SEQUENCE LISTING (178 sequences)

SEQ ID NO: 1 - protein, 652 aa, Homo sapiens
  REGION 1..652: note = extracellular domain of human Receptor tyrosine-protein kinase erbB-2 (HER2)
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLT

SEQ ID NO: 2 - protein, 20 aa, synthetic construct
  REGION 1..20: note = heavy chain CDR3 consensus sequence
YXXYXMXXYGSWXXMXXXDY

SEQ ID NO: 3 - protein, 5 aa, synthetic construct
  REGION 1..5: note = heavy chain CDR1 for antibody 11-1
SSSIH

SEQ ID NO: 4 - protein, 5 aa, synthetic construct
  REGION 1..5: note = heavy chain CDR1 for antibody CH2
RTSIH

SEQ ID NO: 5 - protein, 5 aa, synthetic construct
  REGION 1..5: note = heavy chain CDR1 for antibody CH10
TTSIH

SEQ ID NO: 6 - protein, 5 aa, synthetic construct
  REGION 1..5: note = heavy chain CDR1 for antibodies CH15, CH15V, TL21, TL22, TL23, TL24
GNYIH

SEQ ID NO: 7 - protein, 5 aa, synthetic construct
  REGION 1..5: note = heavy chain CDR1 for antibody CL1
TTAIH

SEQ ID NO: 8 - protein, 5 aa, synthetic construct
  REGION 1..5: note = heavy chain CDR1 for antibody CL3
RTDIH

SEQ ID NO: 9 - protein, 5 aa, synthetic construct
  REGION 1..5: note = heavy chain CDR1 for antibody CL6
GSAIH

SEQ ID NO: 10 - protein, 5 aa, synthetic construct
  REGION 1..5: note = heavy chain CDR1 for antibody CL11
ATSIH

SEQ ID NO: 11 - protein, 5 aa, synthetic construct
  REGION 1..5: note = heavy chain CDR1 for antibody 7-20
YSSIH

SEQ ID NO: 12 - protein, 5 aa, synthetic construct
  REGION 1..5: note = heavy chain CDR1 for antibody 10-20
SSSIH

SEQ ID NO: 13 - protein, 5 aa, synthetic construct
  REGION 1..5: note = heavy chain CDR1 for antibody 25-20
SSYIH

SEQ ID NO: 14 - protein, 17 aa, synthetic construct
  REGION 1..17: note = heavy chain CDR2 for antibody 11-1
SISSYYGYTSYADSVKG

SEQ ID NO: 15 - protein, 17 aa, synthetic construct
  REGION 1..17: note = heavy chain CDR2 for antibody CH2
EISSYDGYTDYADSVKG

SEQ ID NO: 16 - protein, 17 aa, synthetic construct
  REGION 1..17: note = heavy chain CDR2 for antibody CH10
EISSYNGDTDYADSVKG

SEQ ID NO: 17 - protein, 17 aa, synthetic construct
  REGION 1..17: note = heavy chain CDR2 for antibodies CH15, CH15V, TL1, TL7, TL16, TL18, TL21, TL22, TL23, TL24, LL2, LL5
SIYSAGGYTDYADSVKG

SEQ ID NO: 18 - protein, 17 aa, synthetic construct
  REGION 1..17: note = heavy chain CDR2 for antibody CL1
SIASYNGDTDYADSVKG

SEQ ID NO: 19 - protein, 17 aa, synthetic construct
  REGION 1..17: note = heavy chain CDR2 for antibody CL3
SIDSNNGSTYYADSVKG

SEQ ID NO: 20 - protein, 17 aa, synthetic construct
  REGION 1..17: note = heavy chain CDR2 for antibody CL6
SIDSYDGDTDYADSVKG

SEQ ID NO: 21 - protein, 17 aa, synthetic construct
  REGION 1..17: note = heavy chain CDR2 for antibody CL11
EITSYNGSTDYADSVKG

SEQ ID NO: 22 - protein, 17 aa, synthetic construct
  REGION 1..17: note = heavy chain CDR2 for antibody 7-20
SISSYYGSTSYADSVKG

SEQ ID NO: 23 - protein, 17 aa, synthetic construct
  REGION 1..17: note = heavy chain CDR2 for antibody 10-20
YISPSSGSTYYADSVKG

SEQ ID NO: 24 - protein, 17 aa, synthetic construct
  REGION 1..17: note = heavy chain CDR2 for antibody 25-20
SIYPSYGSTYYADSVKG

SEQ ID NO: 25 - protein, 20 aa, synthetic construct
  REGION 1..20: note = heavy chain CDR3 for antibodies 11-1, CH2, CH10, CH15, CL1, CL3, CL6, CL11
YGNYTMHQYGSWEQMPAFDY

SEQ ID NO: 26 - protein, 19 aa, synthetic construct
  REGION 1..19: note = heavy chain CDR3 for antibody 7-20
GKYSWGYYYMDSSMFAMDY

SEQ ID NO: 27 - protein, 18 aa, synthetic construct
  REGION 1..18: note = heavy chain CDR3 for antibody 10-20
SFYFYAYWSHEWHYAMDY

SEQ ID NO: 28 - protein, 20 aa, synthetic construct
  REGION 1..20: note = heavy chain CDR3 for antibody 25-20
QVGVYQYVAPYWGNVWAMDY

SEQ ID NO: 29 - protein, 11 aa, synthetic construct
  REGION 1..11: note = light chain CDR1 for antibodies 11-1, CH2, CH10, CH15, CL1, CL3, CL6, CL11, 7-20, 10-20, 25-20, 20-20, CH15V, TL1, TL7, TL16, TL18, TL21, TL22, TL23, TL24, LL2, LL5
RASQSVSSAVA

SEQ ID NO: 30 - protein, 7 aa, synthetic construct
  REGION 1..7: note = light chain CDR2 for antibodies 11-1, CH2, CH10, CH15, CL1, CL3, CL6, CL11, 7-20, 10-20, 25-20, 20-20, CH15V, TL1, TL7, TL16, TL18, TL21, TL22, TL23, TL24, LL2, LL5
SASSLYS

SEQ ID NO: 31 - protein, 9 aa, synthetic construct
  REGION 1..9: note = light chain CDR3 for antibodies 11-1, CH2, CH10, CH15, CL1, CL3, CL6, CL11, CH15V
QQSSSSLIT

SEQ ID NO: 32 - protein, 11 aa, synthetic construct
  REGION 1..11: note = light chain CDR3 for antibody 7-20
QQYNLRKDLVT

SEQ ID NO: 33 - protein, 10 aa, synthetic construct
  REGION 1..10: note = light chain CDR3 for antibody 10-20
QQYWDYSLIT

SEQ ID NO: 34 - protein, 10 aa, synthetic construct
  REGION 1..10: note = light chain CDR3 for antibody 25-20
QQSSPGWPVT

SEQ ID NO: 35 - protein, 129 aa, synthetic construct
  REGION 1..129: note = clone 11-1 heavy chain with consensus CDR3
EVQLVESGGGLVQPGGSLRLSCAASGFTVYSSSIHWVRQAPGKGLEWVASISSYYGYTSYADSVKGRFTISADTSKNTAYLQMNSLRAEDTAVYYCARYXXYXMXXYGSWXXMXXXDYWGQGTLVTVSS

SEQ ID NO: 36 - protein, 129 aa, synthetic construct
  REGION 1..129: note = Clone 11-1 heavy chain variable region
EVQLVESGGGLVQPGGSLRLSCAASGFTVYSSSIHWVRQAPGKGLEWVASISSYYGYTSYADSVKGRFTISADTSKNTAYLQMNSLRAEDTAVYYCARYGNYTMHQYGSWEQMPAFDYWGQGTLVTVSS

SEQ ID NO: 37 - protein, 110 aa, synthetic construct
  REGION 1..110: note = Clone 11-1 light chain variable region
DIQMTQSPSSLSASVGDRVTITCRASQSVSSAVAWYQQKPGKAPKLLIYSASSLYSGVPSRFSGSRSGTDFTLTISSLQPEDFATYYCQQSSSSLITFGQGTKVEIKRTV

SEQ ID NO: 38 - protein, 129 aa, synthetic construct
  REGION 1..129: note = clone CH2 heavy chain with consensus CDR3
EVQLVESGGGLVQPGGSLRLSCAASGFTFSRTSIHWVRQAPGKGLEWVAEISSYDGYTDYADSVKGRFTISADTSKNTAYLQMNSLRAEDTAVYYCARYXXYXMXXYGSWXXMXXXDYWGQGTLVTVSS

SEQ ID NO: 39 - protein, 129 aa, synthetic construct
  REGION 1..129: note = Clone CH2 heavy chain variable region
EVQLVESGGGLVQPGGSLRLSCAASGFTFSRTSIHWVRQAPGKGLEWVAEISSYDGYTDYADSVKGRFTISADTSKNTAYLQMNSLRAEDTAVYYCARYGNYTMHQYGSWEQMPAFDYWGQGTLVTVSS

SEQ ID NO: 40 - protein, 110 aa, synthetic construct; identical to SEQ ID NO: 37
  REGION 1..110: note = Clone CH2 light chain variable region

SEQ ID NO: 41 - protein, 129 aa, synthetic construct
  REGION 1..129: note = clone CH10 heavy chain with consensus CDR3
EVQLVESGGGLVQPGGSLRLSCAASGFTFSTTSIHWVRQAPGKGLEWVAEISSYNGDTDYADSVKGRFTISADTSKNTAYLQMNSLRAEDTAVYYCARYXXYXMXXYGSWXXMXXXDYWGQGTLVTVSS

SEQ ID NO: 42 - protein, 129 aa, synthetic construct
  REGION 1..129: note = Clone CH10 heavy chain variable region
EVQLVESGGGLVQPGGSLRLSCAASGFTFSTTSIHWVRQAPGKGLEWVAEISSYNGDTDYADSVKGRFTISADTSKNTAYLQMNSLRAEDTAVYYCARYGNYTMHQYGSWEQMPAFDYWGQGTLVTVSS

SEQ ID NO: 43 - protein, 110 aa, synthetic construct; identical to SEQ ID NO: 37
  REGION 1..110: note = Clone CH10 light chain variable region

SEQ ID NO: 44 - protein, 129 aa, synthetic construct
  REGION 1..129: note = clone CH15 heavy chain with consensus CDR3
EVQLVESGGGLVQPGGSLRLSCAASGFTFSGNYIHWVRQAPGKGLEWVASIYSAGGYTDYADSVKGRFTISADTSKNTAYLQMNSLRAEDTAVYYCARYXXYXMXXYGSWXXMXXXDYWGQGTLVTVSS

SEQ ID NO: 45 - protein, 129 aa, synthetic construct
  REGION 1..129: note = Clone CH15 heavy chain variable region
EVQLVESGGGLVQPGGSLRLSCAASGFTFSGNYIHWVRQAPGKGLEWVASIYSAGGYTDYADSVKGRFTISADTSKNTAYLQMNSLRAEDTAVYYCARYGNYTMHQYGSWEQMPAFDYWGQGTLVTVSS

SEQ ID NO: 46 - protein, 110 aa, synthetic construct; identical to SEQ ID NO: 37
  REGION 1..110: note = Clone CH15 light chain variable region

SEQ ID NO: 47 - protein, 129 aa, synthetic construct
  REGION 1..129: note = clone CL1 heavy chain with consensus CDR3
EVQLVESGGGLVQPGGSLRLSCAASGFTFSTTAIHWVRQAPGKGLEWVASIASYNGDTDYADSVKGRFTISADTSKNTAYLQMNSLRAEDTAVYYCARYXXYXMXXYGSWXXMXXXDYWGQGTLVTVSS

SEQ ID NO: 48 - protein, 129 aa, synthetic construct
  REGION 1..129: note = Clone CL1 heavy chain variable region
EVQLVESGGGLVQPGGSLRLSCAASGFTFSTTAIHWVRQAPGKGLEWVASIASYNGDTDYADSVKGRFTISADTSKNTAYLQMNSLRAEDTAVYYCARYGNYTMHQYGSWEQMPAFDYWGQGTLVTVSS

SEQ ID NO: 49 - protein, 110 aa, synthetic construct; identical to SEQ ID NO: 37
  REGION 1..110: note = Clone CL1 light chain variable region

SEQ ID NO: 50 - protein, 129 aa, synthetic construct
  REGION 1..129: note = clone CL3 heavy chain with consensus CDR3
EVQLVESGGGLVQPGGSLRLSCAASGFTFTRTDIHWVRQAPGKGLEWVASIDSNNGSTYYADSVKGRFTISADTSKNTAYLQMNSLRAEDTAVYYCARYXXYXMXXYGSWXXMXXXDYWGQGTLVTVSS

SEQ ID NO: 51 - protein, 129 aa, synthetic construct
  REGION 1..129: note = Clone CL3 heavy chain variable region
EVQLVESGGGLVQPGGSLRLSCAASGFTFTRTDIHWVRQAPGKGLEWVASIDSNNGSTYYADSVKGRFTISADTSKNTAYLQMNSLRAEDTAVYYCARYGNYTMHQYGSWEQMPAFDYWGQGTLVTVSS

SEQ ID NO: 52 - protein, 110 aa, synthetic construct; identical to SEQ ID NO: 37
  REGION 1..110: note = Clone CL3 light chain variable region

SEQ ID NO: 53 - protein, 129 aa, synthetic construct
  REGION 1..129: note = clone CL6 heavy chain with consensus CDR3
EVQLVESGGGLVQPGGSLRLSCAASGFTFTGSAIHWVRQAPGKGLEWVASIDSYDGDTDYADSVKGRFTISADTSKNTAYLQMNSLRAEDTAVYYCARYXXYXMXXYGSWXXMXXXDYWGQGTLVTVSS

SEQ ID NO: 54 - protein, 129 aa, synthetic construct
  REGION 1..129: note = Clone CL6 heavy chain variable region
EVQLVESGGGLVQPGGSLRLSCAASGFTFTGSAIHWVRQAPGKGLEWVASIDSYDGDTDYADSVKGRFTISADTSKNTAYLQMNSLRAEDTAVYYCARYGNYTMHQYGSWEQMPAFDYWGQGTLVTVSS

SEQ ID NO: 55 - protein, 110 aa, synthetic construct; identical to SEQ ID NO: 37
  REGION 1..110: note = Clone CL6 light chain variable region

SEQ ID NO: 56 - protein, 129 aa, synthetic construct
  REGION 1..129: note = clone CL11 heavy chain with consensus CDR3
EVQLVESGGGLVQPGGSLRLSCAASGFTFSATSIHWVRQAPGKGLEWVAEITSYNGSTDYADSVKGRFTISADTSKNTAYLQMNSLRAEDTAVYYCARYXXYXMXXYGSWXXMXXXDYWGQGTLVTVSS

SEQ ID NO: 57 - protein, 129 aa, synthetic construct
  REGION 1..129: note = Clone CL11 heavy chain variable region
EVQLVESGGGLVQPGGSLRLSCAASGFTFSATSIHWVRQAPGKGLEWVAEITSYNGSTDYADSVKGRFTISADTSKNTAYLQMNSLRAEDTAVYYCARYGNYTMHQYGSWEQMPAFDYWGQGTLVTVSS

SEQ ID NO: 58 - protein, 110 aa, synthetic construct; identical to SEQ ID NO: 37
  REGION 1..110: note = Clone CL11 light chain variable region

SEQ ID NO: 59 - protein, 128 aa, synthetic construct
  REGION 1..128: note = Clone 7-20 heavy chain variable region
EVQLVESGGGLVQPGGSLRLSCAASGFTISYSSIHWVRQAPGKGLEWVASISSYYGSTSYADSVKGRFTISADTSKNTAYLQMNSLRAEDTAVYYCARGKYSWGYYYMDSSMFAMDYWGQGTLVTVSS

SEQ ID NO: 60 - protein, 112 aa, synthetic construct
  REGION 1..112: note = Clone 7-20 light chain variable region
DIQMTQSPSSLSASVGDRVTITCRASQSVSSAVAWYQQKPGKAPKLLIYSASSLYSGVPSRFSGSRSGTDFTLTISSLQPEDFATYYCQQYNLRKDLVTFGQGTKVEIKRTV

SEQ ID NO: 61 - protein, 127 aa, synthetic construct
  REGION 1..127: note = Clone 10-20 heavy chain variable region
EVQLVESGGGLVQPGGSLRLSCAASGFTVSSSSIHWVRQAPGKGLEWVAYISPSSGSTYYADSVKGRFTISADTSKNTAYLQMNSLRAEDTAVYYCARSFYFYAYWSHEWHYAMDYWGQGTLVTVSS

SEQ ID NO: 62 - protein, 111 aa, synthetic construct
  REGION 1..111: note = Clone 10-20 light chain variable region
DIQMTQSPSSLSASVGDRVTITCRASQSVSSAVAWYQQKPGKAPKLLIYSASSLYSGVPSRFSGSRSGTDFTLTISSLQPEDFATYYCQQYWDYSLITFGQGTKVEIKRTV

SEQ ID NO: 63 - protein, 129 aa, synthetic construct
  REGION 1..129: note = Clone 25-20 heavy chain variable region
EVQLVESGGGLVQPGGSLRLSCAASGFTLSSSYIHWVRQAPGKGLEWVASIYPSYGSTYYADSVKGRFTISADTSKNTAYLQMNSLRAEDTAVYYCARQVGVYQYVAPYWGNVWAMDYWGQGTLVTVSS

SEQ ID NO: 64 - protein, 111 aa, synthetic construct
  REGION 1..111: note = Clone 25-20 light chain variable region
DIQMTQSPSSLSASVGDRVTITCRASQSVSSAVAWYQQKPGKAPKLLIYSASSLYSGVPSRFSGSRSGTDFTLTISSLQPEDFATYYCQQSSPGWPVTFGQGTKVEIKRTV

SEQ ID NO: 65 - protein, 123 aa, synthetic construct
  REGION 1..123: note = Clone 20-20 heavy chain variable region
EVQLVESGGGLVQPGGSLRLSCAASGFTLSYYSIHWVRQAPGKGLEWVASIYSSYGYTSYADSVKGRFTISADTSKNTAYLQMNSLRAEDTAVYYCARYWSEYWQYYPAMDYWGQGTLVTVSS

SEQ ID NO: 66 - protein, 112 aa, synthetic construct
  REGION 1..112: note = Clone 20-20 light chain variable region
DIQMTQSPSSLSASVGDRVTITCRASQSVSSAVAWYQQKPGKAPKLLIYSASSLYSGVPSRFSGSRSGTDFTLTISSLQPEDFATYYCQQSLNYSGLITFGQGTKVEIKRTV

SEQ ID NO: 67 - protein, 916 aa, synthetic construct
  REGION 1..916: note = wildtype HER2-Fc construct
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTPGSRSPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLEGGGGLNDIFEAQKIEWHESRHHHHHH

SEQ ID NO: 68 - protein, 916 aa, synthetic construct
  REGION 1..916: note = mutant HER2(S310F)-Fc construct
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGFCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTPGSRSPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLEGGGGLNDIFEAQKIEWHESRHHHHHH

SEQ ID NO: 69 - protein, 916 aa, synthetic construct
  REGION 1..916: note = mutant HER2(S310Y)-Fc construct
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGYCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTPGSRSPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLEGGGGLNDIFEAQKIEWHESRHHHHHH

SEQ ID NO: 70 - protein, 5 aa, synthetic construct
  REGION 1..5: note = heavy chain CDR1 for antibody 20-20
YYSIH

SEQ ID NO: 71 - protein, 17 aa, synthetic construct
  REGION 1..17: note = heavy chain CDR2 for antibody 20-20
SIYSSYGYTSYADSVKG

SEQ ID NO: 72 - protein, 14 aa, synthetic construct
  REGION 1..14: note = heavy chain CDR3 for antibody 20-20
YWSEYWQYYPAMDY

SEQ ID NO: 73 - protein, 11 aa, synthetic construct
  REGION 1..11: note = light chain CDR3 for antibody 20-20
QQSLNYSGLIT

SEQ ID NO: 74 - protein, 20 aa, synthetic construct
  REGION 1..20: note = heavy chain CDR3 for antibodies CH15V
YGVYTMHQYGSWEQMPAFDY

SEQ ID NO: 75 - protein, 5 aa, synthetic construct
  REGION 1..5: note = heavy chain CDR1 for antibodies TL1, TL16
GAYIH

SEQ ID NO: 76 - protein, 5 aa, synthetic construct
  REGION 1..5: note = heavy chain CDR1 for antibodies TL7, TL18, LL2, LL5
GSYIH

SEQ ID NO: 77 - protein, 20 aa, synthetic construct
  REGION 1..20: note = heavy chain CDR3 for antibodies TL1
YGTYELKSYGSWESLPAFDY

SEQ ID NO: 78 - protein, 20 aa, synthetic construct
  REGION 1..20: note = heavy chain CDR3 for antibodies TL7
YGVYTLAAYGSWESLPAFDY

SEQ ID NO: 79 - protein, 20 aa, synthetic construct
  REGION 1..20: note = heavy chain CDR3 for antibodies TL16
YGTYELKAAGSWEQLPAFDY

SEQ ID NO: 80 - protein, 20 aa, synthetic construct
  REGION 1..20: note = heavy chain CDR3 for antibodies TL18
YGVYTLHQYGSWEQLPAFDY

SEQ ID NO: 81 - protein, 20 aa, synthetic construct
  REGION 1..20: note = heavy chain CDR3 for antibodies TL21, TL22, TL23, TL24, LL2, LL5
YGVYTLASAGSWESLPAFDY

SEQ ID NO: 82 - protein, 10 aa, synthetic construct
  REGION 1..10: note = light chain CDR3 for antibody TL1
QQSEWGGLIT

SEQ ID NO: 83 - protein, 9 aa, synthetic construct
  REGION 1..9: note = light chain CDR3 for antibody TL7
QQSDYGLIT

SEQ ID NO: 84 - protein, 9 aa, synthetic construct
  REGION 1..9: note = light chain CDR3 for antibody TL16
QQSGYSLIT

SEQ ID NO: 85 - protein, 9 aa, synthetic construct
  REGION 1..9: note = light chain CDR3 for antibody TL18
QQDDYSLIT

SEQ ID NO: 86 - protein, 9 aa, synthetic construct
  REGION 1..9: note = light chain CDR3 for antibody TL21
QQSSWDLIT

SEQ ID NO: 87 - protein, 10 aa, synthetic construct
  REGION 1..10: note = light chain CDR3 for antibody TL22
QQSSWNGLIT

SEQ ID NO: 88 - protein, 10 aa, synthetic construct
  REGION 1..10: note = light chain CDR3 for antibody TL22
QQSGWGGLIT

SEQ ID NO: 89 - protein, 10 aa, synthetic construct
  REGION 1..10: note = light chain CDR3 for antibody TL24
QQSSYEDPIT

SEQ ID NO: 90 - protein, 11 aa, synthetic construct
  REGION 1..11: note = light chain CDR3 for antibody LL2
QQSSWESPPIT

SEQ ID NO: 91 - protein, 10 aa, synthetic construct
  REGION 1..10: note = light chain CDR3 for antibody LL5
QQSSYDGLLT

SEQ ID NO: 92 - skipped

SEQ ID NO: 93 - protein, 20 aa, synthetic construct
  REGION 1..20: note = heavy chain CDR3
  SITE 1: note = where X at 1 is Tyr (Y) or Phe (F);
  SITE 3: note = X at 3 is Val (V), Glu (E), His (H), Ile (I), Leu (L), Met (M), Pro (P), Gln (Q), or Thy (T);
  SITE 5: note = X at 5 is Thy (T), Ala (A), Glu (E), His (H), Ile (I), Lys (K), Leu (L), Met (M), Asn (N), Gln (Q), Arg (R), or Ser (S);
  SITE 6: note = X at 6 is Met (M) or Leu (L);
  SITE 7: note = X at 7 is His (H), Ala (A), Lys (K), Met (M), Gln (Q), or Arg (R);
  SITE 8: note = X at 8 is Gln (Q), Ala (A), Phe (F), His (H), Ile (I), Lys (K), Leu (L), Met (M), Asn (N), Arg (R), Ser (S), or Thy (T);
  SITE 9: note = X at 9 is Tyr (Y), Ala (A), Phe (F), Gly (G), or Arg (R);
  SITE 13: note = X at 13 is Glu (E), Gln (Q), or Val (V);
  SITE 14: note = X at 14 is Gln (Q), Met (M), or Ser (S);
  SITE 15: note = X at 15 is Met (M), Phe (F), Leu (L), Arg (R), or Thy (T);
  SITE 17: note = X at 17 is Ala (A) or Tyr (Y)
  SITE 18: note = X at 18 is Phe (F), His (H), or Tyr (Y).
XGXYXXXXXGSWXXXPXXDY

SEQ ID NO: 94 - skipped

SEQ ID NO: 95 - protein, 30 aa, synthetic construct
  REGION 1..30: note = heavy chain FR1 region
  SITE 29: note = where X at 29 is Phe (F), Trp (W), or Tyr (Y)
  SITE 30: note = where X at 30 is Ser (S), Ala (A), Phe (F), Gly (G), His (H), Ile (I), Met (M), Asn (N), Gln (Q), Arg (R), Thy (T), Val (V), Trp (W), or Tyr (Y)
EVQLVESGGGLVQPGGSLRLSCAASGFTXX

SEQ ID NO: 96 - protein, 129 aa, synthetic construct
  REGION 1..129: note = clone CH15V heavy chain with consensus CDR1 and CDR3
  SITE 29: note = X at 29 is Phe (F), Trp (W), or Tyr (Y)
  SITE 30: note = X at 30 is Ser (S), Ala (A), Phe (F), Gly (G), His (H), Ile (I), Met (M), Asn (N), Gln (Q), Arg (R), Thy (T), Val (V), Trp (W), or Tyr (Y);
  SITE 32: note = X at 32 is Asn (N), Ala (A), Cys (C), Phe (F), His (H), Leu (L), Met (M), or Ser (S)
  SITE 33: note = X at 33 is Tyr (Y), Glu (E), or Phe (F).
  SITE 99: note = X at 99 is Tyr (Y) or Phe (F);
  SITE 101: note = X at 101 is Val (V), Glu (E), His (H), Ile (I), Leu (L), Met (M), Pro (P), Gln (Q), or Thy (T);
  SITE 103: note = X at 103 is Thy (T), Ala (A), Glu (E), His (H), Ile (I), Lys (K), Leu (L), Met (M), Asn (N), Gln (Q), Arg (R), or Ser (S);
  SITE 104: note = X at 104 is Met (M) or Leu (L);
  SITE 105: note = X at 105 is His (H), Ala (A), Lys (K), Met (M), Gln (Q), or Arg (R);
  SITE 106: note = X at 106 is Gln (Q), Ala (A), Phe (F), His (H), Ile (I), Lys (K), Leu (L), Met (M), Asn (N), Arg (R), Ser (S), or Thy (T);
  SITE 107: note = X at 107 is Tyr (Y), Ala (A), Phe (F), Gly (G), or Arg (R);
  SITE 111: note = X at 111 is Glu (E), Gln (Q), or Val (V);
  SITE 112: note = X at 112 is Gln (Q), Met (M), or Ser (S);
  SITE 113: note = X at 113 is Met (M), Phe (F), Leu (L), Arg (R), or Thy (T);
  SITE 115: note = X at 115 is Ala (A) or Tyr (Y)
  SITE 116: note = X at 116 is Phe (F), His (H), or Tyr (Y).
EVQLVESGGGLVQPGGSLRLSCAASGFTXXGXXIHWVRQAPGKGLEWVASIYSAGGYTDYADSVKGRFTISADTSKNTAYLQMNSLRAEDTAVYYCARXGXYXXXXXGSWXXXPXXDYWGQGTLVTVSS

SEQ ID NO: 97 - protein, 129 aa, synthetic construct
  REGION 1..129: note = Clone CH15V heavy chain variable region
EVQLVESGGGLVQPGGSLRLSCAASGFTFSGNYIHWVRQAPGKGLEWVASIYSAGGYTDYADSVKGRFTISADTSKNTAYLQMNSLRAEDTAVYYCARYGVYTMHQYGSWEQMPAFDYWGQGTLVTVSS

SEQ ID NO: 98 - protein, 111 aa, synthetic construct
  REGION 1..111: note = clone CH15V light chain with consensus CDR3
  SITE 91: note = where X at 91 is Tyr (Y), Ser (S) or Asp (D);
  SITE 92: note = X at 92 is Ser (S), Asn (N), Trp (W), Leu (L), Glu (E), Asp (D), Gly (G),
  SITE 93: note = X at 93 is absent, Trp (W), Tyr (Y), Asp (D), Pro (P), -Trp-Glu- (-WE-), -Leu-Arg- (-LR-), or -Asn-Tyr- (-NY-);
  SITE 94: note = X at 94 is Ser (S), Lys (K), Tyr (Y), Gly (G), Trp (W), Asn (N), or Glu (E);
  SITE 95: note = X at 95 is Ser (S), Asp (D), Trp (W), Gly (G), Asp (D), or Pro (P);
  SITE 96: note = X at 96 is Leu (L) or Pro (P);
  SITE 97: note = X at 97 is Ile (I), Leu (L), or Val (V);
DIQMTQSPSSLSASVGDRVTITCRASQSVSSAVAWYQQKPGKAPKLLIYSASSLYSGVPSRFSGSRSGTDFTLTISSLQPEDFATYYCQQXXXXXXXTFGQGTKVEIKRTV

SEQ ID NO: 99 - protein, 110 aa, synthetic construct; identical to SEQ ID NO: 37
  REGION 1..110: note = Clone CH15V light chain variable region

SEQ ID NO: 100 - protein, 127 aa, synthetic construct
  REGION 1..127: note = clone TL1 heavy chain with consensus CDR1 and CDR3
  SITE 29: note = X at 29 is Phe (F), Trp (W), or Tyr (Y)
  SITE 30: note = X at 30 is Ser (S), Ala (A), Phe (F), Gly (G), His (H), Ile (I), Met (M), Asn (N), Gln (Q), Arg (R), Thy (T), Val (V), Trp (W), or Tyr (Y);
  SITE 32: note = X at 32 is Asn (N), Ala (A), Cys (C), Phe (F), His (H), Leu (L), Met (M), or Ser (S)
  SITE 33: note = X at 33 is Tyr (Y), Glu (E), or Phe (F).
  SITE 99: note = X at 99 is Tyr (Y) or Phe (F);
  SITE 101: note = X at 101 is Val (V), Glu (E), His (H), Ile (I), Leu (L), Met (M), Pro (P), Gln (Q), or Thy (T);
  SITE 103: note = X at 103 is Thy (T), Ala (A), Glu (E), His (H), Ile (I), Lys (K), Leu (L), Met (M), Asn (N), Gln (Q), Arg (R), or Ser (S);
  SITE 104: note = X at 104 is Met (M) or Leu (L);
  SITE 105: note = X at 105 is His (H), Ala (A), Lys (K), Met (M), Gln (Q), or Arg (R);
  SITE 106: note = X at 106 is Gln (Q), Ala (A), Phe (F), His (H), Ile (I), Lys (K), Leu (L), Met (M), Asn (N), Arg (R), Ser (S), or Thy (T);
  SITE 107: note = X at 107 is Tyr (Y), Ala (A), Phe (F), Gly (G), or Arg (R);
  SITE 111: note = X at 111 is Glu (E), Gln (Q), or Val (V);
  SITE 112: note = X at 112 is Gln (Q), Met (M), or Ser (S);
  SITE 113: note = X at 113 is Met (M), Phe (F), Leu (L), Arg (R), or Thy (T);
  SITE 115: note = X at 115 is Ala (A) or Tyr (Y)
  SITE 116: note = X at 116 is Phe (F), His (H), or Tyr (Y).
EVQLVESGGGLVQPGGSLRLSCAASGFTXXGXXIHWVRQAPGKGLEWVASIYSAGGYTDYADSVKGRFTISADTSKNTAYLQMNSLRAEDTAVYYCARXGXYXXXXXGSWXXXPXXDYWGQGTLVTV

SEQ ID NO: 101 - protein, 127 aa, synthetic construct
  REGION 1..127: note = Clone TL1 heavy chain variable region
EVQLVESGGGLVQPGGSLRLSCAASGFTWSGAYIHWVRQAPGKGLEWVASIYSAGGYTDYADSVKGRFTISADTSKNTAYLQMNSLRAEDTAVYYCARYGTYELKSYGSWESLPAFDYWGQGTLVTV

SEQ ID NO: 102 - protein, 111 aa, synthetic construct; identical to SEQ ID NO: 98
  REGION 1..111: note = clone TL1 light chain with consensus CDR3
  SITE 91: note = where X at 91 is Tyr (Y), Ser (S) or Asp (D);
  SITE 92: note = X at 92 is Ser (S), Asn (N), Trp (W), Leu (L), Glu (E), Asp (D), Gly (G),
  SITE 93: note = X at 93 is absent, Trp (W), Tyr (Y), Asp (D), Pro (P), -Trp-Glu- (-WE-), -Leu-Arg- (-LR-), or -Asn-Tyr- (-NY-);
  SITE 94: note = X at 94 is Ser (S), Lys (K), Tyr (Y), Gly (G), Trp (W), Asn (N), or Glu (E);
  SITE 95: note = X at 95 is Ser (S), Asp (D), Trp (W), Gly (G), Asp (D), or Pro (P);
  SITE 96: note = X at 96 is Leu (L) or Pro (P);
  SITE 97: note = X at 97 is Ile (I), Leu (L), or Val (V);

SEQ ID NO: 103 - protein, 111 aa, synthetic construct
  REGION 1..111: note = Clone TL1 light chain variable region
DIQMTQSPSSLSASVGDRVTITCRASQSVSSAVAWYQQKPGKAPKLLIYSASSLYSGVPSRFSGSRSGTDFTLTISSLQPEDFATYYCQQSEWGGLITFGQGTKVEIKRTV

SEQ ID NO: 104 - protein, 127 aa, synthetic construct; identical to SEQ ID NO: 100
  REGION 1..127: note = clone TL7 heavy chain with consensus CDR1 and CDR3
  SITE 29: note = X at 29 is Phe (F), Trp (W), or Tyr (Y)
  SITE 30: note = X at 30 is Ser (S), Ala (A), Phe (F), Gly (G), His (H), Ile (I), Met (M), Asn (N), Gln (Q), Arg (R), Thy (T), Val (V), Trp (W), or Tyr (Y);
  SITE 32: note = X at 32 is Asn (N), Ala (A), Cys (C), Phe (F), His (H), Leu (L), Met (M), or Ser (S)
  SITE 33: note = X at 33 is Tyr (Y), Glu (E), or Phe (F).
  SITE 99: note = X at 99 is Tyr (Y) or Phe (F);
  SITE 101: note = X at 101 is Val (V), Glu (E), His (H), Ile (I), Leu (L), Met (M), Pro (P), Gln (Q), or Thy (T);
  SITE 103: note = X at 103 is Thy (T), Ala (A), Glu (E), His (H), Ile (I), Lys (K), Leu (L), Met (M), Asn (N), Gln (Q), Arg (R), or Ser (S);
  SITE 104: note = X at 104 is Met (M) or Leu (L);
  SITE 105: note = X at 105 is His (H), Ala (A), Lys (K), Met (M), Gln (Q), or Arg (R);
  SITE 106: note = X at 106 is Gln (Q), Ala (A), Phe (F), His (H), Ile (I), Lys (K), Leu (L), Met (M), Asn (N), Arg (R), Ser (S), or Thy (T);
  SITE 107: note = X at 107 is Tyr (Y), Ala (A), Phe (F), Gly (G), or Arg (R);
  SITE 111: note = X at 111 is Glu (E), Gln (Q), or Val (V);
  SITE 112: note = X at 112 is Gln (Q), Met (M), or Ser (S);
  SITE 113: note = X at 113 is Met (M), Phe (F), Leu (L), Arg (R), or Thy (T);
  SITE 115: note = X at 115 is Ala (A) or Tyr (Y)
  SITE 116: note = X at 116 is Phe (F), His (H), or Tyr (Y).

SEQ ID NO: 105 - protein, 127 aa, synthetic construct
  REGION 1..127: note = Clone TL7 heavy chain variable region
EVQLVESGGGLVQPGGSLRLSCAASGFTWGGSYIHWVRQAPGKGLEWVASIYSAGGYTDYADSVKGRFTISADTSKNTAYLQMNSLRAEDTAVYYCARYGVYTLAAYGSWESLPAFDYWGQGTLVTV

SEQ ID NO: 106 - protein, 111 aa, synthetic construct; identical to SEQ ID NO: 98
  REGION 1..111: note = clone TL7 light chain with consensus CDR3
  SITE 91: note = where X at 91 is Tyr (Y), Ser (S) or Asp (D);
  SITE 92: note = X at 92 is Ser (S), Asn (N), Trp (W), Leu (L), Glu (E), Asp (D), Gly (G),
  SITE 93: note = X at 93 is absent, Trp (W), Tyr (Y), Asp (D), Pro (P), -Trp-Glu- (-WE-), -Leu-Arg- (-LR-), or -Asn-Tyr- (-NY-);
  SITE 94: note = X at 94 is Ser (S), Lys (K), Tyr (Y), Gly (G), Trp (W), Asn (N), or Glu (E);
  SITE 95: note = X at 95 is Ser (S), Asp (D), Trp (W), Gly (G), Asp (D), or Pro (P);
  SITE 96: note = X at 96 is Leu (L) or Pro (P);
  SITE 97: note = X at 97 is Ile (I), Leu (L), or Val (V);

SEQ ID NO: 107 - protein, 110 aa, synthetic construct
  REGION 1..110: note = Clone TL7 light chain variable region
DIQMTQSPSSLSASVGDRVTITCRASQSVSSAVAWYQQKPGKAPKLLIYSASSLYSGVPSRFSGSRSGTDFTLTISSLQPEDFATYYCQQSDYGLITFGQGTKVEIKRTV

SEQ ID NO: 108 - protein, 127 aa, synthetic construct; identical to SEQ ID NO: 100
  REGION 1..127: note = clone TL16 heavy chain with consensus CDR1 and CDR3
  SITE 29: note = X at 29 is Phe (F), Trp (W), or Tyr (Y)
  SITE 30: note = X at 30 is Ser (S), Ala (A), Phe (F), Gly (G), His (H), Ile (I), Met (M), Asn (N), Gln (Q), Arg (R), Thy (T), Val (V), Trp (W), or Tyr (Y);
  SITE 32: note = X at 32 is Asn (N), Ala (A), Cys (C), Phe (F), His (H), Leu (L), Met (M), or Ser (S)
  SITE 33: note = X at 33 is Tyr (Y), Glu (E), or Phe (F).
  SITE 99: note = X at 99 is Tyr (Y) or Phe (F);
  SITE 101: note = X at 101 is Val (V), Glu (E), His (H), Ile (I), Leu (L), Met (M), Pro (P), Gln (Q), or Thy (T);
  SITE 103: note = X at 103 is Thy (T), Ala (A), Glu (E), His (H), Ile (I), Lys (K), Leu (L), Met (M), Asn (N), Gln (Q), Arg (R), or Ser (S);
  SITE 104: note = X at 104 is Met (M) or Leu (L);
  SITE 105: note = X at 105 is His (H), Ala (A), Lys (K), Met (M), Gln (Q), or Arg (R);
  SITE 106: note = X at 106 is Gln (Q), Ala (A), Phe (F), His (H), Ile (I), Lys (K), Leu (L), Met (M), Asn (N), Arg (R), Ser (S), or Thy (T);
  SITE 107: note = X at 107 is Tyr (Y), Ala (A), Phe (F), Gly (G), or Arg (R);
  SITE 111: note = X at 111 is Glu (E), Gln (Q), or Val (V);
  SITE 112: note = X at 112 is Gln (Q), Met (M), or Ser (S);
  SITE 113: note = X at 113 is Met (M), Phe (F), Leu (L), Arg (R), or Thy (T);
  SITE 115: note = X at 115 is Ala (A) or Tyr (Y)
  SITE 116: note = X at 116 is Phe (F), His (H), or Tyr (Y).

SEQ ID NO: 109 - protein, 127 aa, synthetic construct
  REGION 1..127: note = Clone TL16 heavy chain variable region
EVQLVESGGGLVQPGGSLRLSCAASGFTFGGAYIHWVRQAPGKGLEWVASIYSAGGYTDYADSVKGRFTISADTSKNTAYLQMNSLRAEDTAVYYCARYGTYELKAAGSWEQLPAFDYWGQGTLVTV

SEQ ID NO: 110 - protein, 111 aa, synthetic construct; identical to SEQ ID NO: 98
  REGION 1..111: note = clone TL16 light chain with consensus CDR3
  SITE 91: note = where X at 91 is Tyr (Y), Ser (S) or Asp (D);
  SITE 92: note = X at 92 is Ser (S), Asn (N), Trp (W), Leu (L), Glu (E), Asp (D), Gly (G),
  SITE 93: note = X at 93 is absent, Trp (W), Tyr (Y), Asp (D), Pro (P), -Trp-Glu- (-WE-), -Leu-Arg- (-LR-), or -Asn-Tyr- (-NY-);
  SITE 94: note = X at 94 is Ser (S), Lys (K), Tyr (Y), Gly (G), Trp (W), Asn (N), or Glu (E);
  SITE 95: note = X at 95 is Ser (S), Asp (D), Trp (W), Gly (G), Asp (D), or Pro (P);
  SITE 96: note = X at 96 is Leu (L) or Pro (P);
  SITE 97: note = X at 97 is Ile (I), Leu (L), or Val (V);

SEQ ID NO: 111 - protein, 110 aa, synthetic construct
  REGION 1..110: note = Clone TL16 light chain variable region
DIQMTQSPSSLSASVGDRVTITCRASQSVSSAVAWYQQKPGKAPKLLIYSASSLYSGVPSRFSGSRSGTDFTLTISSLQPEDFATYYCQQSGYSLITFGQGTKVEIKRTV

SEQ ID NO: 112 - protein, 127 aa, synthetic construct; identical to SEQ ID NO: 100
  REGION 1..127: note = clone TL18 heavy chain with consensus CDR1 and CDR3
  SITE 29: note = X at 29 is Phe (F), Trp (W), or Tyr (Y)
  SITE 30: note = X at 30 is Ser (S), Ala (A), Phe (F), Gly (G), His (H), Ile (I), Met (M), Asn (N), Gln (Q), Arg (R), Thy (T), Val (V), Trp (W), or Tyr (Y);
  SITE 32: note = X at 32 is Asn (N), Ala (A), Cys (C), Phe (F), His (H), Leu (L), Met (M), or Ser (S)
  SITE 33: note = X at 33 is Tyr (Y), Glu (E), or Phe (F).
  SITE 99: note = X at 99 is Tyr (Y) or Phe (F);
  SITE 101: note = X at 101 is Val (V), Glu (E), His (H), Ile (I), Leu (L), Met (M), Pro (P), Gln (Q), or Thy (T);
  SITE 103: note = X at 103 is Thy (T), Ala (A), Glu (E), His (H), Ile (I), Lys (K), Leu (L), Met (M), Asn (N), Gln (Q), Arg (R), or Ser (S);
  SITE 104: note = X at 104 is Met (M) or Leu (L);
  SITE 105: note = X at 105 is His (H), Ala (A), Lys (K), Met (M), Gln (Q), or Arg (R);
  SITE 106: note = X at 106 is Gln (Q), Ala (A), Phe (F), His (H), Ile (I), Lys (K), Leu (L), Met (M), Asn (N), Arg (R), Ser (S), or Thy (T);
  SITE 107: note = X at 107 is Tyr (Y), Ala (A), Phe (F), Gly (G), or Arg (R);
  SITE 111: note = X at 111 is Glu (E), Gln (Q), or Val (V);
  SITE 112: note = X at 112 is Gln (Q), Met (M), or Ser (S);
  SITE 113: note = X at 113 is Met (M), Phe (F), Leu (L), Arg (R), or Thy (T);
  SITE 115: note = X at 115 is Ala (A) or Tyr (Y)
  SITE 116: note = X at 116 is Phe (F), His (H), or Tyr (Y).

SEQ ID NO: 113 - protein, 127 aa, synthetic construct
  REGION 1..127: note = Clone TL18 heavy chain variable region
EVQLVESGGGLVQPGGSLRLSCAASGFTFGGSYIHWVRQAPGKGLEWVASIYSAGGYTDYADSVKGRFTISADTSKNTAYLQMNSLRAEDTAVYYCARYGVYTLHQYGSWEQLPAFDYWGQGTLVTV

SEQ ID NO: 114 - protein, 111 aa, synthetic construct; identical to SEQ ID NO: 98
  REGION 1..111: note = clone TL18 light chain with consensus CDR3
  SITE 91: note = where X at 91 is Tyr (Y), Ser (S) or Asp (D);
  SITE 92: note = X at 92 is Ser (S), Asn (N), Trp (W), Leu (L), Glu (E), Asp (D), Gly (G),
  SITE 93: note = X at 93 is absent, Trp (W), Tyr (Y), Asp (D), Pro (P), -Trp-Glu- (-WE-), -Leu-Arg- (-LR-), or -Asn-Tyr- (-NY-);
  SITE 94: note = X at 94 is Ser (S), Lys (K), Tyr (Y), Gly (G), Trp (W), Asn (N), or Glu (E);
  SITE 95: note = X at 95 is Ser (S), Asp (D), Trp (W), Gly (G), Asp (D), or Pro (P);
  SITE 96: note = X at 96 is Leu (L) or Pro (P);
  SITE 97: note = X at 97 is Ile (I), Leu (L), or Val (V);

SEQ ID NO: 115 - protein, 110 aa, synthetic construct
  REGION 1..110: note = Clone TL18 light chain variable region
DIQMTQSPSSLSASVGDRVTITCRASQSVSSAVAWYQQKPGKAPKLLIYSASSLYSGVPSRFSGSRSGTDFTLTISSLQPEDFATYYCQQDDYSLITFGQGTKVEIKRTV

SEQ ID NO: 116 - protein, 127 aa, synthetic construct; identical to SEQ ID NO: 100
  REGION 1..127: note = clone TL21 heavy chain with consensus CDR1 and CDR3
  SITE 29: note = X at 29 is Phe (F), Trp (W), or Tyr (Y)
  SITE 30: note = X at 30 is Ser (S), Ala (A), Phe (F), Gly (G), His (H), Ile (I), Met (M), Asn (N), Gln (Q), Arg (R), Thy (T), Val (V), Trp (W), or Tyr (Y);
  SITE 32: note = X at 32 is Asn (N), Ala (A), Cys (C), Phe (F), His (H), Leu (L), Met (M), or Ser (S)
  SITE 33: note = X at 33 is Tyr (Y), Glu (E), or Phe (F).
  SITE 99: note = X at 99 is Tyr (Y) or Phe (F);
  SITE 101: note = X at 101 is Val (V), Glu (E), His (H), Ile (I), Leu (L), Met (M), Pro (P), Gln (Q), or Thy (T);
  SITE 103: note = X at 103 is Thy (T), Ala (A), Glu (E), His (H), Ile (I), Lys (K), Leu (L), Met (M), Asn (N), Gln (Q), Arg (R), or Ser (S);
  SITE 104: note = X at 104 is Met (M) or Leu (L);
  SITE 105: note = X at 105 is His (H), Ala (A), Lys (K), Met (M), Gln (Q), or Arg (R);
  SITE 106: note = X at 106 is Gln (Q), Ala (A), Phe (F), His (H), Ile (I), Lys (K), Leu (L), Met (M), Asn (N), Arg (R), Ser (S), or Thy (T);
  SITE 107: note = X at 107 is Tyr (Y), Ala (A), Phe (F), Gly (G), or Arg (R);
  SITE 111: note = X at 111 is Glu (E), Gln (Q), or Val (V);
  SITE 112: note = X at 112 is Gln (Q), Met (M), or Ser (S);
  SITE 113: note = X at 113 is Met (M), Phe (F), Leu (L), Arg (R), or Thy (T);
  SITE 115: note = X at 115 is Ala (A) or Tyr (Y)
  SITE 116: note = X at 116 is Phe (F), His (H), or Tyr (Y).

SEQ ID NO: 117 - protein, 127 aa, synthetic construct
  REGION 1..127: note = Clone TL21 heavy chain variable region
EVQLVESGGGLVQPGGSLRLSCAASGFTFSGNYIHWVRQAPGKGLEWVASIYSAGGYTDYADSVKGRFTISADTSKNTAYLQMNSLRAEDTAVYYCARYGVYTLASAGSWESLPAFDYWGQGTLVTV

SEQ ID NO: 118 - protein, 111 aa, synthetic construct; identical to SEQ ID NO: 98
  REGION 1..111: note = clone TL21 light chain with consensus CDR3
  SITE 91: note = where X at 91 is Tyr (Y), Ser (S) or Asp (D);
  SITE 92: note = X at 92 is Ser (S), Asn (N), Trp (W), Leu (L), Glu (E), Asp (D), Gly (G),
  SITE 93: note = X at 93 is absent, Trp (W), Tyr (Y), Asp (D), Pro (P), -Trp-Glu- (-WE-), -Leu-Arg- (-LR-), or -Asn-Tyr- (-NY-);
  SITE 94: note = X at 94 is Ser (S), Lys (K), Tyr (Y), Gly (G), Trp (W), Asn (N), or Glu (E);
  SITE 95: note = X at 95 is Ser (S), Asp (D), Trp (W), Gly (G), Asp (D), or Pro (P);
  SITE 96: note = X at 96 is Leu (L) or Pro (P);
  SITE 97: note = X at 97 is Ile (I), Leu (L), or Val (V);

SEQ ID NO: 119 - protein, 110 aa, synthetic construct
  REGION 1..110: note = Clone TL21 light chain variable region
DIQMTQSPSSLSASVGDRVTITCRASQSVSSAVAWYQQKPGKAPKLLIYSASSLYSGVPSRFSGSRSGTDFTLTISSLQPEDFATYYCQQSSWDLITFGQGTKVEIKRTV

SEQ ID NO: 120 - protein, 127 aa, synthetic construct; identical to SEQ ID NO: 100
  REGION 1..127: note = clone TL22 heavy chain with consensus CDR1 and CDR3
  SITE 29: note = X at 29 is Phe (F), Trp (W), or Tyr (Y)
  SITE 30: note = X at 30 is Ser (S), Ala (A), Phe (F), Gly (G), His (H), Ile (I), Met (M), Asn (N), Gln (Q), Arg (R), Thy (T), Val (V), Trp (W), or Tyr (Y);
  SITE 32: note = X at 32 is Asn (N), Ala (A), Cys (C), Phe (F), His (H), Leu (L), Met (M), or Ser (S)
  SITE 33: note = X at 33 is Tyr (Y), Glu (E), or Phe (F).
  SITE 99: note = X at 99 is Tyr (Y) or Phe (F);
  SITE 101: note = X at 101 is Val (V), Glu (E), His (H), Ile (I), Leu (L), Met (M), Pro (P), Gln (Q), or Thy (T);
  SITE 103: note = X at 103 is Thy (T), Ala (A), Glu (E), His (H), Ile (I), Lys (K), Leu (L), Met (M), Asn (N), Gln (Q), Arg (R), or Ser (S);
  SITE 104: note = X at 104 is Met (M) or Leu (L);
  SITE 105: note = X at 105 is His (H), Ala (A), Lys (K), Met (M), Gln (Q), or Arg (R);
  SITE 106: note = X at 106 is Gln (Q), Ala (A), Phe (F), His (H), Ile (I), Lys (K), Leu (L), Met (M), Asn (N), Arg (R), Ser (S), or Thy (T);
  SITE 107: note = X at 107 is Tyr (Y), Ala (A), Phe (F), Gly (G), or Arg (R);
  SITE 111: note = X at 111 is Glu (E), Gln (Q), or Val (V);
  SITE 112: note = X at 112 is Gln (Q), Met (M), or Ser (S);
  SITE 113: note = X at 113 is Met (M), Phe (F), Leu (L), Arg (R), or Thy (T);
  SITE 115: note = X at 115 is Ala (A) or Tyr (Y)
  SITE 116: note = X at 116 is Phe (F), His (H), or Tyr (Y).

SEQ ID NO: 121 - protein, 127 aa, synthetic construct; identical to SEQ ID NO: 117
  REGION 1..127: note = Clone TL22 heavy chain variable region

SEQ ID NO: 122 - protein, 111 aa, synthetic construct; identical to SEQ ID NO: 98
  REGION 1..111: note = clone TL22 light chain with consensus CDR3
  SITE 91: note = where X at 91 is Tyr (Y), Ser (S) or Asp (D);
  SITE 92: note = X at 92 is Ser (S), Asn (N), Trp (W), Leu (L), Glu (E), Asp (D), Gly (G),
  SITE 93: note = X at 93 is absent, Trp (W), Tyr (Y), Asp (D), Pro (P), -Trp-Glu- (-WE-), -Leu-Arg- (-LR-), or -Asn-Tyr- (-NY-);
  SITE 94: note = X at 94 is Ser (S), Lys (K), Tyr (Y), Gly (G), Trp (W), Asn (N), or Glu (E);
  SITE 95: note = X at 95 is Ser (S), Asp (D), Trp (W), Gly (G), Asp (D), or Pro (P);
  SITE 96: note = X at 96 is Leu (L) or Pro (P);
  SITE 97: note = X at 97 is Ile (I), Leu (L), or Val (V);

SEQ ID NO: 123 - protein, 111 aa, synthetic construct
  REGION 1..111: note = Clone TL22 light chain variable region
DIQMTQSPSSLSASVGDRVTITCRASQSVSSAVAWYQQKPGKAPKLLIYSASSLYSGVPSRFSGSRSGTDFTLTISSLQPEDFATYYCQQSSWNGLITFGQGTKVEIKRTV

SEQ ID NO: 124 - protein, 127 aa, synthetic construct; identical to SEQ ID NO: 100
  REGION 1..127: note = clone TL23 heavy chain with consensus CDR1 and CDR3
  SITE 29: note = X at 29 is Phe (F), Trp (W), or Tyr (Y)
  SITE 30: note = X at 30 is Ser (S), Ala (A), Phe (F), Gly (G), His (H), Ile (I), Met (M), Asn (N), Gln (Q), Arg (R), Thy (T), Val (V), Trp (W), or Tyr (Y);
  SITE 32: note = X at 32 is Asn (N), Ala (A), Cys (C), Phe (F), His (H), Leu (L), Met (M), or Ser (S)
  SITE 33: note = X at 33 is Tyr (Y), Glu (E), or Phe (F).
  SITE 99: note = X at 99 is Tyr (Y) or Phe (F);
  SITE 101: note = X at 101 is Val (V), Glu (E), His (H), Ile (I), Leu (L), Met (M), Pro (P), Gln (Q), or Thy (T);
  SITE 103: note = X at 103 is Thy (T), Ala (A), Glu (E), His (H), Ile (I), Lys (K), Leu (L), Met (M), Asn (N), Gln (Q), Arg (R), or Ser (S);
  SITE 104: note = X at 104 is Met (M) or Leu (L);
  SITE 105: note = X at 105 is His (H), Ala (A), Lys (K), Met (M), Gln (Q), or Arg (R);
  SITE 106: note = X at 106 is Gln (Q), Ala (A), Phe (F), His (H), Ile (I), Lys (K), Leu (L), Met (M), Asn (N), Arg (R), Ser (S), or Thy (T);
  SITE 107: note = X at 107 is Tyr (Y), Ala (A), Phe (F), Gly (G), or Arg (R);
  SITE 111: note = X at 111 is Glu (E), Gln (Q), or Val (V);
  SITE 112: note = X at 112 is Gln (Q), Met (M), or Ser (S);
  SITE 113: note = X at 113 is Met (M), Phe (F), Leu (L), Arg (R), or Thy (T);
  SITE 115: note = X at 115 is Ala (A) or Tyr (Y)
  SITE 116: note = X at 116 is Phe (F), His (H), or Tyr (Y).

SEQ ID NO: 125 - protein, 127 aa, synthetic construct; identical to SEQ ID NO: 117
  REGION 1..127: note = Clone TL23 heavy chain variable region

SEQ ID NO: 126 - protein, 111 aa, synthetic construct; identical to SEQ ID NO: 98
  REGION 1..111: note = clone TL23 light chain with consensus CDR3
  SITE 91: note = where X at 91 is Tyr (Y), Ser (S) or Asp (D);
  SITE 92: note = X at 92 is Ser (S), Asn (N), Trp (W), Leu (L), Glu (E), Asp (D), Gly (G),
  SITE 93: note = X at 93 is absent, Trp (W), Tyr (Y), Asp (D), Pro (P), -Trp-Glu- (-WE-), -Leu-Arg- (-LR-), or -Asn-Tyr- (-NY-);
  SITE 94: note = X at 94 is Ser (S), Lys (K), Tyr (Y), Gly (G), Trp (W), Asn (N), or Glu (E);
  SITE 95: note = X at 95 is Ser (S), Asp (D), Trp (W), Gly (G), Asp (D), or Pro (P);
  SITE 96: note = X at 96 is Leu (L) or Pro (P);
  SITE 97: note = X at 97 is Ile (I), Leu (L), or Val (V);

SEQ ID NO: 127 - protein, 111 aa, synthetic construct
  REGION 1..111: note = Clone TL23 light chain variable region
DIQMTQSPSSLSASVGDRVTITCRASQSVSSAVAWYQQKPGKAPKLLIYSASSLYSGVPSRFSGSRSGTDFTLTISSLQPEDFATYYCQQSGWGGLITFGQGTKVEIKRTV

SEQ ID NO: 128 - protein, 127 aa, synthetic construct; identical to SEQ ID NO: 100
  REGION 1..127: note = clone TL24 heavy chain with consensus CDR1 and CDR3
  SITE 29: note = X at 29 is Phe (F), Trp (W), or Tyr (Y)
  SITE 30: note = X at 30 is Ser (S), Ala (A), Phe (F), Gly (G), His (H), Ile (I), Met (M), Asn (N), Gln (Q), Arg (R), Thy (T), Val (V), Trp (W), or Tyr (Y);
  SITE 32: note = X at 32 is Asn (N), Ala (A), Cys (C), Phe (F), His (H), Leu (L), Met (M), or Ser (S)
  SITE 33: note = X at 33 is Tyr (Y), Glu (E), or Phe (F).
  SITE 99: note = X at 99 is Tyr (Y) or Phe (F);
  SITE 101: note = X at 101 is Val (V), Glu (E), His (H), Ile (I), Leu (L), Met (M), Pro (P), Gln (Q), or Thy (T);
  SITE 103: note = X at 103 is Thy (T), Ala (A), Glu (E), His (H), Ile (I), Lys (K), Leu (L), Met (M), Asn (N), Gln (Q), Arg (R), or Ser (S);
  SITE 104: note = X at 104 is Met (M) or Leu (L);
  SITE 105: note = X at 105 is His (H), Ala (A), Lys (K), Met (M), Gln (Q), or Arg (R);
  SITE 106: note = X at 106 is Gln (Q), Ala (A), Phe (F), His (H), Ile (I), Lys (K), Leu (L), Met (M), Asn (N), Arg (R), Ser (S), or Thy (T);
  SITE 107: note = X at 107 is Tyr (Y), Ala (A), Phe (F), Gly (G), or Arg (R);
  SITE 111: note = X at 111 is Glu (E), Gln (Q), or Val (V);
  SITE 112: note = X at 112 is Gln (Q), Met (M), or Ser (S);
  SITE 113: note = X at 113 is Met (M), Phe (F), Leu (L), Arg (R), or Thy (T);
  SITE 115: note = X at 115 is Ala (A) or Tyr (Y)
  SITE 116: note = X at 116 is Phe (F), His (H), or Tyr (Y).

SEQ ID NO: 129 - protein, 127 aa, synthetic construct; identical to SEQ ID NO: 117
  REGION 1..127: note = Clone TL24 heavy chain variable region

SEQ ID NO: 130 - protein, 111 aa, synthetic construct; identical to SEQ ID NO: 98
  REGION 1..111: note = clone TL24 light chain with consensus CDR3
  SITE 91: note = where X at 91 is Tyr (Y), Ser (S) or Asp (D);
  SITE 92: note = X at 92 is Ser (S), Asn (N), Trp (W), Leu (L), Glu (E), Asp (D), Gly (G),
  SITE 93: note = X at 93 is absent, Trp (W), Tyr (Y), Asp (D), Pro (P), -Trp-Glu- (-WE-), -Leu-Arg- (-LR-), or -Asn-Tyr- (-NY-);
  SITE 94: note = X at 94 is Ser (S), Lys (K), Tyr (Y), Gly (G), Trp (W), Asn (N), or Glu (E);
  SITE 95: note = X at 95 is Ser (S), Asp (D), Trp (W), Gly (G), Asp (D), or Pro (P);
  SITE 96: note = X at 96 is Leu (L) or Pro (P);
  SITE 97: note = X at 97 is Ile (I), Leu (L), or Val (V);

SEQ ID NO: 131 - protein, 111 aa, synthetic construct
  REGION 1..111: note = Clone TL24 light chain variable region
DIQMTQSPSSLSASVGDRVTITCRASQSVSSAVAWYQQKPGKAPKLLIYSASSLYSGVPSRFSGSRSGTDFTLTISSLQPEDFATYYCQQSSYEDPITFGQGTKVEIKRTV

SEQ ID NO: 132 - protein, 127 aa, synthetic construct; identical to SEQ ID NO: 100
  REGION 1..127: note = clone LL2 heavy chain with consensus CDR1 and CDR3
  SITE 29: note = X at 29 is Phe (F), Trp (W), or Tyr (Y)
  SITE 30: note = X at 30 is Ser (S), Ala (A), Phe (F), Gly (G), His (H), Ile (I), Met (M), Asn (N), Gln (Q), Arg (R), Thy (T), Val (V), Trp (W), or Tyr (Y);
  SITE 32: note = X at 32 is Asn (N), Ala (A), Cys (C), Phe (F), His (H), Leu (L), Met (M), or Ser (S)
  SITE 33: note = X at 33 is Tyr (Y), Glu (E), or Phe (F).
  SITE 99: note = X at 99 is Tyr (Y) or Phe (F);
  SITE 101: note = X at 101 is Val (V), Glu (E), His (H), Ile (I), Leu (L), Met (M), Pro (P), Gln (Q), or Thy (T);
  SITE 103: note = X at 103 is Thy (T), Ala (A), Glu (E), His (H), Ile (I), Lys (K), Leu (L), Met (M), Asn (N), Gln (Q), Arg (R), or Ser (S);
  SITE 104: note = X at 104 is Met (M) or Leu (L);
  SITE 105: note = X at 105 is His (H), Ala (A), Lys (K), Met (M), Gln (Q), or Arg (R);
  SITE 106: note = X at 106 is Gln (Q), Ala (A), Phe (F), His (H), Ile (I), Lys (K), Leu (L), Met (M), Asn (N), Arg (R), Ser (S), or Thy (T);
  SITE 107: note = X at 107 is Tyr (Y), Ala (A), Phe (F), Gly (G), or Arg (R);
  SITE 111: note = X at 111 is Glu (E), Gln (Q), or Val (V);
  SITE 112: note = X at 112 is Gln (Q), Met (M), or Ser (S);
  SITE 113: note = X at 113 is Met (M), Phe (F), Leu (L), Arg (R), or Thy (T);
  SITE 115: note = X at 115 is Ala (A) or Tyr (Y)
  SITE 116: note = X at 116 is Phe (F), His (H), or Tyr (Y).

SEQ ID NO: 133 - protein, 127 aa, synthetic construct
  REGION 1..127: note = Clone LL2 heavy chain variable region
EVQLVESGGGLVQPGGSLRLSCAASGFTWSGSYIHWVRQAPGKGLEWVASIYSAGGYTDYADSVKGRFTISADTSKNTAYLQMNSLRAEDTAVYYCARYGVYTLASAGSWESLPAFDYWGQGTLVTV

SEQ ID NO: 134 - protein, 111 aa, synthetic construct; identical to SEQ ID NO: 98
  REGION 1..111: note = clone LL2 light chain with consensus CDR3
  SITE 91: note = where X at 91 is Tyr (Y), Ser (S) or Asp (D);
  SITE 92: note = X at 92 is Ser (S), Asn (N), Trp (W), Leu (L), Glu (E), Asp (D), Gly (G),
  SITE 93: note = X at 93 is absent, Trp (W), Tyr (Y), Asp (D), Pro (P), -Trp-Glu- (-WE-), -Leu-Arg- (-LR-), or -Asn-Tyr- (-NY-);
  SITE 94: note = X at 94 is Ser (S), Lys (K), Tyr (Y), Gly (G), Trp (W), Asn (N), or Glu (E);
  SITE 95: note = X at 95 is Ser (S), Asp (D), Trp (W), Gly (G), Asp (D), or Pro (P);
  SITE 96: note = X at 96 is Leu (L) or Pro (P);
  SITE 97: note = X at 97 is Ile (I), Leu (L), or Val (V);

SEQ ID NO: 135 - protein, 112 aa, synthetic construct
  REGION 1..112: note = Clone LL2 light chain variable region
DIQMTQSPSSLSASVGDRVTITCRASQSVSSAVAWYQQKPGKAPKLLIYSASSLYSGVPSRFSGSRSGTDFTLTISSLQPEDFATYYCQQSSWESPPITFGQGTKVEIKRTV

SEQ ID NO: 136 - protein, 127 aa, synthetic construct; identical to SEQ ID NO: 100
  REGION 1..127: note = clone LL5 heavy chain with consensus CDR1 and CDR3
  SITE 29: note = X at 29 is Phe (F), Trp (W), or Tyr (Y)
  SITE 30: note = X at 30 is Ser (S), Ala (A), Phe (F), Gly (G), His (H), Ile (I), Met (M), Asn (N), Gln (Q), Arg (R), Thy (T), Val (V), Trp (W), or Tyr (Y);
  SITE 32: note = X at 32 is Asn (N), Ala (A), Cys (C), Phe (F), His (H), Leu (L), Met (M), or Ser (S)
  SITE 33: note = X at 33 is Tyr (Y), Glu (E), or Phe (F).
  SITE 99: note = X at 99 is Tyr (Y) or Phe (F);
  SITE 101: note = X at 101 is Val (V), Glu (E), His (H), Ile (I), Leu (L), Met (M), Pro (P), Gln (Q), or Thy (T);
  SITE 103: note = X at 103 is Thy (T), Ala (A), Glu (E), His (H), Ile (I), Lys (K), Leu (L), Met (M), Asn (N), Gln (Q), Arg (R), or Ser (S);
  SITE 104: note = X at 104 is Met (M) or Leu (L);
  SITE 105: note = X at 105 is His (H), Ala (A), Lys (K), Met (M), Gln (Q), or Arg (R);
  SITE 106: note = X at 106 is Gln (Q), Ala (A), Phe (F), His (H), Ile (I), Lys (K), Leu (L), Met (M), Asn (N), Arg (R), Ser (S), or Thy (T);
  SITE 107: note = X at 107 is Tyr (Y), Ala (A), Phe (F), Gly (G), or Arg (R);
  SITE 111: note = X at 111 is Glu (E), Gln (Q), or Val (V);
  SITE 112: note = X at 112 is Gln (Q), Met (M), or Ser (S);
  SITE 113: note = X at 113 is Met (M), Phe (F), Leu (L), Arg (R), or Thy (T);
  SITE 115: note = X at 115 is Ala (A) or Tyr (Y)
  SITE 116: note = X at 116 is Phe (F), His (H), or Tyr (Y).

SEQ ID NO: 137 - protein, 127 aa, synthetic construct; identical to SEQ ID NO: 133
  REGION 1..127: note = Clone LL5 heavy chain variable region

SEQ ID NO: 138 - protein, 111 aa, synthetic construct; identical to SEQ ID NO: 98
  REGION 1..111: note = clone LL5 light chain with consensus CDR3
  SITE 91: note = where X at 91 is Tyr (Y), Ser (S) or Asp (D);
  SITE 92: note = X at 92 is Ser (S), Asn (N), Trp (W), Leu (L), Glu (E), Asp (D), Gly (G),
  SITE 93: note = X at 93 is absent, Trp (W), Tyr (Y), Asp (D), Pro (P), -Trp-Glu- (-WE-), -Leu-Arg- (-LR-), or -Asn-Tyr- (-NY-);
  SITE 94: note = X at 94 is Ser (S), Lys (K), Tyr (Y), Gly (G), Trp (W), Asn (N), or Glu (E);
  SITE 95: note = X at 95 is Ser (S), Asp (D), Trp (W), Gly (G), Asp (D), or Pro (P);
  SITE 96: note = X at 96 is Leu (L) or Pro (P);
  SITE 97: note = X at 97 is Ile (I), Leu (L), or Val (V);

SEQ ID NO: 139 - protein, 111 aa, synthetic construct
  REGION 1..111: note = Clone LL5 light chain variable region
DIQMTQSPSSLSASVGDRVTITCRASQSVSSAVAWYQQKPGKAPKLLIYSASSLYSGVPSRFSGSRSGTDFTLTISSLQPEDFATYYCQQSSYDGLLTFGQGTKVEIKRTV

SEQ ID NO: 140 - protein, 652 aa, Homo sapiens
  REGION 1..652: note = extracellular domain of human HER2 S310F
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGFCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLT

SEQ ID NO: 141 - protein, 652 aa, Homo sapiens
  REGION 1..652: note = extracellular domain of human HER2 S310Y
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGYCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLT

SEQ ID NO: 142 - protein, 459 aa, synthetic construct
  REGION 1..459: note = heavy chain of antibody CH15
EVQLVESGGGLVQPGGSLRLSCAASGFTFSGNYIHWVRQAPGKGLEWVASIYSAGGYTDYADSVKGRFTISADTSKNTAYLQMNSLRAEDTAVYYCARYGNYTMHQYGSWEQMPAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 143 - protein, 214 aa, synthetic construct
  REGION 1..214: note = light chain of antibody CH15
DIQMTQSPSSLSASVGDRVTITCRASQSVSSAVAWYQQKPGKAPKLLIYSASSLYSGVPSRFSGSRSGTDFTLTISSLQPEDFATYYCQQSSSSLITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 144 - protein, 523 aa, synthetic construct
  REGION 1..523: note = single-chain diabody formed from CH15V-CD3 antibody fragments
DIVRSDIQMTQSPSSLSASVGDRVTITCRASQSVSSAVAWYQQKPGKAPKLLIYSASSLYSGVPSRFSGSRSGTDFTLTISSLQPEDFATYYCQQSSSSLITFGQGTKVEIKGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVSSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSEVQLVESGGGLVQPGGSLRLSCAASGFTFSGNYIHWVRQAPGKGLEWVASIYSAGGYTDYADSVKGRFTISADTSKNTAYLQMNSLRAEDTAVYYCARYGVYTMHQYGSWEQMPAFDYWGQGTLVTVSSLEGGGGLNDIFEAQKIEWHESRHHHHHH

SEQ ID NO: 145 - protein, 523 aa, synthetic construct
  REGION 1..523: note = single-chain diabody formed from LL2-CD3 antibody fragments
DIVRSDIQMTQSPSSLSASVGDRVTITCRASQSVSSAVAWYQQKPGKAPKLLIYSASSLYSGVPSRFSGSRSGTDFTLTISSLQPEDFATYYCQQSSSSLITFGQGTKVEIKGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVSSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSEVQLVESGGGLVQPGGSLRLSCAASGFTWSGSYIHWVRQAPGKGLEWVASIYSAGGYTDYADSVKGRFTISADTSKNTAYLQMNSLRAEDTAVYYCARYGVYTLASAGSWESLPAFDYWGQGTLVTVSSLEGGGGLNDIFEAQKIEWHESRHHHHHH

SEQ ID NO: 146 - protein, 523 aa, synthetic construct
  REGION 1..523: note = single-chain diabody formed from sTL1-CD3 antibody fragments
DIVRSDIQMTQSPSSLSASVGDRVTITCRASQSVSSAVAWYQQKPGKAPKLLIYSASSLYSGVPSRFSGSRSGTDFTLTISSLQPEDFATYYCQQSSSSLITFGQGTKVEIKGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVSSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSEVQLVESGGGLVQPGGSLRLSCAASGFTWSGAYIHWVRQAPGKGLEWVASIYSAGGYTDYADSVKGRFTISADTSKNTAYLQMNSLRAEDTAVYYCARYGTYELKSYGSWESLPAFDYWGQGTLVTVSSLEGGGGLNDIFEAQKIEWHESRHHHHHH

SEQ ID NO: 147 - protein, 523 aa, synthetic construct
  REGION 1..523: note = single-chain diabody formed from sTL18-CD3 antibody fragments
DIVRSDIQMTQSPSSLSASVGDRVTITCRASQSVSSAVAWYQQKPGKAPKLLIYSASSLYSGVPSRFSGSRSGTDFTLTISSLQPEDFATYYCQQSSSSLITFGQGTKVEIKGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVSSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSEVQLVESGGGLVQPGGSLRLSCAASGFTFGGSYIHWVRQAPGKGLEWVASIYSAGGYTDYADSVKGRFTISADTSKNTAYLQMNSLRAEDTAVYYCARYGVYTLHQYGSWEQLPAFDYWGQGTLVTVSSLEGGGGLNDIFEAQKIEWHESRHHHHHH

SEQ ID NO: 148 - protein, 529 aa, synthetic construct
  REGION 1..529: note = bi-specific T-cell engager sTL18_BiTE_HL
EVQLVESGGGLVQPGGSLRLSCAASGFTFGGSYIHWVRQAPGKGLEWVASIYSAGGYTDYADSVKGRFTISADTSKNTAYLQMNSLRAEDTAVYYCARYGVYTLHQYGSWEQLPAFDYWGQGTLVTVSSGGGGSGGGGSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQSSSSLITFGQGTKVEIKSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVSSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKLEGGGGLNDIFEAQKIEWHESRHHHHHH

SEQ ID NO: 149 - protein, 529 aa, synthetic construct
  REGION 1..529: note = bi-specific T-cell engager sTL18_BiTE_LH
DIQMTQSPSSLSASVGDRVTITCRASQSVSSAVAWYQQKPGKAPKLLIYSASSLYSGVPSRFSGSRSGTDFTLTISSLQPEDFATYYCQQSSSSLITFGQGTKVEIKGGGGSGGGGSGGGGSEVQLVESGGGLVQPGGSLRLSCAASGFTFGGSYIHWVRQAPGKGLEWVASIYSAGGYTDYADSVKGRFTISADTSKNTAYLQMNSLRAEDTAVYYCARYGVYTLHQYGSWEQLPAFDYWGQGTLVTVSSSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVSSLEGGGGLNDIFEAQKIEWHESRHHHHHH

SEQ ID NO: 150 - protein, 229 aa, synthetic construct
  REGION 1..229: note = bi-specific sTL18_CrossMab, Chain A UCHT1VHCL
EVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 151 - protein, 680 aa, synthetic construct
  REGION 1..680: note = bi-specific sTL18_CrossMab, Chain B UCHT1VLCH1_Fcknob
EVQLVESGGGLVQPGGSLRLSCAASGFTFGGSYIHWVRQAPGKGLEWVASIYSAGGYTDYADSVKGRFTISADTSKNTAYLQMNSLRAEDTAVYYCARYGVYTLHQYGSWEQLPAFDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 152 - protein, 457 aa, synthetic construct
  REGION 1..457: note = bi-specific sTL18_CrossMab, Chain C sTL18VHCH1_Fchole
EVQLVESGGGLVQPGGSLRLSCAASGFTFGGSYIHWVRQAPGKGLEWVASIYSAGGYTDYADSVKGRFTISADTSKNTAYLQMNSLRAEDTAVYYCARYGVYTLHQYGSWEQLPAFDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 153 - protein, 214 aa, synthetic construct
  REGION 1..214: note = bi-specific sTL18_CrossMab, Chain D sTL18VLCL
DIQMTQSPSSLSASVGDRVTITCRASQSVSSAVAWYQQKPGKAPKLLIYSASSLYSGVPSRFSGSRSGTDFTLTISSLQPEDFATYYCQQSSSSLITFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 154 - protein, 481 aa, synthetic construct
  REGION 1..481: note = bi-specific sTL18VHCH1_UCHT1scFv
EVQLVESGGGLVQPGGSLRLSCAASGFTFGGSYIHWVRQAPGKGLEWVASIYSAGGYTDYADSVKGRFTISADTSKNTAYLQMNSLRAEDTAVYYCARYGVYTLHQYGSWEQLPAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVSSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIK

SEQ ID NO: 155 - protein, 214 aa, synthetic construct; identical to SEQ ID NO: 143
  REGION 1..214: note = bi-specific sTL18VLCL

SEQ ID NO: 156 - protein, 232 aa, synthetic construct
  REGION 1..232: note = bi-specific sTL18VHCH1
EVQLVESGGGLVQPGGSLRLSCAASGFTFGGSYIHWVRQAPGKGLEWVASIYSAGGYTDYADSVKGRFTISADTSKNTAYLQMNSLRAEDTAVYYCARYGVYTLHQYGSWEQLPAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 157 - protein, 463 aa, synthetic construct
  REGION 1..463: note = bi-specific sTL18VLCL_UCHT1scFv
DIQMTQSPSSLSASVGDRVTITCRASQSVSSAVAWYQQKPGKAPKLLIYSASSLYSGVPSRFSGSRSGTDFTLTISSLQPEDFATYYCQQSSSSLITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVSSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIK

SEQ ID NO: 158 - protein, 129 aa, synthetic construct
  REGION 1..129: note = Clone 11_Y95S VH
EVQLVESGGGLVQPGGSLRLSCAASGFTVYSSSIHWVRQAPGKGLEWVASISSYYGYTSYADSVKGRFTISADTSKNTAYLQMNSLRAEDTAVYYCARSGNYTMHQYGSWEQMPAFDYWGQGTLVTVSS

SEQ ID NO: 159 - protein, 129 aa, synthetic construct
  REGION 1..129: note = Clone 11_ G96S VH
EVQLVESGGGLVQPGGSLRLSCAASGFTVYSSSIHWVRQAPGKGLEWVASISSYYGYTSYADSVKGRFTISADTSKNTAYLQMNSLRAEDTAVYYCARYSNYTMHQYGSWEQMPAFDYWGQGTLVTVSS

SEQ ID NO: 160 - protein, 129 aa, synthetic construct
  REGION 1..129: note = Clone 11 N97S VH
EVQLVESGGGLVQPGGSLRLSCAASGFTVYSSSIHWVRQAPGKGLEWVASISSYYGYTSYADSVKGRFTISADTSKNTAYLQMNSLRAEDTAVYYCARYGSYTMHQYGSWEQMPAFDYWGQGTLVTVSS

SEQ ID NO: 161 - protein, 129 aa, synthetic construct
  REGION 1..129: note = Clone 11 Y98S VH
EVQLVESGGGLVQPGGSLRLSCAASGFTVYSSSIHWVRQAPGKGLEWVASISSYYGYTSYADSVKGRFTISADTSKNTAYLQMNSLRAEDTAVYYCARYGNSTMHQYGSWEQMPAFDYWGQGTLVTVSS

SEQ ID NO: 162 - protein, 129 aa, synthetic construct
  REGION 1..129: note = Clone 11_T99S VH
EVQLVESGGGLVQPGGSLRLSCAASGFTVYSSSIHWVRQAPGKGLEWVASISSYYGYTSYADSVKGRFTISADTSKNTAYLQMNSLRAEDTAVYYCARYGNYSMHQYGSWEQMPAFDYWGQGTLVTVSS

SEQ ID NO: 163 - protein, 129 aa, synthetic construct
  REGION 1..129: note = Clone 11_ M100S VH
EVQLVESGGGLVQPGGSLRLSCAASGFTVYSSSIHWVRQAPGKGLEWVASISSYYGYTSYADSVKGRFTISADTSKNTAYLQMNSLRAEDTAVYYCARYGNYTSHQYGSWEQMPAFDYWGQGTLVTVSS

SEQ ID NO: 164 - protein, 129 aa, synthetic construct
  REGION 1..129: note = Clone 11_H100aS VH
EVQLVESGGGLVQPGGSLRLSCAASGFTVYSSSIHWVRQAPGKGLEWVASISSYYGYTSYADSVKGRFTISADTSKNTAYLQMNSLRAEDTAVYYCARYGNYTMSQYGSWEQMPAFDYWGQGTLVTVSS

SEQ ID NO: 165 - protein, 129 aa, synthetic construct
  REGION 1..129: note = Clone 11 Q100bS VH
EVQLVESGGGLVQPGGSLRLSCAASGFTVYSSSIHWVRQAPGKGLEWVASISSYYGYTSYADSVKGRFTISADTSKNTAYLQMNSLRAEDTAVYYCARYGNYTMHSYGSWEQMPAFDYWGQGTLVTVSS

SEQ ID NO: 166 - protein, 129 aa, synthetic construct
  REGION 1..129: note = Clone 11 Y100cS VH
EVQLVESGGGLVQPGGSLRLSCAASGFTVYSSSIHWVRQAPGKGLEWVASISSYYGYTSYADSVKGRFTISADTSKNTAYLQMNSLRAEDTAVYYCARYGNYTMHQSGSWEQMPAFDYWGQGTLVTVSS

SEQ ID NO: 167 - protein, 129 aa, synthetic construct
  REGION 1..129: note = Clone 11 G100dS VH
EVQLVESGGGLVQPGGSLRLSCAASGFTVYSSSIHWVRQAPGKGLEWVASISSYYGYTSYADSVKGRFTISADTSKNTAYLQMNSLRAEDTAVYYCARYGNYTMHQYSSWEQMPAFDYWGQGTLVTVSS

SEQ ID NO: 168 - protein, 129 aa, synthetic construct
  REGION 1..129: note = Clone 11 S100eG VH
EVQLVESGGGLVQPGGSLRLSCAASGFTVYSSSIHWVRQAPGKGLEWVASISSYYGYTSYADSVKGRFTISADTSKNTAYLQMNSLRAEDTAVYYCARYGNYTMHQYGGWEQMPAFDYWGQGTLVTVSS

SEQ ID NO: 169 - protein, 129 aa, synthetic construct
  REGION 1..129: note = Clone 11 W100fS VH
EVQLVESGGGLVQPGGSLRLSCAASGFTVYSSSIHWVRQAPGKGLEWVASISSYYGYTSYADSVKGRFTISADTSKNTAYLQMNSLRAEDTAVYYCARYGNYTMHQYGSSEQMPAFDYWGQGTLVTVSS

SEQ ID NO: 170 - protein, 129 aa, synthetic construct
  REGION 1..129: note = Clone 11_E100gS VH
EVQLVESGGGLVQPGGSLRLSCAASGFTVYSSSIHWVRQAPGKGLEWVASISSYYGYTSYADSVKGRFTISADTSKNTAYLQMNSLRAEDTAVYYCARYGNYTMHQYGSWSQMPAFDYWGQGTLVTVSS

SEQ ID NO: 171 - protein, 129 aa, synthetic construct
  REGION 1..129: note = Clone 11_Q100hS VH
EVQLVESGGGLVQPGGSLRLSCAASGFTVYSSSIHWVRQAPGKGLEWVASISSYYGYTSYADSVKGRFTISADTSKNTAYLQMNSLRAEDTAVYYCARYGNYTMHQYGSWESMPAFDYWGQGTLVTVSS

SEQ ID NO: 172 - protein, 129 aa, synthetic construct
  REGION 1..129: note = Clone 11_M100iS VH
EVQLVESGGGLVQPGGSLRLSCAASGFTVYSSSIHWVRQAPGKGLEWVASISSYYGYTSYADSVKGRFTISADTSKNTAYLQMNSLRAEDTAVYYCARYGNYTMHQYGSWEQSPAFDYWGQGTLVTVSS

SEQ ID NO: 173 - protein, 129 aa, synthetic construct
  REGION 1..129: note = Clone 11_P100jS VH
EVQLVESGGGLVQPGGSLRLSCAASGFTVYSSSIHWVRQAPGKGLEWVASISSYYGYTSYADSVKGRFTISADTSKNTAYLQMNSLRAEDTAVYYCARYGNYTMHQYGSWEQMSAFDYWGQGTLVTVSS

SEQ ID NO: 174 - protein, 129 aa, synthetic construct
  REGION 1..129: note = Clone 11_A100kS VH
EVQLVESGGGLVQPGGSLRLSCAASGFTVYSSSIHWVRQAPGKGLEWVASISSYYGYTSYADSVKGRFTISADTSKNTAYLQMNSLRAEDTAVYYCARYGNYTMHQYGSWEQMPSFDYWGQGTLVTVSS

SEQ ID NO: 175 - protein, 129 aa, synthetic construct
  REGION 1..129: note = Clone 11 F100lS VH
EVQLVESGGGLVQPGGSLRLSCAASGFTVYSSSIHWVRQAPGKGLEWVASISSYYGYTSYADSVKGRFTISADTSKNTAYLQMNSLRAEDTAVYYCARYGNYTMHQYGSWEQMPASDYWGQGTLVTVSS

SEQ ID NO: 176 - protein, 514 aa, synthetic construct
  REGION 1..514: note = Trastuzumab_scDb
DIVRSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVSSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSLEGGGGLNDIFEAQKIEWHESRHHHHHH

SEQ ID NO: 177 - protein, 725 aa, synthetic construct
  REGION 1..725: note = sTL18_scDb_huFc chain A
DIQMTQSPSSLSASVGDRVTITCRASQSVSSAVAWYQQKPGKAPKLLIYSASSLYSGVPSRFSGSRSGTDFTLTISSLQPEDFATYYCQQSSSSLITFGQGTKVEIKGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVSSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSEVQLVESGGGLVQPGGSLRLSCAASGFTFGGSYIHWVRQAPGKGLEWVASIYSAGGYTDYADSVKGRFTISADTSKNTAYLQMNSLRAEDTAVYYCARYGVYTLHQYGSWEQLPAFDYWGQGTLVTVSSDKTYTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPSSSSHHHHHH

SEQ ID NO: 178 - protein, 225 aa, synthetic construct
  REGION 1..225: note = sTL18_scDb_huFc chain B
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

(19) United States
(12) Patent Application Publication
(10) Pub. No.: US 2026/0234287 A1
(43) Pub. Date: Aug. 13, 2026

(54) HUMAN SYNTHETIC ANTIBODIES TARGETING HUMAN EPIDERMAL GROWTH FACTOR RECEPTOR 2 (HER2) MUTANTS

(71) Applicant: NEW YORK UNIVERSITY, New York, NY (US)

(72) Inventors: Elizabeth BUCK, Huntington Bay, NY (US); Kenneth G. GELES, Nyack, NY (US); Shohei KOIDE, New York, NY (US); Akiko KOIDE, New York, NY (US); Nadia LELOUP, Ixelles (BE); Takamitsu HATTORI, Fort Lee, NJ (US); Injin BANG, Redmond, WA (US)

(21) Appl. No.: 19/156,390

(22) PCT Filed: Feb. 14, 2024
(86) PCT No.: PCT/US2024/015812

§ 371 Date: Aug. 13, 2025

Related U.S. Application Data
(60) Provisional application No. 63/445,702, filed on Feb. 14, 2023.
(60) Provisional application No. 63/586,811, filed on Sep. 29, 2023.

(51) Int. Cl. C07K 16/32; A61K 39/00; A61P 35/00; G01N 33/57515

(52) U.S. Cl. CPC C07K 16/32; A61P 35/00; G01N 33/57515; A61K 2039/505; C07K 2317/52; C07K 2317/55; C07K 2317/565; C07K 2317/732; C07K 2317/92; G01N 2458/00

(57) ABSTRACT

Disclosed are synthetic antibodies and binding fragments thereof that target mutant forms of HER2, specifically mutant HER2 containing S310F/Y, nucleic acid molecules and vectors encoding such antibodies and binding fragments, as well as pharmaceutical compositions containing the same, and the use thereof for treatment or diagnosis of such mutant HER2-mediated cancers.

This application claims the priority benefit of U.S. Provisional Patent Application Ser. Nos. 63/586,811, filed Sep. 29, 2023, and 63/445,702, filed Feb. 14, 2023, each of which is hereby incorporated by reference in its entirety.

This application contains a computer readable Sequence Listing, which has been submitted electronically in XML format and is hereby incorporated by reference in its entirety. Said XML copy, created on Feb. 14, 2024, is named 147462_002343.xml and is 360,055 bytes in size.

### FIELD

This invention relates to synthetic antibodies and binding fragments thereof that target mutant forms of HER2, nucleic acid molecules and vectors encoding such antibodies and binding fragments, as well as pharmaceutical compositions containing the same, and the use thereof for treatment or diagnosis of HER2-mediated cancers.

### BACKGROUND

As a cell-surface antigen that also regulates key signaling pathway, human epidermal growth factor receptor 2 (HER2) is an attractive drug target. Indeed, there are several FDA-approved drugs including antibodies, trastuzumab and pertuzumab, that target the extracellular region of HER2 (Parakh et al., “Evolution of Anti-HER2 Therapies for Cancer Treatment,” Cancer Treatment Reviews 59:1-21 (2017); Capelan et al., “Pertuzumab: New Hope for Patients with HER2-positive Breast Cancer,” Annals of Oncology 24:273-282 (2013); Albanell et al., “Trastuzumab, a Humanized Anti-HER2 Monoclonal Antibody, for the Treatment of Breast Cancer,” Drugs of Today (Barcelona, Spain: 1998) 35:931-946 (1999)). These antibodies are effective against cancers with unusually increased levels of HER2 on their cell surface, commonly termed HER2-positive cancers, and are now a part of the routine therapy. Nevertheless, overexpression of an antigen is a cancer-associated, but not cancer-specific, feature and targeting a cancer-associated antigen carries a risk of adverse effects, as the same antigen is also present on the healthy tissues albeit at a lower level. Indeed, a major drawback of trastuzumab is the risk of heart problems as the basal level of HER2 expressed on cardiomyocytes is enough to make them targetable by trastuzumab for antibody-mediated cellular cytotoxicity (Chen et al., “Risk of Cardiac Dysfunction with Trastuzumab in Breast Cancer Patients: A Meta-analysis,” Cancer Treatment Reviews 37:312-320 (2011); Telli et al., “Trastuzumab-related Cardiotoxicity: Calling Into Question the Concept of Reversibility,” J. Clinical Oncology 25:3525-3533 (2007); Hussain et al., “Cardiac Outcomes of Trastuzumab Therapy in Patients with HER2-positive Breast Cancer and Reduced Left Ventricular Ejection Fraction,” Breast Cancer Res Treat 175:239-246 (2019)). Although there remain the needs for the development of more specific drug that only targets HER2 on cancer cells, the approved drugs and those under development represent effective therapies against cancers with high levels of wild-type HER2.

Overexpression and genetic amplification of HER2 are not the only mechanisms by which HER2 promote the development of cancer. Cancer genomics efforts have identified numerous oncogenic mutations in various cell-surface proteins including HER2 (Zeng et al., “Targeting HER2 Genomic Alterations in Non-small Cell Lung Cancer,” J. National Cancer Center 1:58-73 (2021); Greulich et al., “Functional Analysis of Receptor Tyrosine Kinase Mutations in Lung Cancer Identifies Oncogenic Extracellular Domain Mutations of ERBB2,” Proc. Nat'l Acad. Sci. USA 109:14476-14481 (2012); Sun et al., “Analysis of Different HER-2 Mutations in Breast Cancer Progression and Drug Resistance,” J. Cell. Mol. Med. 19:2691-2701 (2015)). HER2 harboring an oncogenic mutation can promote tumorigenesis through hyperactive signaling without overexpression (Bose et al., “Activating HER2 Mutations in HER2 Gene Amplification Negative Breast Cancer,” Cancer Discovery 3:224-237 (2013)). Such HER2-low cancers have been neglected as a subject of HER2-targeted therapy, as the focus has been skewed towards treating HER2-positive cancers. In addition, the oncogenic mutations sometimes occur in response to cancer therapy, as tumors acquire resistance against administered HER2-targeting drugs (Nayar et al., “Acquired HER2 Mutations in ER+ Metastatic Breast Cancer Confer Resistance to Estrogen Receptor-directed Therapies,” Nature Genetics 51:207-216 (2019)). Development of a therapy specific to HER2 mutant-driven cancers could effectively treat HER2-low cancer, thereby addressing the current gap in HER2-targeting immunotherapy.

Among the hyperactivating mutations identified, S310F and S310Y are among the most common mutations found in the extracellular region (Petrelli et al., “Clinical and Pathological Characterization of HER2 Mutations in Human Breast Cancer: A Systematic Review of the Literature,” Breast Cancer Research and Treatment 166:339-349 (2017)). S310F/Y have been found in multiple types of cancers, including lung, colon, breast, ovarian and urinary bladder cancer (Greulich et al., “Functional Analysis of Receptor Tyrosine Kinase Mutations in Lung Cancer Identifies Oncogenic Extracellular Domain Mutations of ERBB2,” Proc. Nat'l Acad. Sci. USA 109:14476-14481 (2012); Kan et al., “Diverse Somatic Mutation Patterns and Pathway Alterations in Human Cancers,” Nature 466:869-873 (2010); Eng et al., “Outcomes of Chemotherapies and HER2 Directed Therapies in Advanced HER2-mutant Lung Cancers,” Lung Cancer 99:53-56 (2016); Ding et al., “Somatic Mutations Affect Key Pathways in Lung Adenocarcinoma,” Nature 455:1069-1075 (2008); Shah et al., “The Clonal and Mutational Evolution Spectrum of Primary Triple-negative Breast Cancers,” Nature 486:395-399 (2012); Jasra et al., “A Rare Case of S310F Somatic ERBB2 Mutation in a HER2-nonamplified Breast Cancer,” Clinical Breast Cancer 17:e37-e41 (2017); Han et al., “Targeted Sequencing of Cancer-related Genes in Colorectal Cancer Using Next-Generation Sequencing,” PloS One 8:e64271 (2013); Ross et al., “A High Frequency of Activating Extracellular Domain ERBB2 (HER2) Mutation in Micropapillary Urothelial Carcinoma,” Clinical Cancer Research 20:68-75 (2014)). S310 is located in the dimerization arm binding pocket and the mutations have been linked to the elevated level of HER2 dimerization (Greulich et al., “Functional Analysis of Receptor Tyrosine Kinase Mutations in Lung Cancer Identifies Oncogenic Extracellular Domain Mutations of ERBB2,” Proc. Nat'l Acad. Sci. USA 109:14476-14481 (2012); Shin et al., “The HER2 S310F Mutant Can Form an Active Heterodimer with the EGFR, Which Can Be Inhibited by Cetuximab but Not by Trastuzumab as well as Pertuzumab,” Biomolecules 9(10):629 (2019)). Homo- and hetero-dimerization are critical regulatory mechanism of the HER family receptor tyrosine kinases, as it is a crucial early step in the activation of downstream signaling. HER2 is unique among the four subtypes of HERs found in human in that it has no known endogenous ligands but it regulates the activity of the other HER subtypes through heterodimerization (Yarden and Sliwkowski, “Untangling the ErbB Signalling Network,” Nature Reviews Molecular Cell Biology 2:127-137 (2001); Cho et al., “Structure of the Extracellular Region of HER2 Alone and in Complex with the Herceptin Fab,” Nature 421:756-760 (2003)). Homodimerization of wild type (WT) HER2 is not favored, but its overexpression can drive homodimerization via mass action, leading to signal activation (Di Fiore et al., “erbB-2 Is a Potent Oncogene When Overexpressed in NIH/3T3 Cells,” Science 237:178-182 (1987); Hudziak et al., “Increased Expression of the Putative Growth Factor Receptor p185HER2 Causes Transformation and Tumorigenesis of NIH 3T3 Cells,” Proc. Nat'l Acad. Sci. USA 84:7159-7163 (1987); Alvarado et al., “ErbB2 Resembles an Autoinhibited Invertebrate Epidermal Growth Factor Receptor,” Nature 461:287-291 (2009)). By contrast, HER2 S310F/Y enhances the dimerization even when it is not overexpressed, promoting oncogenesis. A recent structure of HER2 S310F in complex with HER3/neuregulin-1ß revealed molecular interactions contributing to stabilizing dimerization (Diwanji et al., “Structures of the HER2-HER3-NRG1β Complex Reveal a Dynamic Dimer Interface,” Nature 600:339-343 (2021)). However, the structure of the HER2 S310F/Y homodimer is yet to be determined.

Although HER2 S310F/Y are single-point mutation hot-spots that are cancer drivers, which make them potentially attractive drug targets, there have been no reported therapeutics directly targeting them. More generally, there is a rather surprising paucity of therapeutics selective to oncogenic mutations of cell-surface antigens. It is generally challenging to develop an antibody that recognizes a single point mutation with high selectivity.

It would be desirable to identify antibody or antibody binding fragments that have the ability to bind selectively to HER2 mutants possessing a point mutation at Ser310, particularly Ser310Phe/Tyr, while displaying low binding affinity for wildtype HER2.

The claimed invention is directed to overcoming this and other deficiencies in the art.

### SUMMARY

A first aspect relates to an antibody-based molecule that binds mutant, but not wild type, Receptor tyrosine-protein kinase erbB-2 (HER2), the mutant HER2 including a substitution of serine corresponding to position 310 (S310) of SEQ ID NO: 1.

In preferred embodiments, the antibody-based molecule is capable of selectively binding to a mutant HER2 having a serine to phenylalanine substitution at a position corresponding to serine310 (S310F) of SEQ ID NO: 1 or a serine to tyrosine substitution at a position corresponding to serine310 (S310Y) of SEQ ID NO: 1.

A second aspect relates to a polynucleotide encoding the antibody-based molecule according to the first aspect. Also covered by this aspect are a vector that includes the polynucleotide as well as host cells containing the vector (and the polynucleotide).

A third aspect relates to a chimeric antigen receptor molecule that includes an antibody an antibody-based molecule according to the first aspect, a transmembrane domain, and an activation domain.

A fourth aspect relates to a polynucleotide encoding the chimeric receptor molecule according to the third aspect. Also covered by this aspect are a vector that includes the polynucleotide as well as host cells that express the polynucleotide or contain the vector (and the polynucleotide). Preferred host cells include natural killer cells, T cells, and macrophages.

A fifth aspect relates to an immunoconjugate that includes the antibody-based molecule according to the first aspect, and a cytotoxic agent that is coupled to the antibody-based molecule.

A sixth aspect relates to a multi-specific antibody or multi-specific binding fragment thereof that includes a first antigen-binding arm that includes the antibody-based molecule according to the first aspect, and a second antigen-binding arm that binds to a surface antigen selectively expressed on an immune cell surface. The targeting of surface antigen selectively expressed on natural killer cells, T cells, and macrophages is contemplated.

A seventh aspect relates to a polynucleotide encoding the multi-specific antibody or multi-specific binding fragment thereof according to the sixth aspect. Also covered by this aspect are a vector that includes the polynucleotide as well as host cells that express the polynucleotide or contain the vector (and the polynucleotide).

An eighth aspect relates to a pharmaceutical composition that includes the antibody-based molecule according to the first aspect, the immunoconjugate according to the fifth aspect, or the multi-specific antibody or multi-specific binding fragment thereof according to the sixth aspect, or one (or more) of the polynucleotides or vectors according to the second, fourth, or seventh aspects; and a pharmaceutically acceptable carrier.

A ninth aspect relates to a delivery vehicle that includes one (or more) of the polynucleotides or one (or more) of vectors according to the second, fourth, or seventh aspects. Any of a variety of delivery vehicles are contemplated including, without limitation, nanoparticle, polymer-based particles, and lipid-based particles.

A tenth aspect relates to a method of treating a subject having a HER2-positive cancer. This method includes the step of administering to the subject a pharmaceutical composition according to the ninth aspect in an amount effective to treat the subject having the HER2-positive cancer.

An eleventh aspect relates to a method of treating a subject having a cancer expressing a mutant HER-2. This method includes the step of administering to the subject a population of autologous immune cells expressing the chimeric antigen receptor according to the third aspect in an amount effective to treat the subject having the mutant HER-2 expressing cancer.

A twelfth aspect relates to a method of selectively killing of HER2-mutant cells in a population of cells. This method includes the step of contacting a population of cells comprising HER2-mutant cells with the antibody-based molecule according to the first aspect, the immunoconjugate according to the fifth aspect, or the multi-specific antibody or multi-specific binding fragment according to the sixth aspect in an amount effective to selectively kill the HER2-mutant cells in the population of cells. The targeting of HER2-mutant cells that express an S310F/Y mutant HER2 is contemplated.

A thirteenth aspect relates to a diagnostic agent that includes the antibody-based molecule according to the first aspect, and a detectable label that is coupled to the antibody-based molecule.

A fourteenth aspect relates to a method of detecting a cancer expressing mutant HER2 in a subject. This method includes the steps of contacting a biological sample from the subject with an antibody-based molecule according to the first aspect or the diagnostic agent according to the thirteenth aspect, and detecting a complex between HER2 mutant cancer cells and the antibody-based molecule or a complex between the HER2 mutant cancer cells and the diagnostic agent in the biological sample.

A fifteenth aspect relates to an HER2-Fc conjugate protein that includes the amino acid sequence of a mutant HER2 amino acid sequence fused to an Fc region amino acid sequence.

The accompanying Examples demonstrate the development of antibodies that are capable of recognizing a single-point mutation in the context of a large antigen. Specifically, antibodies that bind potently to both HER2 S310F and S310Y with high selectivity over WT HER2 are identified. When assembled into a multi-specific antibody format, exemplified by a T-cell engager format, these multi-specific antibodies can effectively kill cancer cells expressing HER2 S310F/Y. Using a bi-specific T-cell engager for S310F/Y and CD3, the T-cell engager demonstrated efficacy in inhibiting tumor growth or causing tumor shrinkage depending on the dose. In addition, these antibodies can be used as mechanistic probes and for structural analyses using cryo-electron microscopy (cryoEM), which offers insight into the dynamics of HER2 homo- and hetero-dimerization and effects of the S310F/Y mutations on them.

### BRIEF DESCRIPTION OF THE DRAWINGS

FIGS. 1A-1D illustrate affinity improvement of CH15 from deep mutational scanning. In FIG. 1A, the top panel shows the flow cytometry profile of CH15 scFv with 1 μM HER2 S310F (23-652)-Fc and the bottom panel shows that of the mutated library. The horizontal axis shows the surface display level, and the vertical axis shows the target binding level. Clones exhibiting higher affinity are indicated with the gray circle. FIG. 1B is a comparison of the CDR-H3 sequence of isolated clones from the higher affinity pool. Mutated residues are highlighted in gray. The clone marked as 5 is named as CH15V and further characterized. In FIG. 1C-1D, binding titration curves of identified clones for HER2 S310F (23-652)-Fc and HER2 S310Y (23-652)-Fc in the scFv format using yeast display are shown.

FIGS. 2A-2D illustrate characterization of CH15V as purified protein samples. FIGS. 2A-2B are BLI sensorgrams of purified CH15V Fab towards HER2 S310F (23-652)-Fc and HER2 S310Y (23-652)-Fc immobilized on the sensor surface, respectively. The white curves indicate the best fit of a single exponential to the dissociation phase. The dissociation rate was measured to be 5.02×10⁻³ s⁻¹ and 8.39×10⁻³ s⁻¹ respectively to HER2 S310F and S310Y. In FIG. 2C-2D, binding of the indicated antibodies in hIgG1 format to HEK293T expressing WT HER2 or to HEK293T expressing HER2 S310F are shown. The histograms for flow cytometry data are shown in the upper panels of FIGS. 2C-2D, and the median fluorescence intensities of these histograms are plotted as bar graphs below.

FIGS. 3A-3B illustrate the effects of the indicated antibodies in the hIgG1 format on the proliferation of Ba/F3 cells expressing either HER2 S310F or Her2 S310Y, respectively. The cells were seeded at 100,000 cells/ml with the indicated antibody over the range of concentration. Cells were counted after 3 days of culture. Experiments were performed in triplicate. The curves show nonlinear fitting of a 1:1 binding model. IC₅₀ values derived from the fitting are shown.

FIGS. 4A-4F illustrate Assessment of binding capacity of antibodies selective to HER2 S310F/Y mutants. FIG. 4A shows schematics of antigen constructs used in the Examples. FIG. 4B shows BLI sensorgrams of the indicated antibodies in the Fab format to HER2 S310F-Fc and HER2 S310Y-Fc immobilized on AHC biosensors. The white lines show the fitted curves of a local 1-to-1 binding model. The deduced dissociation rate constants are given in Table A. (C) FIG. 4C shows BLI sensorgrams of the indicated antibodies in the Fab format (200 nM) to immobilized HER2 WT-Fc. In FIGS. 4D-4F, the mean fluorescence intensities (MFI) of the indicated antibodies in the IgG format (50 nM) to HEK293T S310F^{Low} cells (4D), HEK293T S310Y^{Low} cells (4E), and) HEK293T WT^{High} cells (4F), detected with flow cytometry. The graphs show the mean of MFI with error bars indicating SEM (n=3). The dotted lines indicate the basal signal given by the control samples with no primary antibody, but with a secondary antibody conjugate with a fluorophore.

FIGS. 5A-5I illustrate cryoEM structures of HER2 S310F-Fc alone and in complex with TL1 Fab. FIG. 5A shows the density map and built-in structure of HER2 S310F-Fc alone. One protomer is shown in light gray and the other in dark gray. FIG. 5B is a closeup view of the structure of the dimerization arm (white) and its interaction partners (blue in color version). FIGS. 5C-5D show the density map and structure, respectively, of the HER2 S310F-TL1 Fab complex. TL1 VH is shown in dark blue in color version, VL in light blue in color version, and HER2 in light gray. S310F is shown in yellow in color version. The detailed workflow is provided in FIGS. 14, 15A-15E. FIG. 5E shows TL1 residues that interact with HER2. Residues of TL1 that are within 4 Å of HER2 are shown as stick models. The region of HER2 in proximity of TL1 VH (≤4 Å) is highlighted in pink in color version. FIGS. 5F-5I show the molecular interactions between TL1 and HER2. Interaction between TL1 CDR-H3 and HER2 are shown in FIGS. 5F, 5G. Interaction between TL1 CDR-H1 and CDR L3 and HER2 are shown in FIGS. 5H and 5I, respectively. Dotted lines indicate polar interaction with a distance less than 3.2 Å.

FIGS. 6A-6I illustrate the assessment of binding capacity of fourth-generation antibodies to HER2 mutant and the impact of HER2 dimerization on antibody-HER2 interaction. FIGS. 6A-6B show BLI sensorgrams of the indicated antibodies in the Fab format to monomeric HER2 S310F-Fc (6A) and HER2 S310Y-Fc (6B) antigens immobilized on AHC biosensors. The white curves show the best fits of a global 1-to-1 binding model determined with the Octet data analysis software. The deduced parameters are given in Table B. FIGS. 6C-6D show BLI sensorgrams of the indicated antibodies to immobilized HER2 WT-Fc. Antibodies in the Fab format at 200 nM was used in FIG. 6C, and those in the IgG format at 100 nM were used in FIG. 6D. FIGS. 6E-6F show the MFI of the indicated antibodies in the IgG format (50 nM) to HEK293T WT^{High} (6E) and HTB9 (6F). The graphs show the mean and SEM (n=3). FIG. 6G shows BLI sensorgrams of 20 nM sTL18 Fab to monomeric or dimeric constructs of HER2 S310F-Fc (left) and HER2 S310Y-Fc (right). FIGS. 6H-6I show BLI sensorgrams of 20 nM of sTL18 Fab to immobilized HER heterodimeric constructs, HER1/HER2 S310F (6H) and HER3/HER2 S310F (6I) in the presence or absence of the respective ligands of HER1 and HER3.

FIGS. 7A-7K illustrate the cytotoxic potency of developed antibodies in different formats. FIGS. 7A-7D show the efficacy of ADC constructs on the indicated cells. Cell viability was measured with PrestoBlue after three days of incubation with an ADC construct. FIGS. 7E-7J show the efficacy of bispecific T cell engager constructs on the indicated cells. FIG. 7K shows the effect of adding EGF (10 nM) on the efficacy of sTL18 scDb. All experiments in this figure were done at least in triplicate and error bars indicate the SEM. All the graphs were prepared and fitted with Prism 9 and deduced IC₅₀ and EC₅₀ values are given in Table C.

FIG. 8 shows size exclusion chromatograms and SDS-PAGE gel profiles of purified antigens.

FIG. 9 illustrates a comparison of HER2 expression levels among different cell lines and engineered cells as probed with 50 nM trastuzumab and detected with flow cytometry. The median florescence intensities (MFI) are shown.

FIG. 10 illustrates binding titrations of the indicated antibodies to HTB9 as detected using flow cytometry.

FIG. 11 illustrates binding titrations of Clone 11 (eft) and CH15 in the IgG format to HEK293T S310F^{High} cells as detected using flow cytometry.

FIG. 12 illustrates binding of the indicated antibodies to HEK293T WT^{High} cells (left graph) and to HEK293T S310F^{High} cells (right graph) as detected using flow cytometry.

FIGS. 13A-13B illustrate a heatmap presentation of deep mutational scanning (DMS) data on CH15V. FIG. 13A shows the results from the positive selection pool, i.e., clones that bound to HER2 S310F. FIG. 13B shows the results from negative selection, i.e., clones that did not bind to HER2 S310F. The x-axis of the plot shows the residue number (VH) and original amino acid, while y-axis indicated screened 20 amino acids. The enrichment levels are indicated with the color code.

FIG. 14 illustrates CryoEM workflow for HER2 S310F-Fc in complex with TL1 Fab.

FIG. 15A-15E illustrate FSC plot, resolution estimations and map quality from cryoEM data analyses for the HER2 S310F/TL1 complex. FIG. 15A shows the local resolution of the cryoEM map in the range of 2.2 to 3.4 Å with a scale bar at the bottom. FIG. 15B shows gold-standard Fourier shell correlation (GSFSC) curves. FIG. 15C shows angular distribution of the particles used in the final reconstruction. FIG. 15D shows Fourier shell correlation plot calculated based on the fitted structure model and cryoEM map. FIG. 15E shows representative regions of the fitted model and cryoEM map.

FIG. 16 illustrates the structural comparisons of TL1 VH with other proteins that bind to the dimerization arm-binding pocket of HER2. HER2 is shown in light grey with S310F marked in yellow. TL1 VH is in dark blue. On left is superposition with HER2/pertuzumab structure (Hao et al., “Cryo-EM Structure of Her2 Extracellular Domain-Trastuzumab Fab-Pertuzumab Fab Complex,” PLoS One 14:e0216095-e0216095 (2019), which is hereby incorporated by reference in its entirety) where pertuzumab is in orange in color version. On right is superimposition with HER3/HER2 S310F (Diwanji et al., “Structures of the HER2-HER3-NRG1β Complex Reveal a Dynamic Dimer Interface,” Nature 600:339-343 (2021), which is hereby incorporated by reference in its entirety) where the HER3 dimerization arm is shown in green in color version.

FIG. 17 illustrates residues of CDR H1 and H3 of TL1, which are colored based on the DMS analyses of FIGS. 13A-13B6. In a colored version of this image, green indicates residues that can be mutated to <5 amino acid types, and blue indicates residues can be mutated to ≥5 amino acid types. Residues of HER2 within 4 Å of TL1 CDR H1 and H3 are displayed in pink in a colored version.

FIG. 18 illustrates BLI sensorgrams of sTL18 Fab (20, 40, and 80 nM) against homodimeric HER2 S310F/Y-Fc constructs immobilized on sensor tips.

FIG. 19 illustrates the HER2 S310F homodimer structure determined in this study (top) and published structures of HER homo-/heterodimers. The identities of the receptors and the PDB IDs are indicated.

FIGS. 20A-201 illustrate cell killing assays. FIG. 20A shows ADCC assay with HEK293T S310F^{High} cells. FIGS. 20B-20C show ADC assays done using sTL1 and sLL2 with HEK293T S310F^{High} cells (20B) and with HEK293T S310Y^{High} cells (20C). FIG. 20D shows ADC assay done on HTB-9 cells with 1 nM of indicated antibodies. FIGS. 20E-20F show results from cell killing assay with T-cell engagers (scDbs) using, in the order of, HEK293T S310F^{High}, HEK293T S310Y^{High} and HTB-9 cells. FIG. 20G shows cell killing assays with CH15V and sTL1, sLL2 and trastuzumab in the scDb format against HTB-9 cells. Control indicates a negative-control scDb construct. FIG. 20H shows cell killing assay with sTL18 scDb on HTB-9 cells in the presence of 0.1 and 1 ng/ml of EGF. FIG. 20I shows cell killing assay with sTL18 scDb-Fc to determine the effect of having Fc domain fused to scDb. All assays were done in at least triplicate and the error bars indicate SEM. Nonlinear fit was performed using Prism 9 (GraphPad).

FIG. 21 illustrates cell killing assay with sTL18 scDb with and without Fc to determine the effect of having Fc domain fused to scDb.

FIG. 22 illustrates a mouse xenograft model using sTL18 scDb-Fc. The left panel shows the scheme of the sTL18 scDb-Fc efficacy study. The right panel shows relative changes in the tumor volume over the treatment period. Each treatment group started with 7 mice and error bars indicate SEM.

### DETAILED DESCRIPTION

The epidermal growth factor receptor (EGFR) family of transmembrane receptor tyrosine kinases activates signaling pathways regulating cellular proliferation and survival. Receptor tyrosine-protein kinase erbB-2 (HER2/ErbB2/Neu) is a member of this EGFR family. Although there is no known ligand for HER2, it exerts its activity through heterodimerization with other EGFR family members to affect the downstream signaling of those receptors. Increased HER2 expression and activation of its tyrosine kinase is known to promote cell transformation and oncogenesis. This has been well characterized in HER2 gene amplification in breast and gastro-esophageal cancers. Recently, somatic HER2 gene mutations have been detected in a range of human cancer types. Preclinical data suggest that functionally activating HER2 mutations may drive and maintain cancers in a manner analogous to HER2 gene amplification (see Connell and Doherty, “Activating HER2 Mutations as Emerging Targets in Multiple Solid Cancers,” ESMO Open 2:e000279 (2017), which is hereby incorporated by reference in its entirety). Mutations of ERBB2 encoding HER2 are found in many types of cancers. HER mutations are found across all exons of the HER2 gene. Mutations affecting the extracellular domain and transmembrane domains are capable of activating HER2 signaling.

One such mutation is at the S310 position which resides in the HER2 extracellular domain II and causes receptor activation. The S310 mutation is clinically one of the most frequent HER2 extracellular domain mutations (see Shin et al., “The HER2 S310F Mutant Can Form an Active Heterodimer with the EGFR, Which Can Be Inhibited by Cetuximab but Not by Trastuzumab as well as Pertuzumab,” Biomolecules 9(10):629 (2019), which is hereby incorporated by reference in its entirety). Mutations at S310 are among the most frequent HER2 mutations, and the S310F and S310Y mutations are considered hotspot mutations (Hyman et al., “HER Kinase Inhibition in Patients with HER2- and HER3-mutant Cancers,” Nature 554(7691):189-94 (2018), which is hereby incorporated by reference in its entirety). The HER2 S310F mutation is strongly activating (Greulich et al., “Functional Analysis of Receptor Tyrosine Kinase Mutations in Lung Cancer Identifies Oncogenic Extracellular Domain Mutations of ERBB2,” Proc. Nat'l Acad. Sci. USA 109:14476-14481 (2012), which is hereby incorporated by reference in its entirety), and it was found in patients with HER2-nonamplified cancer (see Connell and Doherty, “Activating HER2 Mutations as Emerging Targets in Multiple Solid Cancers,” ESMO Open 2:e000279 (2017); Chumsri et al., “Prolonged Response to Trastuzumab in a Patient With HER2-Nonamplified Breast Cancer with Elevated HER2 Dimerization Harboring an ERBB2 S310F Mutation,” J. Natl Compr Canc Netw. 13(9):1066-70 (2015), each of which is hereby incorporated by reference in its entirety).

Because position S310 is located within the extracellular domain (ECD) of HER2, it is accessible to biologics therapeutics such as antibodies. Current anti-HER2 therapeutic antibodies, Trastuzumab and Pertuzumab, are not selective to HER2 mutants, and thus they are not expected to selectively target cancer cells harboring a HER2 mutation in a patient. Therefore, antibodies that selectively recognize S310 mutations can have therapeutic efficacy through ADCC (antibody-mediated cellular cytotoxicity) or ADCP (antibody-mediated cellular phagocytosis). Such antibodies can be further engineered into biologic therapeutics and cellular therapeutics that effectively engage immune cells for cytotoxic effect, for examples, T-cell engagers, NK cell engagers, chimeric antigen receptor (CAR)-T cells, CAR-NK cells and CAR-macrophages.

Thus, the present disclosure relates to antibody-based molecules, including antibodies, epitope-binding domains thereof, and antibody derivatives as described herein, that are capable of selectively binding mutant, but not wildtype HER2. The mutant HER2 bound by the antibody-based molecules described herein comprises a substitution of serine at position 310 (S310) of HER2 (SEQ ID NO: 1). Such antibody-based molecules are useful for the treatment and diagnosis of cancers expressing the mutant HER2.

The amino acid sequence of human HER2 extracellular domain is provided below as SEQ ID NO: 1. The location of the S310 is bolded and underlined.

(SEQ ID NO: 1)
MELAALCRWGLLLALLPPGAASTQVCTGTDMKLRLPASPETHLDMLRH
LYQGCQVVQGNLELTYLPTNASLSFLQDIQEVQGYVLIAHNQVRQVPL
QRLRIVRGTQLFEDNYALAVLDNGDPLNNTTPVTGASPGGLRELQLRS
LTEILKGGVLIQRNPQLCYQDTILWKDIFHKNNQLALTLIDTNRSRAC
HPCSPMCKGSRCWGESSEDCQSLTRTVCAGGCARCKGPLPTDCCHEQC
AAGCTGPKHSDCLACLHFNHSGICELHCPALVTYNTDTFESMPNPEGR
YTFGASCVTACPYNYLSTDVGSCTLVCPLHNQEVTAEDGTQRCEKCSK
PCARVCYGLGMEHLREVRAVTSANIQEFAGCKKIFGSLAFLPESFDGD
PASNTAPLQPEQLQVFETLEEITGYLYISAWPDSLPDLSVFQNLQVIR
GRILHNGAYSLTLQGLGISWLGLRSLRELGSGLALIHHNTHLCFVHTV
PWDQLFRNPHQALLHTANRPEDECVGEGLACHQLCARGHCWGPGPTQC
VNCSQFLRGQECVEECRVLQGLPREYVNARHCLPCHPECQPQNGSVTC
FGPEADQCVACAHYKDPPFCVARCPSGVKPDLSYMPIWKFPDEEGACQ
PCPINCTHSCVDLDDKGCPAEQRASPLT

The amino acid sequence of human HER2 S310F extracellular domain is provided below as SEQ ID NO: 140. The location of the S310F is bolded and underlined.

(SEQ ID NO: 140)
MELAALCRWGLLLALLPPGAASTQVCTGTDMKLRLPASPETHLDMLRH
LYQGCQVVQGNLELTYLPTNASLSFLQDIQEVQGYVLIAHNQVRQVPL
QRLRIVRGTQLFEDNYALAVLDNGDPLNNTTPVTGASPGGLRELQLRS
LTEILKGGVLIQRNPQLCYQDTILWKDIFHKNNQLALTLIDTNRSRAC
HPCSPMCKGSRCWGESSEDCQSLTRTVCAGGCARCKGPLPTDCCHEQC
AAGCTGPKHSDCLACLHFNHSGICELHCPALVTYNTDTFESMPNPEGR
YTFGASCVTACPYNYLSTDVGFCTLVCPLHNQEVTAEDGTQRCEKCSK
PCARVCYGLGMEHLREVRAVTSANIQEFAGCKKIFGSLAFLPESFDGD
PASNTAPLQPEQLQVFETLEEITGYLYISAWPDSLPDLSVFQNLQVIR
GRILHNGAYSLTLQGLGISWLGLRSLRELGSGLALIHHNTHLCFVHTV
PWDQLFRNPHQALLHTANRPEDECVGEGLACHQLCARGHCWGPGPTQC
VNCSQFLRGQECVEECRVLQGLPREYVNARHCLPCHPECQPQNGSVTC
FGPEADQCVACAHYKDPPFCVARCPSGVKPDLSYMPIWKFPDEEGACQ
PCPINCTHSCVDLDDKGCPAEQRASPLT

The amino acid sequence of human HER2 S310Y extracellular domain is provided below as SEQ ID NO: 141. The location of the S310Y is bolded and underlined.

(SEQ ID NO: 141)
MELAALCRWGLLLALLPPGAASTQVCTGTDMKLRLPASPETHLDMLRH
LYQGCQVVQGNLELTYLPTNASLSFLQDIQEVQGYVLIAHNQVRQVPL
QRLRIVRGTQLFEDNYALAVLDNGDPLNNTTPVTGASPGGLRELQLRS
LTEILKGGVLIQRNPQLCYQDTILWKDIFHKNNQLALTLIDTNRSRAC
HPCSPMCKGSRCWGESSEDCQSLTRTVCAGGCARCKGPLPTDCCHEQC
AAGCTGPKHSDCLACLHFNHSGICELHCPALVTYNTDTFESMPNPEGR
YTFGASCVTACPYNYLSTDVGYCTLVCPLHNQEVTAEDGTQRCEKCSK
PCARVCYGLGMEHLREVRAVTSANIQEFAGCKKIFGSLAFLPESFDGD
PASNTAPLQPEQLQVFETLEEITGYLYISAWPDSLPDLSVFQNLQVIR
GRILHNGAYSLTLQGLGISWLGLRSLRELGSGLALIHHNTHLCFVHTV
PWDQLFRNPHQALLHTANRPEDECVGEGLACHQLCARGHCWGPGPTQC
VNCSQFLRGQECVEECRVLQGLPREYVNARHCLPCHPECQPQNGSVTC
FGPEADQCVACAHYKDPPFCVARCPSGVKPDLSYMPIWKFPDEEGACQ
PCPINCTHSCVDLDDKGCPAEQRASPLT

Although the above-identified polypeptides containing the extracellular domain of wildtype and mutant HER2 are shown above, it should be appreciated that full length wildtype and mutant HER2 proteins or polypeptides can also be used.

In accordance with the present invention, the HER2 antibody-based molecules described herein bind to an epitope of SEQ ID NO: 140 and SEQ ID NO: 141, which is not present in the amino acid sequence of SEQ ID NO: 1. The term “epitope” as used herein refers to an antigenic determinant capable of being bound to an antibody. Epitopes usually comprise surface groupings of molecules such as amino acids or sugar side chains and usually have specific three-dimensional structural characteristics, as well as specific charge characteristics. Conformational and non-conformational epitopes are distinguished in that the binding to the former, but not the latter, is lost in the presence of denaturing solvents. An epitope may comprise amino acid residues directly involved in the binding (also called the immunodominant component of the epitope) and other amino acid residues, which are not directly involved in the binding, such as amino acid residues that are effectively blocked by the specific antigen-binding peptide (in other words, the amino acid residue is within the footprint of the specific antigen-binding peptide). An epitope typically includes at least 3, and more usually, at least 5, 6, 7, 8, 9, 10, 11, 12, 13, 14, 15, 16, 17, 18, 19, 20 or more amino acids in a unique spatial conformation.

The HER2 antibody-based molecules of the present disclosure immunospecifically bind an epitope within the S310F/Y HER2 sequence of SEQ ID NO: 140 and SEQ ID NO: 141 more frequently, with greater duration and/or with greater affinity or avidity (i.e., dissociate more slowly) than an alternative epitope. In an embodiment, the HER2 antibody-based molecules described herein bind immunospecifically to any 2, 3, 4, 5, 6, 7, 8, 9, 10 or more amino acid residues of SEQ ID NO: 140 or SEQ ID NO: 141. The term “affinity”, “specific binding”, “binding”, “immunospecific binding”, “binding activity” or “specific binding activity”, as used herein, refers to the degree to which an antibody or an antibody fragment as defined herein binds to an epitope within the HER2 S310F sequence of SEQ ID NO: 140 and HER2 S310Y sequence of SEQ ID NO: 141, which epitope includes S310F/Y.

Antibody-based molecules include, without limitation full antibodies, epitope binding fragments of whole antibodies, and antibody derivatives. An epitope binding fragment of an antibody can be obtained through the actual fragmenting of a parental antibody (for example, a Fab or (Fab) 2 fragment). Alternatively, the epitope binding fragment is an amino acid sequence that comprises a portion of the amino acid sequence of such parental antibody. As used herein, a molecule is said to be a “derivative” of an antibody (or relevant portion thereof) if it is obtained through the actual chemical modification of a parent antibody or portion thereof, or if it comprises an amino acid sequence that is substantially similar to the amino acid sequence of such parental antibody or relevant portion thereof (for example, differing by less than 30%, less than 20%, less than 10%, or less than 5% from such parental molecule or such relevant portion thereof, or by 10 amino acid residues, or by fewer than 10, 9, 8, 7, 6, 5, 4, 3 or 2 amino acid residues from such parental molecule or relevant portion thereof).

In an embodiment, an antibody-based molecule of the present disclosure is an intact immunoglobulin or a molecule having an epitope-binding fragment thereof. As used herein, the terms “fragment”, “region”, “portion”, and “domain” are generally intended to be synonymous, unless the context of their use indicates otherwise. Naturally occurring antibodies typically comprise a tetramer, which is usually composed of at least two heavy (H) chains and at least two light (L) chains. Each heavy chain is comprised of a heavy chain variable (V_{H}) region and a heavy chain constant (C_{H}) region, usually comprised of three domains (C_{H}1, C_{H}2 and C_{H}3 domains). Heavy chains can be of any isotype, including IgG (IgG1, IgG2, IgG3 and IgG4 subtypes), IgA (IgAQ1 and IgA2 subtypes), IgM and IgE. Each light chain is comprised of a light chain variable (V_{L}) region and a light chain constant (C_{L}) region. Light chains include kappa chains and lambda chains. The heavy and light chain variable regions are typically responsible for antigen recognition, while the heavy and light chain constant regions may mediate the binding of the immunoglobulin to host tissues or factors, including various cells of the immune system (e.g., effector cells) and the first component (Clq) of the classical complement system. The V_{H} and V_{L} regions can be further subdivided into regions of hypervariability, termed “complementarity determining regions,” or “CDRs,” that are interspersed with regions of more conserved sequence, termed “framework regions” (FR). Each V_{H} and V_{L} region is composed of three CDR domains and four FR domains arranged from amino-terminus to carboxy-terminus in the following order: FR1-CDR1-FR2-CDR2-FR3-CDR3-FR4. The variable regions of the heavy and light chains contain a binding domain that interacts with an antigen. Of particular relevance are antibodies and their epitope-binding fragments that have been “isolated” so as to exist in a physical milieu distinct from that in which it may occur in nature or that have been modified so as to differ from a naturally occurring antibody in amino acid sequence.

Fragments of antibodies (including Fab and (Fab)₂ fragments) that exhibit epitope-binding ability can be obtained, for example, by protease cleavage of intact antibodies. Single domain antibody fragments possess only one variable domain (e.g., V_{L} or V_{H}). Examples of the epitope-binding fragments encompassed within the present invention include (i) Fab′ or Fab fragments, which are monovalent fragments containing the V_{L}, V_{H}, C_{L} and C_{H}1 domains; (ii) F(ab′)₂ fragments, which are bivalent fragments comprising two Fab fragments linked by a disulfide bridge at the hinge region; (iii) Fd fragments consisting essentially of the V_{H} and C_{H}1 domains; (iv) Fv fragments consisting essentially of a V_{L} and V_{H} domain, (v) dAb fragments (Ward et al. “Binding Activities Of A Repertoire Of Single Immunoglobulin Variable Domains Secreted From Escherichia coli,” Nature 341:544-546 (1989), which is hereby incorporated by reference in its entirety), which consist essentially of a V_{H} or V_{L} domain and also called domain antibodies (Holt et al. “Domain Antibodies: Proteins For Therapy,” Trends Biotechnol. 21(11):484-490 (2003), which is hereby incorporated by reference in its entirety); (vi) nanobodies (Revets et al. “Nanobodies as Novel Agents for Cancer Therapy,” Expert Opin. Biol. Ther. 5(1):111-124 (2005), which is hereby incorporated by reference in its entirety), and (vii) isolated complementarity determining regions (CDR). An epitope-binding fragment may contain 1, 2, 3, 4, 5 or all 6 of the CDR domains of such antibody. In an embodiment, a fragment (or region or portion or domain) of an antibody comprises, essentially consists of, or consists of 30 to 100 amino acids or 50 to 150 amino acids or 70 to 200 amino acids. In an embodiment, the length of a fragment (or region or portion or domain) of an antibody is at least 40%, 50%, 60%, 70%, 80%, 90% or 95% of the length of the antibody (full length antibody). In an embodiment, a fragment is an epitope binding fragment or a functional fragment of said antibody meaning it is expected it will elicit an activity of the antibody at least to some extent. “At least to some extent” may mean at least 20%, 30%, 40%, 50%, 60%, 70%, 80%, 90%, 95%, 100%, 150%, 200% or more. In an embodiment, the fragment of the antibody or the antibody should elicit a detectable activity of the antibody. An activity of the antibody has been earlier defined herein.

Such antibody fragments may be obtained using conventional techniques known to those of skill in the art. For example, F(ab′)₂ fragments may be generated by treating a full-length antibody with pepsin. The resulting F(ab′)₂ fragment may be treated to reduce disulfide bridges to produce Fab′ fragments. Fab fragments may be obtained by treating an IgG antibody with papain and Fab′ fragments may be obtained with pepsin digestion of IgG antibody. A Fab′ fragment may be obtained by treating an F(ab′)₂ fragment with a reducing agent, such as dithiothreitol. Antibody fragments may also be generated by expression of nucleic acids encoding such fragments in recombinant cells (see e.g., Evans et al. “Rapid Expression of An Anti-Human C5 Chimeric Fab Utilizing a Vector That Replicates in COS And 293 Cells,” J. Immunol. Meth. 184:123-38 (1995), which is hereby incorporated by reference in its entirety). For example, a chimeric gene encoding a portion of a F(ab′)₂ fragment could include DNA sequences encoding the CH1 domain and hinge region of the heavy chain, followed by a translational stop codon to yield such a truncated antibody fragment molecule. Suitable fragments capable of binding to a desired epitope may be readily screened for utility in the same manner as an intact antibody.

Antibody derivatives include those molecules that contain at least one epitope-binding domain of an antibody, and are typically formed using recombinant techniques. One exemplary antibody derivative includes a single chain Fv (scFv). A scFv is formed from the two domains of the Fv fragment, the V_{L} region and the V_{H} region, which may be encoded by separate genes. Such gene sequences or their encoding cDNA are joined, using recombinant methods, by a flexible linker (typically of about 10, 12, 15 or more amino acid residues) that enables them to be made as a single protein chain in which the V_{L} and V_{H} regions associate to form monovalent epitope-binding molecules (see e.g., Bird et al. “Single-Chain Antigen-Binding Proteins,” Science 242:423-426 (1988); and Huston et al. “Protein Engineering Of Antibody Binding Sites: Recovery Of Specific Activity In An Anti-Digoxin Single-Chain Fv Analogue Produced In Escherichia coli,” Proc. Natl. Acad. Sci. (U.S.A.) 85:5879-5883 (1988), each of which is hereby incorporated by reference in its entirety). Alternatively, by employing a flexible linker that is not too short (e.g., not less than about 9 residues) to enable the V_{L} and V_{H} regions of a different single polypeptide chains to associate together, one can form a bispecific antibody, having binding specificity for two different epitopes.

In another embodiment, the antibody derivative is a divalent or bivalent single-chain variable fragment, engineered by linking two scFvs together either in tandem (i.e., tandem scFv), or such that they dimerize to form a diabody (Holliger et al. “‘Diabodies’: Small Bivalent And Bispecific Antibody Fragments,” Proc. Natl. Acad. Sci. (U.S.A.) 90(14), 6444-8 (1993), which is hereby incorporated by reference in its entirety). In yet another embodiment, the antibody is a triabody, i.e., a trivalent single chain variable fragment, engineered by linking three scFvs together, either in tandem or in a trimer formation to form a triabody. In another embodiment, the antibody is a tetrabody of four single chain variable fragments. In another embodiment, the antibody is a “linear antibody” which is an antibody comprising a pair of tandem Fd segments (V_{H}-C_{H}1-V_{H}-C_{H}1) that form a pair of antigen binding regions (see Zapata et al. Protein Eng. 8(10):1057-1062 (1995), which is hereby incorporated by reference in its entirety). In another embodiment, the antibody derivative is a minibody, consisting of the single-chain Fv regions coupled to the C_{H}3 region (i.e., scFv-C_{H}3). Antibody-based molecules as described herein also includes multi-specific antibodies, e.g., bi-specific antibodies and tri-specific antibodies and antibody conjugates.

Additional exemplary bi-specific antibody fragments include, without limitation, CrossMab (Surowka et al., “Ten Years in the Making: Application of CrossMab Technology for the Development of Therapeutic bispecific Antibodies and Antibody Fusion Protein,” MAbs 13(1):1967714 (2021), which is hereby incorporated by reference in its entirety), ART-Ig (Igawa, “Next Generation Antibody Therapeutics Using Bispecific Antibody Technology,” Yakugaku Zasshi 137(7):831-36 (2017), which is hereby incorporated by reference in its entirety), BEAT (Skegro et al., “Immunoglobulin Domain Interface Exchange as a Platform Technology for the Generation of Fc Heterodimers and Bispecific Antibodies,” J Biol Chem. 292(23):9745-59 (2017), which is hereby incorporated by reference in its entirety), BiTE (Einsele et al., “The BiTE (Bispecific T-cell Engager) Platform: Development and Future Potential of a Targeted Immuno-oncology Therapy Across Tumor Types,” Cancer 126(14):3192-201 (2020), which is hereby incorporated by reference in its entirety), common light chains (De Nardis et al., “A New Approach for Generating Bispecific Antibodies Based on a Common Light Chain Format and the Stable Architecture of Human Immunoglobulin G1,” J Biol Chem. 292(35):14706-17 (2017), which is hereby incorporated by reference in its entirety), DAF (Bostrom et al., “Variants of the Antibody Herceptin that Interact with HER2 and VEGF at the Antigen Binding Site,” Science 323(5921):1610-14 (2009), which is hereby incorporated by reference in its entirety), DART (Johnson et al., “Effector Cell Recruitment with Novel Fv-based Dual-affinity Re-targeting Protein Leads to Potent Tumor Cytolysis and in vivo B-cell Depletion,” J Mol Biol. 399(3):436-49 (2010), which is hereby incorporated by reference in its entirety), DuoBody (Labrijn et al., “Efficient Generation of Stable Bispecific IgG1 by Controlled Fab-arm Exchange,” Proc. Natl. Acad. Sci. USA 110(13):5145-50 (2013), which is hereby incorporated by reference in its entirety), DutaFab (Beckmann et al., “DutaFabs Are Engineered Therapeutic Fab Fragments that Can Bind Two Targets Simultaneously,” Nat Commun. 12(1):708 (2021), which is hereby incorporated by reference in its entirety), DVD-Ig (Wu et al., “Simultaneous Targeting of Multiple Disease Mediators by a Dual-variable-domain Immunoglobulin,” Nat Biotechnol. 25(11):1290-97 (2007), which is hereby incorporated by reference in its entirety), Fab arm exchange (Strop et al., “Generating Bispecific Human IgG1 and IgG2 Antibodies from any Antibody Pair,” J Mol Biol. 420(3):204-19 (2012), which is hereby incorporated by reference in its entirety), Fcab (Leung et al., “A HER2-specific Modified Fc Fragment (Fcab) Induces Antitumor Effects Through Degradation of HER2 and Apoptosis,” Mol Ther. 23(11):1722-33 (2015), which is hereby incorporated by reference in its entirety), FORCEm (Dengl et al., “Format Chain Exchange (FORCE) for High-throughput Generation of Bispecific Antibodies in Combinatorial Binder-format Matrices,” Nat Commun. 11(1):4974 (2020), which is hereby incorporated by reference in its entirety), half antibody assembly (Spiess et al., “Bispecific Antibodies with Natural Architecture Produced by Co-culture of Bacteria Expressing Two Distinct Half-antibodies,” Nat Biotechnol. 31(8):753-58 (2013), which is hereby incorporated by reference in its entirety), Hetero-Ig (Liu et al., “A Novel Antibody Engineering Strategy for Making Monovalent Bispecific Heterodimeric IgG Antibodies by Electrostatic Steering Mechanism,” J Biol Chem. 290(12):7535-62 (2015), which is hereby incorporated by reference in its entirety), IgG-scFvv (Coloma et al., Design and Production of Novel Tetravalent Bispecific Antibodies,” Nat Biotechnol. 15(2):159-63 (1997); Dong et al., “A Stable IgG-like Bispecific Antibody Targeting the Epidermal Growth Factor Receptor and the type I Insulin-like Growth Factor Receptor Demonstrates Superior Anti-tumor Activity,” MAbs 3(3):273-88 (2011), each of which is hereby incorporated by reference in its entirety), KA-bodies (Fischer et al., “Exploiting Light Chains for the Scalable Generation and Platform Purification of Native Human Bispecific IgG,” Nat Commun. 6:6113 (2015), which is hereby incorporated by reference in its entirety), Multiclonics (De Nardis et al., “A New Approach for Generating Bispecific Antibodies Based on a Common Light Chain Format and the Stable Architecture of Human Immunoglobulin G1,” J Biol Chem. 292(35):14706-17 (2017), which is hereby incorporated by reference in its entirety), orthogonal Fab interface (Lewis et al., “Generation of Bispecific IgG Antibodies by Structure-based Design of an Orthogonal Fab Interface,” Nat Biotechnol. 32(2):191-98 (2014), which is hereby incorporated by reference in its entirety), Tandab (Cochlovius et al., “Cure of Burkitt's Lymphoma in Severe Combined Immunodeficiency Mice by T Cells, Tetravalent CD3×CD19 Tandem Diabody, and CD28 Costimulation,” Cancer Res. 60(16):4336-41 (2000), which is hereby incorporated by reference in its entirety), XmAb (Moore et al., “A Robust Heterodimeric Fc Platform Engineered for Efficient Development of Bispecific Antibodies of Multiple Formats,” Methods 154:38-50 (2019), which is hereby incorporated by reference in its entirety), VELOCI-Bi (Smith et al., “A Novel, Native-format Bispecific Antibody Triggering T-cell Killing of B-cells Is Robustly Active in Mouse Tumor Models and Cynomolgus Monkeys,” Sci Rep. 5(1):17943 (2015), which is hereby incorporated by reference in its entirety), and WuxiBODY (Guo et al., “A Potential Downstream Platform Approach for WuXiBody-based IgG-like Bispecific Antibodies,” Protein Expr Purif. 173:105647 (2020), which is hereby incorporated by reference in its entirety).

These and other useful antibody fragments and derivatives in the context of the present invention are discussed further herein. It also should be understood that the term antibody-based molecule, unless specified otherwise, also includes antibody-like polypeptides, such as chimeric antibodies and humanized antibodies, and antibody fragments retaining the ability to specifically bind to the antigen (epitope-binding fragments or functional fragment) provided by any known technique, such as enzymatic cleavage, peptide synthesis, and recombinant techniques. In an embodiment, the wording “antibody-based molecule” may be replaced by the word “antibody” or by the expression “antibody or a functional fragment thereof”

An antibody as generated herein may be of any isotype. As used herein, “isotype” refers to the immunoglobulin class (for instance IgG1, IgG2, IgG3, IgG4, IgD, IgA, IgE, or IgM) that is encoded by heavy chain constant region genes. The choice of isotype typically will be guided by the desired effector functions, such as antibody-dependent cellular cytotoxicity (ADCC) induction. Exemplary isotypes are IgG1, IgG2, IgG3, and IgG4. Particularly useful isotypes of the HER2 antibodies disclosed herein include IgG1 and IgG2.

Either of the human light chain constant regions, kappa or lambda, may be used. If desired, the class of a HER2 antibody of the present invention may be switched by known methods. For example, an antibody of the present invention that was originally IgM may be class switched to an IgG antibody of the present invention. Further, class switching techniques may be used to convert one IgG subclass to another, for instance from IgG1 to IgG2. Thus, the effector function of the antibodies of the present invention may be changed by isotype switching to, e.g., an IgG1, IgG2, IgG3, IgG4, IgD, IgA, IgE, or IgM antibody for various therapeutic uses.

In an embodiment, the antibody-based molecules of the present invention are “humanized,” particularly if they are to be employed for therapeutic purposes. The term “humanized” refers to a chimeric molecule, generally prepared using recombinant techniques, having an antigen-binding site derived from an immunoglobulin from a non-human species and a remaining immunoglobulin structure based upon the structure and/or sequence of a human immunoglobulin. The antigen-binding site may comprise either complete non-human antibody variable domains fused to human constant domains, or only the complementarity determining regions (CDRs) of such variable domains grafted to appropriate human framework regions of human variable domains. The framework residues of such humanized molecules may be wild-type (e.g., fully human) or they may be modified to contain one or more amino acid substitutions not found in the human antibody whose sequence has served as the basis for humanization. Humanization lessens or eliminates the likelihood that a constant region of the molecule will act as an immunogen in human individuals, but the possibility of an immune response to the foreign variable region remains (LoBuglio, A.F. et al. “Mouse/Human Chimeric Monoclonal Antibody In Man: Kinetics And Immune Response,” Proc. Natl. Acad. Sci. USA 86:4220-4224 (1989), which is hereby incorporated by reference in its entirety).

Phage display technology can alternatively be used to increase (or decrease) CDR affinity of the antibody-based molecules of the present invention. This technology, referred to as affinity maturation, employs mutagenesis or “CDR walking” and re-selection using the target antigen or an antigenic fragment thereof to identify antibodies having CDRs that bind with higher (or lower) affinity to the antigen when compared with the initial or parental antibody (see, e.g. Glaser et al., “Antibody Engineering By Codon-Based Mutagenesis In A Filamentous Phage Vector System,” J. Immunology 149:3903-3913 (1992), which is hereby incorporated by reference in its entirety). Mutagenizing entire codons rather than single nucleotides results in a semi-randomized repertoire of amino acid mutations. Libraries can be constructed consisting of a pool of variant clones each of which differs by a single amino acid alteration in a single CDR from another member of such library and which contain variants potentially representing each possible amino acid substitution for each CDR residue. Mutants with increased (or decreased) binding affinity for the antigen can be screened by contacting the immobilized mutants with labeled antigen. Any screening method known in the art can be used to identify variant antibody-based binding molecules with increased or decreased affinity to the antigen (e.g., ELISA) (See Wu, H. et al., “Stepwise In Vitro Affinity Maturation Of Vitaxin, An Alphav Beta3-Specific Humanized mAb,” Proc. Natl. Acad. Sci. USA 95:6037-6042 (1998); Yelton et al., “Affinity Maturation Of The BR96 Anti-Carcinoma Antibody By Codon-Based Mutagenesis,” J. Immunology 155:1994 (1995), each of which is hereby incorporated by reference in its entirety). CDR walking, which randomizes the light chain may be used (see, Schier et al., “Isolation of Picomolar Affinity Anti-c-erbB-2 Single-Chain Fv by Molecular Evolution Of The Complementarity Determining Regions In The Center Of The Antibody Binding Site,” J. Mol. Biol. 263:551-567 (1996), which is hereby incorporated by reference in its entirety).

Methods for affinity maturation of the HER2 antibody molecule are described herein and disclosed for example, in Krause et al., “An Insertion Mutation That Distorts Antibody Binding Site Architecture Enhances Function of a Human Antibody,” MBio. 2(1): e00345-10 (2011); Kuan et al., “Affinity-Matured Anti-Glycoprotein NMB Recombinant Immunotoxins Targeting Malignant Gliomas And Melanomas,” Int. J. Cancer 10.1002/ijc.25645 (2010); Hackel et al., “Stability And CDR Composition Biases Enrich Binder Functionality Landscapes,” J. Mol. Biol. 401(1):84-96 (2010); Montgomery et al., “Affinity Maturation And Characterization Of A Human Monoclonal Antibody Against HIV-1 gp41,” MAbs 1(5):462-474 (2009); Gustchina et al., “Affinity Maturation By Targeted Diversification Of The CDR-H2 Loop Of A Monoclonal Fab Derived From A Synthetic Naïve Human Antibody Library And Directed Against The Internal Trimeric Coiled-Coil Of Gp41 Yields A Set Of Fabs With Improved HIV-1 Neutralization Potency And Breadth,” Virology 393(1):112-119 (2009); Finlay et al., “Affinity Maturation Of A Humanized Rat Antibody For Anti-RAGE Therapy: Comprehensive Mutagenesis Reveals A High Level Of Mutational Plasticity Both Inside And Outside The Complementarity-Determining Regions,” J. Mol. Biol. 388(3):541-558 (2009); Bostrom et al., “Improving Antibody Binding Affinity And Specificity For Therapeutic Development,” Methods Mol. Biol. 525:353-376 (2009); Steidl et al., “In Vitro Affinity Maturation Of Human GM-CSF Antibodies By Targeted CDR-Diversification,” Mol. Immunol. 46(1):135-144 (2008); and Barderas et al., “Affinity Maturation of Antibodies Assisted by In Silico Modeling,” Proc. Natl. Acad. Sci. USA 105(26):9029-9034 (2008), each of which is hereby incorporated by reference in its entirety.

In an aspect of the present invention, the HER-antibody based molecule as described herein comprises the amino acid sequence of any one, any two, any three, any four, any five, or any six CDRs as provided in Tables 1 and 2 herein.

TABLE 1
HER2 Ab Heavy Chain CDRs

| Clone | CDRH1 | SEQ | CDRH2 | SEQ | CDRH3 | SEQ |
|---|---|---|---|---|---|---|
| 11-1 | SSSIH | 3 | SISSYYGYTSYADSVKG | 14 | YGNYTMHQYGSWEQMPAFDY | 25 |
| CH2 | RTSIH | 4 | EISSYDGYTDYADSVKG | 15 | YGNYTMHQYGSWEQMPAFDY | 25 |
| CH10 | TTSIH | 5 | EISSYNGDTDYADSVKG | 16 | YGNYTMHQYGSWEQMPAFDY | 25 |
| CH15 | GNYIH | 6 | SIYSAGGYTDYADSVKG | 17 | YGNYTMHQYGSWEQMPAFDY | 25 |
| CL1 | TTAIH | 7 | SIASYNGDTDYADSVKG | 18 | YGNYTMHQYGSWEQMPAFDY | 25 |
| CL3 | RTDIH | 8 | SIDSNNGSTYYADSVKG | 19 | YGNYTMHQYGSWEQMPAFDY | 25 |
| CL6 | GSAIH | 9 | SIDSYDGDTDYADSVKG | 20 | YGNYTMHQYGSWEQMPAFDY | 25 |
| CL11 | ATSIH | 10 | EITSYNGSTDYADSVKG | 21 | YGNYTMHQYGSWEQMPAFDY | 25 |
| Genericized HC CDR 3 based on | | | | | YXXYXMXXYGSWXXMXXXDY | 2 |
| serine substitution analysis | | | | | | |
| 7-20 | YSSIH | 11 | SISSYYGSTSYADSVKG | 22 | GKYSWGYYYMDSSMFAMDY | 26 |
| 10-20 | SSSIH | 12 | YISPSSGSTYYADSVKG | 23 | SFYFYAYWSHEWHYAMDY | 27 |
| 25-20 | SSYIH | 13 | SIYPSYGSTYYADSVKG | 24 | QVGVYQYVAPYWGNVWAMDY | 28 |
| 20-20* | YYSIH | 70 | SIYSSYGYTSYADSVKG | 71 | YWSEYWQYYPAMDY | 72 |
| CH15V | GNYIH | 6 | SIYSAGGYTDYADSVKG | 17 | YGVYTMHQYGSWEQMPAFDY | 74 |
| TL1 | GAYIH | 75 | SIYSAGGYTDYADSVKG | 17 | YGTYELKSYGSWESLPAFDY | 77 |
| TL7 | GSYIH | 76 | SIYSAGGYTDYADSVKG | 17 | YGVYTLAAYGSWESLPAFDY | 78 |
| TL16 | GAYIH | 75 | SIYSAGGYTDYADSVKG | 17 | YGTYELKAAGSWEQLPAFDY | 79 |
| TL18 | GSYIH | 76 | SIYSAGGYTDYADSVKG | 17 | YGVYTLHQYGSWEQLPAFDY | 80 |
| TL21 | GNYIH | 6 | SIYSAGGYTDYADSVKG | 17 | YGVYTLASAGSWESLPAFDY | 81 |
| TL22 | GNYIH | 6 | SIYSAGGYTDYADSVKG | 17 | YGVYTLASAGSWESLPAFDY | 81 |
| TL23 | GNYIH | 6 | SIYSAGGYTDYADSVKG | 17 | YGVYTLASAGSWESLPAFDY | 81 |
| TL24 | GNYIH | 6 | SIYSAGGYTDYADSVKG | 17 | YGVYTLASAGSWESLPAFDY | 81 |
| LL2 | GSYIH | 76 | SIYSAGGYTDYADSVKG | 17 | YGVYTLASAGSWESLPAFDY | 81 |
| LL5 | GSYIH | 76 | SIYSAGGYTDYADSVKG | 17 | YGVYTLASAGSWESLPAFDY | 81 |

*20-20 is does not selectively bind mutant HER2

TABLE 2
HER2 Ab Light Chain CDRs

| Clone | CDRL1 | SEQ | CDRL2 | SEQ | CDRL3 | SEQ |
|---|---|---|---|---|---|---|
| 11-1 | RASQSVSSAVA | 29 | SASSLYS | 30 | QQSSSSLIT | 31 |
| CH2 | RASQSVSSAVA | 29 | SASSLYS | 30 | QQSSSSLIT | 31 |
| CH10 | RASQSVSSAVA | 29 | SASSLYS | 30 | QQSSSSLIT | 31 |
| CH15 | RASQSVSSAVA | 29 | SASSLYS | 30 | QQSSSSLIT | 31 |
| CL1 | RASQSVSSAVA | 29 | SASSLYS | 30 | QQSSSSLIT | 31 |
| CL3 | RASQSVSSAVA | 29 | SASSLYS | 30 | QQSSSSLIT | 31 |
| CL6 | RASQSVSSAVA | 29 | SASSLYS | 30 | QQSSSSLIT | 31 |
| CL11 | RASQSVSSAVA | 29 | SASSLYS | 30 | QQSSSSLIT | 31 |
| 7-20 | RASQSVSSAVA | 29 | SASSLYS | 30 | QQYNLRKDLVT | 32 |
| 10-20 | RASQSVSSAVA | 29 | SASSLYS | 30 | QQYWDYSLIT | 33 |
| 25-20 | RASQSVSSAVA | 29 | SASSLYS | 30 | QQSSPGWPVT | 34 |
| 20-20* | RASQSVSSAVA | 29 | SASSLYS | 30 | QQSLNYSGLIT | 73 |
| CH15V | RASQSVSSAVA | 29 | SASSLYS | 30 | QQSSSSLIT | 31 |
| TL1 | RASQSVSSAVA | 29 | SASSLYS | 30 | QQSEWGGLIT | 82 |
| TL7 | RASQSVSSAVA | 29 | SASSLYS | 30 | QQSDYGLIT | 83 |
| TL 16 | RASQSVSSAVA | 29 | SASSLYS | 30 | QQSGYSLIT | 84 |
| TL 18 | RASQSVSSAVA | 29 | SASSLYS | 30 | QQDDYSLIT | 85 |
| TL21 | RASQSVSSAVA | 29 | SASSLYS | 30 | QQSSWDLIT | 86 |
| TL22 | RASQSVSSAVA | 29 | SASSLYS | 30 | QQSSWNGLIT | 87 |
| TL23 | RASQSVSSAVA | 29 | SASSLYS | 30 | QQSGWGGLIT | 88 |
| TL24 | RASQSVSSAVA | 29 | SASSLYS | 30 | QQSSYEDPIT | 89 |
| LL2 | RASQSVSSAVA | 29 | SASSLYS | 30 | QQSSWESPPIT | 90 |
| LL5 | RASQSVSSAVA | 29 | SASSLYS | 30 | QQSSYDGLLT | 91 |

*20-20 is does not selectively bind mutant HER2

In certain embodiments, the residues of heavy chain CDR3 include tyrosine at positions 1, 4, 9 and 20, methionine at positions 6 and 15, glycine at position 10, serine at position 11, tryptophan at position 12, and aspartic acid at position 19. Thus, in these embodiments, the antibody-based molecule may comprise a variable heavy region (V_{H}) comprising a complementarity-determining region 3 (CDR-H3) comprising an amino acid sequence of Y₁X₂X₃Y₄X₅M₆X₇X₈Y₉G₁₀S₁₁W₁₂X₁₃X₁₄M₁₅X₁₆X₁₇X₁₈D₁₉Y₂₀ (SEQ ID NO: 2), wherein X is any amino acid residue.

Based on deep mutational scanning of CH15V, variability of amino acid residues in heavy chain CDR1 and CDR3, as well as adjacent framework region residues, were evaluated for binding specificity to the HER2 S310F mutant. Based on the results reported in the Examples hereinafter, in certain embodiments the heavy chain CDR1 comprises the amino acid sequence of G₁X₂X₃I₄H₅ (SEQ ID NO: 92), where
X₂ can be any of Asn (N), Ala (A), Cys (C), Phe (F), His (H), Leu (L), Met (M), or Ser(S); and
X₃ can be any of Tyr (Y), Glu (E), or Phe (F).
   In certain embodiments, the antibody heavy chain includes a CDR1 containing one or two amino acid changes relative to SEQ ID NO: 6.

In certain embodiments, the FR1 region adjacent to CDR1 includes a pair of residues selected from [Phe (F)/Trp (W)/Tyr (Y)]-[Ser(S)/Ala (A)/Phe (F)/Gly (G)/His (H)/Ile (I)/Met (M)/Asn (N)/Gln (Q)/Arg (R)/Thy (T)/Val (V)/Trp (W)/Tyr (Y)].

In certain embodiments, the heavy chain FR1 region contains the amino acid sequence of EVOLVESGGGLVQPGGSLRLSCAASGFTX₂₉X₃₀ (SEQ ID NO: 95) where
X₂₉ can be any of Phe (F), Trp (W), or Tyr (Y); and
X₃₀ can be any of Ser(S), Ala (A), Phe (F), Gly (G), His (H), Ile (I), Met (M), Asn (N), Gln (Q), Arg (R), Thy (T), Val (V), Trp (W), or Tyr (Y).

In certain embodiments, the antibody-based molecule or binding fragment thereof comprises a heavy chain V_{H} domain that contains an FR1 region according to SEQ ID NO: 95 and an adjacent CDR1 according to SEQ ID NO: 92.

Based on the results reported in the Examples hereinafter, in certain embodiments the heavy chain CDR3 comprises the amino acid sequence of X₁G₂X₃Y₄X₅X₆X₇X₈X₉G₁₀S₁₁W₁₂X₁₃X₁₄X₁₅P₁₆X₁₇X₁₈D₁₉Y₂₀ (SEQ ID NO: 93), where
X₁ can be any of Tyr (Y) or Phe (F);
X₃ can be any of Val (V), Glu (E), His (H), Ile (I), Leu (L), Met (M), Pro (P), Gln (Q), or Thy (T);
X₅ can be any of Thy (T), Ala (A), Glu (E), His (H), Ile (I), Lys (K), Leu (L), Met (M), Asn (N), Gln (Q), Arg (R), or Ser(S);
X₆ can be any of Met (M) or (L);
X₇ can be any of His (H), Ala (A), Lys (K), Met (M), Gln (Q), or Arg (R);
X₈ can be any of Gln (Q), Ala (A), Phe (F), His (H), Ile (I), Lys (K), Leu (L), Met (M), Asn (N), Arg (R), Ser(S), or Thy (T);
X₉ can be any of Tyr (Y), Ala (A), Phe (F), Gly (G), or Arg (R);
X₁₃ can be any of Glu (E), Gln (Q), or (V);
X₁₄ can be any of Gln (Q), Met (M), or Ser(S);
X₁₅ can be any of Met (M), Phe (F), Leu (L), Arg (R), or Thy (T);
X₁₇ can be any of Ala (A) or Tyr (Y); and
X₁₈ can be any of Phe (F), His (H), or Tyr (Y).
   In certain embodiments, the antibody heavy chain includes a CDR3 containing one, two, three, four, five, six, seven, eight, nine, ten, eleven, or twelve amino acid changes relative to SEQ ID NO: 93.

In certain embodiments, the antibody-based molecule or binding fragment thereof comprises a heavy chain V_{H} domain that contains an FR1 region according to SEQ ID NO: 95, an adjacent CDR1 according to SEQ ID NO: 92, and a CDR3 according to SEQ ID NO: 93.

Based on the mutational studies described in the accompanying Examples, variability of amino acid residues in light chain CDR3 has been demonstrated for binding to HER2 mutants. In certain embodiments the light chain CDR3 comprises the amino acid sequence of Q₁Q₂X₃X₄-Z₅-X₆X₇X₈X₉T₁₀ (SEQ ID NO: 94) where
X₃ can be any of Tyr (Y), Ser(S) or Asp (D);
X₄ can be any of Ser(S), Asn (N), Trp (W), Leu (L), Glu (E), Asp (D), Gly (G),
Z₅ can be absent, in which case there is a direct bond between X₄ and X₆, or Z₅ can be a single residue or dipeptide selected from: Trp (W), Tyr (Y), Asp (D), Pro (P), -Trp-Glu-(-WE-), -Leu-Arg-(-LR-), or -Asn-Tyr-(-NY-);
X₆ can be any of Ser(S), Lys (K), Tyr (Y), Gly (G), Trp (W), Asn (N), or Glu (E);
X₇ can be any of Ser(S), Asp (D), Trp (W), Gly (G), Asp (D), or Pro (P);
X₈ can be any of Leu (L) or Pro (P);
X₉ can be any of Ile (I), Leu (L), or Val (V);
   In certain embodiments, the antibody light chain includes a CDR3 containing one, two, three, four, five, six, or seven amino acid changes relative to SEQ ID NO: 31.

In certain embodiments, the antibody-based molecule or binding fragment thereof comprises a heavy chain V_{H} domain that contains an FR1 region according to SEQ ID NO: 95, an adjacent CDR1 according to SEQ ID NO: 92, and/or a CDR3 according to SEQ ID NO: 93; and a light chain V_{L} domain that contains a CDR3 amino acid sequence according to SEQ ID NO: 94.

In any embodiment, the V_{H} of the antibody-based molecule further comprises a complementarity-determining region 1 (CDR-H1) comprising an amino acid sequence of any one of SEQ ID NOs: 3-13, 75, or 76, or a modified amino acid sequence of any one of SEQ ID NOs: 3-13, 75, or 76, said modified sequences having at least 80% sequence identity to any one of SEQ ID NOs: 3-13, 75, or 76; and a complementarity-determining region 2 (CDR-H2) comprising an amino acid sequence of any one of SEQ ID NOs: 14-24, or a modified amino acid sequence of any one of SEQ ID NOs: 14-24, said modified sequences having at least 80% sequence identity to any one of SEQ ID NOs: 14-24.

In any embodiment, antibody-based molecule has a V_{H} selected from the group consisting of: a V_{H} comprising the CDR-H1 of SEQ ID NO: 3, the CDR-H2 of SEQ ID NO: 14, and the CDR-H3 of SEQ ID NO: 2 (11-1); a V_{H} comprising the CDR-H1 of SEQ ID NO: 4, the CDR-H2 of SEQ ID NO: 15, and the CDR-H3 of SEQ ID NO: 2 (CH2); a V_{H} comprising the CDR-H1 of SEQ ID NO: 5, the CDR-H2 of SEQ ID NO: 16, and the CDR-H3 of SEQ ID NO: 2 (CH10); a V_{H} comprising the CDR-H1 of SEQ ID NO: 6, the CDR-H2 of SEQ ID NO: 17, and the CDR-H3 of SEQ ID NO: 2 (CH15); a V_{H} comprising the CDR-H1 of SEQ ID NO: 7, the CDR-H2 of SEQ ID NO: 18, and the CDR-H3 of SEQ ID NO: 2 (CL1); a V_{H} comprising the CDR-H1 of SEQ ID NO: 8, the CDR-H2 of SEQ ID NO: 19, and the CDR-H3 of SEQ ID NO: 2 (CL3); a V_{H} comprising the CDR-H1 of SEQ ID NO: 9, the CDR-H2 of SEQ ID NO: 20, and the CDR-H3 of SEQ ID NO: 2 (CL6); a V_{H} comprising the CDR-H1 of SEQ ID NO: 10, the CDR-H2 of SEQ ID NO: 21, and the CDR-H3 of SEQ ID NO: 2 (CL11); a V_{H} comprising the CDR-H1 of SEQ ID NO: 70, the CDR-H2 of SEQ ID NO: 71, and the CDR-H3 of SEQ ID NO: 93 (CH15V); a V_{H} comprising the CDR-H1 of SEQ ID NO: 75, the CDR-H2 of SEQ ID NO: 17, and the CDR-H3 of SEQ ID NO: 93 (TL1); a V_{H} comprising the CDR-H1 of SEQ ID NO: 76, the CDR-H2 of SEQ ID NO: 17, and the CDR-H3 of SEQ ID NO: 93 (TL7); a V_{H} comprising the CDR-H1 of SEQ ID NO: 75, the CDR-H2 of SEQ ID NO: 17, and the CDR-H3 of SEQ ID NO: 93 (TL16); a V_{H} comprising the CDR-H1 of SEQ ID NO: 76, the CDR-H2 of SEQ ID NO: 17, and the CDR-H3 of SEQ ID NO: 93 (TL18); a V_{H} comprising the CDR-H1 of SEQ ID NO: 6, the CDR-H2 of SEQ ID NO: 17, and the CDR-H3 of SEQ ID NO: 93 (TL21-24); and a V_{H} comprising the CDR-H1 of SEQ ID NO: 76, the CDR-H2 of SEQ ID NO: 17, and the CDR-H3 of SEQ ID NO: 93 (LL2, LL5).

In any embodiment, the antibody-based molecule comprises a V_{H} is selected from the group consisting: a V_{H} comprising the CDR-H1 of SEQ ID NO: 3, the CDR-H2 of SEQ ID NO: 14, and the CDR-H3 of SEQ ID NO: 25 (11-1); a V_{H} comprising the CDR-H1 of SEQ ID NO: 4, the CDR-H2 of SEQ ID NO: 15, and the CDR-H3 of SEQ ID NO: 25 (CH₂); a V_{H} comprising the CDR-H1 of SEQ ID NO: 5, the CDR-H2 of SEQ ID NO: 16, and the CDR-H3 of SEQ ID NO: 25 (CH10); a V_{H} comprising the CDR-H1 of SEQ ID NO: 6, the CDR-H2 of SEQ ID NO: 17, and the CDR-H3 of SEQ ID NO: 25 (CH15); a V_{H} comprising the CDR-H1 of SEQ ID NO: 7, the CDR-H2 of SEQ ID NO: 18, and the CDR-H3 of SEQ ID NO: 25 (CL1); a V_{H} comprising the CDR-H1 of SEQ ID NO: 8, the CDR-H2 of SEQ ID NO: 19, and the CDR-H3 of SEQ ID NO: 25 (CL3); a V_{H} comprising the CDR-H1 of SEQ ID NO: 9, the CDR-H2 of SEQ ID NO: 20, and the CDR-H3 of SEQ ID NO: 25 (CL6); a V_{H} comprising the CDR-H1 of SEQ ID NO: 10, the CDR-H2 of SEQ ID NO: 21, and the CDR-H3 of SEQ ID NO: 25 (CL11); a V_{H} comprising the CDR-H1 of SEQ ID NO: 11, the CDR-H2 of SEQ ID NO: 22, and the CDR-H3 of SEQ ID NO: 26 (7-20); a V_{H} comprising the CDR-H1 of SEQ ID NO: 12, the CDR-H2 of SEQ ID NO: 23, and the CDR-H3 of SEQ ID NO: 27 (10-20); a V_{H} comprising the CDR-H1 of SEQ ID NO: 13, the CDR-H2 of SEQ ID NO: 24, and the CDR-H3 of SEQ ID NO: 28 (25-20); a V_{H} comprising the CDR-H1 of SEQ ID NO: 6, the CDR-H2 of SEQ ID NO: 17, and the CDR-H3 of SEQ ID NO: 74 (CH15V); a V_{H} comprising the CDR-H1 of SEQ ID NO: 75, the CDR-H2 of SEQ ID NO: 17, and the CDR-H3 of SEQ ID NO: 77 (TL1, sTL1); a V_{H} comprising the CDR-H1 of SEQ ID NO: 76, the CDR-H2 of SEQ ID NO: 17, and the CDR-H3 of SEQ ID NO: 78 (TL7); a V_{H} comprising the CDR-H1 of SEQ ID NO: 75, the CDR-H2 of SEQ ID NO: 17, and the CDR-H3 of SEQ ID NO: 79 (TL16); a Vu comprising the CDR-H1 of SEQ ID NO: 76, the CDR-H2 of SEQ ID NO: 17, and the CDR-H3 of SEQ ID NO: 80 (TL18, sTL18); a V_{H} comprising the CDR-H1 of SEQ ID NO: 6, the CDR-H2 of SEQ ID NO: 17, and the CDR-H3 of SEQ ID NO: 81 (TL21-24); and a V_{H} comprising the CDR-H1 of SEQ ID NO: 76, the CDR-H2 of SEQ ID NO: 17, and the CDR-H3 of SEQ ID NO: 78 (LL2, sLL2, LL5).

The antibody-based molecule described herein can further comprises a variable light region (V_{L}), wherein said V_{L} comprises: a complementarity-determining region 1 (CDR-L1) having an amino acid sequence of SEQ ID NO: 29 or a modified amino acid sequence of SEQ ID NO: 29, said modified sequence having at least 80% sequence identity to SEQ ID NO: 29; a complementarity-determining region 2 (CDR-L2) having an amino acid sequence of SEQ ID NO: 30 or a modified amino acid sequence of SEQ ID NO: 30, said modified sequence having at least 80% sequence identity to SEQ ID NO: 30; and a complementarity-determining region 1 (CDR-L1) having an amino acid sequence of any one of SEQ ID NOs: 31-34 or 82-91, or a modified amino acid sequence of any one of SEQ ID NOs: 31-34 or 82-91, said modified sequence having at least 80% sequence identity to SEQ ID NOs: 31-34 or 82-91.

In any embodiment, the antibody-based molecule comprises a V_{L} comprising the CDR-L1 of SEQ ID NO: 29, the CDR-L2 of SEQ ID NO: 30, and the CDR-L3 of SEQ ID NO: 94.

In any embodiment, the antibody-based molecule comprises a V_{L} is selected from the group consisting of: a V_{L} comprising the CDR-L1 of SEQ ID NO: 29, the CDR-L2 of SEQ ID NO: 30, and the CDR-L3 of SEQ ID NO: 31; a V_{L} comprising the CDR-L1 of SEQ ID NO: 29, the CDR-L2 of SEQ ID NO: 30, and the CDR-L3 of SEQ ID NO: 32; a V_{L} comprising the CDR-L1 of SEQ ID NO: 29, the CDR-L2 of SEQ ID NO: 30, and the CDR-L3 of SEQ ID NO: 33; a V_{L} comprising the CDR-L1 of SEQ ID NO: 29, the CDR-L2 of SEQ ID NO: 30, and the CDR-L3 of SEQ ID NO: 34; a V_{L} comprising the CDR-L1 of SEQ ID NO: 29, the CDR-L2 of SEQ ID NO: 30, and the CDR-L3 of SEQ ID NO: 82; a V_{L} comprising the CDR-L1 of SEQ ID NO: 29, the CDR-L2 of SEQ ID NO: 30, and the CDR-L3 of SEQ ID NO: 83; a V_{L} comprising the CDR-L1 of SEQ ID NO: 29, the CDR-L2 of SEQ ID NO: 30, and the CDR-L3 of SEQ ID NO: 84; a V_{L} comprising the CDR-L1 of SEQ ID NO: 29, the CDR-L2 of SEQ ID NO: 30, and the CDR-L3 of SEQ ID NO: 85; a V_{L} comprising the CDR-L1 of SEQ ID NO: 29, the CDR-L2 of SEQ ID NO: 30, and the CDR-L3 of SEQ ID NO: 86; a V_{L} comprising the CDR-L1 of SEQ ID NO: 29, the CDR-L2 of SEQ ID NO: 30, and the CDR-L3 of SEQ ID NO: 87; a V_{L} comprising the CDR-L1 of SEQ ID NO: 29, the CDR-L2 of SEQ ID NO: 30, and the CDR-L3 of SEQ ID NO: 88; a V_{L} comprising the CDR-L1 of SEQ ID NO: 29, the CDR-L2 of SEQ ID NO: 30, and the CDR-L3 of SEQ ID NO: 89; a V_{L} comprising the CDR-L1 of SEQ ID NO: 29, the CDR-L2 of SEQ ID NO: 30, and the CDR-L3 of SEQ ID NO: 90; and a V_{L} comprising the CDR-L1 of SEQ ID NO: 29, the CDR-L2 of SEQ ID NO: 30, and the CDR-L3 of SEQ ID NO: 91.

In any embodiment, the antibody-based molecule or binding fragment thereof of the present disclosure comprises:
a V_{H} comprising the CDR-H1 of SEQ ID NO: 3, the CDR-H2 of SEQ ID NO: 14, and the CDR-H3 of SEQ ID NO: 2, and a V_{L} comprising the CDR-L1 of SEQ ID NO: 29, the CDR-L2 of SEQ ID NO: 30, and the CDR-L3 of SEQ ID NO: 31 (11-1);
a V_{H} comprising the CDR-H1 of SEQ ID NO: 4, the CDR-H2 of SEQ ID NO: 15, and the CDR-H3 of SEQ ID NO: 2, and a V_{L} comprising the CDR-L1 of SEQ ID NO: 29, the CDR-L2 of SEQ ID NO: 30, and the CDR-L3 of SEQ ID NO: 31 (CH2);
a V_{H} comprising the CDR-H1 of SEQ ID NO: 5, the CDR-H2 of SEQ ID NO: 16, and the CDR-H3 of SEQ ID NO: 2, and a V_{L} comprising the CDR-L1 of SEQ ID NO: 29, the CDR-L2 of SEQ ID NO: 30, and the CDR-L3 of SEQ ID NO: 31 (CH10);
a V_{H} comprising the CDR-H1 of SEQ ID NO: 6, the CDR-H2 of SEQ ID NO: 17, and the CDR-H3 of SEQ ID NO: 2, and a V_{L} comprising the CDR-L1 of SEQ ID NO: 29, the CDR-L2 of SEQ ID NO: 30, and the CDR-L3 of SEQ ID NO: 31 (CH15);
a V_{H} comprising the CDR-H1 of SEQ ID NO: 7, the CDR-H2 of SEQ ID NO: 18, and the CDR-H3 of SEQ ID NO: 2, and a V_{L} comprising the CDR-L1 of SEQ ID NO: 29, the CDR-L2 of SEQ ID NO: 30, and the CDR-L3 of SEQ ID NO: 31 (CL1);
a V_{H} comprising the CDR-H1 of SEQ ID NO: 8, the CDR-H2 of SEQ ID NO: 19, and the CDR-H3 of SEQ ID NO: 2, and a V_{L} comprising the CDR-L1 of SEQ ID NO: 29, the CDR-L2 of SEQ ID NO: 30, and the CDR-L3 of SEQ ID NO: 31 (CL3);
a V_{H} comprising the CDR-H1 of SEQ ID NO: 9, the CDR-H2 of SEQ ID NO: 20, and the CDR-H3 of SEQ ID NO: 2, and a V_{L} comprising the CDR-L1 of SEQ ID NO: 29, the CDR-L2 of SEQ ID NO: 30, and the CDR-L3 of SEQ ID NO: 31 (CL6);
a V_{H} comprising the CDR-H1 of SEQ ID NO: 10, the CDR-H2 of SEQ ID NO: 21, and the CDR-H3 of SEQ ID NO: 2, and a V_{L} comprising the CDR-L1 of SEQ ID NO: 29, the CDR-L2 of SEQ ID NO: 30, and the CDR-L3 of SEQ ID NO: 31 (CL11).

In any embodiment, the antibody-based molecule or binding fragment thereof comprises:
a V_{H} comprising the CDR-H1 of SEQ ID NO: 3, the CDR-H2 of SEQ ID NO: 14, and the CDR-H3 of SEQ ID NO: 25, and a V_{L} comprising the CDR-L1 of SEQ ID NO: 29, the CDR-L2 of SEQ ID NO: 30, and the CDR-L3 of SEQ ID NO: 31 (11-1);
a V_{H} comprising the CDR-H1 of SEQ ID NO: 4, the CDR-H2 of SEQ ID NO: 15, and the CDR-H3 of SEQ ID NO: 25, and a V_{L} comprising the CDR-L1 of SEQ ID NO: 29, the CDR-L2 of SEQ ID NO: 30, and the CDR-L3 of SEQ ID NO: 31 (CH2);
a V_{H} comprising the CDR-H1 of SEQ ID NO: 5, the CDR-H2 of SEQ ID NO: 16, and the CDR-H3 of SEQ ID NO: 25, and a V_{L} comprising the CDR-L1 of SEQ ID NO: 29, the CDR-L2 of SEQ ID NO: 30, and the CDR-L3 of SEQ ID NO: 31 (CH10);
a V_{H} comprising the CDR-H1 of SEQ ID NO: 6, the CDR-H2 of SEQ ID NO: 17, and the CDR-H3 of SEQ ID NO: 25, and a V_{L} comprising the CDR-L1 of SEQ ID NO: 29, the CDR-L2 of SEQ ID NO: 30, and the CDR-L3 of SEQ ID NO: 31 (CH15);
a V_{H} comprising the CDR-H1 of SEQ ID NO: 7, the CDR-H2 of SEQ ID NO: 18, and the CDR-H3 of SEQ ID NO: 25, and a V_{L} comprising the CDR-L1 of SEQ ID NO: 29, the CDR-L2 of SEQ ID NO: 30, and the CDR-L3 of SEQ ID NO: 31 (CL1);
a V_{H} comprising the CDR-H1 of SEQ ID NO: 8, the CDR-H2 of SEQ ID NO: 19, and the CDR-H3 of SEQ ID NO: 25, and a V_{L} comprising the CDR-L1 of SEQ ID NO: 29, the CDR-L2 of SEQ ID NO: 30, and the CDR-L3 of SEQ ID NO: 31 (CL3);
a V_{H} comprising the CDR-H1 of SEQ ID NO: 9, the CDR-H2 of SEQ ID NO: 20, and the CDR-H3 of SEQ ID NO: 25, and a V_{L} comprising the CDR-L1 of SEQ ID NO: 29, the CDR-L2 of SEQ ID NO: 30, and the CDR-L3 of SEQ ID NO: 31 (CL6);
a V_{H} comprising the CDR-H1 of SEQ ID NO: 10, the CDR-H2 of SEQ ID NO: 21, and the CDR-H3 of SEQ ID NO: 25, and a V_{L} comprising the CDR-L1 of SEQ ID NO: 29, the CDR-L2 of SEQ ID NO: 30, and the CDR-L3 of SEQ ID NO: 31 (CL11)
a V_{H} comprising the CDR-H1 of SEQ ID NO: 11, the CDR-H2 of SEQ ID NO: 22, and the CDR-H3 of SEQ ID NO: 26, and a V_{L} comprising the CDR-L1 of SEQ ID NO: 29, the CDR-L2 of SEQ ID NO: 30, and the CDR-L3 of SEQ ID NO: 32 (7-20)
a V_{H} comprising the CDR-H1 of SEQ ID NO: 12, the CDR-H2 of SEQ ID NO: 23, and the CDR-H3 of SEQ ID NO: 27, and a V_{L} comprising the CDR-L1 of SEQ ID NO: 29, the CDR-L2 of SEQ ID NO: 30, and the CDR-L3 of SEQ ID NO: 33 (10-20);
a V_{H} comprising the CDR-H1 of SEQ ID NO: 13, the CDR-H2 of SEQ ID NO: 24, and the CDR-H3 of SEQ ID NO: 28, and a V_{L} comprising the CDR-L1 of SEQ ID NO: 29, the CDR-L2 of SEQ ID NO: 30, and the CDR-L3 of SEQ ID NO: 34 (25-20);
a V_{H} comprising the CDR-H1 of SEQ ID NO: 6, the CDR-H2 of SEQ ID NO: 17, and the CDR-H3 of SEQ ID NO: 74, and a V_{L} comprising the CDR-L1 of SEQ ID NO: 29, the CDR-L2 of SEQ ID NO: 30, and the CDR-L3 of SEQ ID NO: 31 (CH15V);
a V_{H} comprising the CDR-H1 of SEQ ID NO: 75, the CDR-H2 of SEQ ID NO: 17, and the CDR-H3 of SEQ ID NO: 77, and a V_{L} comprising the CDR-L1 of SEQ ID NO: 29, the CDR-L2 of SEQ ID NO: 30, and the CDR-L3 of SEQ ID NO: 82 (TL1);
a V_{H} comprising the CDR-H1 of SEQ ID NO: 76, the CDR-H2 of SEQ ID NO: 17, and the CDR-H3 of SEQ ID NO: 78, and a V_{L} comprising the CDR-L1 of SEQ ID NO: 29, the CDR-L2 of SEQ ID NO: 30, and the CDR-L3 of SEQ ID NO: 83 (TL7);
a V_{H} comprising the CDR-H1 of SEQ ID NO: 75, the CDR-H2 of SEQ ID NO: 17, and the CDR-H3 of SEQ ID NO: 79, and a V_{L} comprising the CDR-L1 of SEQ ID NO: 29, the CDR-L2 of SEQ ID NO: 30, and the CDR-L3 of SEQ ID NO: 84 (TL16);
a V_{H} comprising the CDR-H1 of SEQ ID NO: 76, the CDR-H2 of SEQ ID NO: 17, and the CDR-H3 of SEQ ID NO: 80 and a V_{L} comprising the CDR-L1 of SEQ ID NO: 29, the CDR-L2 of SEQ ID NO: 30, and the CDR-L3 of SEQ ID NO: 85 (TL18);
a V_{H} comprising the CDR-H1 of SEQ ID NO: 6, the CDR-H2 of SEQ ID NO: 17, and the CDR-H3 of SEQ ID NO: 81, and a V_{L} comprising the CDR-L1 of SEQ ID NO: 29, the CDR-L2 of SEQ ID NO: 30, and the CDR-L3 of SEQ ID NO: 86 (TL21);
a V_{H} comprising the CDR-H1 of SEQ ID NO: 6, the CDR-H2 of SEQ ID NO: 17, and the CDR-H3 of SEQ ID NO: 81, and a V_{L} comprising the CDR-L1 of SEQ ID NO: 29, the CDR-L2 of SEQ ID NO: 30, and the CDR-L3 of SEQ ID NO: 87 (TL22);
a V_{H} comprising the CDR-H1 of SEQ ID NO: 6, the CDR-H2 of SEQ ID NO: 17, and the CDR-H3 of SEQ ID NO: 81, and a V_{L} comprising the CDR-L1 of SEQ ID NO: 29, the CDR-L2 of SEQ ID NO: 30, and the CDR-L3 of SEQ ID NO: 88 (TL23);
a V_{H} comprising the CDR-H1 of SEQ ID NO: 6, the CDR-H2 of SEQ ID NO: 17, and the CDR-H3 of SEQ ID NO: 81, and a V_{L} comprising the CDR-L1 of SEQ ID NO: 29, the CDR-L2 of SEQ ID NO: 30, and the CDR-L3 of SEQ ID NO: 89 (TL24);
a V_{H} comprising the CDR-H1 of SEQ ID NO: 76, the CDR-H2 of SEQ ID NO: 17, and the CDR-H3 of SEQ ID NO: 78, and a V_{L} comprising the CDR-L1 of SEQ ID NO: 29, the CDR-L2 of SEQ ID NO: 30, and the CDR-L3 of SEQ ID NO: 90 (LL2); and
a V_{H} comprising the CDR-H1 of SEQ ID NO: 76, the CDR-H2 of SEQ ID NO: 17, and the CDR-H3 of SEQ ID NO: 78, and a V_{L} comprising the CDR-L1 of SEQ ID NO: 29, the CDR-L2 of SEQ ID NO: 30, and the CDR-L3 of SEQ ID NO: 91 (LL25);
a V_{H} comprising the CDR-H1 of SEQ ID NO: 75, the CDR-H2 of SEQ ID NO: 17, and the CDR-H3 of SEQ ID NO: 77, and a V_{L} comprising the CDR-L1 of SEQ ID NO: 29, the CDR-L2 of SEQ ID NO: 30, and the CDR-L3 of SEQ ID NO: 31 (sTL1);
a V_{H} comprising the CDR-H1 of SEQ ID NO: 76, the CDR-H2 of SEQ ID NO: 17, and the CDR-H3 of SEQ ID NO: 80 and a V_{L} comprising the CDR-L1 of SEQ ID NO: 29, the CDR-L2 of SEQ ID NO: 30, and the CDR-L3 of SEQ ID NO: 31 (sTL18); and
a V_{H} comprising the CDR-H1 of SEQ ID NO: 76, the CDR-H2 of SEQ ID NO: 17, and the CDR-H3 of SEQ ID NO: 78, and a V_{L} comprising the CDR-L1 of SEQ ID NO: 29, the CDR-L2 of SEQ ID NO: 30, and the CDR-L3 of SEQ ID NO: 31 (sLL2).

Exemplary amino acid sequences of the variable heavy and variable light regions of the HER2 antibodies described herein are provided in Tables 3 below. In any embodiment, the HER2 antibody-based molecule comprises a V_{H} and/or V_{L} sequence having least 80% identity to the V_{H} and V_{L} sequences provided in Table 3 below.

TABLE 3
HER2 Ab Variable Region Sequences

| | | | SEQ ID |
|---|---|---|---|
| Clone | Region | Sequence | NO |
| 11-1 | V_{H(consensus)} | EVQLVESGGGLVQPGGSLRLSCAASGFTVYSSSIHWVRQAPGKGLEW | 35 |
| | | VASISSYYGYTSYADSVKGRFTISADTSKNTAYLQMNSLRAEDTAVY | |
| | | YCARYXXYXMXXYGSWXXMXXXDYWGQGTLVTVSS | |
| | V_{H} | EVQLVESGGGLVQPGGSLRLSCAASGFTVYSSSIHWVRQAPGKGLEW | 36 |
| | | VASISSYYGYTSYADSVKGRFTISADTSKNTAYLQMNSLRAEDTAVY | |
| | | YCARYGNYTMHQYGSWEQMPAFDYWGQGTLVTVSS | |
| | V_{L} | DIQMTQSPSSLSASVGDRVTITCRASQSVSSAVAWYQQKPGKAPKLL | 37 |
| | | IYSASSLYSGVPSRFSGSRSGTDFTLTISSLQPEDFATYYCQQSSSS | |
| | | LITFGQGTKVEIKRTV | |
| CH2 | V_{H(consensus)} | EVQLVESGGGLVQPGGSLRLSCAASGFTFSRTSIHWVRQAPGKGLEW | 38 |
| | | VAEISSYDGYTDYADSVKGRFTISADTSKNTAYLQMNSLRAEDTAVY | |
| | | YCARYXXYXMXXYGSWXXMXXXDYWGQGTLVTVSS | |
| | V_{H} | EVQLVESGGGLVQPGGSLRLSCAASGFTFSRTSIHWVRQAPGKGLEW | 39 |
| | | VAEISSYDGYTDYADSVKGRFTISADTSKNTAYLQMNSLRAEDTAVY | |
| | | YCARYGNYTMHQYGSWEQMPAFDYWGQGTLVTVSS | |
| | V_{L} | DIQMTQSPSSLSASVGDRVTITCRASQSVSSAVAWYQQKPGKAPKLL | 40 |
| | | IYSASSLYSGVPSRFSGSRSGTDFTLTISSLQPEDFATYYCQQSSSS | |
| | | LITFGQGTKVEIKRTV | |
| CH10 | V_{H(consensus)} | EVQLVESGGGLVQPGGSLRLSCAASGFTFSTTSIHWVRQAPGKGLEW | 41 |
| | | VAEISSYNGDTDYADSVKGRFTISADTSKNTAYLQMNSLRAEDTAVY | |
| | | YCARYXXYXMXXYGSWXXMXXXDYWGQGTLVTVSS | |
| | V_{H} | EVQLVESGGGLVQPGGSLRLSCAASGFTFSTTSIHWVRQAPGKGLEW | 42 |
| | | VAEISSYNGDTDYADSVKGRFTISADTSKNTAYLQMNSLRAEDTAVY | |
| | | YCARYGNYTMHQYGSWEQMPAFDYWGQGTLVTVSS | |
| | V_{L} | DIQMTQSPSSLSASVGDRVTITCRASQSVSSAVAWYQQKPGKAPKLL | 43 |
| | | IYSASSLYSGVPSRFSGSRSGTDFTLTISSLQPEDFATYYCQQSSSS | |
| | | LITFGQGTKVEIKRTV | |
| CH15 | V_{H(consensus)} | EVQLVESGGGLVQPGGSLRLSCAASGFTFSGNYIHWVRQAPGKGLEW | 44 |
| | | VASIYSAGGYTDYADSVKGRFTISADTSKNTAYLQMNSLRAEDTAVY | |
| | | YCARYXXYXMXXYGSWXXMXXXDYWGQGTLVTVSS | |
| | V_{H} | EVQLVESGGGLVQPGGSLRLSCAASGFTFSGNYIHWVRQAPGKGLEW | 45 |
| | | VASIYSAGGYTDYADSVKGRFTISADTSKNTAYLQMNSLRAEDTAVY | |
| | | YCARYGNYTMHQYGSWEQMPAFDYWGQGTLVTVSS | |
| | V_{L} | DIQMTQSPSSLSASVGDRVTITCRASQSVSSAVAWYQQKPGKAPKLL | 46 |
| | | IYSASSLYSGVPSRFSGSRSGTDFTLTISSLQPEDFATYYCQQSSSS | |
| | | LITFGQGTKVEIKRTV | |
| CH15 | HC | EVQLVESGGGLVQPGGSLRLSCAASGFTFSGNYIHWVRQAPGKGLEW | 142 |
| | | VASIYSAGGYTDYADSVKGRFTISADTSKNTAYLQMNSLRAEDTAVY | |
| | | YCARYGNYTMHQYGSWEQMPAFDYWGQGTLVTVSSASTKGPSVFPLA | |
| | | PSSKSTSGGTAALGCLVKDYFPEPVTVSWNSGALTSGVHTFPAVLQS | |
| | | SGLYSLSSVVTVPSSSLGTQTYICNVNHKPSNTKVDKKVEPKSCDKT | |
| | | HTCPPCPAPELLGGPSVFLFPPKPKDTLMISRTPEVTCVVVDVSHED | |
| | | PEVKFNWYVDGVEVHNAKTKPREEQYNSTYRVVSVLTVLHQDWLNGK | |
| | | EYKCKVSNKALPAPIEKTISKAKGQPREPQVYTLPPSREEMTKNQVS | |
| | | LTCLVKGFYPSDIAVEWESNGQPENNYKTTPPVLDSDGSFFLYSKLT | |
| | | VDKSRWQQGNVFSCSVMHEALHNHYTQKSLSLSPGK | |
| | LC | DIQMTQSPSSLSASVGDRVTITCRASQSVSSAVAWYQQKPGKAPKLL | 143 |
| | | IYSASSLYSGVPSRFSGSRSGTDFTLTISSLQPEDFATYYCQQSSSS | |
| | | LITFGQGTKVEIKRTVAAPSVFIFPPSDEQLKSGTASVVCLLNNFYP | |
| | | REAKVQWKVDNALQSGNSQESVTEQDSKDSTYSLSSTLTLSKADYEK | |
| | | HKVYACEVTHQGLSSPVTKSENRGEC | |
| CL1 | V_{H(consensus)} | EVQLVESGGGLVQPGGSLRLSCAASGFTFSTTAIHWVRQAPGKGLEW | 47 |
| | | VASIASYNGDTDYADSVKGRFTISADTSKNTAYLQMNSLRAEDTAVY | |
| | | YCARYXXYXMXXYGSWXXMXXXDYWGQGTLVTVSS | |
| | V_{H} | EVQLVESGGGLVQPGGSLRLSCAASGFTFSTTAIHWVRQAPGKGLEW | 48 |
| | | VASIASYNGDTDYADSVKGRFTISADTSKNTAYLQMNSLRAEDTAVY | |
| | | YCARYGNYTMHQYGSWEQMPAFDYWGQGTLVTVSS | |
| | V_{L} | DIQMTQSPSSLSASVGDRVTITCRASQSVSSAVAWYQQKPGKAPKLL | 49 |
| | | IYSASSLYSGVPSRFSGSRSGTDFTLTISSLQPEDFATYYCQQSSSS | |
| | | LITFGQGTKVEIKRTV | |
| CL3 | V_{H(consensus)} | EVQLVESGGGLVQPGGSLRLSCAASGFTFTRTDIHWVRQAPGKGLEW | 50 |
| | | VASIDSNNGSTYYADSVKGRFTISADTSKNTAYLQMNSLRAEDTAVY | |
| | | YCARYXXYXMXXYGSWXXMXXXDYWGQGTLVTVSS | |
| | V_{H} | EVQLVESGGGLVQPGGSLRLSCAASGFTFTRTDIHWVRQAPGKGLEW | 51 |
| | | VASIDSNNGSTYYADSVKGRFTISADTSKNTAYLQMNSLRAEDTAVY | |
| | | YCARYGNYTMHQYGSWEQMPAFDYWGQGTLVTVSS | |
| | V_{L} | DIQMTQSPSSLSASVGDRVTITCRASQSVSSAVAWYQQKPGKAPKLL | 52 |
| | | IYSASSLYSGVPSRFSGSRSGTDFTLTISSLQPEDFATYYCQQSSSS | |
| | | LITFGQGTKVEIKRTV | |
| CL6 | V_{H(consensus)} | EVQLVESGGGLVQPGGSLRLSCAASGFTFTGSAIHWVRQAPGKGLEW | 53 |
| | | VASIDSYDGDTDYADSVKGRFTISADTSKNTAYLQMNSLRAEDTAVY | |
| | | YCARYXXYXMXXYGSWXXMXXXDYWGQGTLVTVSS | |
| | V_{H} | EVQLVESGGGLVQPGGSLRLSCAASGFTFTGSAIHWVRQAPGKGLEW | 54 |
| | | VASIDSYDGDTDYADSVKGRFTISADTSKNTAYLQMNSLRAEDTAVY | |
| | | YCARYGNYTMHQYGSWEQMPAFDYWGQGTLVTVSS | |
| | V_{L} | DIQMTQSPSSLSASVGDRVTITCRASQSVSSAVAWYQQKPGKAPKLL | 55 |
| | | IYSASSLYSGVPSRFSGSRSGTDFTLTISSLQPEDFATYYCQQSSSS | |
| | | LITFGQGTKVEIKRTV | |
| CL11 | V_{H(consensus)} | EVQLVESGGGLVQPGGSLRLSCAASGFTFSATSIHWVRQAPGKGLEW | 56 |
| | | VAEITSYNGSTDYADSVKGRFTISADTSKNTAYLQMNSLRAEDTAVY | |
| | | YCARYXXYXMXXYGSWXXMXXXDYWGQGTLVTVSS | |
| | V_{H} | EVQLVESGGGLVQPGGSLRLSCAASGFTFSATSIHWVRQAPGKGLEW | 57 |
| | | VAEITSYNGSTDYADSVKGRFTISADTSKNTAYLQMNSLRAEDTAVY | |
| | | YCARYGNYTMHQYGSWEQMPAFDYWGQGTLVTVSS | |
| | V_{L} | DIQMTQSPSSLSASVGDRVTITCRASQSVSSAVAWYQQKPGKAPKLL | 58 |
| | | IYSASSLYSGVPSRFSGSRSGTDFTLTISSLQPEDFATYYCQQSSSS | |
| | | LITFGQGTKVEIKRTV | |
| 7-20 | V_{H} | EVQLVESGGGLVQPGGSLRLSCAASGFTISYSSIHWVRQAPGKGLEW | 59 |
| | | VASISSYYGSTSYADSVKGRFTISADTSKNTAYLQMNSLRAEDTAVY | |
| | | YCARGKYSWGYYYMDSSMFAMDYWGQGTLVTVSS | |
| | V_{L} | DIQMTQSPSSLSASVGDRVTITCRASQSVSSAVAWYQQKPGKAPKLL | 60 |
| | | IYSASSLYSGVPSRFSGSRSGTDFTLTISSLQPEDFATYYCQQYNLR | |
| | | KDLVTFGQGTKVEIKRTV | |
| 10-20 | V_{H} | EVQLVESGGGLVQPGGSLRLSCAASGFTVSSSSIHWVRQAPGKGLEW | 61 |
| | | VAYISPSSGSTYYADSVKGRFTISADTSKNTAYLQMNSLRAEDTAVY | |
| | | YCARSFYFYAYWSHEWHYAMDYWGQGTLVTVSS | |
| | V_{L} | DIQMTQSPSSLSASVGDRVTITCRASQSVSSAVAWYQQKPGKAPKLL | 62 |
| | | IYSASSLYSGVPSRFSGSRSGTDFTLTISSLQPEDFATYYCQQYWDY | |
| | | SLITFGQGTKVEIKRTV | |
| 25-20 | V_{H} | EVQLVESGGGLVQPGGSLRLSCAASGFTLSSSYIHWVRQAPGKGLEW | 63 |
| (2) | | VASIYPSYGSTYYADSVKGRFTISADTSKNTAYLQMNSLRAEDTAVY | |
| | | YCARQVGVYQYVAPYWGNVWAMDYWGQGTLVTVSS | |
| | V_{L} | DIQMTQSPSSLSASVGDRVTITCRASQSVSSAVAWYQQKPGKAPKLL | 64 |
| | | IYSASSLYSGVPSRFSGSRSGTDFTLTISSLQPEDFATYYCQQSSPG | |
| | | WPVTFGQGTKVEIKRTV | |
| 20-20 | V_{H} | EVQLVESGGGLVQPGGSLRLSCAASGFTLSYYSIHWVRQAPGKGLEW | 65 |
| | | VASIYSSYGYTSYADSVKGRFTISADTSKNTAYLQMNSLRAEDTAVY | |
| | | YCARYWSEYWQYYPAMDYWGQGTLVTVSS | |
| | V_{L} | DIQMTQSPSSLSASVGDRVTITCRASQSVSSAVAWYQQKPGKAPKLL | 66 |
| | | IYSASSLYSGVPSRFSGSRSGTDFTLTISSLQPEDFATYYCQQSLNY | |
| | | SGLITFGQGTKVEIKRTV | |
| CH15V | V_{H(consensus)} | EVQLVESGGGLVQPGGSLRLSCAASGFTXXGXXIHWVRQAPGKGLEW | 96 |
| | | VASIYSAGGYTDYADSVKGRFTISADTSKNTAYLQMNSLRAEDTAVY | |
| | | YCARXGXYXXXXXGSWXXXPXXDYWGQGTLVTVSS | |
| | V_{H} | EVQLVESGGGLVQPGGSLRLSCAASGFTFSGNYIHWVRQAPGKGLEW | 97 |
| | | VASIYSAGGYTDYADSVKGRFTISADTSKNTAYLQMNSLRAEDTAVY | |
| | | YCARYGVYTMHQYGSWEQMPAFDYWGQGTLVTVSS | |
| | V_{L(consensus)} | DIQMTQSPSSLSASVGDRVTITCRASQSVSSAVAWYQQKPGKAPKLL | 98 |
| | | IYSASSLYSGVPSRFSGSRSGTDFTLTISSLQPEDFATYYCQQSSSS | |
| | | LITFGQGTKVEIKRTV | |
| | V_{L} | DIQMTQSPSSLSASVGDRVTITCRASQSVSSAVAWYQQKPGKAPKLL | 99 |
| | | IYSASSLYSGVPSRFSGSRSGTDFTLTISSLQPEDFATYYCQQSSSS | |
| | | LITFGQGTKVEIKRTV | |
| TL1 | V_{H(consensus)} | EVQLVESGGGLVQPGGSLRLSCAASGFTXXGXXIHWVRQAPGKGLEW | 100 |
| | | VASIYSAGGYTDYADSVKGRFTISADTSKNTAYLQMNSLRAEDTAVY | |
| | | YCARXGXYXXXXXGSWXXXPXXDYWGQGTLVTV | |
| | V_{H} | EVQLVESGGGLVQPGGSLRLSCAASGFTWSGAYIHWVRQAPGKGLEW | 101 |
| | | VASIYSAGGYTDYADSVKGRFTISADTSKNTAYLQMNSLRAEDTAVY | |
| | | YCARYGTYELKSYGSWESLPAFDYWGQGTLVTV | |
| | V_{L(consensus)} | DIQMTQSPSSLSASVGDRVTITCRASQSVSSAVAWYQQKPGKAPKLL | 102 |
| | | IYSASSLYSGVPSRFSGSRSGTDFTLTISSLQPEDFATYYCQQSEWG | |
| | | GLITFGQGTKVEIKRTV | |
| | V_{L} | DIQMTQSPSSLSASVGDRVTITCRASQSVSSAVAWYQQKPGKAPKLL | 103 |
| | | IYSASSLYSGVPSRFSGSRSGTDFTLTISSLQPEDFATYYCQQXXZX | |
| | | XXXTFGQGTKVEIKRTV | |
| TL7 | V_{H(consensus)} | EVQLVESGGGLVQPGGSLRLSCAASGFTXXGXXIHWVRQAPGKGLEW | 104 |
| | | VASIYSAGGYTDYADSVKGRFTISADTSKNTAYLQMNSLRAEDTAVY | |
| | | YCARXGXYXXXXXGSWXXXPXXDYWGQGTLVTV | |
| | V_{H} | EVQLVESGGGLVQPGGSLRLSCAASGFTWGGSYIHWVRQAPGKGLEW | 105 |
| | | VASIYSAGGYTDYADSVKGRFTISADTSKNTAYLQMNSLRAEDTAVY | |
| | | YCARYGVYTLAAYGSWESLPAFDYWGQGTLVTV | |
| | V_{L(consensus)} | DIQMTQSPSSLSASVGDRVTITCRASQSVSSAVAWYQQKPGKAPKLL | 106 |
| | | IYSASSLYSGVPSRFSGSRSGTDFTLTISSLQPEDFATYYCQQXXZX | |
| | | XXXTFGQGTKVEIKRTV | |
| | V_{L} | DIQMTQSPSSLSASVGDRVTITCRASQSVSSAVAWYQQKPGKAPKLL | 107 |
| | | IYSASSLYSGVPSRFSGSRSGTDFTLTISSLQPEDFATYYCQQSDYG | |
| | | LITFGQGTKVEIKRTV | |
| TL16 | V_{H(consensus)} | EVQLVESGGGLVQPGGSLRLSCAASGFTXXGXXIHWVRQAPGKGLEW | 108 |
| | | VASIYSAGGYTDYADSVKGRFTISADTSKNTAYLQMNSLRAEDTAVY | |
| | | YCARXGXYXXXXXGSWXXXPXXDYWGQGTLVTV | |
| | V_{H} | EVQLVESGGGLVQPGGSLRLSCAASGFTFGGAYIHWVRQAPGKGLEW | 109 |
| | | VASIYSAGGYTDYADSVKGRFTISADTSKNTAYLQMNSLRAEDTAVY | |
| | | YCARYGTYELKAAGSWEQLPAFDYWGQGTLVTV | |
| | V_{L(consensus)} | DIQMTQSPSSLSASVGDRVTITCRASQSVSSAVAWYQQKPGKAPKLL | 110 |
| | | IYSASSLYSGVPSRFSGSRSGTDFTLTISSLQPEDFATYYCQQXXZX | |
| | | XXXTFGQGTKVEIKRTV | |
| | V_{L} | DIQMTQSPSSLSASVGDRVTITCRASQSVSSAVAWYQQKPGKAPKLL | 111 |
| | | IYSASSLYSGVPSRFSGSRSGTDFTLTISSLQPEDFATYYCQQSGYS | |
| | | LITFGQGTKVEIKRTV | |
| TL18 | V_{H(consensus)} | EVQLVESGGGLVQPGGSLRLSCAASGFTXXGXXIHWVRQAPGKGLEW | 112 |
| | | VASIYSAGGYTDYADSVKGRFTISADTSKNTAYLQMNSLRAEDTAVY | |
| | | YCARXGXYXXXXXGSWXXXPXXDYWGQGTLVTV | |
| | V_{H} | EVQLVESGGGLVQPGGSLRLSCAASGFTFGGSYIHWVRQAPGKGLEW | 113 |
| | | VASIYSAGGYTDYADSVKGRFTISADTSKNTAYLQMNSLRAEDTAVY | |
| | | YCARYGVYTLHQYGSWEQLPAFDYWGQGTLVTV | |
| | _{VL(consensus)} | DIQMTQSPSSLSASVGDRVTITCRASQSVSSAVAWYQQKPGKAPKLL | 114 |
| | | IYSASSLYSGVPSRFSGSRSGTDFTLTISSLQPEDFATYYCQQXXZX | |
| | | XXXTFGQGTKVEIKRTV | |
| | V_{L} | DIQMTQSPSSLSASVGDRVTITCRASQSVSSAVAWYQQKPGKAPKLL | 115 |
| | | IYSASSLYSGVPSRFSGSRSGTDFTLTISSLQPEDFATYYCQQDDYS | |
| | | LITFGQGTKVEIKRTV | |
| TL21 | V_{H(consensus)} | EVQLVESGGGLVQPGGSLRLSCAASGFTXXGXXIHWVRQAPGKGLEW | 116 |
| | | VASIYSAGGYTDYADSVKGRFTISADTSKNTAYLQMNSLRAEDTAVY | |
| | | YCARXGXYXXXXXGSWXXXPXXDYWGQGTLVTV | |
| | V_{H} | EVQLVESGGGLVQPGGSLRLSCAASGFTFSGNYIHWVRQAPGKGLEW | 117 |
| | | VASIYSAGGYTDYADSVKGRFTISADTSKNTAYLQMNSLRAEDTAVY | |
| | | YCARYGVYTLASAGSWESLPAFDYWGQGTLVTV | |
| | V_{L(consensus)} | DIQMTQSPSSLSASVGDRVTITCRASQSVSSAVAWYQQKPGKAPKLL | 118 |
| | | IYSASSLYSGVPSRFSGSRSGTDFTLTISSLQPEDFATYYCQQXXZX | |
| | | XXXTFGQGTKVEIKRTV | |
| | V_{L} | DIQMTQSPSSLSASVGDRVTITCRASQSVSSAVAWYQQKPGKAPKLL | 119 |
| | | IYSASSLYSGVPSRFSGSRSGTDFTLTISSLQPEDFATYYCQQSSWD | |
| | | LITFGQGTKVEIKRTV | |
| TL22 | V_{H(consensus)} | EVQLVESGGGLVQPGGSLRLSCAASGFTXXGXXIHWVRQAPGKGLEW | 120 |
| | | VASIYSAGGYTDYADSVKGRFTISADTSKNTAYLQMNSLRAEDTAVY | |
| | | YCARXGXYXXXXXGSWXXXPXXDYWGQGTLVTV | |
| | V_{H} | EVQLVESGGGLVQPGGSLRLSCAASGFTFSGNYIHWVRQAPGKGLEW | 121 |
| | | VASIYSAGGYTDYADSVKGRFTISADTSKNTAYLQMNSLRAEDTAVY | |
| | | YCARYGVYTLASAGSWESLPAFDYWGQGTLVTV | |
| | V_{L(consensus)} | DIQMTQSPSSLSASVGDRVTITCRASQSVSSAVAWYQQKPGKAPKLL | 122 |
| | | IYSASSLYSGVPSRFSGSRSGTDFTLTISSLQPEDFATYYCQQXXZX | |
| | | XXXTFGQGTKVEIKRTV | |
| | V_{L} | DIQMTQSPSSLSASVGDRVTITCRASQSVSSAVAWYQQKPGKAPKLL | 123 |
| | | IYSASSLYSGVPSRFSGSRSGTDFTLTISSLQPEDFATYYCQQSSWN | |
| | | GLITFGQGTKVEIKRTV | |
| TL23 | V_{H(consensus)} | EVQLVESGGGLVQPGGSLRLSCAASGFTXXGXXIHWVRQAPGKGLEW | 124 |
| | | VASIYSAGGYTDYADSVKGRFTISADTSKNTAYLQMNSLRAEDTAVY | |
| | | YCARXGXXXXXXXGSWXXXPXXDYWGQGTLVTV | |
| | V_{H} | EVQLVESGGGLVQPGGSLRLSCAASGFTFSGNYIHWVRQAPGKGLEW | 125 |
| | | VASIYSAGGYTDYADSVKGRFTISADTSKNTAYLQMNSLRAEDTAVY | |
| | | YCARYGVYTLASAGSWESLPAFDYWGQGTLVTV | |
| | V_{L(consensus)} | DIQMTQSPSSLSASVGDRVTITCRASQSVSSAVAWYQQKPGKAPKLL | 126 |
| | | IYSASSLYSGVPSRFSGSRSGTDFTLTISSLQPEDFATYYCQQXXZX | |
| | | XXXTFGQGTKVEIKRTV | |
| | V_{L} | DIQMTQSPSSLSASVGDRVTITCRASQSVSSAVAWYQQKPGKAPKLL | 127 |
| | | IYSASSLYSGVPSRFSGSRSGTDFTLTISSLQPEDFATYYCQQSGWG | |
| | | GLITFGQGTKVEIKRTV | |
| TL24 | V_{H(consensus)} | EVQLVESGGGLVQPGGSLRLSCAASGFTXXGXXIHWVRQAPGKGLEW | 128 |
| | | VASIYSAGGYTDYADSVKGRFTISADTSKNTAYLQMNSLRAEDTAVY | |
| | | YCARXGXXXXXXXGSWXXXPXXDYWGQGTLVTV | |
| | V_{H} | EVQLVESGGGLVQPGGSLRLSCAASGFTFSGNYIHWVRQAPGKGLEW | 129 |
| | | VASIYSAGGYTDYADSVKGRFTISADTSKNTAYLQMNSLRAEDTAVY | |
| | | YCARYGVYTLASAGSWESLPAFDYWGQGTLVTV | |
| | V_{L(consensus)} | DIQMTQSPSSLSASVGDRVTITCRASQSVSSAVAWYQQKPGKAPKLL | 130 |
| | | IYSASSLYSGVPSRFSGSRSGTDFTLTISSLQPEDFATYYCQQXXZX | |
| | | XXXTFGQGTKVEIKRTV | |
| | V_{L} | DIQMTQSPSSLSASVGDRVTITCRASQSVSSAVAWYQQKPGKAPKLL | 131 |
| | | IYSASSLYSGVPSRFSGSRSGTDFTLTISSLQPEDFATYYCQQSSYE | |
| | | DPITFGQGTKVEIKRTV | |
| LL2 | V_{H(consensus)} | EVQLVESGGGLVQPGGSLRLSCAASGFTXXGXXIHWVRQAPGKGLEW | 132 |
| | | VASIYSAGGYTDYADSVKGRFTISADTSKNTAYLQMNSLRAEDTAVY | |
| | | YCARXGXXXXXXXGSWXXXPXXDYWGQGTLVTV | |
| | V_{H} | EVQLVESGGGLVQPGGSLRLSCAASGFTWSGSYIHWVRQAPGKGLEW | 133 |
| | | VASIYSAGGYTDYADSVKGRFTISADTSKNTAYLQMNSLRAEDTAVY | |
| | | YCARYGVYTLASAGSWESLPAFDYWGQGTLVTV | |
| | V_{L(consensus)} | DIQMTQSPSSLSASVGDRVTITCRASQSVSSAVAWYQQKPGKAPKLL | 134 |
| | | IYSASSLYSGVPSRFSGSRSGTDFTLTISSLQPEDFATYYCQQXXZX | |
| | | XXXTFGQGTKVEIKRTV | |
| | V_{L} | DIQMTQSPSSLSASVGDRVTITCRASQSVSSAVAWYQQKPGKAPKLL | 135 |
| | | IYSASSLYSGVPSRFSGSRSGTDFTLTISSLQPEDFATYYCQQSSWE | |
| | | SPPITFGQGTKVEIKRTV | |
| LL5 | V_{H(consensus)} | EVQLVESGGGLVQPGGSLRLSCAASGFTXXGXXIHWVRQAPGKGLEW | 136 |
| | | VASIYSAGGYTDYADSVKGRFTISADTSKNTAYLQMNSLRAEDTAVY | |
| | | YCARXGXYXXXXXGSWXXXPXXDYWGQGTLVTV | |
| | V_{H} | EVQLVESGGGLVQPGGSLRLSCAASGFTWSGSYIHWVRQAPGKGLEW | 137 |
| | | VASIYSAGGYTDYADSVKGRFTISADTSKNTAYLQMNSLRAEDTAVY | |
| | | YCARYGVYTLASAGSWESLPAFDYWGQGTLVTV | |
| | V_{L(consensus)} | DIQMTQSPSSLSASVGDRVTITCRASQSVSSAVAWYQQKPGKAPKLL | 138 |
| | | IYSASSLYSGVPSRFSGSRSGTDFTLTISSLQPEDFATYYCQQXXZX | |
| | | XXXTFGQGTKVEIKRTV | |
| | V_{L} | DIQMTQSPSSLSASVGDRVTITCRASQSVSSAVAWYQQKPGKAPKLL | 139 |
| | | IYSASSLYSGVPSRFSGSRSGTDFTLTISSLQPEDFATYYCQQSSYD | |
| | | GLLTFGQGTKVEIKRTV | |
| sTL1 | V_{H} | EVQLVESGGGLVQPGGSLRLSCAASGFTWSGAYIHWVRQAPGKGLEW | 101 |
| | | VASIYSAGGYTDYADSVKGRFTISADTSKNTAYLQMNSLRAEDTAVY | |
| | | YCARYGTYELKSYGSWESLPAFDYWGQGTLVTV | |
| | V_{L} | DIQMTQSPSSLSASVGDRVTITCRASQSVSSAVAWYQQKPGKAPKLL | 99 |
| | | IYSASSLYSGVPSRFSGSRSGTDFTLTISSLQPEDFATYYCQQSSSS | |
| | | LITFGQGTKVEIKRTV | |
| sTL18 | V_{H} | EVQLVESGGGLVQPGGSLRLSCAASGFTFGGSYIHWVRQAPGKGLEW | 113 |
| | | VASIYSAGGYTDYADSVKGRFTISADTSKNTAYLQMNSLRAEDTAVY | |
| | | YCARYGVYTLHQYGSWEQLPAFDYWGQGTLVTV | |
| | V_{L} | DIQMTQSPSSLSASVGDRVTITCRASQSVSSAVAWYQQKPGKAPKLL | 99 |
| | | IYSASSLYSGVPSRFSGSRSGTDFTLTISSLQPEDFATYYCQQSSSS | |
| | | LITFGQGTKVEIKRTV | |
| sLL2 | V_{H} | EVQLVESGGGLVQPGGSLRLSCAASGFTWSGSYIHWVRQAPGKGLEW | 133 |
| | | VASIYSAGGYTDYADSVKGRFTISADTSKNTAYLQMNSLRAEDTAVY | |
| | | YCARYGVYTLASAGSWESLPAFDYWGQGTLVTV | |
| | V_{L} | DIQMTQSPSSLSASVGDRVTITCRASQSVSSAVAWYQQKPGKAPKLL | 99 |
| | | IYSASSLYSGVPSRFSGSRSGTDFTLTISSLQPEDFATYYCQQSSSS | |
| | | LITFGQGTKVEIKRTV | |

Another aspect of the present disclosure is directed to polynucleotides encoding the HER2 antibody-based molecules described herein. The nucleic acid molecules of the present disclosure include isolated polynucleotides, portions of expression vectors or portions of linear DNA sequences, including linear DNA sequences used for in vitro transcription/translation, vectors compatible with prokaryotic, eukaryotic or filamentous phage expression, secretion and/or display of the compositions or directed mutagens thereof.

In any embodiments, the nucleic acid molecules encoding the HER2 antibody-based molecules as described herein are codon optimized for expression in mammalian cells, preferably human cells. Methods of codon-optimization are known and have been described previously (e.g. International Patent Application Publication No. WO1996/09378 to Seed, which is hereby incorporated by reference in its entirety). A sequence is considered codon optimized if at least one non-preferred codon as compared to a wild-type sequence is replaced by a codon that is more preferred. Herein, a non-preferred codon is a codon that is used less frequently in an organism than another codon coding for the same amino acid, and a codon that is more preferred is a codon that is used more frequently in an organism than a non-preferred codon. The frequency of codon usage for a specific organism can be found in codon frequency tables that are well known and available in the art. Preferably more than one non-preferred codon, e.g. more than 10%, 40%, 60%, 80% of non-preferred codons, preferably most (e.g. at least 90%) or all non-preferred codons, are replaced by codons that are more preferred. Preferably the most frequently used codons in an organism are used in a codon-optimized sequence. Replacement by preferred codons generally leads to higher expression.

Polynucleotide sequences of the present disclosure can be cloned using routine molecular biology techniques, or generated de novo by DNA synthesis, which can be performed using routine procedures by service companies having business in the field of DNA synthesis and/or molecular cloning (e.g. GeneArt, GenScript, Invitrogen, Eurofins).

Another aspect of the present disclosure is directed a vector comprising the polynucleotides encoding the mutant HER2 antibody-based molecules described herein. Such vectors may be plasmid vectors, viral vectors, vectors for baculovirus expression, transposon-based vectors or any other vector suitable for introduction of the polynucleotides of the invention into a given host cell, organism or genetic background by any means. Such vectors may be expression vectors comprising nucleic acid sequence elements that can control, regulate, cause or permit expression of a polypeptide encoded by such a vector. Such elements may comprise transcriptional enhancer binding sites, RNA polymerase initiation sites, ribosome binding sites, and other sites that facilitate the expression of encoded polypeptides in a given expression system. Such expression systems may be cell-based, or cell-free systems well known in the art.

Suitable vectors include, without limitation, DNA vectors, plasmid vectors, a linear nucleic acid, and a viral vector, e.g., an adeno-associated virus (AAV) vector (see, e.g., Krause et al., “Delivery of Antigens by Viral Vectors for Vaccination,” Ther. Deliv. 2(1):51-70 (2011); Ura et al., “Developments in Viral Vector-Based Vaccines,” Vaccines 2: 624-641 (2014); Buning et al, “Recent Developments in Adeno-associated Virus Vector Technology,” J. Gene Med. 10:717-733 (2008), each of which is incorporated herein by reference in its entirety), a lentivirus vector (see, e.g., U.S. Pat. No. 748,529 to Fang et al.; Ura et al., “Developments in Viral Vector-Based Vaccines,” Vaccines 2: 624-641 (2014); and Hu et al., “Immunization Delivered by Lentiviral Vectors for Cancer and Infection Diseases,” Immunol. Rev. 239: 45-61 (2011), each of which is hereby incorporated by reference in its entirety), a retrovirus vector (see e.g., U.S. Pat. No. 748,529 to Fang et al., and Ura et al., “Developments in Viral Vector-Based Vaccines,” Vaccines 2: 624-641 (2014), each of which is hereby incorporated by reference in its entirety), a vaccinia virus, a replication deficient adenovirus vector, and a gutless adenovirus vector (see e.g., U.S. Pat. No. 5,872,005, which is incorporated herein by reference in its entirety). Methods for generating and isolating adeno-associated viruses (AAVs) suitable for use as vectors are known in the art (see, e.g., Grieger & Samulski, “Adeno-associated Virus as a Gene Therapy Vector: Vector Development, Production and Clinical Applications,” Adv. Biochem. Engin/Biotechnol. 99: 119-145 (2005); Buning et al, “Recent Developments in Adeno-associated Virus Vector Technology,” J. Gene Med. 10:717-733 (2008), each of which is incorporated herein by reference in its entirety).

The nucleic acid molecules encoding the mutant HER2 antibody-based molecules described herein are typically combined with sequences of a promoter, translation initiation, 3′ untranslated region, polyadenylation, and transcription termination in the expression vector constructs to achieve maximal expression. Promoter sequences suitable for driving expression of the mutant HER2 antibody-based molecules include, without limitation, the elongation factor 1-alpha (EF1a) promoter, a phosphoglycerate kinase-1 (PGK) promoter, a cytomegalovirus immediate early gene promoter (CMV), a chimeric liver-specific promoter (LSP), a cytomegalovirus enhancer/chicken beta-actin promoter (CAG), a tetracycline responsive promoter (TRE), a transthyretin promoter (TTR), a simian virus 40 promoter (SV40) and a CK6 promoter. Promoters suitable for recombinant T cells are identified infra. Other promoters suitable for driving gene expression in host cells that are known in the art are also suitable for incorporation into the expression constructs disclosed herein.

Another aspect of the present disclosure is directed to a host cell comprising a vector containing a polynucleotide encoding the mutant HER2 antibody-based molecules as described herein. The mutant HER2 antibody-based molecules as described herein can optionally be produced by a cell line, a mixed cell line, an immortalized cell or clonal population of immortalized cells, as well known in the art (see e.g., Ausubel et al., ed., Current Protocols in Molecular Biology, John Wiley & Sons, Inc., NY, N.Y. (1987-2001); Sambrook et al., Molecular Cloning: A Laboratory Manual, 2^{nd} Edition, Cold Spring Harbor, N.Y. (1989); Harlow and Lane, Antibodies, a Laboratory Manual, Cold Spring Harbor, N.Y. (1989); Colligan et al., eds., Current Protocols in Immunology, John Wiley & Sons, Inc., NY (1994-2001); Colligan et al., Current Protocols in Protein Science, John Wiley & Sons, NY, N.Y., (1997-2001), which are hereby incorporated by reference in their entirety). Such host cells may be eukaryotic cells, bacterial cells, plant cells or archaeal cells.

In any embodiments, the mutant HER2 antibody-based molecules are produced in a eukaryotic cell. Exemplary eukaryotic cells may be of mammalian, insect, avian or other animal origins. Mammalian eukaryotic cells include immortalized cell lines such as hybridomas or myeloma cell lines such as SP2/0 (American Type Culture Collection (ATCC), Manassas, Va., CRL-1581), NSO (European Collection of Cell Cultures (ECACC), Salisbury, Wiltshire, UK, ECACC No. 85110503), FO (ATCC CRL-1646) and Ag653 (ATCC CRL-1580) murine cell lines. An exemplary human myeloma cell line is U266 (ATTC CRL-TIB-196). Other useful cell lines include those derived from Chinese Hamster Ovary (CHO) cells such as CHO-KISV (Lonza Biologics, Walkersville, Md.), CHO-K1 (ATCC CRL-61) or DG44.

The mutant HER2 antibody-based molecules as described herein can be prepared by any of a variety of techniques using the isolated polynucleotides, vectors, and host cells described supra. In general, proteins are produced by standard cloning and cell culture techniques commonly used to prepare the recombinant expression vector, transfect the host cells, select for transformants, culture the host cells, and recover the proteins or polypeptides from the culture medium. Transfecting the host cell can be carried out using a variety of techniques commonly used for the introduction of exogenous DNA into a prokaryotic or eukaryotic host cell, e.g., by electroporation, calcium-phosphate precipitation, DEAE-dextran transfection and the like.

In any embodiment, the polynucleotides and/or vector encoding the HER2 antibody-based molecules described herein, or the antibody-based molecules are coupled to or packaged within a delivery vehicle. In accordance with this aspect of the disclosure, any suitable drug delivery vehicle known in the art can be utilized for delivery of the polynucleotides and/or vector encoding the HER2 antibody-based molecules described herein. In any embodiment, the drug delivery vehicle is a nanoparticle delivery vehicle, a polymer-based particle, or a lipid-based particle delivery vehicle known in the art (see, e.g., Xiao et al., “Engineering Nanoparticles for Targeted Delivery of Nucleic Acid Therapeutics in Tumor,” Mol. Ther. Meth. Clin. Dev. 12: 1-18 (2019); Ni et al., “Synthetic Approaches for Nucleic Acid Delivery: Choosing the Right Carriers,” Life 9(3):59 (2019), each of which is hereby incorporated by reference in its entirety), can be employed in the methods as described herein.

Suitable nanoparticle delivery vehicles comprise, without limitation, gold nanoparticles, calcium phosphate nanoparticles, cadmium (quantum dots) nanoparticles, iron oxide nanoparticles, as well as particles derived from any other solid inorganic materials as known in the art.

Suitable polymer-based particles or polyplex carriers comprise cationic polymers such as polyethylenimine (PEI), and/or cationic polymers conjugated to neutral polymers, like polyethylene glycol (PEG) and cyclodextrin. Other suitable PEI conjugates to facilitate nucleic acid molecule or expression vector delivery in accordance with the methods described herein include, without limitation, PEI-salicylamide conjugates and PEI-steric acid conjugate. Other synthetic cationic polymers suitable for use as a delivery vehicle material include, without limitation, poly-L-lysine (PLL), polyacrylic acid (PAA), polyamideamine-epichlorohydrin (PAE) and poly[2-(dimethylamino)ethyl methacrylate] (PDMAEMA). Natural cationic polymers suitable for use as delivery vehicle material include, without limitation, chitosan, poly(lactic-co-glycolic acid) (PLGA), gelatin, dextran, cellulose, and cyclodextrin.

Suitable lipid-based vehicles include cationic lipid based lipoplexes (e.g., 1,2-dioleoyl-3-trimethylammonium-propane (DOTAP)), neutral lipids based lipoplexes (e.g., cholesterol and dioleoylphosphatidyl ethanolamine (DOPE)), anionic lipid based lipoplexes (e.g., cholesteryl hemisuccinate (CHEMS)), and pH-sensitive lipid lipoplexes (e.g., 2,3-dioleyloxy-N-[2 (sperminecarboxamido)ethyl]-N,N-dimethyl-1-propanaminium trifluoroacetate (DOSPA)). Other suitable lipid-based delivery particles incorporate ionizable DOSPA in lipofectamine and DLin-MC3-DMA ((6Z,9Z,28Z,31Z)-heptatriaconta-6,9,28,31-tetraen-19-yl-4-(dimethylamino)butanoate).

Another aspect of the present disclosure is directed to an immunoconjugate comprising: the HER2 antibody-based molecule as described herein, and a cytotoxic agent, wherein said cytotoxic agent is coupled to said antibody-based molecule. In any embodiment, the cytotoxic agent is a chemotherapeutic drug. In any embodiment, the cytotoxic agent of the immunoconjugate is selected from auristatin, a maytansinoid, a calicheamicin, a pyrrolobenzodiazepine, a nemorubicin derivative, and a 1-(chloromethyl)-2,3-dihydro-1H-benzo[e]indole (CBI). Other cytotoxic agents can also be utilized.

Another aspect of the present disclosure is directed to a chimeric antigen receptor (CAR) molecule comprising the HER2 antibody-based molecule as described herein; a transmembrane domain; and an activation domain. In any embodiment, the antibody-based molecule comprises of the chimeric antigen receptor is a scFv.

The term “chimeric antigen receptors (CARs)” as used herein may be referred to as artificial T-cell receptors, chimeric T-cell receptors, or chimeric immune-receptors, for example, and encompass engineered receptors that graft an artificial specificity onto a particular immune effector cell. The CARs may be employed to impart the specificity of a monoclonal antibody onto a T cell, thereby allowing a large number of specific T cells to be generated, for example, in use for adoptive cell therapy. The CARs described herein direct specificity of the cell to the HER2 S310F/Y antigen. In particular aspects, CARs comprise fusions of single-chain variable fragments (scFv) derived from the HER2 S310F/Y monoclonal antibodies, fused to CD3-zeta transmembrane and endodomain. In particular embodiments, one can target malignant or solid tumor cells by redirecting the specificity of T cells using a chimeric immunoreceptor specific for the HER2 S310F/Y antigen. In some cases, molecules can be co-expressed with the CAR. These include co-stimulatory molecules, reporter genes for imaging (e.g., for positron emission tomography), gene products that conditionally ablate the T cells upon addition of a pro-drug, homing receptors, cytokines, and cytokine receptors.

As used herein, the term “extracellular domain,” refers to the part of a CAR that is located outside of the cell membrane and is capable of binding to the HER2 S310F/Y antigen. Any suitable antibody-based molecule as described herein, with binding specificity to the HER2 S310F/Y antigen, can be used as the extracellular domain.

As used herein, the term “transmembrane domain” refers to the portion of a CAR that extends across the cell membrane and anchors the CAR to cell membrane. Any suitable transmembrane domain known to be effective in CARs can be utilized.

As used herein, the term “intracellular signaling domain” refers to the part of a CAR that is located inside of the cell membrane and is capable of transducing an effector signal. Any suitable intracellular signaling domain known to be effective in CARs can be utilized.

CARs afford a useful anti-tumor approach to eradicate tumor cells by adoptive transfer of T cells expressing chimeric antigen receptors to recognize specific antigens presented on tumor cells and activate T cells to specifically lyse these tumor cells. One aspect of this CAR strategy is the selection of target epitopes that are specifically or selectively expressed on tumors, are present on all tumor cells, and are membrane epitopes not prone to shed or modulate from the cell surface. HER2 S310F/Y meets these criteria. To employ the cells in such a manner, one must prevent their rejection in a graft-versus-host response without compromising CAR-dependent effector functions.

Various approaches for the development of CAR T cells are described, for example, in U.S Application Publ. No. 20200332255A1 to Lee et al., which is hereby incorporated by reference in its entirety. In particular, Lee et al., describes the generation of universal T cell-based immunotherapies by redirecting T-cell specificity using immortalized T cells that can serve as “off-the-shelf reagents.” In other words, engineered immortalized T cells can be pre-prepared and then infused into multiple recipients. This will facilitate “centralized” manufacturing of the universal T cells and subsequent pre-positioning of the T cells at regional facilities for infusion on demand, enable clinical trials to be undertaken that are powered for efficacy, and facilitate combination therapies in which the universal T cells can be administered with other biologies and therapeutics. To achieve this, one can eliminate endogenous TCR and B2M expression, which causes unwanted allogeneic immune reactions. Such steps can occur by any suitable manner, including by introducing a Cas9/CRISPR complex, for example, targeting TCR a constant region or β constant region. Embodiments of the invention are unique as they combine (i) redirecting the specificity of immortalized T cells by introducing a CAR and (ii) eliminating expression of endogenous TCR and B2M to generate a desired T-cell product. In certain embodiments, the introduction of CAR and elimination of TCR/B2M are accomplished by electroporation to stably express CAR and desired transient transfection of in vitro-transcribed mRNA. In embodiments of the invention, infusing specific engineered immortalized CAR-T cells are pre-prepared and thawed to be infused on demand as an off-the-shelf reagent.

Alternatively, autologous CAR T cells can be utilized to treat a patient by recovering T cells from a patient and using the recovered T cells for ex vivo generation of CAR-T cells from patient.

As used herein, the term “T cell” refers to a type of lymphocyte that matures in the thymus. T cells play an important role in cell-mediated immunity and are distinguished from other lymphocytes, such as B cells, by the presence of a T-cell receptor on the cell surface. T cells may either be isolated or obtained from a commercially available source. “T cell” includes all types of immune cells expressing CD3 including T-helper cells (CD4+ cells), cytotoxic T-cells (CD8+ cells), natural killer T-cells, T-regulatory cells (Treg) and gamma-delta T cells. A “cytotoxic cell” includes CD8+ T cells, natural-killer (NK) cells, and neutrophils, which cells are capable of mediating cytotoxicity responses.

Another aspect of the present disclosure is directed to a multi-specific antibody or multi-specific binding fragment thereof comprising: a first antigen-binding arm comprising the HER2 antibody-based molecule described herein, and a second antigen-binding arm that binds to a surface antigen selectively expressed on an immune cell surface.

In any embodiment, the second antigen-binding arm binds to a surface antigen selectively expressed on natural killer (NK) cells. In any embodiment, the NK cell specific surface antigen is selected from the group consisting of CD16A, NKG2D, CD94/NKG2C, NKp30, NKp44, and NKp46. Antibodies capable of binding NK cell specific surface antigens are known in the art and suitable for use in the multi-specific antibody describe herein. See e.g., U.S. Pat. No. 11,001,633 to Affimed GmbH (anti-CD16A); U.S. Patent Appl. Publ. No. 20200231678 (anti-NKG2D); U.S. Patent Appl. Publ. No. 20190055315 to Innate Pharma SAS (anti-NKp46), each of which is hereby incorporated by reference in their entirety.

In any embodiment, the second antigen-binding arm of the multispecific antibody or multi-specific binding fragment thereof binds to a surface antigen selectively expressed on T cells. In any embodiment, the T cell surface antigen is CD3. Antibodies capable of binding NK cell specific surface antigens are known in the art and suitable for use in the multispecific antibody describe herein. See e.g., WO2013026837 to Roche; US20170327579 to Engmab SARL; U.S. Pat. No. 9,315,567 to IBC Pharmaceuticals; WO2017055391 to Hoffmann-La Roche Ag; and WO2012055961 to Micromet GmBh, each of which is hereby incorporated by reference in its entirety.

Exemplary bispecific antibody constructs, in the form of single-chain diabodies, include a first antigen-binding arm comprising a HER2-binding antibody fragment described herein and a second antigen-binding arm comprising a CD3-binding antibody fragment. Several embodiments are provided below:

CH15V_scDb
(SEQ ID NO: 144)
DIVRSDIQMTQSPSSLSASVGDRVTITCRASQSVSSAVAWYQQKPGKAPKLLIYSASSLYSGVPSRFS
GSRSGTDFTLTISSLQPEDFATYYCQQSSSSLITFGQGTKVEIKGGGGSEVQLQQSGPELVKPGASMK
ISCKASGYSFTGYTMNWVKQSHGKNLEWMGLINPYKGVSTYNQKFKDKATLTVDKSSSTAYMELLSLT
SEDSAVYYCARSGYYGDSDWYFDVWGQGTTLTVSSGGGGSGGGGSGGGGSDIQMTQTTSSLSASLGDR
VTISCRASQDIRNYLNWYQQKPDGTVKLLIYYTSRLHSGVPSKFSGSGSGTDYSLTISNLEQEDIATY
FCQQGNTLPWTFAGGTKLEIKGGGGSEVQLVESGGGLVQPGGSLRLSCAASGFTFSGNYIHWVRQAPG
KGLEWVASIYSAGGYTDYADSVKGRFTISADTSKNTAYLQMNSLRAEDTAVYYCARYGVYTMHQYGSW
EQMPAFDYWGQGTLVTVSSLEGGGGLNDIFEAQKIEWHESRHHHHHH
sLL2_scDb
(SEQ ID NO: 145)
DIVRSDIQMTQSPSSLSASVGDRVTITCRASQSVSSAVAWYQQKPGKAPKLLIYSASSLYSGVPSRFS
GSRSGTDFTLTISSLQPEDFATYYCQQSSSSLITFGQGTKVEIKGGGGSEVQLQQSGPELVKPGASMK
ISCKASGYSFTGYTMNWVKQSHGKNLEWMGLINPYKGVSTYNQKFKDKATLTVDKSSSTAYMELLSLT
SEDSAVYYCARSGYYGDSDWYFDVWGQGTTLTVSSGGGGSGGGGSGGGGSDIQMTQTTSSLSASLGDR
VTISCRASQDIRNYLNWYQQKPDGTVKLLIYYTSRLHSGVPSKFSGSGSGTDYSLTISNLEQEDIATY
FCQQGNTLPWTFAGGTKLEIKGGGGSEVQLVESGGGLVQPGGSLRLSCAASGFTWSGSYIHWVRQAPG
KGLEWVASIYSAGGYTDYADSVKGRFTISADTSKNTAYLQMNSLRAEDTAVYYCARYGVYTLASAGSW
ESLPAFDYWGQGTLVTVSSLEGGGGLNDIFEAQKIEWHESRHHHHHH
sTL1_scDb
(SEQ ID NO: 146)
DIVRSDIQMTQSPSSLSASVGDRVTITCRASQSVSSAVAWYQQKPGKAPKLLIYSASSLYSGVPSRFS
GSRSGTDFTLTISSLQPEDFATYYCQQSSSSLITFGQGTKVEIKGGGGSEVQLQQSGPELVKPGASMK
ISCKASGYSFTGYTMNWVKQSHGKNLEWMGLINPYKGVSTYNQKFKDKATLTVDKSSSTAYMELLSLT
SEDSAVYYCARSGYYGDSDWYFDVWGQGTTLTVSSGGGGSGGGGSGGGGSDIQMTQTTSSLSASLGDR
VTISCRASQDIRNYLNWYQQKPDGTVKLLIYYTSRLHSGVPSKFSGSGSGTDYSLTISNLEQEDIATY
FCQQGNTLPWTFAGGTKLEIKGGGGSEVQLVESGGGLVQPGGSLRLSCAASGFTWSGAYIHWVRQAPG
KGLEWVASIYSAGGYTDYADSVKGRFTISADTSKNTAYLQMNSLRAEDTAVYYCARYGTYELKSYGSW
ESLPAFDYWGQGTLVTVSSLEGGGGLNDIFEAQKIEWHESRHHHHHH
sTL18_scDb
(SEQ ID NO: 147)
DIVRSDIQMTQSPSSLSASVGDRVTITCRASQSVSSAVAWYQQKPGKAPKLLIYSASSLYSGVPSRFS
GSRSGTDFTLTISSLQPEDFATYYCQQSSSSLITFGQGTKVEIKGGGGSEVQLQQSGPELVKPGASMK
ISCKASGYSFTGYTMNWVKQSHGKNLEWMGLINPYKGVSTYNQKFKDKATLTVDKSSSTAYMELLSLT
SEDSAVYYCARSGYYGDSDWYFDVWGQGTTLTVSSGGGGSGGGGSGGGGSDIQMTQTTSSLSASLGDR
VTISCRASQDIRNYLNWYQQKPDGTVKLLIYYTSRLHSGVPSKFSGSGSGTDYSLTISNLEQEDIATY
FCQQGNTLPWTFAGGTKLEIKGGGGSEVQLVESGGGLVQPGGSLRLSCAASGFTFGGSYIHWVRQAPG
KGLEWVASIYSAGGYTDYADSVKGRFTISADTSKNTAYLQMNSLRAEDTAVYYCARYGVYTLHQYGSW
EQLPAFDYWGQGTLVTVSSLEGGGGLNDIFEAQKIEWHESRHHHHHH

In each of the constructs shown above, the AviTag and HisTag sequences are underlined.

Additional exemplary bispecific antibody constructs, in addition to the form of single-chain diabodies, include a first antigen-binding arm comprising a HER2-binding antibody fragment described herein and a second antigen-binding arm comprising a CD3-binding antibody fragment. Several embodiments, with a CD3-binding antibody UCHT1 as an example for the second antigen-binding arm (although other anti-CD3-binding antibodies can be used instead of UCHT1), are provided below:

sTL18_BiTE_HL (Goebeler et al., “T Cell-engaging Therapies-BiTEs and Beyond,”
Nat Rev Clin Oncol. 17(7): 418-434 (2020), which is hereby incorporated by
reference in its entirety).
(SEQ ID NO: 148)
EVQLVESGGGLVQPGGSLRLSCAASGFTFGGSYIHWVRQAPGKGLEWVASIYSAGGYTDYADSVKGRFTISADT
SKNTAYLQMNSLRAEDTAVYYCARYGVYTLHQYGSWEQLPAFDYWGQGTLVTVSSGGGGSGGGGSGGGGSDIQM
TQSPSSLSASVGDRVTITCRASQSVSSAVAWYQQKPGKAPKLLIYSASSLYSGVPSRFSGSRSGTDFTLTISSL
QPEDFATYYCQQSSSSLITFGQGTKVEIKSGGGGSEVQLQQSGPELVKPGASMKISCKASGYSFTGYTMNWVKQ
SHGKNLEWMGLINPYKGVSTYNQKFKDKATLTVDKSSSTAYMELLSLTSEDSAVYYCARSGYYGDSDWYFDVWG
QGTTLTVSSGGGGSGGGGSGGGGSDIQMTQTTSSLSASLGDRVTISCRASQDIRNYLNWYQQKPDGTVKLLIYY
TSRLHSGVPSKFSGSGSGTDYSLTISNLEQEDIATYFCQQGNTLPWTFAGGTKLEIKLEGGGGLNDIFEAQKIE
WHESRHHHHHH
sTL18_BITE_LH
(SEQ ID NO: 149)
DIQMTQSPSSLSASVGDRVTITCRASQSVSSAVAWYQQKPGKAPKLLIYSASSLYSGVPSRFSGSRSGTDFTLT
ISSLQPEDFATYYCQQSSSSLITFGQGTKVEIKGGGGSGGGGSGGGGSEVQLVESGGGLVQPGGSLRLSCAASG
FTFGGSYIHWVRQAPGKGLEWVASIYSAGGYTDYADSVKGRFTISADTSKNTAYLQMNSLRAEDTAVYYCARYG
VYTLHQYGSWEQLPAFDYWGQGTLVTVSSSGGGGSDIQMTQTTSSLSASLGDRVTISCRASQDIRNYLNWYQQK
PDGTVKLLIYYTSRLHSGVPSKFSGSGSGTDYSLTISNLEQEDIATYFCQQGNTLPWTFAGGTKLEIKGGGGSG
GGGSGGGGSEVQLQQSGPELVKPGASMKISCKASGYSFTGYTMNWVKQSHGKNLEWMGLINPYKGVSTYNQKFK
DKATLTVDKSSSTAYMELLSLTSEDSAVYYCARSGYYGDSDWYFDVWGQGTTLTVSSLEGGGGLNDIFEAQKIE
WHESRHHHHHH

In each of the constructs shown above, the AviTag and HisTag sequences are underlined.

sTL18_CrossMab, where the CrossMab molecule includes four chains A, B, C and D (Surowka et al., “Ten Years in the Making: Application of CrossMab Technology for the Development of Therapeutic bispecific Antibodies and Antibody Fusion Protein,” MAbs 13(1):1967714 (2021), which is hereby incorporated by reference in its entirety).

Chain_A (UCHT1VHCL)
(SEQ ID NO: 150)
EVQLQQSGPELVKPGASMKISCKASGYSFTGYTMNWVKQSHGKNLEWMGLINPYKGVSTYNQKFKDKATLTVDK
SSSTAYMELLSLTSEDSAVYYCARSGYYGDSDWYFDVWGQGTTLTVSSASVAAPSVFIFPPSDEQLKSGTASVV
CLLNNFYPREAKVQWKVDNALQSGNSQESVTEQDSKDSTYSLSSTLTLSKADYEKHKVYACEVTHQGLSSPVTK
SFNRGEC
Chain_B (sTL18VHCH1_UCHT1VLCH1_Fcknob)
(SEQ ID NO: 151)
EVQLVESGGGLVQPGGSLRLSCAASGFTEGGSYIHWVRQAPGKGLEWVASIYSAGGYTDYADSVKGRFTISADT
SKNTAYLQMNSLRAEDTAVYYCARYGVYTLHQYGSWEQLPAFDYWGQGTLVTVSSASTKGPSVFPLAPSSKSTS
GGTAALGCLVEDYFPEPVTVSWNSGALTSGVHTFPAVLQSSGLYSLSSVVTVPSSSLGTQTYICNVNHKPSNTK
VDEKVEPKSCDGGGGGGGGSDIQMTQTTSSLSASLGDRVTISCRASQDIRNYLNWYQQKPDGTVKLLIYYTSR
LHSGVPSKFSGSGSGTDYSLTISNLEQEDIATYFCQQGNTLPWTFAGGTKLEIKSSASTKGPSVFPLAPSSKST
SGGTAALGCLVKDYFPEPVTVSWNSGALTSGVHTFPAVLQSSGLYSLSSVVTVPSSSLGTQTYICNVNHKPSNT
KVDKKVEPKSCDKTHTCPPCPAPEAAGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALGAPIEKTISKAKGQPREPQVYTLPPCRDE
LTKNQVSLWCLVKGFYPSDIAVEWESNGQPENNYKTTPPVLDSDGSFFLYSKLTVDKSRWQQGNVFSCSVMHEA
LHNHYTQKSLSLSP
Chain_C (sTL18VHCH1_Fchole)
(SEQ ID NO: 152)
EVQLVESGGGLVQPGGSLRLSCAASGFTEGGSYIHWVRQAPGKGLEWVASIYSAGGYTDYADSVKGRFTISADT
SKNTAYLQMNSLRAEDTAVYYCARYGVYTLHQYGSWEQLPAFDYWGQGTLVTVSSASTKGPSVFPLAPSSKSTS
GGTAALGCLVEDYFPEPVTVSWNSGALTSGVHTFPAVLQSSGLYSLSSVVTVPSSSLGTQTYICNVNHKPSNTK
VDEKVEPKSCDKTHTCPPCPAPEAAGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALGAPIEKTISKAKGQPREPQVCTLPPSRDEL
TKNQVSLSCAVKGFYPSDIAVEWESNGQPENNYKTTPPVLDSDGSFFLVSKLTVDKSRWQQGNVFSCSVMHEAL
HNHYTQKSLSLSP
Chain_D (sTL18VLCL)
(SEQ ID NO: 153)
DIQMTQSPSSLSASVGDRVTITCRASQSVSSAVAWYQQKPGKAPKLLIYSASSLYSGVPSRFSGSRSGTDFTLT
ISSLQPEDFATYYCQQSSSSLITFGQGTKVEIKRTVAAPSVFIFPPSDRKLKSGTASVVCLLNNFYPREAKVQW
KVDNALQSGNSQESVTEQDSKDSTYSLSSTLTLSKADYEKHKVYACEVTHQGLSSPVTKSFNRGEC

STL18 FabH-UCHT1 scFv (equivalent to ImmTAC) that contains two chains A and B (Liddy et al., “Monoclonal TCR-redirected Tumor Cell Killing,” Nat Med. 18(6):980-7 (2012), which is hereby incorporated by reference in its entirety).

sTL18VHCH1_UCHT1scFv
(SEQ ID NO: 154)
EVQLVESGGGLVQPGGSLRLSCAASGFTFGGSYIHWVRQAPGKGLEWVASIYSAGGYTDYADSVKGRFTISADT
SKNTAYLQMNSLRAEDTAVYYCARYGVYTLHQYGSWEQLPAFDYWGQGTLVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKPSNTK
VDKRVEPKSCGGGGSEVQLQQSGPELVKPGASMKISCKASGYSFTGYTMNWVKQSHGKNLEWMGLINPYKGVST
YNQKFKDKATLTVDKSSSTAYMELLSLTSEDSAVYYCARSGYYGDSDWYFDVWGQGTTLTVSSGGGGSGGGGSG
GGGSDIQMTQTTSSLSASLGDRVTISCRASQDIRNYLNWYQQKPDGTVKLLIYYTSRLHSGVPSKFSGSGSGTD
YSLTISNLEQEDIATYFCQQGNTLPWTFAGGTKLEIK
sTL18VLCL
(SEQ ID NO: 155)
DIQMTQSPSSLSASVGDRVTITCRASQSVSSAVAWYQQKPGKAPKLLIYSASSLYSGVPSRFSGSRSGTDFTLT
ISSLQPEDFATYYCQQSSSSLITFGQGTKVEIKRTVAAPSVFIFPPSDEQLKSGTASVVCLLNNFYPREAKVQW
KVDNALQSGNSQESVTEQDSKDSTYSLSSTLTLSKADYEKHKVYACEVTHQGLSSPVTKSFNRGEC

STL18 FabL-UCHT1 scFv (equivalent to ImmTAC) that contains two chains, A and B (Liddy et al., “Monoclonal TCR-redirected Tumor Cell Killing,” Nat Med. 18(6):980-7 (2012), which is hereby incorporated by reference in its entirety).

sTIL18VHCH1
(SEQ ID NO: 156)
EVQLVESGGGLVQPGGSLRLSCAASGFTFGGSYIHWVRQAPGKGLEWVASIYSAGGYTDYADSVKGRFTISADT
SKNTAYLQMNSLRAEDTAVYYCARYGVYTLHQYGSWEQLPAFDYWGQGTLVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKPSNTK
VDKRVEPKSC
sTL18VLCL_UCHT1scFv)
(SEQ ID NO: 157)
DIQMTQSPSSLSASVGDRVTITCRASQSVSSAVAWYQQKPGKAPKLLIYSASSLYSGVPSRFSGSRSGTDFTLT
ISSLQPEDFATYYCQQSSSSLITFGQGTKVEIKRTVAAPSVFIFPPSDEQLKSGTASVVCLLNNFYPREAKVQW
KVDNALQSGNSQESVTEQDSKDSTYSLSSTLTLSKADYEKHKVYACEVTHQGLSSPVTKSFNRGECGGGGSEVQ
LQQSGPELVKPGASMKISCKASGYSFTGYTMNWVKQSHGKNLEWMGLINPYKGVSTYNQKFKDKATLTVDKSSS
TAYMELLSLTSEDSAVYYCARSGYYGDSDWYFDVWGQGTTLTVSSGGGGSGGGGSGGGGSDIQMTQTTSSLSAS
LGDRVTISCRASQDIRNYLNWYQQKPDGTVKLLIYYTSRLHSGVPSKFSGSGSGTDYSLTISNLEQEDIATYFC
QQGNTLPWTFAGGTKLEIK

Another aspect of the present disclosure is directed to a pharmaceutical composition comprising: the HER2 antibody-based molecule as describe herein, the HER-antibody immunoconjugate as described herein, the multi-specific antibody or multi-specific binding fragment thereof as described herein, the polynucleotides and vectors encoding the HER-2 antibodies and multi-specific antibodies and antibody conjugates as described herein and a pharmaceutically acceptable carrier.

The formulation of pharmaceutically active ingredients with pharmaceutically acceptable carriers is known in the art, e.g., Remington: The Science and Practice of Pharmacy (e.g. 21st edition (2005), and any later editions). Non-limiting examples of additional ingredients include: buffers, diluents, solvents, tonicity regulating agents, preservatives, stabilizers, and chelating agents. One or more pharmaceutically acceptable carrier can be used in formulating the pharmaceutical compositions of the invention.

As used herein, the terms “pharmaceutically acceptable carrier” and “pharmaceutically acceptable excipient” (e.g., additives such as diluents, immunostimulants, adjuvants, antioxidants, preservatives and solubilizing agents) are non-toxic to the subject administered the composition at the dosages and concentrations employed. Examples of pharmaceutically acceptable carriers include water, e.g., buffered with phosphate, citrate and another organic acid. Representative examples of pharmaceutically acceptable excipients that may be useful in the present disclosure include antioxidants such as ascorbic acid; low molecular weight (less than about 10 residues) polypeptides; proteins, such as serum albumin, gelatin, or other immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt forming counterions such as sodium; and/or nonionic surfactants.

In any embodiment, the pharmaceutical composition as described herein is a liquid formulation. A preferred example of a liquid formulation is an aqueous formulation, i.e., a formulation comprising water. The liquid formulation can comprise a solution, a suspension, an emulsion, a microemulsion, a gel, and the like. An aqueous formulation typically comprises at least 50% w/w water, or at least 60%, 70%, 75%, 80%, 85%, 90%, or at least 95% w/w of water.

The pH in an aqueous formulation of the pharmaceutical composition can be between pH 3 and pH 10. In one embodiment, the pH of the pharmaceutical composition is from about 7.0 to about 9.5. In another embodiment, the pH of the pharmaceutical composition is from about 3.0 to about 7.0.

In any embodiment, the pharmaceutical compositions can be provided in a unit dosage form wherein each dosage unit, e.g., an injection, contains a predetermined therapeutically effective amount of the composition, alone or in appropriate combination with other active agents. The term unit dosage form as used herein refers to physically discrete units suitable as unitary dosages for human and animal subjects, each unit containing a predetermined quantity of the composition of the present invention, alone or in combination with other active agents, calculated in an amount sufficient to produce the desired effect, in association with a pharmaceutically acceptable diluent, carrier, or vehicle, where appropriate. The specifications for the novel unit dosage forms of the present invention depend on the particular pharmacodynamics associated with the pharmaceutical composition in the particular subject.

The term “therapeutically effective amount” refers to an amount of an active ingredient or component that elicits the desired biological or medicinal response in a subject. A therapeutically effective amount can be determined empirically and in a routine manner, in relation to the stated purpose. Suitable doses can be calculated in consideration of target attributes (baseline and turnovers), antibody pharmacokinetics (PK), target-binding properties, modes of action (MoAs) and patient characteristics. See, e.g., Tang et al., “Which Factors Matter the Most? Revisiting and Dissecting Antibody Therapeutic Doses,” Drug Discov Today 26(8):1980-1990 (2021), which is hereby incorporated by reference in its entirety. In general, receptor occupancy (RO) is the most common indicator for dose adequacy, which is often used to predict antibody efficacy and safety profiles. The measurement of RO in vivo or ex vivo reveals an antibody's key properties for engaging with its target. After the safety profile has been cleared and the risk of hyper-inflammation in the early clinical stages has been minimized, a nearly saturated RO (close to 100%) is usually desired for the highest efficacy potential. In consideration of these principles, the amount of the HER2 antibody-based molecule per unit dose is typically between about 0.1 mg/kg and about 20 mg/kg, such as between about 0.1 mg/kg and about 5 mg/kg, or about 5 mg/kg to about 10 mg/kg.

In various embodiments, the pharmaceutical compositions as described herein are suitable for delivery parenterally, peritoneally, intravenously, intraarterially, intratumorally, peritumorally, intramuscularly, subcutaneously, intradermally, intranasally, by inhalation, intravitreal, intranodally, intraportally, intrahepatically, and intra-CNS routes. Other routes are described by Pitiot et al., “Alternative Routes of Administration for Therapeutic Antibodies—State of the Art,” Antibodies (Basel) 11(3):56 (2022), which is hereby incorporated by reference in its entirety.

Another aspect of the present disclosure is directed to a method of treating a subject having a HER2-positive cancer, the method comprising administering to the subject the pharmaceutical composition as described herein in an amount effective to treat the subject having the HER2-positive cancer.

Another aspect of the present disclosure is directed to a method of treating a subject having a cancer expressing a mutant HER-2, the method comprising administering to the subject a population of autologous immune cells expressing the chimeric antigen receptor of as described herein in an amount effective to treat the subject having the mutant HER-2 expressing cancer.

The methods described herein are suitable for treating a cancer expressing a HER2 mutant, in particular a HER2 S310 mutant. The cancer expressing mutant HER2 is selected from breast cancer, colorectal cancer, lung squamous cell carcinoma, lung small cell cancer, lung adenocarcinoma, bladder cancer, gastric cancer, glioblastoma, cutaneous squamous carcinoma, gallbladder cancer, head and neck squamous cell carcinoma, endometrial cancer, cholangiocarcinoma, cervical cancer, uterine cancer, glioma, prostate cancer, salivary gland cancer, and testicular cancer.

As used herein, the terms “treat,” “treating,” and “treatment” are all intended to refer to an amelioration or reversal of at least one measurable physical parameter related to a cancer, which is not necessarily discernible in the subject, but can be discernible in the subject. The terms “treat,” “treating,” and “treatment,” can also refer to causing regression, preventing the progression, or at least slowing down the progression of the disease, disorder, or condition. In a particular embodiment, “treat,” “treating,” and “treatment” refer to an alleviation, prevention of the development or onset, or reduction in the duration of one or more symptoms associated with the disease, disorder, or condition, such as a tumor or more preferably a cancer. In a particular embodiment, “treat,” “treating,” and “treatment” refer to prevention of the recurrence of the disease, disorder, or condition. In a particular embodiment, “treat,” “treating,” and “treatment” refer to an increase in the survival of a subject having the disease, disorder, or condition. In a particular embodiment, “treat,” “treating,” and “treatment” refer to elimination of the disease, disorder, or condition in the subject.

Another aspect of the present disclosure is directed to a diagnostic agent comprising: the HER2 antibody-based molecule as describe herein, and a detectable label, wherein said detectable label is coupled to the antibody-based molecule.

Any of a variety of detectable label can be used to facilitate detection of a label-linked HER2 antibody-based molecule. Exemplary detectable labels include, without limitation, fluorophores, radioisotopes, paramagnetic beads, CT contrasting agents, microbubbles, and combinations thereof.

The detectable label is preferably covalently linked to the antibody at very low molar ratios to prevent interference with the antigen-binding site and to prevent a high rate of hepatic clearance. Furthermore, the imaging efficiency of the diagnostic probe provides a strong signal even at these relatively low molar ratios. Several strategies for antibody-tracer conjugates (ATCs) are currently in clinical practice. For nuclear imaging purposes, a modality-specific radionuclide is conjugated to the antibody and combined with either SPECT or PET imaging. Paramagnetic or superparamagnetic particles for antibody labelling are used in combination with magnetic resonance imaging. Microbubbles can be used in combination with ultrasound imaging. Optical dyes are dependent on the properties of light and as a result, have limited depth of tissue penetration, but high resolution when imaged at the surface. These agents are considered optimal for the surgical setting where the tissue planes are exposed and wide-field, high-resolution imaging can be applied in real-time. Furthermore, optical probes can be designed to emit fluorescence and toxic reactive oxygen species after light based activation. This imaging strategy is referred to as photoimmunotherapy (PIT) and has concurrent diagnostic and therapeutic application. These and other forms of imaging cancers are described in Warram et al., “Antibody-based Imaging Strategies for Cancer,” Cancer Metastasis Rev. 33(2-3):809-22 (2014), which is hereby incorporated by reference in its entirety.

In vitro diagnostics are also contemplated, including immunohistochemical detection of anti-HER2 S310F/Y labeled cancer cells in tissue samples. The labeling of tissue samples and counter-staining, if applied, are well known in the literature. Various labeling and detection protocols are described by Gremel et al., “In Situ Protein Detection for Companion Diagnostics,” Front. Oncol. 3: Article 271 (2013), which is hereby incorporated by reference in its entirety.

### EXAMPLES

The examples below are intended to exemplify the practice of embodiments of the disclosure but are by no means intended to limit the scope thereof.

### Example 1—Identification of Antibody Clones Selective to HER2 Mutants S310F and S310Y

Surface plasmon resonance (SPR) measurements showed that Trastuzumab bound to both S310F and WT HER2 samples, but Pertuzumab had markedly diminished binding to S310F relative to WT HER2. These binding characteristics can be rationalized based on the crystal structures of HER2 ECD in complex with these antibodies. Pertuzumab binds close to S310F (Franklin et al., “Insights into ErbB Signaling from the Structure of the ErbB2-Pertuzumab Complex,” Cancer Cell. 5(4):317-28 (2004), which is hereby incorporated by reference in its entirety), whereas Trastuzumab binds distal to S310F (Choo et al., “Structure of the Extracellular Region of HER2 Alone and in Complex with the Herceptin Fab,” Nature 421(6924):756-60 (2003), which is hereby incorporated by reference in its entirety).

Sorting of a phage-display library of synthetic antibodies was similar to those previously described (Miller et al., “T Cell Receptor-like Recognition of Tumor in vivo by Synthetic Antibody Fragment,” PLoS One 7(8):e43746 (2012); Paduch et al., “Generating Conformation-specific Synthetic Antibodies to Trap Proteins in Selected Functional States,” Methods 60(1):3-14 (2013), each of which is hereby incorporated by reference in its entirety). To enrich antibody clones that are selective to the HER2 mutants, negative selection with the WT HER2 sample and positive selection using the S310F and S310Y samples were used. Four clones exhibiting stronger binding to the HER2 mutants than to WT HER2 (7-20, 11-1, 10-20 and 25-20) were identified, and one clone binding to both WT and mutants with similar strength (20-20) was identified.

These antibody clones were first produced in the Fab format with a C-terminal Avi-tag. Using a bead binding assay (Hattori et al., “Multiplex Bead Binding Assays Using Off-the-shelf Components and Common Flow Cytometers,” J Immunol Methods 490:112952 (2021), which is hereby incorporated by reference in its entirety), their binding specificity was confirmed. The binding specificity of clone 11 (aka 11-1) was further confirmed using SPR.

Ser substitutions of clone 11 were tested at a few positions. None of these mutations showed strong decrease in binding, suggesting that these residues may be substituted with another amino acid without detrimental effect.

A subset of the identified clones was produced in the format of human IgG1. Flow cytometry measurements showed that they selective bound to HER2 S310F in the low-level expressing cells. A bead binding assay showed that the new clones have increased affinity compared with clone 11, as expected. Similar results were obtained using binding titration using HER2-expressing HEK293T cells. Biolayer interferometry (BLI) measurements showed that CH15 bound to S310F and S310Y mutants.

ADCC assay showed that the CH15 clone did not kill BaF3 cells expressing WT HER2, whereas both Trastuzumab and Pertuzumab killed the cells. In contrast, CH15 killed BaF3 cells expressing S310Y. As expected, the effectiveness of Pertuzumab was decreased in S310Y killing, because Pertuzumab binds less well to S310 mutants than WT. These results indicate that CH15 can selectively kill HER2 mutant cells.

### HER2 ECD-Fc Constructs:

In the WT HER2-Fc shown below, the secretion signal is shown in italics, an interdomain linker and C-terminal tag are underlined, and the S310 residue is bold/underlined.

(SEQ ID NO: 67)
MELAALCRWGLLLALLPPGAAS
TQVCTGTDMKLRLPASPETHLDMLRHLYQGCQVVQGNLELTYLPTNASLSFLQDIQEVQGYVLIAHNQVRQVPLQRL
RIVRGTQLFEDNYALAVLDNGDPLNNTTPVTGASPGGLRELQLRSLTEILKGGVLIQRNPQLCYQDTILWKDIFHKN
NQLALTLIDTNRSRACHPCSPMCKGSRCWGESSEDCQSLTRTVCAGGCARCKGPLPTDCCHEQCAAGCTGPKHSDCL
ACLHFNHSGICELHCPALVTYNTDTFESMPNPEGRYTFGASCVTACPYNYLSTDVGSCTLVCPLHNQEVTAEDGTQR
CEKCSKPCARVCYGLGMEHLREVRAVTSANIQEFAGCKKIFGSLAFLPESFDGDPASNTAPLQPEQLQVFETLEEIT
GYLYISAWPDSLPDLSVFQNLQVIRGRILHNGAYSLTLQGLGISWLGLRSLRELGSGLALIHHNTHLCFVHTVPWDQ
LERNPHQALLHTANRPEDECVGEGLACHQLCARGHCWGPGPTQCVNCSQFLRGQECVEECRVLQGLPREYVNARHCL
PCHPECQPQNGSVTCFGPEADQCVACAHYKDPPFCVARCPSGVKPDLSYMPIWKEPDEEGACQPCPINCTHSCVDLD
DKGCPAEQRASPLT
PGSRS
PKSCDKTHTCPPCPAPELLGGPSVFLFPPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQGNVFSCSVMHEALHNHYTQKSLSLSPGK
LEGGGGLNDIFEAQKIEWHESRHHHHHH

In the HER2 S310F-Fc shown below, the secretion signal is shown in italics, an interdomain linker and C-terminal tag are underlined, and the S310F substitution is bold/underlined.

(SEQ ID NO: 68)
MELAALCRWGLLLALLPPGAAS
TQVCTGTDMKLRLPASPETHLDMLRHLYQGCQVVQGNLELTYLPTNASLSFLQDIQEVQGYVLIAHNQVRQVPLQRL
RIVRGTQLFEDNYALAVLDNGDPLNNTTPVTGASPGGLRELQLRSLTEILKGGVLIQRNPQLCYQDTILWKDIFHKN
NQLALTLIDTNRSRACHPCSPMCKGSRCWGESSEDCQSLTRTVCAGGCARCKGPLPTDCCHEQCAAGCTGPKHSDCL
ACLHFNHSGICELHCPALVTYNTDTFESMPNPEGRYTFGASCVTACPYNYLSTDVGFCTLVCPLHNQEVTAEDGTQR
CEKCSKPCARVCYGLGMEHLREVRAVTSANIQEFAGCKKIFGSLAFLPESEDGDPASNTAPLQPEQLQVFETLEEIT
GYLYISAWPDSLPDLSVFQNLQVIRGRILHNGAYSLTLQGLGISWLGLRSLRELGSGLALIHHNTHLCFVHTVPWDQ
LERNPHQALLHTANRPEDECVGEGLACHQLCARGHCWGPGPTQCVNCSQFLRGQECVEECRVLQGLPREYVNARHCL
PCHPECQPQNGSVTCFGPEADQCVACAHYKDPPFCVARCPSGVKPDLSYMPIWKEPDEEGACQPCPINCTHSCVDLD
DKGCPAEQRASPLT
PGSRS
PKSCDKTHTCPPCPAPELLGGPSVFLFPPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQGNVFSCSVMHEALHNHYTQKSLSLSPGK
LEGGGGLNDIFEAQKIEWHESRHHHHHH

In the HER2 S310Y-Fc shown below, the secretion signal is shown in italics, an interdomain linker and C-terminal tag are underlined, and the S310Y substitution is bold/underlined.

(SEQ ID NO: 69)
MELAALCRWGLLLALLPPGAAS
TQVCTGTDMKLRLPASPETHLDMLRHLYQGCQVVQGNLELTYLPTNASLSFLQDIQEVQGYVLIAHNQVRQVPLQRL
RIVRGTQLFEDNYALAVLDNGDPLNNTTPVTGASPGGLRELQLRSLTEILKGGVLIQRNPQLCYQDTILWKDIFHKN
NQLALTLIDTNRSRACHPCSPMCKGSRCWGESSEDCQSLTRTVCAGGCARCKGPLPTDCCHEQCAAGCTGPKHSDCL
ACLHFNHSGICELHCPALVTYNTDTFESMPNPEGRYTFGASCVTACPYNYLSTDVGYCTLVCPLHNQEVTAEDGTQR
CEKCSKPCARVCYGLGMEHLREVRAVTSANIQEFAGCKKIFGSLAFLPESEDGDPASNTAPLQPEQLQVFETLEEIT
GYLYISAWPDSLPDLSVFQNLQVIRGRILHNGAYSLTLQGLGISWLGLRSLRELGSGLALIHHNTHLCFVHTVPWDQ
LERNPHQALLHTANRPEDECVGEGLACHQLCARGHCWGPGPTQCVNCSQFLRGQECVEECRVLQGLPREYVNARHCL
PCHPECQPQNGSVTCFGPEADQCVACAHYKDPPFCVARCPSGVKPDLSYMPIWKEPDEEGACQPCPINCTHSCVDLD
DKGCPAEQRASPLT
PGSRS
PKSCDKTHTCPPCPAPELLGGPSVFLFPPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQGNVFSCSVMHEALHNHYTQKSLSLSPGK
LEGGGGLNDIFEAQKIEWHESRHHHHHH

Exemplary Antibody Clones Binding to the HER2 S310F mutant: CDR residues (Kabat scheme) in bold. The actual sequences used in the examples contain additional residues at the N-terminus of VL and VH, i.e., the VL sequence starts with SDIQM, and the VH sequences starts with EISEVQL. These cloning artifacts are not expected to alter the functions of antibodies.

•Clone 11 (aka 11-1) (SEQ ID NOS: 37 and 36, respectively)
VL:
DIQMTQSPSSLSASVGDRVTITCRASQSVSSAVAWYQQKPGKAPKLLIYSASSLYSGVPSRFSGSRSG
TDFTLTISSLQPEDFATYYCQQSSSSLITFGQGTKVEIKRTV
VH:
EVQLVESGGGLVQPGGSLRLSCAASGFTVYSSSIHWVRQAPGKGLEWVASISSYYGYTSYADSVKGRF
TISADTSKNTAYLQMNSLRAEDTAVYYCARYGNYTMHQYGSWEQMPAFDYWGQGTLVTVSS
•Clone 7-20 (SEQ ID NOS: 60 and 59, respectively)
VL:
DIQMTQSPSSLSASVGDRVTITCRASQSVSSAVAWYQQKPGKAPKLLIYSASSLYSGVPSRFSGSRSG
TDFTLTISSLQPEDFATYYCQQYNLRKDLVTFGQGTKVEIKRTV
VH:
EVQLVESGGGLVQPGGSLRLSCAASGFTISYSSIHWVRQAPGKGLEWVASISSYYGSTSYADSVKGRF
TISADT SKNTAYLQMNSLRAEDTAVYYCARGKYSWGYYYMDSSMFAMDYWGQGTLVTVSS
•Clone 10-20 (SEQ ID NOS: 62 and 61, respectively)
VL:
DIQMTQSPSSLSASVGDRVTITCRASQSVSSAVAWYQQKPGKAPKLLIYSASSLYSGVPSRFSGSRSG
TDFTLTISSLQPEDFATYYCQQYWDYSLITFGQGTKVEIKRTV
VH:
EVQLVESGGGLVQPGGSLRLSCAASGFTVSSSSIHWVRQAPGKGLEWVAYISPSSGSTYYADSVKGRF
TISADTSKNTAYLQMNSLRAEDTAVYYCARSFYFYAYWSHEWHYAMDYWGQGTLVTVSS
•Clone 20-20 (SEQ ID NOSL 66 and 65, respectively)
VL:
DIQMTQSPSSLSASVGDRVTITCRASQSVSSAVAWYQQKPGKAPKLLIYSASSLYSGVPSRFSGSRSG
TDFTLTISSLQPEDFATYYCQQSLNYSGLITFGQGTKVEIKRTV
VH:
EVQLVESGGGLVQPGGSLRLSCAASGFTLSYYSIHWVRQAPGKGLEWVASIYSSYGYTSYADSVKGRF
TISADTSKNTAYLQMNSLRAEDTAVYYCARYWSEYWQYYPAMDYWGQGTLVTVSS
•Clone 25-20 (SEQ ID NOS: 64 and 63, respectively)
VL:
DIQMTQSPSSLSASVGDRVTITCRASQSVSSAVAWYQQKPGKAPKLLIYSASSLYSGVPSRFSGSRSG
TDFTLTISSLQPEDFATYYCQQSSPGWPVTFGQGTKVEIKRTV
VH:
EVQLVESGGGLVQPGGSLRLSCAASGFTLSSSYIHWVRQAPGKGLEWVASIYPSYGSTYYADSVKGRF
TISADTSKNTAYLQMNSLRAEDTAVYYCARQVGVYQYVAPYWGNVWAMDYWGQGTLVTVSS
•CH2 (SEQ ID NOS: 40 and 39, respectively)
VL:
DIQMTQSPSSLSASVGDRVTITCRASQSVSSAVAWYQQKPGKAPKLLIYSASSLYSGVPSRFSGSRSG
TDFTLTISSLQPEDFATYYCQQSSSSLITFGQGTKVEIKRTV
VH:
EVQLVESGGGLVQPGGSLRLSCAASGFTFSRTSIHWVRQAPGKGLEWVAEISSYDGYTDYADSVKGRF
TISADTSKNTAYLQMNSLRAEDTAVYYCARYGNYTMHQYGSWEQMPAFDYWGQGTLVTVSS
•CH10v (SEQ ID NOS: 43 and 42, respectively)
VL:
DIQMTQSPSSLSASVGDRVTITCRASQSVSSAVAWYQQKPGKAPKLLIYSASSLYSGVPSRFSGSRSG
TDFTLTISSLQPEDFATYYCQQSSSSLITFGQGTKVEIKRTV
VH:
EVQLVESGGGLVQPGGSLRLSCAASGFTFSTTSIHWVRQAPGKGLEWVAEISSYNGDTDYADSVKGRF
TISADTSKNTAYLQMNSLRAEDTAVYYCARYGNYTMHQYGSWEQMPAFDYWGQGTLVTVSS
•CH15 (SEQ ID NOS: 46 and 45, respectively)
VL:
DIQMTQSPSSLSASVGDRVTITCRASQSVSSAVAWYQQKPGKAPKLLIYSASSLYSGVPSRFSGSRSG
TDFTLTISSLQPEDFATYYCQQSSSSLITFGQGTKVEIKRTV
VH:
EVQLVESGGGLVQPGGSLRLSCAASGFTFSGNYIHWVRQAPGKGLEWVASIYSAGGYTDYADSVKGRF
TISADTSKNTAYLQMNSLRAEDTAVYYCARYGNYTMHQYGSWEQMPAFDYWGQGTLVTVSS
•CL1 (SEQ ID NOS: 49 and 48, respectively)
VL:
DIQMTQSPSSLSASVGDRVTITCRASQSVSSAVAWYQQKPGKAPKLLIYSASSLYSGVPSRFSGSRSG
TDFTLTISSLQPEDFATYYCQQSSSSLITFGQGTKVEIKRTV
VH:
EVQLVESGGGLVQPGGSLRLSCAASGFTFSTTAIHWVRQAPGKGLEWVASIASYNGDTDYADSVKGRF
TISADTSKNTAYLQMNSLRAEDTAVYYCARYGNYTMHQYGSWEQMPAFDYWGQGTLVTVSS
•CL3 (SEQ ID NOS: 52 and 51, respectively)
VL:
DIQMTQSPSSLSASVGDRVTITCRASQSVSSAVAWYQQKPGKAPKLLIYSASSLYSGVPSRFSGSRSG
TDFTLTISSLQPEDFATYYCQQSSSSLITFGQGTKVEIKRTV
VH:
EVQLVESGGGLVQPGGSLRLSCAASGFTFTRTDIHWVRQAPGKGLEWVASIDSNNGSTYYADSVKGRF
TISADTSKNTAYLQMNSLRAEDTAVYYCARYGNYTMHQYGSWEQMPAFDYWGQGTLVTVSS
•CL6 (SEQ ID NOS: 55 and 54, respectively)
VL:
DIQMTQSPSSLSASVGDRVTITCRASQSVSSAVAWYQQKPGKAPKLLIYSASSLYSGVPSRFSGSRSG
TDFTLTISSLQPEDFATYYCQQSSSSLITFGQGTKVEIKRTV
VH:
EVQLVESGGGLVQPGGSLRLSCAASGFTFTGSAIHWVRQAPGKGLEWVASIDSYDGDTDYADSVKGRF
TISADTSKNTAYLQMNSLRAEDTAVYYCARYGNYTMHQYGSWEQMPAFDYWGQGTLVTVSS
•CL11 (SEQ ID NOS: 58 and 57, respectively)
VL:
DIQMTQSPSSLSASVGDRVTITCRASQSVSSAVAWYQQKPGKAPKLLIYSASSLYSGVPSRFSGSRSG
TDFTLTISSLQPEDFATYYCQQSSSSLITFGQGTKVEIKRTV
VH:
EVQLVESGGGLVQPGGSLRLSCAASGFTFSATSIHWVRQAPGKGLEWVAEITSYNGSTDYADSVKGRF
TISADTSKNTAYLQMNSLRAEDTAVYYCARYGNYTMHQYGSWEQMPAFDYWGQGTLVTVSS
•CH15 hIgG1 (SEQ ID NOS: 143 and 142, respectively)
Light chain:
DIQMTQSPSSLSASVGDRVTITCRASQSVSSAVAWYQQKPGKAPKLLIYSASSLYSGVPSRFSGSRSG
TDFTLTISSLQPEDFATYYCQQSSSSLITFGQGTKVEIKRTVAAPSVFIFPPSDEQLKSGTASVVCLL
NNFYPREAKVQWKVDNALQSGNSQESVTEQDSKDSTYSLSSTLTLSKADYEKHKVYACEVTHQGLSSP
VTKSFNRGEC
Heavy chain:
EVQLVESGGGLVQPGGSLRLSCAASGFTFSGNYIHWVRQAPGKGLEWVASIYSAGGYTDYADSVKGRF
TISADTSKNTAYLQMNSLRAEDTAVYYCARYGNYTMHQYGSWEQMPAFDYWGQGTLVTVSSASTKGPS
VFPLAPSSKSTSGGTAALGCLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPSVFLFPPKPKDTLMISRTPEVT
CVVVDVSHEDPEVKFNWYVDGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK

Exemplary Clone 11 Mutants: CDR residues (Kabat scheme) are shown in bold and mutations are underlined. For each mutant, the V_{L} chain is SEQ ID NO: 40, and the V_{H} chain is shown below.

•Clone 11 Y95S V_{H} (SEQ ID NO: 158)
EVQLVESGGGLVQPGGSLRLSCAASGFTVYSSSIHWVRQAPGKGLEWVASISSYYGYTSYADSVKGRF
TISADTSKNTAYLQMNSLRAEDTAVYYCARSGNYTMHQYGSWEQMPAFDYWGQGTLVTVSS
•Clone 11_G96S V_{H} (SEQ ID NO: 159)
EVQLVESGGGLVQPGGSLRLSCAASGFTVYSSSIHWVRQAPGKGLEWVASISSYYGYTSYADSVKGRF
TISADTSKNTAYLQMNSLRAEDTAVYYCARYSNYTMHQYGSWEQMPAFDYWGQGTLVTVSS
•Clone 11 N97S V_{H} (SEQ ID NO: 160)
EVQLVESGGGLVQPGGSLRLSCAASGFTVYSSSIHWVRQAPGKGLEWVASISSYYGYTSYADSVKGRF
TISADTSKNTAYLQMNSLRAEDTAVYYCARYGSYTMHQYGSWEQMPAFDYWGQGTLVTVSS
•Clone 11 Y98S V_{H} (SEQ ID NO: 161)
EVQLVESGGGLVQPGGSLRLSCAASGFTVYSSSIHWVRQAPGKGLEWVASISSYYGYTSYADSVKGRF
TISADTSKNTAYLQMNSLRAEDTAVYYCARYGNSTMHQYGSWEQMPAFDYWGQGTLVTVSS
•Clone 11_T99S V_{H} (SEQ ID NO: 162)
EVQLVESGGGLVQPGGSLRLSCAASGFTVYSSSIHWVRQAPGKGLEWVASISSYYGYTSYADSVKGRF
TISADTSKNTAYLQMNSLRAEDTAVYYCARYGNYSMHQYGSWEQMPAFDYWGQGTLVTVSS
•Clone 11_M100S V_{H} (SEQ ID NO: 163)
EVQLVESGGGLVQPGGSLRLSCAASGFTVYSSSIHWVRQAPGKGLEWVASISSYYGYTSYADSVKGRF
TISADTSKNTAYLQMNSLRAEDTAVYYCARYGNYTSHQYGSWEQMPAFDYWGQGTLVTVSS
•Clone 11_H100aS V_{H} (SEQ ID NO: 164)
EVQLVESGGGLVQPGGSLRLSCAASGFTVYSSSIHWVRQAPGKGLEWVASISSYYGYTSYADSVKGRF
TISADTSKNTAYLQMNSLRAEDTAVYYCARYGNYTMSQYGSWEQMPAFDYWGQGTLVTVSS
•Clone 11_Q100bS V_{H} (SEQ ID NO: 165)
EVQLVESGGGLVQPGGSLRLSCAASGFTVYSSSIHWVRQAPGKGLEWVASISSYYGYTSYADSVKGRF
TISADTSKNTAYLQMNSLRAEDTAVYYCARYGNYTMHSYGSWEQMPAFDYWGQGTLVTVSS
•Clone 11 Y100cS V_{H} (SEQ ID NO: 166)
EVQLVESGGGLVQPGGSLRLSCAASGFTVYSSSIHWVRQAPGKGLEWVASISSYYGYTSYADSVKGRF
TISADTSKNTAYLQMNSLRAEDTAVYYCARYGNYTMHQSGSWEQMPAFDYWGQGTLVTVSS
•Clone 11_G100dS V_{H} (SEQ ID NO: 167)
EVQLVESGGGLVQPGGSLRLSCAASGFTVYSSSIHWVRQAPGKGLEWVASISSYYGYTSYADSVKGRF
TISADTSKNTAYLQMNSLRAEDTAVYYCARYGNYTMHQYSSWEQMPAFDYWGQGTLVTVSS
•Clone 11 S100eG V_{H} (SEQ ID NO: 168)
EVQLVESGGGLVQPGGSLRLSCAASGFTVYSSSIHWVRQAPGKGLEWVASISSYYGYTSYADSVKGRF
TISADTSKNTAYLQMNSLRAEDTAVYYCARYGNYTMHQYGGWEQMPAFDYWGQGTLVTVSS
•Clone 11 W100fS V_{H} (SEQ ID NO: 169)
EVQLVESGGGLVQPGGSLRLSCAASGFTVYSSSIHWVRQAPGKGLEWVASISSYYGYTSYADSVKGRF
TISADTSKNTAYLQMNSLRAEDTAVYYCARYGNYTMHQYGSSEQMPAFDYWGQGTLVTVSS
•Clone 11 E100gS V_{H} (SEQ ID NO: 170)
EVQLVESGGGLVQPGGSLRLSCAASGFTVYSSSIHWVRQAPGKGLEWVASISSYYGYTSYADSVKGRF
TISADTSKNTAYLQMNSLRAEDTAVYYCARYGNYTMHQYGSWSQMPAFDYWGQGTLVTVSS
•Clone 11_Q100hS V_{H} (SEQ ID NO: 171)
EVQLVESGGGLVQPGGSLRLSCAASGFTVYSSSIHWVRQAPGKGLEWVASISSYYGYTSYADSVKGRF
TISADTSKNTAYLQMNSLRAEDTAVYYCARYGNYTMHQYGSWESMPAFDYWGQGTLVTVSS
•Clone 11_M100iS V_{H} (SEQ ID NO: 172)
EVQLVESGGGLVQPGGSLRLSCAASGFTVYSSSIHWVRQAPGKGLEWVASISSYYGYTSYADSVKGRF
TISADTSKNTAYLQMNSLRAEDTAVYYCARYGNYTMHQYGSWEQSPAFDYWGQGTLVTVSS
•Clone 11 P100jS V_{H} (SEQ ID NO: 173)
EVQLVESGGGLVQPGGSLRLSCAASGFTVYSSSIHWVRQAPGKGLEWVASISSYYGYTSYADSVKGRF
TISADT SKNTAYLQMNSLRAEDTAVYYCARYGNYTMHQYGSWEQMSAFDYWGQGTLVTVSS
•Clone 11 A100KS V_{H} (SEQ ID NO: 174)
EVQLVESGGGLVQPGGSLRLSCAASGFTVYSSSIHWVRQAPGKGLEWVASISSYYGYTSYADSVKGRF
TISADTSKNTAYLQMNSLRAEDTAVYYCARYGNYTMHQYGSWEQMPSFDYWGQGTLVTVSS
•Clone 11 F1001S V_{H} (SEQ ID NO: 175)
EVQLVESGGGLVQPGGSLRLSCAASGFTVYSSSIHWVRQAPGKGLEWVASISSYYGYTSYADSVKGRF
TISADTSKNTAYLQMNSLRAEDTAVYYCARYGNYTMHQYGSWEQMPASDYWGQGTLVTVSS

### Example 2—Development of Antibody Clone CH15V

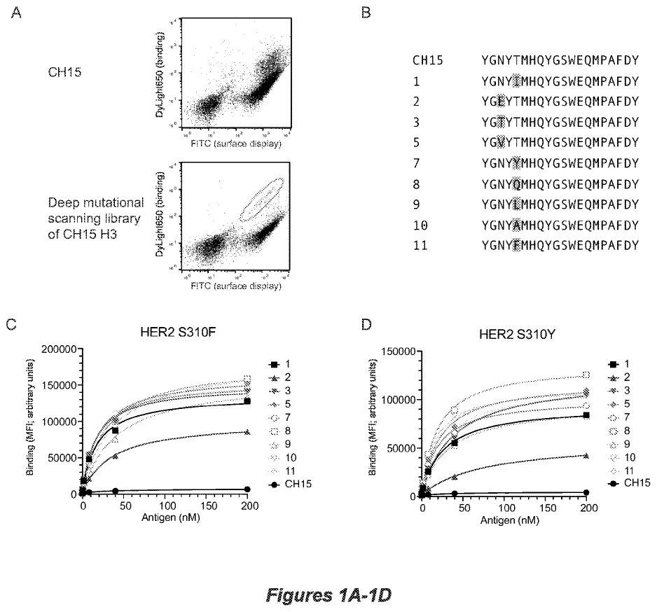

By performing scanning mutagenesis of CDR-H3 of CH15 in the yeast display format, a small subset of mutants was identified that showed substantial increase in antigen binding to HER2 (S310F) (FIGS. 1A, 1C, 1D). These clones contained mutations at N97 or T99 (FIG. 1B). N97 and T99 are a part of a glycosylation consensus, NX(S/T), indicating that N97 is glycosylated when CH15 is produced using Eukaryotic host cells. Such aberrant glycosylation in CDR-H3 is expected to impair antigen binding. Thus, N97 was replaced with Val, resulting in a new clone, CH15V.

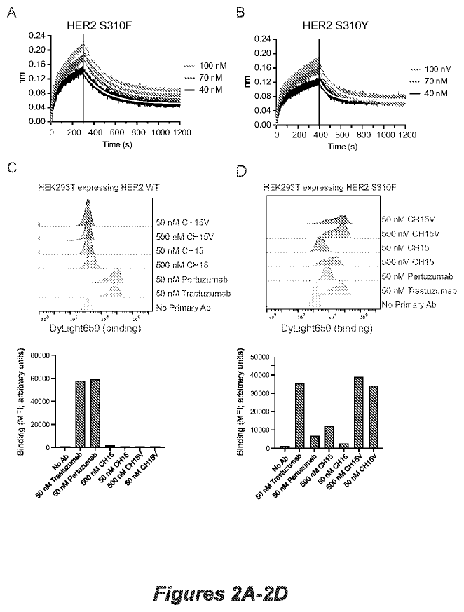

CH15V exhibited high affinity to HER2 mutants as assessed using biolayer interferometry (BLI) (FIG. 2A, 2B). To assess cell binding capability of these antibodies, HEK293T cells overexpressing HER2 WT or HER2 S310F were prepared and utilized for screening. CH15V in the human IgG1 format specifically bound to HEK293T cells expressing HER2 (S310F) but not to the cells expressing HER2 WT (FIG. 2C, 2D) and showed stronger binding signals than CH15.

### Example 3—Deep Mutational Scanning of Antibody Clone CH15V

Deep mutational scanning of CH15V was carried out in which CDR-H1 and CDR-H3 residues of CH15V were mutated to all amino acid types, one residue at a time, to create a yeast-display library. By recovering clones that had binding profiles similar to CH15V and those that showed marginal binding to the antigen (HER2 (S310F)-Fc, mutations that are permissive were deduced (Table 4).

TABLE 4
Permissive mutations in CH15V CDR-H1 and CDR-H3 deduced from deep mutational scanning

| Position | Parental | Allowed residue | |
|---|---|---|---|
| (Heavy | aa | (excluding parental) | Not-allowed residue |
| Chain)¹ | residue | (200 nM target², positive sorting) | (200 nM target², negative sorting) |
| 29 | F | W, Y | A, C, D, I, T, V |
| 30 | S | A, F, G, H, I, M, N, Q, R, T, V, | D, E |
| | | W, Y | |
| 31 | G | | D, E, K, P |
| 32 | N | A, C, F, H, L, M, S | R |
| 33 | Y | E, F | A, C, D, G, I, K, L, M, N, P, R, S, T, V |
| 95 | Y | F | A, C, D, E, H, I, K, L, M, N, P, Q, R, |
| | | | S, T, V |
| 96 | G | | A, C, D, E, H, I, K, L, N, P, Q, R, S, |
| | | | T, W, Y |
| 97 | V | E, H, I, L, M, P, Q, T | G |
| 98 | Y | | A, C, E, G, H, I, K, L, N, P, R, S, T, V |
| 99 | T | A, E, H, I, K, L, M, N, Q, R, S | P |
| 100 | M | L | D, E, F, G, K, N, R, S, T |
| 100A | H | A, K, M, Q, R | D, N, P |
| 100B | Q | A, F, H, I, K, L, M, N, R, S, T | |
| 100C | Y | A, F, G, R | D, E, I, N, P, T, V |
| 100D | G | | C, D, E, F, H, K, L, M, N, P, Q, R, S, |
| | | | T, V, Y |
| 100E | S | | E, F, G, H, I, K, L, M, N, P, Q, R, T, |
| | | | V, W, Y |
| 100F | W | | A, C, D, E, F, G, H, I, K, L, M, N, P, |
| | | | Q, R, S, T, V |
| 100G | E | Q, V | F, P, Y |
| 100H | Q | M, S | D, G, K, R, W |
| 100I | M | F, L, R, T | G, P, Y |
| 100J | P | | G, K, R |
| 100K | A | Y | D, E, K, P, Q, R |
| 100L | F | H, Y | G, K, P, R, W |

¹Positions definition according to Kabat numbering scheme.
²target: bHER2 S310F (23-652)-Fc, i.e., biotinylated HER2 (23-652) harboring the S310F mutation fused with human Fc.

### Example 4—Inhibition of Cell Growth

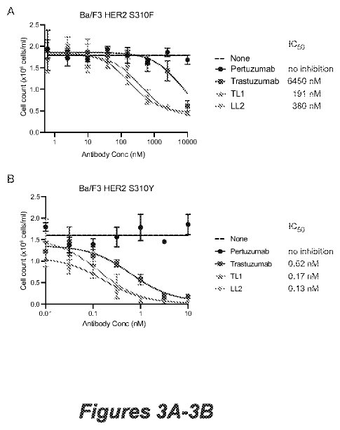

Two antibodies, TL1 and LL2, were tested for efficacy inhibiting cell proliferation driven by the signaling activity of HER2 S310F/Y. Ba/F3 cells engineered to express either HER2 S310F or S310Y were used for this assay. Expression of an activation mutant of HER2 results in proliferation of Ba/F3 cells in the absence of IL-3 supplementation. Cells were seeded at 100,000 cells/ml with TL1, LL2, pertuzumab and trastuzumab at various concentrations, and cell growth was monitored by counting on day 3 (FIGS. 3A-3B). Trastuzumab binds to both HER2 WT and S310F/Y mutants, whereas pertuzumab binds to WT but not to S310F/Y.

TL1 and LL2 showed strong growth inhibition of Ba/F3 cells expressing HER2 S310Y with IC₅₀ values in the subnanomolar range (FIG. 3B), demonstrating the potential of TL1 and LL2 as signaling inhibitors selective to HER2 S310Y. Their IC₅₀ values were ~5-fold smaller than that of trastuzumab. Pertuzumab showed no inhibition as expected.

TL1 and LL2 inhibited the growth of Ba/F3 cells expressing HER2 S310F signaling more potently than trastuzumab (FIG. 3A). Pertuzumab showed no inhibition as expected. Although the inhibition of HER2 S310Y-mediated growth by TL1 and LL2 was weaker than their inhibition of HER2 S310Y, with sub-micromolar IC₅₀ values, it is remarkable that TL1 and LL2 were capable of inhibiting this HER2 mutant more potently than trastuzumab (FIG. 3B). Together, these results demonstrate that TL1 and LL2 are selective inhibitors of cell proliferation driven by HER2 S310F and S310Y.

### Materials and Methods for Examples 5-11

### Expression and Purification of HER Antigens

Synthetic genes encoding the ectodomains of HER isotypes (HER1, HER2 and HER3) were cloned into the plasmid pBCAG in such a way that they are fused C-terminally with hIgG1 Fc either with knob mutations (T366W) or with hole mutations (T366S, L368A, Y407V) (Ridgway et al., “‘Knobs-into-holes’ Engineering of Antibody CH3 Domains for Heavy Chain Heterodimerization,” Protein Engineering, Design and Selection 9:617-621 (1996); Atwell et al., “Stable Heterodimers from Remodeling the Domain Interface of a Homodimer Using a Phage Display Library,” J. Mol. Biol. 270:26-35 (1997), each of which is hereby incorporated by reference in its entirety). Avi-tag and polyhistidine tag were added to the C-terminus of Fc, except for constructs with Fc Knob. A vector containing only the Fc domain with hole mutations was prepared to produce monomeric HER antigens. Altogether, following constructs were produced: Fc Hole, HER1 (1-645)-Fc Hole, HER2 (1-652)-Fc, HER2 S310F (1-652)-Fc Knob, HER2 S310Y (1-652)-Fc Knob, HER3 (1-643)-Fc Hole.

The plasmids encoding the designed antigens were purified using Midiprep kit (Qiagen) and used to transfect Expi293F™ cells following the standard protocol from the vendor (Thermo Fisher Scientific) to 100 ml of Expi293F™ cells at 3×10⁶ cells per ml. Transfected cells were incubated at 37° C. with 8% CO2 and harvested on day 5 post-transfection. After centrifugation at 3,000 rpm to remove cells, Tris HCl buffer (pH 8.0) was added to the culture supernatant at a final concentration of 50 mM, and the supernatant was dialyzed against 20 mM Tris HCl buffer pH 7.5 containing 100 mM NaCl buffer for ≥4 hours. After dialysis, the supernatant was filtered and loaded onto a HisTrap excel column (Cytiva) preequilibrated in 50 mM Tris HCl buffer pH 8.0 containing 150 mM NaCl and eluted with 50 mM Tris HCl buffer pH 8.0 containing 0.5 M imidazole. Elution fractions from the HisTrap column containing the expressed protein were pooled. The biotinylation reaction was initiated by adding 50 mM Bicine, 10 mM magnesium acetate, 10 mM ATP, 0.5 mM biotin and 1 μM BirA (all final concentrations) to the pooled sample. The reaction mixture was incubated at 30° C. for 1 hour and dialyzed against gel filtration buffer (20 mM Tris HCl buffer pH 7.5 containing 150 mM NaCl). Finally, the sample was loaded onto a Superdex S200 Increase 10/300 GL column (Cytiva) for size exclusion chromatography. Eluted proteins were inspected for dimerization by running SDS-PAGE gel in reducing and non-reducing conditions. Fractions containing the intended dimer were pooled, concentrated, aliquoted and stored at −80° C. until needed.

### Expression and Purification of Fab:

Fab were purified following the previously described protocol (Miller et al., “T Cell Receptor-like Recognition of Tumor in vivo by Synthetic Antibody Fragment,” PLoS One 7:e43746 (2012), which is hereby incorporated by reference in its entirety). Briefly, the genes encoding the VH and VL domains of an antibody of interest were subcloned into a bacterial expression vector engineered for Golden Gate Assembly (New England Biolabs). Two BsaI sites were introduced into the RH2.2 Fab expression vector so that the enzyme digests at 9^{th} and 10^{th} position (encoding the SS sequence) of the light chain and after the “elbow” region of the heavy chain (encoding the VF sequence). A pre-existing BsaI site in the bla gene was removed by introducing a silent mutation. Also, a region encoding the heavy chain N-terminal to the hinge region was deleted so that a correctly cloned Fab gene can be easily identified. The rigidified elbow sequence (Bailey et al., “Locking the Elbow: Improved Antibody Fab Fragments as Chaperones for Structure Determination,” J Mol Biol 430:337-347 (2018), which is hereby incorporated by reference in its entirety) was introduced to the Fab gene of interest by PCR and cloned into the recipient vector by using Golden Gate Assembly. The vector was used to transform the 55244 E. coli strain (ATCC). The cells were grown in 800 ml of terrific broth media overnight at 30° C. with shaking. After cell lysis using sonication and centrifugation, Fab in the supernatant was captured on a HiTrap Protein G affinity column (Cytiva) and eluted with 0.2 M glycine pH 2.0. The eluted fractions were neutralized, pooled and dialyzed against 20 mM Tris HCl buffer pH 7.5 containing 150 mM NaCl overnight. The sample was concentrated and aliquoted. The purity of the purified protein was confirmed with SDS-PAGE.

Expression and Purification of hIgG:

pFUSE plasmids (Invivogen) were used to construct expression vectors for the light and heavy chain constructs of our antibodies with the hIgG1 framework. These vectors were used to express antibodies using ExpiCHO cells, following the standard protocol for the ExpiCHO™ expression system (Thermo Fisher Scientific). 100 ml of ExpiCHO cells were transfected at 6×10⁶ cells per ml with 40 μg of heavy chain and 60 μg of light chain plasmid. The supernatant was collected on day 7 post-transfection and passed through a HiTrap Protein G affinity column (Cytiva). The captured hIgG was eluted with 0.2 M glycine pH 2.0 and immediately neutralized with Tris HCl buffer pH 8.0 and dialyzed against 20 mM Tris HCl buffer pH 7.5 containing 150 mM NaCl overnight. Next day, the sample was concentrated, aliquoted and stored at −80° C. until further usage. The purity and correct assembly of heavy and light chains were confirmed with SDS-PAGE in reducing and non-reducing conditions.

Expression and Purification of scDb:

The scDb genes were constructed as described previously (Hattori et al., “Creating MHC-Restricted Neoantigens with Covalent Inhibitors That Can Be Targeted by Immune Therapy,” Cancer Discov 13:132-145 (2023), which is hereby incorporated by reference in its entirety). Briefly, the genes encoding the variable domains of an anti-human CD3ε monoclonal antibody, clone UCHT1 (Beverley and Callard, “Distinctive Functional Characteristics of Human “T” Lymphocytes Defined by E Rosetting or a Monoclonal Anti-T cell Antibody,” Eur J Immunol 11:329-334 (1981), which is hereby incorporated by reference in its entirety), and the developed antibodies were cloned into the pBCAG vector followed by the gene encoding a His₆ tag at the C-terminus. 50 ml of Expi293F cells were transfected with a resulting vector at the cell density of 3×10⁶ cells per ml following the Expi293 Expression System protocol (Thermo Fisher Scientific). Transfected cells were incubated at 37° C. with 8% CO₂ and harvested after 5 days. The supernatant was dialyzed for at least 4 hours against 20 mM Tris HCl buffer pH 7.5 containing 100 mM NaCl before loading onto a HisTrap excel column (Cytiva) followed by size-exclusion chromatography using a Superdex 200 Increase 10/300 GL column (Cytiva). The purity of scDb was assessed by running SDS-PAGE in reducing and non-reducing conditions.

### Antibody Discovery and Affinity Maturation:

The sorting of a synthetic human antibody library was performed as described previously (Oury et al., “Mechanism of Disease and Therapeutic Rescue of Dok7 Congenital Myasthenia,” Nature 595:404-408 (2021); Miller et al., “T Cell Receptor-like Recognition of Tumor in vivo by Synthetic Antibody Fragment,” PLoS One 7:e43746 (2012), each of which is hereby incorporated by reference in its entirety). Briefly, the phage library was incubated with HER2 mutants at concentrations of 20 nM in the first and second rounds, 5 nM for third round and 1 nM for fourth round. In the second and later rounds, biotinylated HER2 WT-Fc was immobilized on the Streptavidin MagneSphere particles (Promega) to pre-clear phages that bind to HER2 WT prior to the selection against HER2 mutants (Teng et al., “Engineering Binders with Exceptional Selectivity,” Methods Mol Biol 2491:143-154 (2022), which is hereby incorporated by reference in its entirety). Phage clones from the sorted library were assessed by phage ELISA (Miller et al., “T Cell Receptor-like Recognition of Tumor in vivo by Synthetic Antibody Fragment,” PLoS One 7:e43746 (2012), which is hereby incorporated by reference in its entirety).

Affinity maturation of clone 11 was performed using phage display as follows. First, a phage library was constructed where the residues in CDR H1 and CDR H2 in Clone 11 were randomized as described previously (Lee et al., “High-affinity Human Antibodies from Phage-displayed Synthetic Fab Libraries with a Single Framework Scaffold,” J Mol Biol 340:1073-1093 (2004), which is hereby incorporated by reference in its entirety). The phage library was sorted against HEK293T cells expressing a high level of S310F (first, second and third rounds) and a low level of S310F (fourth round). In all rounds, phage solutions were first incubated with HEK293T cells expressing a high level of WT HER2 to eliminate clones that cross-react with WT HER2. Phage clones from the sorted library were first characterized by phage ELISA using recombinant HER2 S310F/Y and HER2 WT proteins. Then, a subset of isolated clones was converted into the hIgG format. The hIgG clones were assessed by the cell-based binding assay using HEK293T cells expressing HER2 mutants and WT, and CH15 was identified.

CH15V was generated by mutating Asn at position 101 in the heavy chain of CH15 to Val. Affinity maturation of CH15V was done based on deep mutational scanning analyses on CDR H1, H3 of CH15V. CDR H1 and H3 were mutated one amino acid at a time using the NNK codon, where N is a mixture of A, T, G and C and K is a mixture of G and T. The library, constructed in a yeast-display format as described previously (Hattori et al., “Creating MHC-Restricted Neoantigens with Covalent Inhibitors That Can Be Targeted by Immune Therapy,” Cancer Discov 13:132-145 (2023), which is hereby incorporated by reference in its entirety) was sorted using 200 nM HER2 S310F-Fc for two rounds. Plasmids from the enriched pool and the original library were extracted using a Zymoprep Yeast Plasmid Miniprep II kit (Zymo Research Corporation). The scFv genes were amplified and sequenced on a MiSeq sequencer (Illumina). Sequencing data was analyzed using a custom python script (Hattori et al., “Creating MHC-Restricted Neoantigens with Covalent Inhibitors That Can Be Targeted by Immune Therapy,” Cancer Discov 13:132-145 (2023), which is hereby incorporated by reference in its entirety) to calculate the enrichment of amino acid at each position relative to the original unsorted library. The enrichment was plotted as a heatmap with log₂ fold change and was capped at maximum and minimum of 2 and −2.

An oligo pool was designed by combining permitted mutations in CDR-H1 and -H3 and synthesized (Twist Bioscience). CDR-L3 from the naive synthetic human antibody library was also combined with the oligo pool to make the third library in the yeast-display format. This library was sorted under stringent conditions, 1^{st} round with 5 nM of HER2 S310F-Fc and 2^{nd} round with 2.5 nM of HER2 S310F-Fc. A subset of isolated clones was screened for off-target binding to HER2 WT-Fc and for polyreactivity using biotinylated solubilized membrane protein, prepared following previously published protocol (Xu et al., “Addressing Polyspecificity of Antibodies Selected from an in vitro Yeast Presentation System: A FACS-based, High-throughput Selection and Analytical Tool,” Protein Engineering, Design and Selection 26:663-670 (2013), which is hereby incorporated by reference in its entirety). Clones with high affinity to S310F-Fc and S310Y-Fc were identified by performing binding titration in the yeast-display format. Candidate clones thus identified were converted into the Fab format for further studies.

### BLI Measurement:

BLI measurements were performed on an Octet RED96e instrument (Sartorius). A same solution was used for loading, association, dissociation and baseline steps: 20 mM Tris HCl buffer pH 7.5 containing 100 mM NaCl, 0.5% bovine serum albumin (BSA) and 0.005% Tween 20. Anti-Human Fc Capture 2 (AHC2) biosensors were loaded with 50 nM of HER-Fc antigen to have a final immobilization level in the range of 0.6 to 1.0 nm. Binding kinetics were measured in solutions containing Fab or IgG as the analyte. Data were fitted and analyzed with the Octet Data Analysis software v12.0.2.59 (Sartorius). For kinetic measurements with CH15V, local fitting with 1:1 binding model was applied to the kinetic data collected at 40, 70 and 100 nM of Fab and the values were averaged. For kinetic measurements of the fourth-generation antibodies, global fitting with a 1:1 binding model was applied to the data with the HER2 S310F/Y monomeric constructs with Fab concentrations at 5, 10 and 20 nM. For the HER2 S310F/Y homodimeric constructs, global fitting with a 2:1 binding model was used and Fab concentrations were 20, 40 and 80 nM. Kinetic measurement with HER2 WT was performed with Fab and human IgG at 100 nM. Lastly for kinetic measurements in the presence of a HER ligand, 50 nM of HER1/HER2 and HER3/HER2 heterodimers were pre-incubated with 75 nM of EGF and NRG1β, respectively, before starting the assay. AHC2 biosensors were loaded with a ligand-bound HER heterodimer and then dipped into buffer containing Fab at 40 nM. All the measurements were double referenced.

### Cell-Based Binding Assay:

The cell-based binding assay was performed with 50 nM of an antibody of interest except for the titration assay for which 0.4, 2, 10 and 50 nM antibody was used. Ice-cold PBS supplemented with 2% BSA was used to wash cells and for incubation with an antibody. After 30 min incubation with primary antibodies, cells were washed 3 times. Cells were then incubated for 30 min on ice with anti-human IgG specific against Fcγ fragment, conjugated with Alexa Fluor 647 (Jackson Immunoresearch) and washed 3 times with the buffer. An IntelliCyt iQue Screener PLUS flow cytometer (Sartorius) and FlowJo software (FlowJo) were used to analyze and quantify the fluorescence signals on the cells.

### Cryo-EM Sample Preparation and Data Collection:

To purify TL1 Fab and HER2 S310F (23-652)-Fc complex, the two proteins were mixed 1.2:1 ratio, with TL1 Fab slightly in excess and passed through gel filtration column in buffer 20 mM Tris pH 7.5, 150 mM NaCl. The complex formation was confirmed with SDS-PAGE gel and fractions containing the complex was pooled and concentrated. Fluorinated octyl maltoside (Anatrace) was added immediately before freezing to the final concentration of 0.7 mM. Three microliters of TL1 Fab/HER2 S310F (23-652)-Fc complex at 1.4 g·L⁻¹ were applied on in-house prepared gold foil grids and HER2 S310F (23-652)-Fc alone sample at 1.5 g·L⁻¹. Gold foil grids were prepared following a modified procedure previously described (Russo and Passmore, “Ultrastable Gold Substrates: Properties of a Support for High-Resolution Electron Cryomicroscopy of Biological Specimens,” J Struct Biol 193:33-44 (2016), which is hereby incorporated by reference in its entirety). Briefly, a 35 nm thick gold layer was deposited on Quantifoil 0.6/1 300 mesh grids using a Safematic CCU-010 evaporator (Safematic GmbH, Zizers Switzerland) using the following settings: processing pressure=1.5 e⁻² Torr; process current=30 mA. After coating, grids were flipped and placed in a Gatan Solarus plasma cleaner to remove carbon. Grids were plasma cleaned for 15 min in an O₂/Ar environment. The grids were glow-discharged before use for 30 sec in PELCO easiGlow Glow Discharge Cleaning System (Ted Pella Inc.). A Vitrobot Mark IV (Thermo Fisher Scientific) was used in all freezing with the following setting: wait time 30 s, blot time 4 s, blot force 5, chamber temperature 4° C. and chamber humidity 100%.

All the data collection was done at the NYU Cryo-EM Laboratory using Leginon 3.6 (Suloway et al., “Automated Molecular Microscopy: The New Leginon System,” J Struct Biol 151:41-60 (2005); Cheng et al., “Leginon: New Features and Applications,” Protein Sci 30:136-150 (2021), each of which is hereby incorporated by reference in its entirety). For HER2 S310F (23-652)-Fc alone grid, data were collected on a Talos Arctica (Thermo Fisher Scientific) with a K3 camera (Gatan) at magnification of 45,000×, in the super-resolution mode giving nominal pixel size of 0.430 Å with total dosage of 55.11 e per Å² across 50 frames. The defocus range was set to −1.5 to −2.5 μM. For the Fab and HER2 S310F complex, data were acquired on a Titan Krios (Thermo Fisher Scientific) with a K3 camera (Gatan) at magnification of 105,000×, at super-resolution mode giving nominal pixel size of 0.413 Å with total dosage of 57.43 e⁻ per Å₂ across 50 frames. The defocus range was set to −0.9 to −2.4 μM.

### Mammalian Cell Culture:

HEK293T cells were purchased from ATCC and maintained in Dulbecco's Modified Eagle Medium (Thermo Fisher Scientific) supplemented with 10% fetal bovine serum (FBS, Gemini Bio) and penicillin/streptomycin (Thermo Fisher Scientific) at 37° C. with 5% CO₂. Expi293F cells and ExpiCHO cells (Thermo Fisher Scientific) were maintained in Expi293 and ExpiCHO Expression Medium respectively (Thermo Fisher Scientific) at 37° C. with 8% CO₂. HTB-9 cells were purchased from ATCC and maintained in RPMI 1640 media (Thermo Fisher Scientific) supplemented with 10% fetal bovine serum (FBS, Gemini Bio) and penicillin/streptomycin (Thermo Fisher Scientific) at 37° C. with 5% CO₂.

NK-92 cells were purchased from ATCC and maintained in RPMI 1640 media (Thermo Fisher Scientific) with 10% FBS (Gemini Bio) and penicillin/streptomycin (Thermo Fisher Scientific) in the presence of 33.3 BRMP units per mL of human IL2 (Corning) at 37° C. with 5% CO.

T cells were expanded from purchased peripheral blood mononuclear cells (STEMCELL Technologies) using CTS OpTimizer T-cell Expansion SFM (Thermo Fisher Scientific). T cells were cultured in CTS OpTimizer T-cell Expansion SFM supplemented with L-glutamine (Thermo Fisher Scientific) and penicillin/streptomycin (Thermo Fisher Scientific) or in RPMI 1640 media (Thermo Fisher Scientific) with 10% FBS (Gemini Bio) and penicillin/streptomycin (Thermo Fisher Scientific) in the presence of 10 ng mL-1 of human IL7 and IL5 (Pepro Tech) at 37° C. with 5% CO. All cells were periodically checked for mycoplasma infection.

### Stable Transfection of HEK293T Cells:

Genes encoding HER2 S310F, HER2 S301Y and WT HER2 were cloned into pMXs vectors (Addgene). Production of retrovirus and retroviral transduction of HEK293T cells were performed as described previously (Teng et al., “Selective and Noncovalent Targeting of RAS Mutants for Inhibition and Degradation,” Nat Commun 12:2656 (2021), which is hereby incorporated by reference in its entirety). Briefly, retroviruses were produced by co-transfecting the packaging cell line GP2-293 using Lipofectamine 3000 (Thermo Fisher Scientific) with the pMXs vector derivatives and the virus envelope vector pVSV-G. The filtered viral supernatants were used for transducing HEK293T cells. After transduction, the cells were selected in complete media containing 20 μg mL⁻¹ blasticidin (InvivoGen). After selection, cells were stained with 50 nM Trastuzumab followed by anti-human IgG Fc-Alexa Fluor 647 (Jackson ImmunoResearch). The cells expressing a high or low level of HER2 mutants or WT HER2 were sorted using a FACSAria IIu SORP cell sorter (BD Bioscience). The sorted cells were maintained in complete media containing 20 μg mL⁻¹ blasticidin (InvivoGen). The expression of HER2 mutants and WT HER2 were verified via flow cytometry (FIG. 9)

### Cytotoxicity Assay:

For the ADCC assay, target cells were seeded in poly-D-lysine (Gibco) coated 96-well plates, 10,000 cells per well the day before the initiation of the assay. Next day, cells were stained with 5 μM calcein AM dye (Thermo Fisher Scientific) for 2 hrs. During the staining step, serial dilution of tested IgGs in media containing 2.5 mM probenecid acid, and human IL2 (33.3 BRMP units per mL) were prepared. After staining, the cells were washed and IgG containing media were added together with NK-92 cells. The effector-to-target ratio was set at 8:1, i.e., 80,000 NK-92 cells were added to each well. The plate was incubated at 37° C. for 3 hrs and the supernatant was transferred to a black 96-well plate. To produce a positive control for the maximal fluorescence (complete killing), Triton X-100 was added to the well at a final concentration of 2% and the plate was incubated for 5 min before the transfer to a black 96-well plate. Fluorescence signals were measured with a Synergy Neo2 hybrid multimode reader (BioTek) following the manufacturer's protocol and statistical analyses were performed using Prism 9 (GraphPad software). Cytotoxicity was calculated using the following formula:

$$Cytotoxicity ( \% ) = ( \frac{\begin{matrix}Sample well fluorescence \\ intensity - spontaneous intensity\end{matrix}}{\begin{matrix}maximal fluorescence intensity - \\ spontaneous fluorescence intensity\end{matrix}} ) \times 100 \%$$

The procedure for the cytotoxicity assay using scDb and T cells as the effector was similar to that for the ADCC assay, except for the following. Target cells were seeded at 5,000 cells per well and the effector-to-target ratio was 4 to 1. Incubation time with scDb was either 4 or 6.5 hrs. For the assay where the effect of EGF ligand was tested, EGF was incubated together with calcein AM and also added when media was changed to add scDb and T cells.

Cell Viability Assay with ADC:

Target cells were seeded in white 96-well plates at 2,000 cells per well the day before the initiation of the assay. The next day, media were replaced with fresh media containing 4 nM of Fab-ZAP (Advanced Targeting Systems) and an antibody of interest. Control well with no antibody but 4 nM of Fab-ZAP was also included. Three days post-incubation, cell viability was measured with PrestoBlue (Thermo Fisher Scientific) following the manufacturer's protocol with a Synergy Neo2 hybrid multimode reader (BioTek). Statistical analyses were performed using Prism 9 (GraphPad software). Percentage of cell viability was calculated using the following formula:

$$Cell viability ( \% ) = ( \frac{Sample fluorescence intensity}{Control well fluorescence intensity} ) \times 100 \%$$

### Example 5—Discovery of Antibodies Selective to HER2 S310F/Y

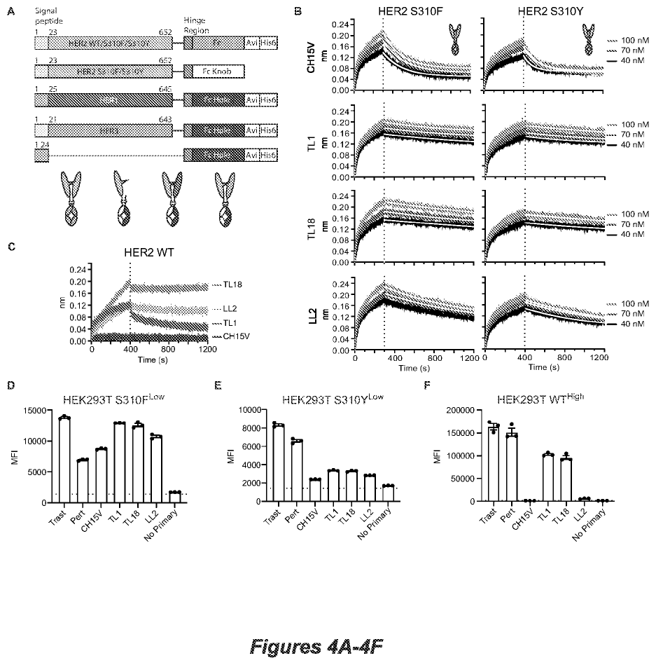

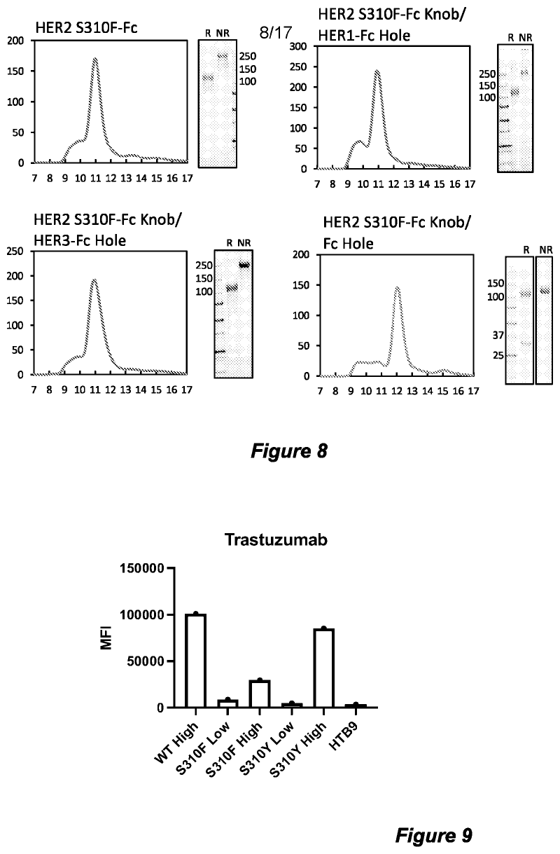

As the first step in antibody discovery, a set of HER2 constructs was designed as antigens (FIG. 4A). Due to general difficulties in handling membrane proteins, constructs were designed with only the water-soluble ectodomain of HER2 (residues 23-652), which were fused to the Fc region of human immunoglobulin G (hIgG). This design tethers the ectodomain to the dimeric Fc, which should stabilize the dimeric form of the ectodomain. This design was chosen to facilitate potentially identifying antibodies that recognize the homodimeric form of HER2. It has been shown that even though the ectodomain has a dimer-forming interface, the transmembrane and kinase region are necessary to stabilize the dimeric conformation (Ferguson et al., “Extracellular Domains Drive Homo-but Not Hetero-dimerization of erbB Receptors,” EMBO J 19:4632-4643 (2000), which is hereby incorporated by reference in its entirety). The forced dimerization with Fc should stabilize the ectodomain homodimer (FIG. 8). All the following work with purified HER2 proteins was done with these constructs, which are referred to as HER2 S310F-Fc, HER2 S310Y-Fc and HER2 WT-Fc for simplicity.

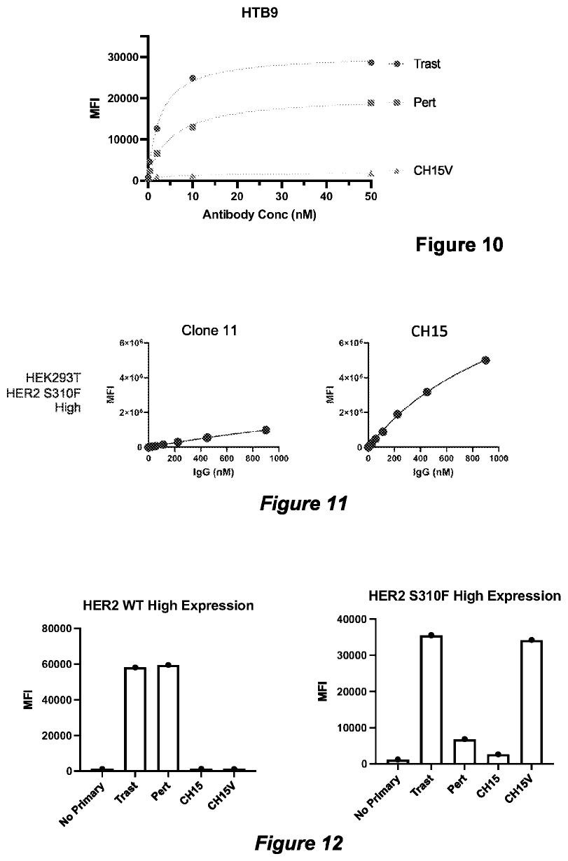

A panel of engineered HEK293T cells was also established to facilitate antibody discovery and characterization. HEK293T cells stably transfected with retroviruses that express HER2 WT or S310F/Y mutant were sorted using trastuzumab to obtain distinct populations of cells with a uniform expression level. Trastuzumab binds to HER2 at a distinct epitope away from S310. The cell populations, arbitrarily termed high and low exhibited more than a 10-fold difference in the HER2 surface level probed with trastuzumab (FIG. 9). For simplicity, the engineered HEK293T cells are termed HEK293T WT/S310F/S310Y^{Low/High} hereafter. A urinary bladder epithelial cancer cell line, HTB9, that endogenously expresses HER2 S310F was also included in the cell line panel employed. HTB9 expressed the least level of HER2 on the surface in the panel (FIG. 9). Furthermore, because HTB9 cells are reported to express both HER2 WT and S310F, the level of HER2 S310F should be even lower than the HER2 level determined from trastuzumab staining (Shin et al., “The HER2 S310F Mutant Can Form an Active Heterodimer with the EGFR, Which Can Be Inhibited by Cetuximab But Not by Trastuzumab as Well as Pertuzumab,” Biomolecules 9(10):629 (2019), which is hereby incorporated by reference in its entirety). Based on the different specificity of trastuzumab, which does not distinguish the HER2 WT and S310F, and pertuzumab, which binds to HER2 WT but not to HER2 S310F (Diwanji et al., “Structures of the HER2-HER3-NRG1β Complex Reveal a Dynamic Dimer Interface,” Nature 600:339-343 (2021); Zhang et al., “Identification of an Activating Mutation in the Extracellular Domain of HER2 Conferring Resistance to Pertuzumab,” Onco Targets Ther 12:11597-11608 (2019); Hao et al., “Cryo-EM Structure of HER2-trastuzumab-pertuzumab Complex,” PLoS One 14:e0216095 (2019), each of which is hereby incorporated by reference in its entirety), it was confirmed that HTB9 indeed express HER2 S310F, although at a lower level than HER2 WT (FIG. 10).

The initial hit was identified from a human synthetic antibody phage library using an established method that incorporated positive selection with HER2 S310F/Y-Fc and negative selection with HER2 WT-Fc (Oury et al., “Mechanism of Disease and Therapeutic Rescue of Dok7 Congenital Myasthenia,” Nature 595:404-408 (2021); Miller et al., “T Cell Receptor-like Recognition of Tumor in vivo by Synthetic Antibody Fragment,” PLoS One 7:e43746 (2012), each of which is hereby incorporated by reference in its entirety). This antibody, termed Clone 11, exhibited high specificity to HER2 S310F/Y, but its affinity was low, particularly to HER2 S310F expressed on the cell surface (FIG. 11). Clone 11 was subjected to affinity maturation by randomizing CDR H1 and H2 and selected clones for binding to HER2 expressed on the cell surface. One clone, termed CH15, was identified that showed improved binding to HER2 S310F on the surface of HEK293T cells (FIG. 11). A potential N-glycosylation site was eliminated in CH15 to produce a new clone, CH15V. CH15V in the hIgG1 format displayed enhanced binding towards HER2 S310F on HEK293T cells compared with CH15 IgG while still specific to the mutant (FIG. 12).

Next, the binding characteristics of CH15V were quantitatively assessed using biolayer interferometry (BLI). CH15V in the monomeric Fab format showed biphasic association to both HER2 S310F and S310Y, which rendered the determination of the K_{D} values challenging. Provisionally estimated K_{D1}=1.52×10⁻⁷ M and K_{D2}=1.38×10⁻⁸ M were obtained from the fitted curves with 95% of contribution coming from K_{D1} for S310F and K_{D1}=3.56×10⁻⁷ M and K_{D2}<1.0×10⁻¹² M from the fitted curves with 88% of contribution coming from K_{D1} for S310Y (FIG. 4B). Despite the binding affinities being in nanomolar range, CH15V did not show binding to HTB9, indicating that its affinity is insufficient for engaging HER2 S310F on these cells (FIG. 10). Thus, one more round of affinity maturation was carried out.

### Example 6—Affinity Maturation of CH15V Aided by Deep Mutational Scanning

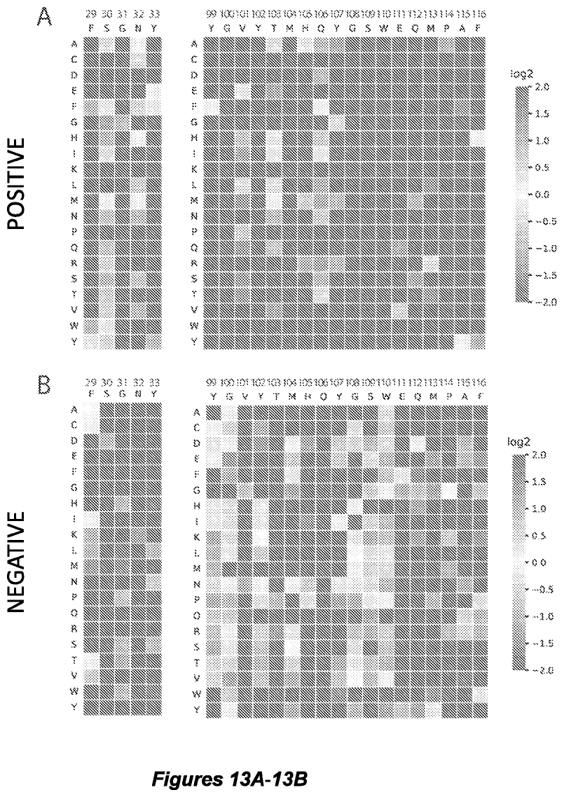

A yeast display system of CH15V in a single-chain Fv format was established, and deep mutational scanning (DMS) of the residues in CDR-H1 and CDR-H3 was performed. These residues were diversified one residue at a time using the degenerate NNK codon that encodes all the 20 amino acids to make a library. The library was then sorted to enrich clones that bind to HER2 S310F-Fc as well as those that are expressed but fail to bind to HER2 S310F-Fc. An analysis of these enriched pools with deep sequencing identified permissible and impermissible mutations (FIGS. 13A-13B). Next, a tailored library that included combinations of permissible mutations was designed. To further enhance the binding affinity, additional diversity was introduced in CDR L3, which was essentially poly-serine in CH15V, by transferring a CDR L3 repertoire from the naïve phage-display library. This yeast-display library was sorted and clones exhibiting improved binding to HER2 S310F/Y were identified. New antibodies were also screened for polyreactivity with biotinylated solubilized membrane following a previously published protocol (Xu et al., “Addressing Polyspecificity of Antibodies Selected from an in vitro Yeast Presentation System: A FACS-based, High-throughput Selection and Analytical Tool,” Protein Engineering, Design and Selection 26:663-670 (2013), which is hereby incorporated by reference in its entirety). Three third-generation clones, named TL1, TL18 and LL2 were identified. BLI analysis of these antibodies in the Fab format showed much improved binding towards HER2 S310F/Y compared with CH15V, with ~5 to 10-fold decreases in k_{off} (FIG. 4B, Table A). They also exhibited tighter binding towards engineered HEK293T S310F^{Low} and S310Y^{Low} (FIGS. 4D, 4E). However, this affinity improvement occurred at a cost of specificity, as the three new clones showed substantial binding to HER2 WT in both BLI and cell binding experiments (FIGS. 4C, 4F). To understand how the new antibodies recognize S310F/Y mutant and to formulate a strategy to enhance specificity, the structure of HER2 S310F bound to one of the antibodies was determined using cryoEM.

TABLE A
The dissociation rate constants of CH15V and the third-generation
antibodies in the Fab format against the homodimeric
HER2-Fc antigens as determined with BLI.
The mean and the SEM of k_{d} values (n = 3) are given.

| Antibody | HER2 S310F (23-652)-Fc | HER2 S310Y (23-652)-Fc |
|---|---|---|
| CH15V | 5.0 × 10⁻³ ± 1.3 × 10⁻⁴ | 8.4 × 10⁻³ ± 1.6 × 10⁻³ |
| TL1 | 1.2 × 10⁻³ ± 1.7 × 10⁻⁴ | 1.1 × 10⁻³ ± 3.3 × 10⁻⁴ |
| TL18 | 5.3 × 10⁻⁴ ± 1.4 × 10⁻⁴ | 4.7 × 10⁻⁴ ± 2.6 × 10⁻⁴ |
| LL2 | 1.10 × 10⁻³ ± 4 × 10⁻⁵ | 1.2 × 10⁻³ ± 1.6 × 10⁻⁴ |

### Example 7—Structures of the Ectodomain of HER2 S310F as a Homodimer and in Complex with TL1

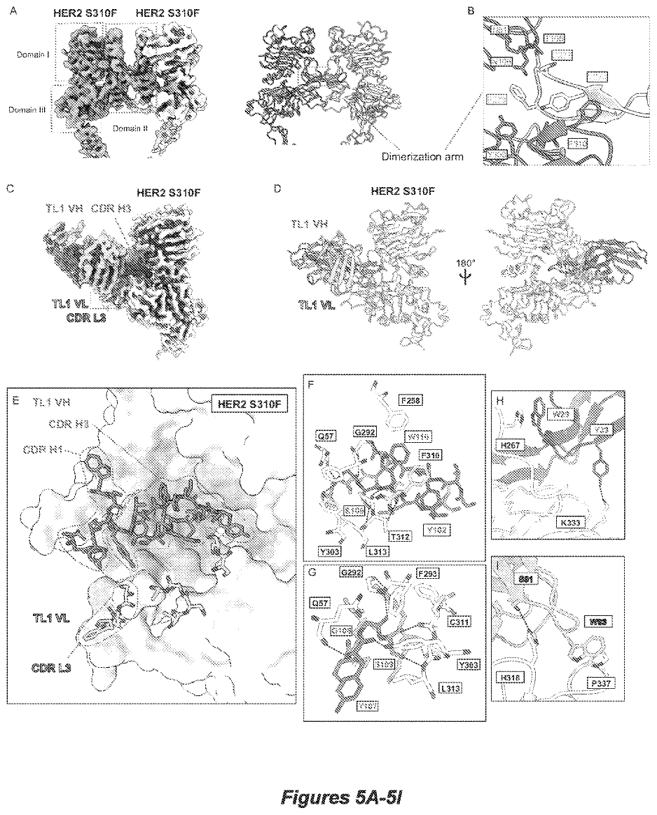

Prior to determining the structure of the TL1-HER2 S310F complex, we first determined the structure of HER2 S310F-Fc without a bound antibody in order to clearly establish the oligomeric state of our HER2-Fc construct. The structure was determined to nominal resolution of 2.01 Å. In accordance with previous reports of enhanced dimerization by S310F/Y mutation (Diwanji et al., “Structures of the HER2-HER3-NRG1β Complex Reveal a Dynamic Dimer Interface,” Nature 600:339-343 (2021); Ishiyama et al., “Computational and Functional Analyses of HER2 Mutations Reveal Allosteric Activation Mechanisms and Altered Pharmacologic Effects,” Cancer Res 83:1531-1542 (2023), each of which is hereby incorporated by reference in its entirety), HER2 S310F ectodomain was observed in the dimeric state but not in the monomeric state (FIG. 5A). The Fc region was completely averaged out, as expected from the flexible linker between the HER2 ectodomain and Fc portions in the construct utilized in these examples. The overall conformation of the HER2 S310F ectodomain is extended and similar to those previously reported (Cho et al., “Structure of the Extracellular Region of HER2 Alone and in Complex with the Herceptin Fab,” Nature 421:756-760 (2003); Diwanji et al., “Structures of the HER2-HER3-NRG1β Complex Reveal a Dynamic Dimer Interface,” Nature 600:339-343 (2021); Bai et al., “Structure and Dynamics of the EGFR/HER2 Heterodimer,” Cell Discov 9:18 (2023), each of which is hereby incorporated by reference in its entirety).

The densities for the HER2 S310F dimerization of both molecules in the dimer were clearly visible. S310F makes π-π interaction with Y274 of its dimeric partner (FIG. 5B), consistent with its role in stabilizing the dimer. This configuration is in contrast to structures of HER1 and HER3 in heterodimeric complex with HER2 WT where the HER1/HER3 dimerization arm is not visible in the cryoEM structure (Diwanji et al., “Structures of the HER2-HER3-NRG1β Complex Reveal a Dynamic Dimer Interface,” Nature 600:339-343 (2021), which is hereby incorporated by reference in its entirety). These results demonstrate that HER2 S310F ectodomain, in the construct utilized, predominantly exists in the noncovalent homodimeric conformation.

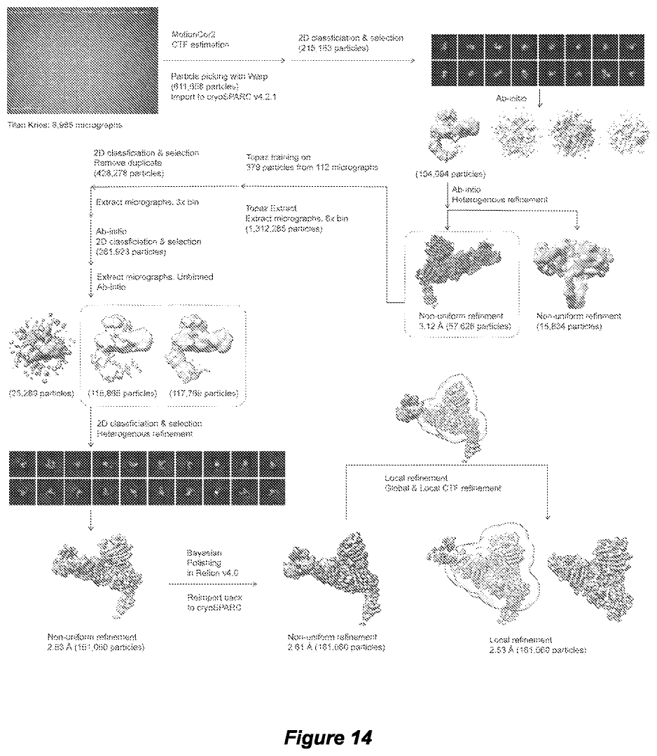

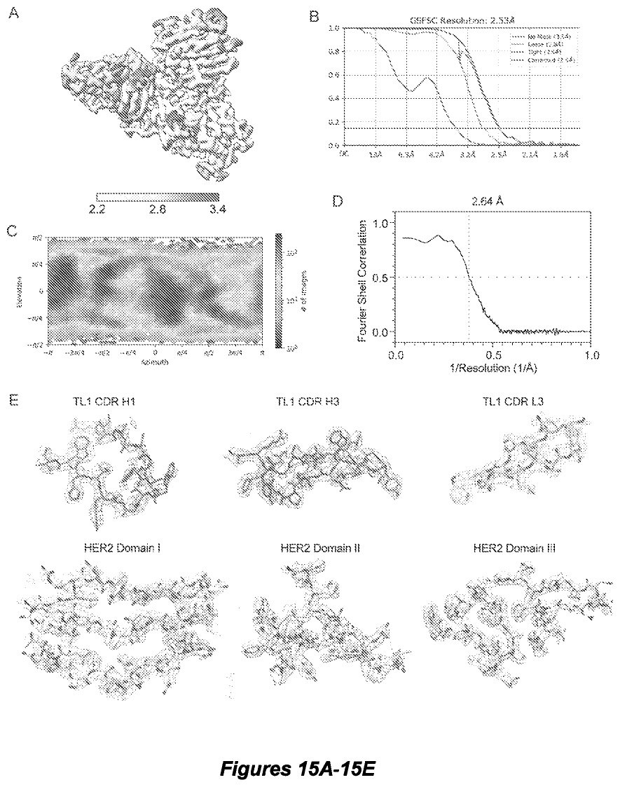

The structure of TL1 Fab bound to HER2 S310F-Fc was determined to the nominal resolution of 2.53 Å (FIGS. 5C, 5D). Again, the density for the Fc portion was not observed. It was initially speculated that this antibody would bind to HER2 in a manner specific to dimer since HER2 S310F-Fc is primarily in dimeric state. Surprisingly, a complex of one TL1 Fab with one HER2 S310F ectodomain was observed. Upon closer inspection, a small number of particles containing two visible copies of the HER2 ectodomain each binding to one Fab (FIG. 14) was observed, indicating that each complex contains two copies of the HER2-Fab complexes but the density of the second complex is averaged out in most particles so that only the 1 to 1 complex is observed. The extended conformation of HER2 S310F is similar to other published structures of HER2. The RMSD for Ca for a pairwise comparison of the structure with HER2 S310F in a heterodimeric complex with HER3/neuregulin-1β (NRG1β) (Diwanji et al., “Structures of the HER2-HER3-NRG1β Complex Reveal a Dynamic Dimer Interface,” Nature 600:339-343 (2021), which is hereby incorporated by reference in its entirety) was 0.728 Å, showing TL1 does not affect the overall conformation of the HER2 ectodomain.

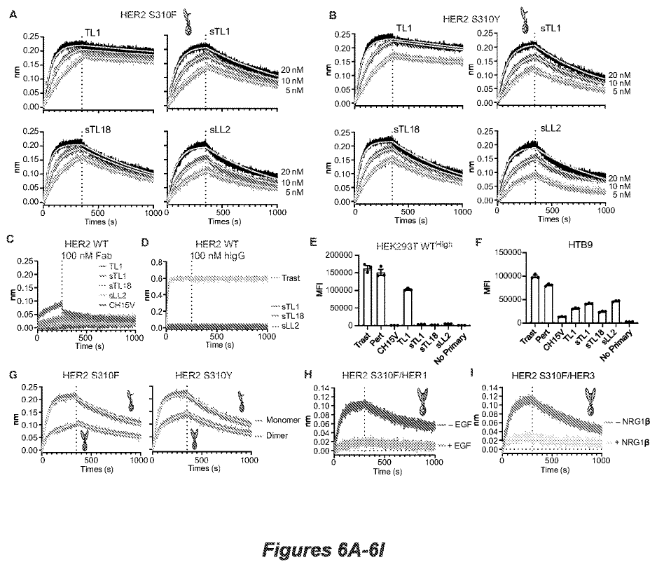

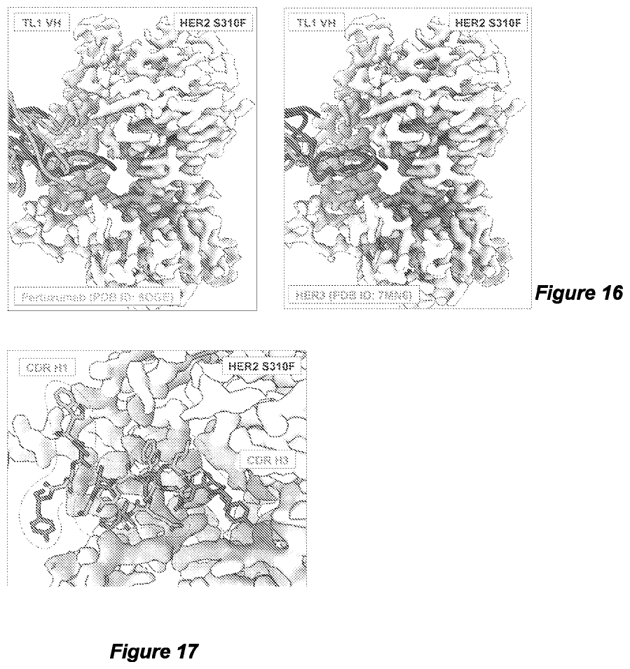

TL1 binds to domain II of HER2 and its epitope overlaps with that of pertuzumab, an antibody that selectively binds to the HER2 WT (FIGS. 5E, 16). However, unlike pertuzumab, TL1 projects its long CDR-H3 much deeper and occupies the dimerization arm-binding pocket of HER2 in a conformation similar to that of the dimerization arm of HER receptors observed in homo- and hetero-dimers (FIG. 16). Taking a closer look at CDR-H3, Y102 in CDR-H3 of TL1 interacts directly with HER2 S310F, forming π-π interaction. Y102 is conserved in TL18 and LL2, and the DMS analyses showed that it can only be replaced by another residue containing the benzene ring, Phe, indicating the importance of this interaction in conferring both affinity and specificity (FIG. 5F). CDR H3 makes a tight turn at Y107 and G108 where G108, which has a positive phi angle that is accommodated uniquely by Gly, cannot be replaced with another amino acid (FIG. 5G). Residues in CDR-H3 C-terminal to this turn form extensive intermolecular polar interactions with HER2 (FIG. 6F, 6G). Few mutations are allowed for the residues in this region facing HER2 (FIGS. 13A-B, 17). In addition to the polar interactions, TL1 W110 mediates hydrophobic interaction with HER2 F258 and S310F (FIG. 5F). These observations indicate the importance of the CDR-H3 conformation that support the placement of the crucial residues. By contrast, the side chains of the residues around Y102 are permissible to mutations according to the DMS analyses, and they do not directly contact HER2 and instead point towards the solvent (FIG. 17). In addition to CDR-H3 residues, W29 in CDR-H1 makes cation-π interaction with HER2 H267 (FIG. 5H). W29 can only be substituted by aromatic residues, F and Y (FIGS. 13A-B, 17). CDR H1 appears to contribute to this interaction by anchoring VH in the observed position. CDR H2 does not make much significant contact with HER2. Overall, this structure revealed that TL1 uses its VH to interact with HER2 mainly through CDR H3 that makes extensive contacts with the HER2 dimerization interface and directly recognizes the S310F mutation (FIG. 5E).

It was discovered that CDR L3 binds to surfaces of HER2 that are distinct from the interaction interface for the VH domain, and also distant from HER2 S310F (FIG. 5I). CH-π interaction between CDR-L3 W93 and HER2 P337 and hydrogen bonding between CDR-L3 S91 and HER2 H318 are observed in this interface (FIG. 5I). Although the sequence of CDR-L3 of TL1, TL18 and LL2 vary, they all have an aromatic residue at position 93 and Ser/Asp at position 91, which should preserve the observed types of interactions mediated by W93 and S91, indicating the importance of these interactions. By contrast, CH15V, which does not interact with HER2 WT, has a CDR-L3 sequence containing Ser residues at these positions, and thus it would be unable to make similar interactions at these sites. The increased binding to HER2 WT exhibited by TL1, TL18 and LL2 can be rationalized by these additional interactions via CDR-L3 (FIG. 4F).

### Example 8—Structure-Guided Improvement of Antibody Selectivity

Because the structural analysis revealed that TL1 interacts with two distinct surfaces of HER2 S310F, one including the S310F mutation and the other shared by HER2 WT and S310F (FIG. 5E), it was believed that a reduction in binding to HER2 WT would be achieved by eliminating the interactions mediated by VL residues. Thus, another set of antibodies was designed by replacing CDR-L3 of TL1, TL18 and LL2 with a poly-serine sequence found in early clones. These fourth-generation antibodies were named as sTL1, sTL18 and sLL2, respectively. Their affinity in the Fab format to HER2 S310F/Y-Fc was measured using BLI (FIGS. 6A, 6B). Because the structural data revealed a 1 to 1 stoichiometry for the Fab/HER2 interaction, affinity of the Fabs was measured towards monomeric ectodomains of HER2 S310F and S310Y to eliminate complications coming from HER2 dimerization. Monomeric HER2 proteins were produced by utilizing the heterodimerizing Fc knobs-in-holes technology (Ridgway et al., “Knobs-into-holes' Engineering of Antibody CH3 Domains for Heavy Chain Heterodimerization,” Protein Engineering, Design and Selection 9:617-621 (1996); Merchant et al., “An Efficient Route to Human Bispecific IgG,” Nature Biotechnol 16:677-681 (1998); Atwell et al., “Stable Heterodimers from Remodeling the Domain Interface of a Homodimer Using a Phage Display Library,” J. Mol. Biol. 270:26-35 (1997), each of which is hereby incorporated by reference in its entirety). sTL1 showed a decrease in affinity compared with TL1, which was expected as an interaction site was lost (Table B). Yet, its K_{D} value still was in the low nanomolar range, and so were the K_{D} values for sTL18 and sLL2. Importantly, these new Fabs showed no detectable association signal at 100 nM unlike TL1 Fab (FIG. 6C). Furthermore, even when tested in the hIgG form, which, due to having two binding arms would exhibit avidity effect in binding, sTL1, sTL18 and sLL2, the hIgGs did not interact with HER2 WT at 100 nM. A comparison to trastuzumab clearly demonstrates how extremely selective these antibodies are towards HER2 S310F/Y (FIG. 6D). These results demonstrate that the structure-guided design selectively reduced the affinity to HER2 WT and consequently improved selectivity toward HER2 S310F/Y.

Next, these antibodies were tested against full-length HER2 expressed on the cell surface. sTL1, sTL18 and sLL2 hIgGs all displayed minimal binding to the HEK293T HER2 WT^{High} cells (FIG. 6E). When tested against HTB9 cells, cancer cells with lowest expression level of HER2 S310F among cell lines that were screened, substantial binding was observed, higher than CH15V, yet comparable to TL1 (FIGS. 9, 6F). Altogether, these results show that all three engineered antibodies, sTL1, sTL18 and sLL2 have high affinity and selectivity toward HER2 S310F/Y and they are capable of recognizing the low level of the HER2 mutant on cancer cells.

TABLE B
The K_{D} values of TL1 and the fourth-generation antibodies in the Fab format
against monomeric or dimeric HER2 mutant constructs as measured with BLI.
The value given are calculated using the Octet data analysis software.

| | HER2 | Oligomeric | | |
|---|---|---|---|---|
| Antibody | mutant | state | K_{D1} (M) | K_{D2} (M) |
| TL1 | S310F | Monomer | 2.15 × 10⁻¹⁰ ± 2 × 10⁻¹² | |
| sTL1 | | Monomer | 1.74 × 10⁻⁹ ± 7 × 10⁻¹² | |
| sTL18 | | Monomer | 1.22 × 10⁻⁹ ± 4 × 10⁻¹² | |
| sLL2 | | Monomer | 2.32 × 10⁻⁹ ± 1.0 × 10⁻¹¹ | |
| sTL18 | | Dimer | 1.8 × 10⁻⁸ ± 1.0 × 10⁻⁹ | 3.05 × 10⁻⁹ ± 6 × 10⁻¹¹ |
| TL1 | S310Y | Monomer | 3.15 × 10⁻¹⁰ ± 2 × 10⁻¹² | |
| sTL1 | | Monomer | 2.71 × 10⁻⁹ ± 1.0 × 10⁻¹¹ | |
| sTL18 | | Monomer | 1.52 × 10⁻⁹ ± 5 × 10⁻¹² | |
| sLL2 | | Monomer | 2.54 × 10⁻⁹ ± 2 × 10⁻¹¹ | |
| sTL18 | | Dimer | 1.56 × 10⁻⁸ ± 7 × 10⁻¹⁰ | 3.14 × 10⁻⁹ ± 4 × 10⁻¹¹ |

### Example 9—Dynamics of HER2 S310F/Y Dimerization Probed with Mutant-Selective Antibodies

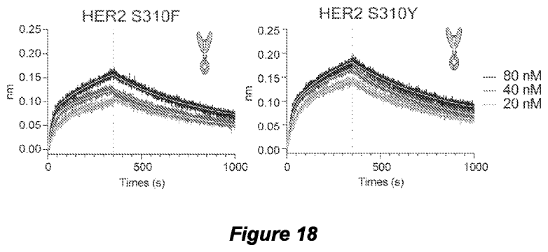

The structural data described in the preceding Examples revealed that the epitope recognized by the described antibodies largely overlaps with the dimeric interface, indicating that Fab binding and HER2 dimerization influence each other. Thus, the binding efficacy of sTL18 to the homodimeric and monomeric HER2 S310F/Y constructs was compared under the same conditions. As expected, the BLI signal intensity was lower for to the homodimer than the monomer, supporting the presence of competition between sTL1 binding and homodimerization (FIG. 6G; Table B). However, the dimerization did not obliterate the binding of sTL18 (FIG. 18). The sensorgram was biphasic, likely reflecting the presence of the monomeric and dimeric states in the HER2 S310F-Fc sample. These findings demonstrate that even when two copies of the HER2 S310F/Y ectodomain are tethered in proximity, they do not maintain a stable dimer as speculated by previous reports, e.g., by forming intermolecular disulfide bonds (Ishiyama et al., “Computational and Functional Analyses of HER2 Mutations Reveal Allosteric Activation Mechanisms and Altered Pharmacologic Effects,” Cancer Res 83:1531-1542 (2023), which is hereby incorporated by reference in its entirety). The high affinity of the disclosed antibodies enables them to overcome HER2 homodimerization and bind to the dimerization interface in each HER2 monomer.

HER2 can form heterodimers with other HER members, in addition to the homodimer. Thus, the effect of heterodimerization on sTL18 binding was also investigated. As with monomeric HER2 preparation, the Fc knob-into-hole technology was used to prepare heterodimers of HER1/HER2 S310F and HER3/HER2 S310F ectodomains fused to Fc (FIG. 4B) (Ridgway et al., “‘Knobs-into-holes’ Engineering of Antibody CH3 Domains for Heavy Chain Heterodimerization,” Protein Engineering, Design and Selection 9:617-621 (1996); Merchant et al., “An Efficient Route to Human Bispecific IgG,” Nature Biotechnol 16:677-681 (1998); Atwell et al., “Stable Heterodimers from Remodeling the Domain Interface of a Homodimer Using a Phage Display Library,” J. Mol. Biol. 270:26-35 (1997), each of which is hereby incorporated by reference in its entirety). Unlike HER2 that always exists in the extended conformation and primed for forming a dimer, HER1 and HER3 exist in a closed, auto-inhibited confirmation in the absence of their ligands (Cho and Leahy, “Structure of the Extracellular Region of HER3 Reveals an Interdomain Tether,” Science 297, 1330-1333 (2002); Ferguson et al., “EGF Activates Its Receptor by Removing Interactions that Autoinhibit Ectodomain Dimerization,” Molecular Cell 11:507-517 (2003), each of which is hereby incorporated by reference in its entirety). When their ligands bind between domain I and domain III, HER1 and HER3 adopt extended conformation, exposing their dimerization arms for interaction (Diwanji et al., “Structures of the HER2-HER3-NRG1β Complex Reveal a Dynamic Dimer Interface,” Nature 600:339-343 (2021); Ogiso et al., “Crystal Structure of the Complex of Human Epidermal Growth Factor and Receptor Extracellular Domains,” Cell 110:775-787 (2002), each of which is hereby incorporated by reference in its entirety). In the absence of the ligands, sTL18 Fab bound to the heterodimers as HER2 S310F remains unassociated (FIGS. 6H, 6G). However, the binding was nearly undetectable in the presence of their corresponding ligands, epidermal growth factor (EGF) for HER1 and NRG1β for HER3, even at a Fab concentration of 20 nM (FIGS. 6H-6I), the concentration at which sTL18 Fab could bind to HER2 S310F/Y homodimers. These results indicate that the HER ligands substantially stabilize the heterodimers, and that such stable heterodimers present a potential challenge in targeting HER2 S310F/Y with the disclosed antibodies if HER2 S310F/Y exists predominantly in such ligand-stabilized heterodimers.

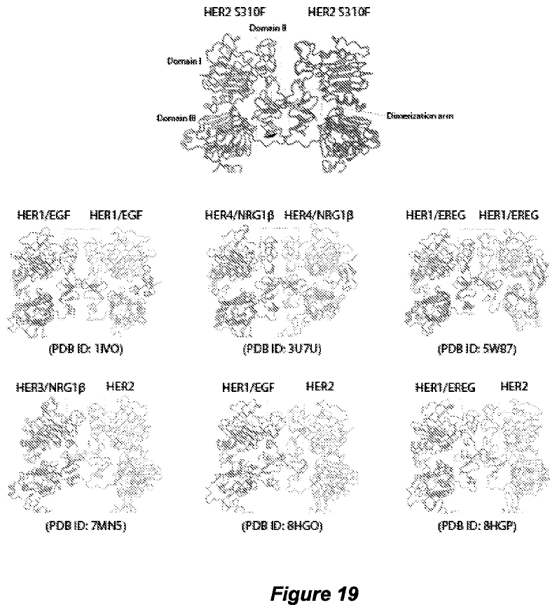

To rationalize the differences in the stability among different HER2 S310 dimers, the dimeric interface of the HER2 S310F homodimer structure was compared with other published HER dimers and the buried surface area of domain II, the major dimeric interface in the ectodomain, was calculated (Table E). One significant difference was that domain II of HER2 takes on a relatively straight conformation as no ligand is bound between domain I and III, HER2 S310F homodimers do not make any contact near the N-terminus of domain II, whereas all the other HER homodimers and heterodimers form conserved interactions involving this region (FIG. 19). Domain II of HER1, HER3 and HER4 are slightly bent and the N-terminal region of their domain II come into contact with their respective dimerization partner, even with HER2 (Diwanji et al., “Structures of the HER2-HER3-NRG1β Complex Reveal a Dynamic Dimer Interface,” Nature 600:339-343 (2021); Bai et al., “Structure and Dynamics of the EGFR/HER2 Heterodimer,” Cell Discov 9:18 (2023); Ogiso et al., “Crystal Structure of the Complex of Human Epidermal Growth Factor and Receptor Extracellular Domains,” Cell 110:775-787 (2002); Lu et al., “Structural Evidence for Loose Linkage Between Ligand Binding and Kinase Activation in the Epidermal Growth Factor Receptor,” Mol Cell Biol 30:5432-5443 (2010); Liu et al., “A Single Ligand Is Sufficient to Activate EGFR Dimers,” Proc Natl Acad Sci USA 109:10861-10866 (2012); Freed et al., “EGFR Ligands Differentially Stabilize Receptor Dimers to Specify Signaling Kinetics,” Cell 171:683-695 (2017), each of which is hereby incorporated by reference in its entirety). However, when two HER2s form a homodimer, it seems the rigidity of domain II does not enable them to make this interaction. The majority of dimeric contact of HER2 S310F comes from the fully resolved dimerization arms of HER2 stabilized by S310F of its counter partner. Despite the absence of contacts involving the N-terminal region of domain II, buried surface area of HER2 S310F homodimer (1481.3 Å²) is comparable to that of other stable hetero- and homodimers of HER and significantly greater than those of HER1/HER1/EREG (1079.8 Å²) and HER2/HER3/NRG1β (1002.2 Å²) that are known to be weak dimers. The buried surface area of HER3/HER2 S310F/NRG1β is 1715.6 Å², the only other dimeric structure solved for HER2 S310F, which is higher than that of HER2 S310F homodimer. Buried surface area specifically in the dimerization arm region was similar between the two, 973.7 Å² for HER2 S310F homodimer and 1045.1 Å² for HER2 S310F/HER3/NRG1β, as both complexes have two dimerization arms stabilized. This analysis indicates that interaction at the N-terminal region of domain II contributes to dimer formation and that the enhanced interaction involving S310F and the dimerization arm partially compensate the lack of the interaction at the N-terminal region in the HER2 homodimer.

TABLE C
Buried surface area calculated between two HER dimers.

| | | | | | Buried |
|---|---|---|---|---|---|
| | | | | | surface |
| | | | | Buried | area in |
| | | | | Surface | dimerization |
| | | | | Area | arm |
| PDB ID | Protein 1 | Protein 2 | Ligand | (Å²) | (Å²) |
| 1IVO | HER1 | HER1 | EGF | 1475.6 | 932.2 |
| 3NJP | HER1 | HER1 | EGF | 1458.6 | 934.2 |
| 8HGS | HER1 | HER1 | EGF | 1490.3 | 999.9 |
| 5WB7 | HER1 | HER1 | EREG | 1079.8 | 626.5 |
| 3U7U | HER4 | HER4 | NRG1B | 1488.1 | 837.5 |
| 8HGP | HER1 | HER2 | EREG | 1428.2 | 691.9 |
| 8HGO | HER1 | HER2 | EGF | 1369.6 | 743.9 |
| 7MN5 | HER3 | HER2 | NRG1B | 1002.2 | 545.6 |
| 7MN6 | HER3 | HER2 S310F | NRG1B | 1715.6 | 1045.1 |
| | HER2 S310F | HER2 S310F | | 1481.3 | 973.7 |
| | HER2 S310F | TL1 VH | | | 930.5 |

For 1IVO, see Ogiso et al., “Crystal Structure of the Complex of Human Epidermal Growth Factor and Receptor Extracellular Domains,” Cell 110(6):775-87 (2002);
for 3NJP, see Lu et al., “Structural Evidence for Loose Linkage Between Ligand Binding and Kinase Activation in the Epidermal Growth Factor Receptor,” Mol. Cell Biol. 30(22):5432-43 (2010);
for 5WB7, see Freed et al., “EGFR Ligands Differentially Stabilize Receptor Dimers to Specify Signaling Kinetics,” Cell 171(3):683-695 (2017);
for 7MN5 and 7MN6, see Diwanji et al., “Structures of the HER2-HER3-NRG1B Complex Reveal a Dynamic Dimer Interface,” Nature 600:339-343 (2021);
for 3U7U, see Liu et al., “A Single Ligand Is Sufficient to Activate EGFR Dimers,” Proc Natl. Acad. Sci. USA 109(27):10861-6 (2012);
for 8HGS, 8HGP, and 8HGO, see Zhang et al., “Identification of an Activating Mutation in the Extracellular Domain of HER2 Conferring Resistance to Pertuzumab,” Onco Targets Ther 12:11597-11608 (2019), each of which is hereby incorporated by reference in its entirety.

### Example 10—Selective Killing of Cells Expressing HER2 S310F/Y

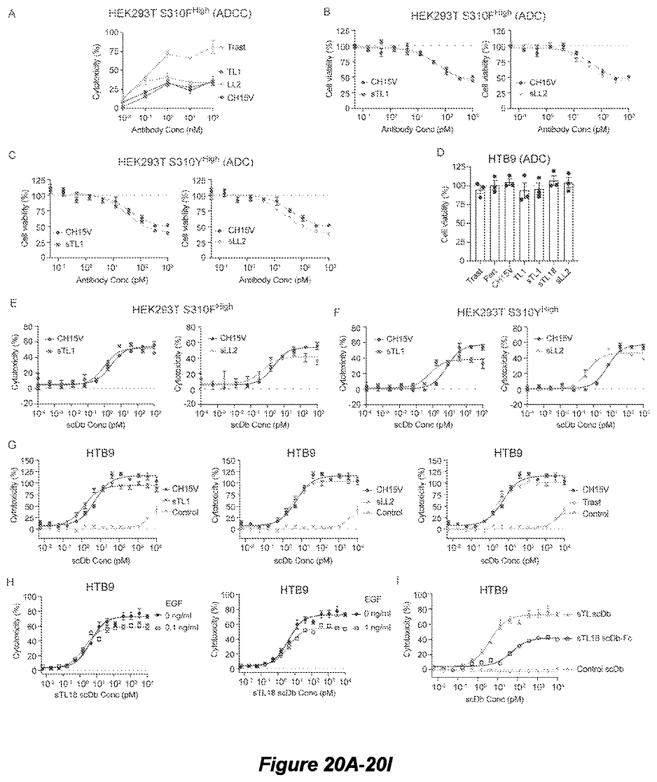

The ability of several antibodies to kill cells expressing HER2 S310F/Y was tested. First, the antibody-dependent cell cytotoxicity (ADCC) of TL1 and LL2, the antibodies with highest affinity to HER2 S310F, was tested. These antibodies in the IgG1 format showed marginal potency in killing HEK293T cells expressing a high level of HER2 S310F (FIGS. 20A-20C). The results indicate that other antibodies would have even lower killing potency mediated by ADCC.

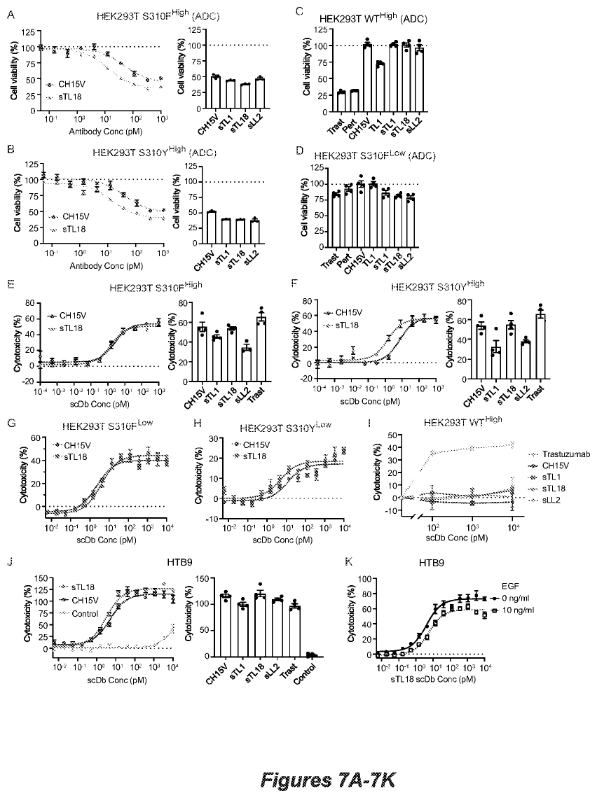

To improve the cytotoxicity of these antibodies, the efficacy of antibody-drug conjugates was examined next. Saporin-conjugated secondary antibody was used as the drug cargo. Saporin is a ribosome inhibitor that once internalized alongside the antibodies, will impede ribosome function and protein synthesis, which will inhibit cell growth and eventually lead to cell death. ADC assay with our fourth-generation antibodies effectively killed HEK293T cells with high expression levels of HER2 S310F/Y mutant with IC50 ranging from 10 to 60 μM (FIG. 7A-7B, FIG. 20B-20C, Table D). sTL18 showed the lowest IC50 value, 13.3 μM for HEK293T S310F^{High} cells and 14.4 μM for the HEK293T S310Y^{High} cells. Most importantly, at 1 nM of ADC, the highest concentration point tested, none of these ADCs exhibited cytotoxicity to HER2 WT high expression cells, indicating their exquisite selectivity (FIG. 7C). However, ADC was not as effective against the HEK293T S310F^{Low} as HEK293T S310F/Y^{High} cells, and similarly they showed no cytotoxicity against HTB9 at 1 nM of hIgG (FIGS. 7D, 20D). These results suggest that the internalization efficiency of our antibodies was not sufficiently high to deliver cytotoxic cargo above effective threshold into the cells with low expression level of HER2 mutants.

TABLE D
IC₅₀ calculated from ADC assay and the EC₅₀ values from the T-cell engager assay.
The mean and SEM of three to four replicates are given.

| | | IC₅₀/EC₅₀ (pM) | | | | |
|---|---|---|---|---|---|---|
| Modality | Target | CH15V | STL1 | STL 18 | sLL2 | Trastuzumab |
| ADC | HEK293T | 52 ± 15 | 59 ± 20 | 13 ± 1 | 21 ± 5 | |
| | S310F^{High} | | | | | |
| ADC | HEK293T | 48 ± 23 | 26.8 ± 0.8 | 15 ± 3 | 23 ± 3 | |
| | S310Y^{High} | | | | | |
| scDb | HEK293T | 3.0 ± 1.2 | 1.17 ± 0.3 | 1.5 ± 0.3 | 0.49 ± 0.13 | |
| | S310F^{High} | | | | | |
| scDb | HEK293T | 2.3 ± 0.4 | | 3.9 ± 0.3 | | |
| | S310F^{Low} | | | | | |
| scDb | HEK293T | 7.0 ± 0.7 | 1.05 ± 0.46 | 1.7 ± 0.5 | 0.45 ± 0.10 | |
| | S310Y^{High} | | | | | |
| scDb | HEK293T | 37.3 ± 28.8 | | 6.4 ± 3.2 | | |
| | S310Y^{Low} | | | | | |
| scDb | HTB9 | 5.02 ± 0.17 | 1.3 ± 0.3 | 3.2 ± 0.2 | 2.87 ± 0.14 | 2.1 ± 0.2 |

To further enhance cytotoxicity in a manner independent of antibody internalization, bispecific T cell engagers were designed and tested. Single-chain diabodies (scDbs) (Brüsselbach et al., “Enzyme Recruitment and Tumor Cell Killing in vitro by a Secreted Bispecific Single-chain Diabody,” Tumor Targeting 4:115-123 (1999), which is hereby incorporated by reference in its entirety), with the first Fv unit consisting of CH15V, sTL1, sTL18, sLL2 or trastuzumab and the other unit with the UCHT1 that recognizes CD3ε on T lymphocytes (Beverley and Callard, “Distinctive Functional Characteristics of Human “T” Lymphocytes Defined by E Rosetting or a Monoclonal Anti-T cell Antibody,” Eur J Immunol 11:329-334 (1981), which is hereby incorporated by reference in its entirety). The structures of the scDbs are shown below:

CH15V_scDb
(SEQ ID NO: 144)
DIVRSDIQMTQSPSSLSASVGDRVTITCRASQSVSSAVAWYQQKPGKAPKLLIYSASSLYSGVPSRFS
GSRSGTDFTLTISSLQPEDFATYYCQQSSSSLITFGQGTKVEIKGGGGSEVQLQQSGPELVKPGASMK
ISCKASGYSFTGYTMNWVKQSHGKNLEWMGLINPYKGVSTYNQKFKDKATLTVDKSSSTAYMELLSLT
SEDSAVYYCARSGYYGDSDWYFDVWGQGTTLTVSSGGGGSGGGGSGGGGSDIQMTQTTSSLSASLGDR
VTISCRASQDIRNYLNWYQQKPDGTVKLLIYYTSRLHSGVPSKFSGSGSGTDYSLTISNLEQEDIATY
FCQQGNTLPWTFAGGTKLEIKGGGGSEVQLVESGGGLVQPGGSLRLSCAASGFTFSGNYIHWVRQAPG
KGLEWVASIYSAGGYTDYADSVKGRFTISADTSKNTAYLQMNSLRAEDTAVYYCARYGVYTMHQYGSW
EQMPAFDYWGQGTLVTVSSLEGGGGLNDIFEAQKIEWHESRHHHHHH
sLL2_scDb
(SEQ ID NO: 145)
DIVRSDIQMTQSPSSLSASVGDRVTITCRASQSVSSAVAWYQQKPGKAPKLLIYSASSLYSGVPSRFS
GSRSGTDFTLTISSLQPEDFATYYCQQSSSSLITFGQGTKVEIKGGGGSEVQLQQSGPELVKPGASMK
ISCKASGYSFTGYTMNWVKQSHGKNLEWMGLINPYKGVSTYNQKFKDKATLTVDKSSSTAYMELLSLT
SEDSAVYYCARSGYYGDSDWYFDVWGQGTTLTVSSGGGGSGGGGSGGGGSDIQMTQTTSSLSASLGDR
VTISCRASQDIRNYLNWYQQKPDGTVKLLIYYTSRLHSGVPSKFSGSGSGTDYSLTISNLEQEDIATY
FCQQGNTLPWTFAGGTKLEIKGGGGSEVQLVESGGGLVQPGGSLRLSCAASGFTWSGSYIHWVRQAPG
KGLEWVASIYSAGGYTDYADSVKGRFTISADTSKNTAYLQMNSLRAEDTAVYYCARYGVYTLASAGSW
ESLPAFDYWGQGTLVTVSSLEGGGGLNDIFEAQKIEWHESRHHHHHH
sTL1_scDb
(SEQ ID NO: 146)
DIVRSDIQMTQSPSSLSASVGDRVTITCRASQSVSSAVAWYQQKPGKAPKLLIYSASSLYSGVPSRFS
GSRSGTDFTLTISSLQPEDFATYYCQQSSSSLITFGQGTKVEIKGGGGSEVQLQQSGPELVKPGASMK
ISCKASGYSFTGYTMNWVKQSHGKNLEWMGLINPYKGVSTYNQKFKDKATLTVDKSSSTAYMELLSLT
SEDSAVYYCARSGYYGDSDWYFDVWGQGTTLTVSSGGGGSGGGGSGGGGSDIQMTQTTSSLSASLGDR
VTISCRASQDIRNYLNWYQQKPDGTVKLLIYYTSRLHSGVPSKFSGSGSGTDYSLTISNLEQEDIATY
FCQQGNTLPWTFAGGTKLEIKGGGGSEVQLVESGGGLVQPGGSLRLSCAASGFTWSGAYIHWVRQAPG
KGLEWVASIYSAGGYTDYADSVKGRFTISADTSKNTAYLQMNSLRAEDTAVYYCARYGTYELKSYGSW
ESLPAFDYWGQGTLVTVSSLEGGGGLNDIFEAQKIEWHESRHHHHHH
sTL18_scDb
(SEQ ID NO: 147)
DIVRSDIQMTQSPSSLSASVGDRVTITCRASQSVSSAVAWYQQKPGKAPKLLIYSASSLYSGVPSRFS
GSRSGTDFTLTISSLQPEDFATYYCQQSSSSLITFGQGTKVEIKGGGGSEVQLQQSGPELVKPGASMK
ISCKASGYSFTGYTMNWVKQSHGKNLEWMGLINPYKGVSTYNQKFKDKATLTVDKSSSTAYMELLSLT
SEDSAVYYCARSGYYGDSDWYFDVWGQGTTLTVSSGGGGSGGGGSGGGGSDIQMTQTTSSLSASLGDR
VTISCRASQDIRNYLNWYQQKPDGTVKLLIYYTSRLHSGVPSKFSGSGSGTDYSLTISNLEQEDIATY
FCQQGNTLPWTFAGGTKLEIKGGGGSEVQLVESGGGLVQPGGSLRLSCAASGFTEGGSYIHWVRQAPG
KGLEWVASIYSAGGYTDYADSVKGRFTISADTSKNTAYLQMNSLRAEDTAVYYCARYGVYTLHQYGSW
EQLPAFDYWGQGTLVTVSSLEGGGGLNDIFEAQKIEWHESRHHHHHH
Trastuzumab_scDb
(SEQ ID NO: 176)
DIVRSDIQMTQSPSSLSASVGDRVTITCRASQDVNTAVAWYQQKPGKAPKLLIYSASFLYSGVPSRFS
GSRSGTDFTLTISSLQPEDFATYYCQQHYTTPPTFGQGTKVEIKGGGGSEVQLQQSGPELVKPGASMK
ISCKASGYSFTGYTMNWVKQSHGKNLEWMGLINPYKGVSTYNQKFKDKATLTVDKSSSTAYMELLSLT
SEDSAVYYCARSGYYGDSDWYFDVWGQGTTLTVSSGGGGSGGGGSGGGGSDIQMTQTTSSLSASLGDR
VTISCRASQDIRNYLNWYQQKPDGTVKLLIYYTSRLHSGVPSKFSGSGSGTDYSLTISNLEQEDIATY
FCQQGNTLPWTFAGGTKLEIKGGGGSEVQLVESGGGLVQPGGSLRLSCAASGFNIKDTYIHWVRQAPG
KGLEWVARIYPTNGYTRYADSVKGRFTISADTSKNTAYLQMNSLRAEDTAVYYCSRWGGDGFYAMDYW
GQGTLVTVSSLEGGGGLNDIFEAQKIEWHESRHHHHHH

STL18_scDb_huFc (sTL18_scDb, which contains two chains (A and B), is attached to human Fc so as to improve the blood half-life.)

sTL18_scDb_huFc_A
(SEQ ID NO: 177)
DIQMTQSPSSLSASVGDRVTITCRASQSVSSAVAWYQQKPGKAPKLLIYSASSLYSGVPSRFSGS
RSGTDFTLTISSLQPEDFATYYCQQSSSSLITFGQGTKVEIKGGGGSEVQLQQSGPELVKPGASM
KISCKASGYSFTGYTMNWVKQSHGKNLEWMGLINPYKGVSTYNQKFKDKATLTVDKSSSTAYMEL
LSLTSEDSAVYYCARSGYYGDSDWYFDVWGQGTTLTVSSGGGGSGGGGSGGGGSDIQMTQTTSSL
SASLGDRVTISCRASQDIRNYLNWYQQKPDGTVKLLIYYTSRLHSGVPSKFSGSGSGTDYSLTIS
NLEQEDIATYFCQQGNTLPWTFAGGTKLEIKGGGGSEVQLVESGGGLVQPGGSLRLSCAASGFTF
GGSYIHWVRQAPGKGLEWVASIYSAGGYTDYADSVKGRFTISADTSKNTAYLQMNSLRAEDTAVY
YCARYGVYTLHQYGSWEQLPAFDYWGQGTLVTVSSDKTYTCPPCPAPEAAGGPSVFLFPPKPKDT
LMISRTPEVTCVVVDVSHEDPEVKFNWYVDGVEVHNAKTKPREEQYNSTYRVVSVLTVLHQDWLN
GKEYKCKVSNKALGAPIEKTISKAKGQPREPQVYTLPPCRDELTKNQVSLWCLVKGFYPSDIAVE
WESNGQPENNYKTTPPVLDSDGSFFLYSKLTVDKSRWQQGNVFSCSVMHEALHNHYTQKSLSLSP
SSSSHHHHHH
sTL18_scDb_huFc_B
(SEQ ID NO: 178)
DKTHTCPPCPAPEAAGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALGAPIEKTISKAKGQPREPQVCT
LPPSRDELTKNQVSLSCAVKGFYPSDIAVEWESNGQPENNYKTTPPVLDSDGSFFLVSKLTVDKS
RWQQGNVFSCSVMHEALHNHYTQKSLSLSP

All the S310F/Y-directed scDbs, including CH15V, potently killed the S310F/Y^{High} cells with EC₅₀ in the low picomolar range, much lower than those for the ADCs (FIGS. 7E-7F, 20E-20F, Table D). The effect of the improved affinity of sTL1, sTL18 and sLL2 compared with CH15V was more pronounced for the S310Y^{High} cells, as they had 2~6 times lower EC₅₀ values than CH15V (Table D). sTL18-scDb and CH15V-scDb were also tested against the S310F/Y^{Low} cells. Unlike their ADC counterparts, these scDbs exhibited cytotoxicity against the S310F/Y^{Low} cells with EC50 in the low pM range, although maximal killing was reduced compared with that against the S310F/Y^{High} cells (FIG. 7G-H). Furthermore, all the four scDbs tested exhibited no cytotoxicity against HEK293T cells expressing a high level of HER2 WT even at 10 nM, whereas the scDb constructed with trastuzumab potently killed these cells (FIG. 71). Thus, these antibodies in the bispecific T cell engager format achieved extremely selective and potent cytotoxicity toward HER2 S310F/Y.

### Example 11—Selective Killing of HTB9 Cancer Cells In Vitro and In Vivo

Having identified an effective format with selective and potent cytotoxicity toward HER2 S310F/Y cells, the scDbs were subsequently tested against HTB9 cells. All four scDbs killed HTB9 cells with EC₅₀ in the low picomolar range (FIG. 7J, Table E). A negative control scDb that does not bind to HER2 showed no significant cytotoxicity, eliminating the possibility of non-specific cell killing.

TABLE E
The EC₅₀ values and the maximum cytotoxicity calculated
from T-cell engager assays with sTL18 scDb in the absence
and presence of 0.1, 1, 10 ng/ml EGF. The mean and
SEM (n = 4) are given.

| | | | | |
|---|---|---|---|---|
| EGF (ng/mL) | 0 | 0.1 | 1 | 10 |
| EC50 (pM) | 4.3 ± 0.5 | 1.8 ± 0.2 | 4.4 ± 1.2 | 6.2 ± 0.9 |
| Maximum | 73 ± 4 | 59 ± 3 | 55.4 ± 1.2 | 59.1 ± 1.8 |
| Cytotoxicity (%) | | | | |

The structural and BLI analyses have shown that HER2 heterodimerization in the presence of a HER1 or HER3 ligand can hinder binding of the tested antibodies. HTB9 cells express HER1 at a high level, higher than HER2 (Shin et al., “The HER2 S310F Mutant Can Form an Active Heterodimer with the EGFR, Which Can Be Inhibited by Cetuximab but Not by Trastuzumab as well as Pertuzumab,” Biomolecules 9(10):629 (2019), which is hereby incorporated by reference in its entirety), suggesting that the efficacy of the tested scDbs is potentially limited by ligand-stabilized heterodimerization. The cytotoxicity of sTL18-scDb was tested in the presence of EGF at 0.1 ng/ml, 1 ng/ml, and 10 ng/ml covering a physiologically relevant range (Joh et al., “Physiological Concentrations of Human Epidermal Growth Factor in Biological Fluids: Use of a Sensitive Enzyme Immunoassay,” Clinica Chimica Acta 158:81-90 (1986); Meybosch et al., “Epidermal Growth Factor and Its Influencing Variables in Healthy Children and Adults,” PLoS ONE 14:e0211212 (2019), each of which is hereby incorporated by reference in its entirety). Unlike the in vitro binding data (FIG. 6H-I), the addition of EGF only marginally affected the cytotoxicity of the scDbs. EC50 did not shift much, and the maximum cytotoxicity decreased only by small percentage even at 10 ng/ml, a concentration above than normal physiological range (FIG. 7K, Table E). These results confirm that these scDb molecules are efficacious against cancer cells even in the presence of high concentrations of HER1 and HER3 ligands. This attribute is encouraging, because EGF and NRG are often upregulated in cancer.

Interestingly, trast-scDb and CH15V-scDb had similar EC₅₀, despite trastuzumab having much higher binding to HTB9 as assessed using cell staining (FIG. 20G). Similarly, it is surprising that CH15V has a comparable EC₅₀ to those for sTL1, sTL18 and sLL2-scDbs. These observations indicate that affinity to the cell surface antigen or the copy number of the antigen plays less role in scDb cytotoxicity. One possible explanation is that T cells do not require many scDbs bound on the cell surface to be triggered. Alternatively, the membrane-proximal position of the trastuzumab epitope on HER2 may be less effective for the scDb to simultaneously bind CD3 on T cells.

Based on the above results, sTL18 was selected as a lead candidate for in vivo testing, because it showed least binding to HER2 WT but still showed high cytotoxic potency in both ADC and T cell engager assay (FIG. 4F, FIG. 7, FIG. 8).

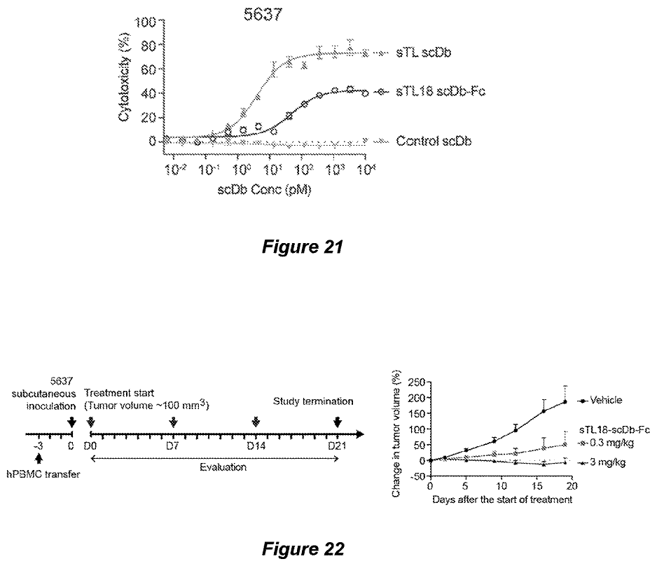

To increase its blood half-life, the Fc segment was fused to sTL18 scDb. This scDb-Fc construct was effective in killing 5637 cells in vitro, although it exhibited reduced efficacy, with higher EC₅₀ (61±17 pM) and reduced cytotoxicity (43±2%) compared with the scDb without Fc (FIG. 21). A mouse xenograft model was used where the 5637 cells were subcutaneously grafted in the NOG mice reconstituted with human peripheral blood mononuclear cells (hPBMC). sTL18 scDb-Fc significantly inhibited tumor growth at the dosage of 0.3 mg/kg and resulted in tumor shrinkage at 3 mg/kg (FIG. 22).

Altogether these data indicate that the tested antibodies, particularly sTL18, possess significant potential as a therapeutic drug, demonstrating exceptional selectivity and cytotoxic efficacy.

### Discussion of Examples 5-11

It is believed the antibodies described here are currently the only antibodies that are specific to HER2 S310F/Y mutant. Although it is a challenging task to develop a single point mutation specific antibody against a large molecular weight protein, oncogenic mutations generate cancer-specific neoantigens and promise safer drug-targets. The preceding Examples proved the viability of such approach. The disclosed antibodies were extensively tested for their off-target binding and cytotoxicity, especially towards HER2 WT, but no significant off-target response was observed. This remarkable selectivity promises a large therapeutic window and an optimistic outlook on developing these antibodies as an immunotherapy drug against cancer harboring this mutation. With improvement in sequencing technology and computational analyses, there is a vast mutational information that are readily available. Developing antibodies against key mutations that are easy to target and commonly found across many different cancers gives tactical advantage in minimizing side-effects arising from non-specificity in the era of personalized medicine.

In the preceding Examples, the cytotoxic potency of mutant HER2 binders in scDb format as T cell engagers, but ADC assays have also shown some promising results. With further optimization including change of toxin used and conjugation methods, the performance of ADC assay may improve. Even for T cell route, further optimization would be desirable for ultimate development of a drug, including for example using a different format of bispecific engager or employing a CAR-T system. However, with our mutant HER2-specific antibodies tested, it was demonstrated that the disclosed binders have a strong potential in targeting HER2-low cancers. Enhertu, a drug (deruxtecan)-conjugated trastuzumab, is the only available drug for HER2-low class breast cancer, approved recently by FDA in August 2022 (Modi et al., “Trastuzumab Deruxtecan in Previously Treated HER2-Low Advanced Breast Cancer,” N Engl J Med 387:9-20 (2022); Grinda et al., “Antibody-Drug Conjugate Revolution in Breast Cancer: The Road Ahead,” Current Treatment Options in Oncology 24:442-465 (2023), each of which is hereby incorporated by reference in its entirety). Although trastuzumab has an advantage in a sense that it can be used for with anyone with HER2-low cancer, as mentioned before it comes at a cost. Enhertu is reported to have several side effects including nausea, decrease in white and red blood cell counts and fatigue and more seriously a risk of interstitial lung disease, heart problem and embryo-fetal toxicity. The disclosed antibodies offer exciting opportunity as they would theoretically have less side-effects resulting from interaction with HER2 WT in healthy tissues.

While characterizing the binding property of the designed antibodies, the dynamic behavior of HER2 S310F/Y dimers was discovered. Prior research had indicated that HER2 S310F facilitates the formation of a covalent intermolecular disulfide bond, ‘locking’ the dimeric state and thereby constantly activating the signaling. The structure reinstates the power of S310F/Y in enhancing the dimeric interaction as both dimerization arms are clearly visible in the cryoEM map disclosed herein. However, the binding assays show homodimerization to be dynamic, allowing the disclosed Fabs to bind. HER2 is known to homodimerize in overexpressed condition, but the cell killing experiment with HER2 S310F/Y high expressing HEK293T cells showed potent cytotoxicity. It is possible that in cellular context, a minute portion of HER2 mutant gets covalently dimerized and become constitutively active in signaling.

The structural study of HER2 S310F/Y homodimer and binding assays with HER2 homodimer and heterodimers also led to the discovery that HER2 homodimer and heterodimer have different dynamics, even though all three of HER1, HER2, HER3 have the key tyrosine in dimerization arm, Y270, Y274, and Y265 respectively, that can make the π-π interaction with S310F/Y. A very strong dimeric interaction in heterodimeric setting was observed, and this actually impaired Fab binding much more than it did for homodimer. Comparison of different HER homo- and heterodimer structures also revealed the molecular basis behind this difference; the N-terminal of domain II in HER2 homodimers do not come in contact with each other. HER2 does not have any endogenous ligand to wedge between domains I and III and bend domain II to make the intermolecular contact. Indeed, the rigidity of HER2 ectodomain has been proposed to contribute to keeping HER2 in an autoinhibited state (Alvarado et al., “ErbB2 Resembles an Autoinhibited Invertebrate Epidermal Growth Factor Receptor,” Nature 461:287-291 (2009), which is hereby incorporated by reference in its entirety). This view also explains why HER2 WT would prefer to heterodimerize than homodimerize. In the absence of S310F/Y, stabilizing the dimerization arm of the partner, the effect of missing N-terminal domain II contact site would be even more crucial.

Due to the overlap in the epitope of the disclosed antibodies and the dimeric interface, ligands pose a risk to diminish the efficacy of the disclosed antibodies. However, it was also shown that even in the presence of EGF at concentration speculated to be 10 times higher than that in physiological serum, the cytotoxicity of the developed scDb is only marginally reduced. These results confirm that HER receptors are fully saturated with ligands and that all HER2 S310F/Y are fully engaged in heterodimeric or homodimeric state in actual physiological condition. Overall, the developed antibodies show great promise as candidates for therapeutics as well as tools for advancing mechanistic studies of the HER family receptors.

Although preferred embodiments have been depicted and described in detail herein, it will be apparent to those skilled in the relevant art that various modifications, additions, substitutions, and the like can be made without departing from the spirit of the disclosure and these are therefore considered to be within the scope of the disclosure as defined in the claims which follow.

1. An antibody-based molecule that binds mutant, but not wild type, Receptor tyrosine-protein kinase erbB-2 (HER2), said mutant HER2 comprising a substitution of serine at position 310 (S310) of SEQ ID NO: 1.

2. The antibody-based molecule of claim 1, wherein the mutant HER2 comprises a serine to phenylalanine substitution at position 310 (S310F) of SEQ ID NO: 1 or a serine to tyrosine substitution at position 310 (S310Y) of SEQ ID NO: 1.

3. The antibody-based molecule of claim 1 or claim 2, wherein said antibody-based molecule comprises a variable heavy region (V_{H}) comprising:
a complementarity-determining region 3 (CDR-H3) comprising an amino acid sequence of Y₁X₂X₃Y₄X₅M₆X₇X₈Y₉G₁₀S₁₁W₁₂X₁₃X₁₄M₁₅X₁₆X₁₇X₁₈D₁₉Y₂₀ (SEQ ID NO: 2), wherein X is any amino acid residue.

4. The antibody-based molecule of claim 1 or claim 2, wherein said antibody-based molecule comprises a variable heavy region (V_{H}) comprising:
a complementarity-determining region 3 (CDR-H3) comprising an amino acid X₁G₂X₃Y₄X₅X₆X₇X₈X₉G₁₀S₁₁W₁₂X₁₃X₁₄X₁₅P₁₆X₁₇X₁₈D₁₉Y₂₀ (SEQ ID NO: 93), where
X₁ can be any of Tyr (Y) or Phe (F);
X₃ can be any of Val (V), Glu (E), His (H), Ile (I), Leu (L), Met (M), Pro (P), Gln (Q), or Thy (T);
X₅ can be any of Thy (T), Ala (A), Glu (E), His (H), Ile (I), Lys (K), Leu (L), Met (M), Asn (N), Gln (Q), Arg (R), or Ser(S);
X₆ can be any of Met (M) or (L);
X₇ can be any of His (H), Ala (A), Lys (K), Met (M), Gln (Q), or Arg (R);
X₈ can be any of Gln (Q), Ala (A), Phe (F), His (H), Ile (I), Lys (K), Leu (L), Met (M), Asn (N), Arg (R), Ser(S), or Thy (T);
X₉ can be any of Tyr (Y), Ala (A), Phe (F), Gly (G), or Arg (R);
X₁₃ can be any of Glu (E), Gln (Q), or (V);
X₁₄ can be any of Gln (Q), Met (M), or Ser(S);
X₁₅ can be any of Met (M), Phe (F), Leu (L), Arg (R), or Thy (T);
X₁₇ can be any of Ala (A) or Tyr (Y); and
X₁₈ can be any of Phe (F), His (H), or Tyr (Y).

5. The antibody-based molecule of any one of claims 1 to 4, wherein said antibody-based molecule comprises a variable heavy region (V_{H}) comprising:
a complementarity-determining region 1 (CDR-H1) comprising an amino acid sequence of G₁X₂X₃I₄H₅ (SEQ ID NO: 92), where
X₂ can be any of Asn (N), Ala (A), Cys (C), Phe (F), His (H), Leu (L), Met (M), or Ser(S); and
X₃ can be any of Tyr (Y), Glu (E), or Phe (F).

6. The antibody-based molecule of claim 5, wherein said antibody-based molecule comprises a variable heavy region (V_{H}) comprising:
a framework region 1 (FR1) adjacent to CDR1 that includes a pair of residues selected from [Phe (F)/Trp (W)/Tyr (Y)]-[Ser(S)/Ala (A)/Phe (F)/Gly (G)/His (H)/Ile (I)/Met (M)/Asn (N)/Gln (Q)/Arg (R)/Thy (T)/Val (V)/Trp (W)/Tyr (Y)].

7. The antibody-based molecule of claim 5, wherein said antibody-based molecule comprises a variable heavy region (V_{H}) comprising:
a framework region 1 (FR1) adjacent to CDR1 that includes the amino acid sequence of EVQLVESGGGLVQPGGSLRLSCAASGFTX₂₉X₃₀ (SEQ ID NO: 95) where
X₂₉ can be any of Phe (F), Trp (W), or Tyr (Y); and
X₃₀ can be any of Ser(S), Ala (A), Phe (F), Gly (G), His (H), Ile (I), Met (M), Asn (N), Gln (Q), Arg (R), Thy (T), Val (V), Trp (W), or Tyr (Y).

8. The antibody-based molecule of any one of claims 1 to 7, wherein said antibody-based molecule comprises a variable light region (V_{L}) comprising:
a complementarity-determining region 3 (CDR-L3) comprising an amino acid comprises the amino acid sequence of Q₁Q₂X₃X₄-Z₅-X₆X₇X₈X₉T₁₀ (SEQ ID NO: 94) where
X₃ can be any of Tyr (Y), Ser(S) or Asp (D);
X₄ can be any of Ser(S), Asn (N), Trp (W), Leu (L), Glu (E), Asp (D), Gly (G),
Z₅ can be absent, in which case there is a direct bond between X₄ and X₆, or Z₅ can be a single residue or dipeptide selected from: Trp (W), Tyr (Y), Asp (D), Pro (P), -Trp-Glu-(-WE-), -Leu-Arg-(-LR-), or -Asn-Tyr-(-NY-);
X₆ can be any of Ser(S), Lys (K), Tyr (Y), Gly (G), Trp (W), Asn (N), or Glu (E);
X₇ can be any of Ser(S), Asp (D), Trp (W), Gly (G), Asp (D), or Pro (P);
X8 can be any of Leu (L) or Pro (P); and
X₉ can be any of Ile (I), Leu (L), or Val (V).

9. The antibody-based molecule of any one of claims 1 to 7, wherein said V_{H} further comprises:
a complementarity-determining region 1 (CDR-H1) comprising an amino acid sequence of any one of SEQ ID NOs: 3-13, 75, or 76 or a modified amino acid sequence of any one of SEQ ID NOs: 3-13, 75, or 76, said modified sequences having at least 80% sequence identity to any one of SEQ ID NOs: 3-13, 75, or 76; and
a complementarity-determining region 2 (CDR-H2) comprising an amino acid sequence of any one of SEQ ID NOs: 14-24, or a modified amino acid sequence of any one of SEQ ID NOs: 14-24, said modified sequences having at least 80% sequence identity to any one of SEQ ID NOs: 14-24.

10. The antibody-based molecule of any one of claim 1-7 or 9, wherein said V_{H} is selected from the group consisting of:
a V_{H} comprising the CDR-H1 of SEQ ID NO: 3, the CDR-H2 of SEQ ID NO: 14, and the CDR-H3 of SEQ ID NO: 2 (11-1);
a V_{H} comprising the CDR-H1 of SEQ ID NO: 4, the CDR-H2 of SEQ ID NO: 15, and the CDR-H3 of SEQ ID NO: 2 (CH2);
a V_{H} comprising the CDR-H1 of SEQ ID NO: 5, the CDR-H2 of SEQ ID NO: 16, and the CDR-H3 of SEQ ID NO: 2 (CH10);
a V_{H} comprising the CDR-H1 of SEQ ID NO: 6, the CDR-H2 of SEQ ID NO: 17, and the CDR-H3 of SEQ ID NO: 2 (CH15);
a V_{H} comprising the CDR-H1 of SEQ ID NO: 7, the CDR-H2 of SEQ ID NO: 18, and the CDR-H3 of SEQ ID NO: 2 (CL1);
a V_{H} comprising the CDR-H1 of SEQ ID NO: 8, the CDR-H2 of SEQ ID NO: 19, and the CDR-H3 of SEQ ID NO: 2 (CL3);
a V_{H} comprising the CDR-H1 of SEQ ID NO: 9, the CDR-H2 of SEQ ID NO: 20, and the CDR-H3 of SEQ ID NO: 2 (CL6);
a V_{H} comprising the CDR-H1 of SEQ ID NO: 10, the CDR-H2 of SEQ ID NO: 21, and the CDR-H3 of SEQ ID NO: 2 (CL11);
a V_{H} comprising the CDR-H1 of SEQ ID NO: 70, the CDR-H2 of SEQ ID NO: 71, and the CDR-H3 of SEQ ID NO: 93 (CH15V);
a V_{H} comprising the CDR-H1 of SEQ ID NO: 75, the CDR-H2 of SEQ ID NO: 17, and the CDR-H3 of SEQ ID NO: 93 (TL1);
a V_{H} comprising the CDR-H1 of SEQ ID NO: 76, the CDR-H2 of SEQ ID NO: 17, and the CDR-H3 of SEQ ID NO: 93 (TL7);
a V_{H} comprising the CDR-H1 of SEQ ID NO: 75, the CDR-H2 of SEQ ID NO: 17, and the CDR-H3 of SEQ ID NO: 93 (TL16);
a V_{H} comprising the CDR-H1 of SEQ ID NO: 76, the CDR-H2 of SEQ ID NO: 17, and the CDR-H3 of SEQ ID NO: 93 (TL18);
a V_{H} comprising the CDR-H1 of SEQ ID NO: 6, the CDR-H2 of SEQ ID NO: 17, and the CDR-H3 of SEQ ID NO: 93 (TL21-24); and
a V_{H} comprising the CDR-H1 of SEQ ID NO: 76, the CDR-H2 of SEQ ID NO: 17, and the CDR-H3 of SEQ ID NO: 93 (LL2, LL5).

11. The antibody-based molecule of any one of claim 1-7 or 9, wherein said V_{H} is selected from the group consisting of:
a V_{H} comprising the CDR-H1 of SEQ ID NO: 3, the CDR-H2 of SEQ ID NO: 14, and the CDR-H3 of SEQ ID NO: 25 (11-1);
a V_{H} comprising the CDR-H1 of SEQ ID NO: 4, the CDR-H2 of SEQ ID NO: 15, and the CDR-H3 of SEQ ID NO: 25 (CH₂);
a V_{H} comprising the CDR-H1 of SEQ ID NO: 5, the CDR-H2 of SEQ ID NO: 16, and the CDR-H3 of SEQ ID NO: 25 (CH10);
a V_{H} comprising the CDR-H1 of SEQ ID NO: 6, the CDR-H2 of SEQ ID NO: 17, and the CDR-H3 of SEQ ID NO: 25 (CH15);
a V_{H} comprising the CDR-H1 of SEQ ID NO: 7, the CDR-H2 of SEQ ID NO: 18, and the CDR-H3 of SEQ ID NO: 25 (CL1);
a V_{H} comprising the CDR-H1 of SEQ ID NO: 8, the CDR-H2 of SEQ ID NO: 19, and the CDR-H3 of SEQ ID NO: 25 (CL3);
a V_{H} comprising the CDR-H1 of SEQ ID NO: 9, the CDR-H2 of SEQ ID NO: 20, and the CDR-H3 of SEQ ID NO: 25 (CL6);
a V_{H} comprising the CDR-H1 of SEQ ID NO: 10, the CDR-H2 of SEQ ID NO: 21, and the CDR-H3 of SEQ ID NO: 25 (CL11);
a V_{H} comprising the CDR-H1 of SEQ ID NO: 11, the CDR-H2 of SEQ ID NO: 22, and the CDR-H3 of SEQ ID NO: 26 (7-20);
a V_{H} comprising the CDR-H1 of SEQ ID NO: 12, the CDR-H2 of SEQ ID NO: 23, and the CDR-H3 of SEQ ID NO: 27 (10-20);
a V_{H} comprising the CDR-H1 of SEQ ID NO: 13, the CDR-H2 of SEQ ID NO: 24, and the CDR-H3 of SEQ ID NO: 28 (25-20);
a V_{H} comprising the CDR-H1 of SEQ ID NO: 6, the CDR-H2 of SEQ ID NO: 17, and the CDR-H3 of SEQ ID NO: 74 (CH15V);
a V_{H} comprising the CDR-H1 of SEQ ID NO: 75, the CDR-H2 of SEQ ID NO: 17, and the CDR-H3 of SEQ ID NO: 77 (TL1);
a V_{H} comprising the CDR-H1 of SEQ ID NO: 76, the CDR-H2 of SEQ ID NO: 17, and the CDR-H3 of SEQ ID NO: 78 (TL7);
a V_{H} comprising the CDR-H1 of SEQ ID NO: 75, the CDR-H2 of SEQ ID NO: 17, and the CDR-H3 of SEQ ID NO: 79 (TL16);
a V_{H} comprising the CDR-H1 of SEQ ID NO: 76, the CDR-H2 of SEQ ID NO: 17, and the CDR-H3 of SEQ ID NO: 80 (TL18);
a V_{H} comprising the CDR-H1 of SEQ ID NO: 6, the CDR-H2 of SEQ ID NO: 17, and the CDR-H3 of SEQ ID NO: 81 (TL21-24); and
a V_{H} comprising the CDR-H1 of SEQ ID NO: 76, the CDR-H2 of SEQ ID NO: 17, and the CDR-H3 of SEQ ID NO: 78 (LL2, LL5).

12. The antibody-based molecule of any one of claims 1-11, wherein said antibody-based molecule is a single-domain antibody.

13. The antibody-based molecule of any one of claims 3-12, wherein said antibody-based molecule comprises:
a V_{H} comprising an amino acid sequence that is at least 80% identical to SEQ ID NO: 35;
a V_{H} comprising an amino acid sequence that is at least 80% identical to SEQ ID NO: 36;
a V_{H} comprising an amino acid sequence that is at least 80% identical to SEQ ID NO: 38;
a V_{H} comprising an amino acid sequence that is at least 80% identical to SEQ ID NO: 39;
a V_{H} comprising an amino acid sequence that is at least 80% identical to SEQ ID NO: 41;
a V_{H} comprising an amino acid sequence that is at least 80% identical to SEQ ID NO: 42;
a V_{H} comprising an amino acid sequence that is at least 80% identical to SEQ ID NO: 44;
a V_{H} comprising an amino acid sequence that is at least 80% identical to SEQ ID NO: 45;
a V_{H} comprising an amino acid sequence that is at least 80% identical to SEQ ID NO: 47;
a V_{H} comprising an amino acid sequence that is at least 80% identical to SEQ ID NO: 48;
a V_{H} comprising an amino acid sequence that is at least 80% identical to SEQ ID NO: 50;
a V_{H} comprising an amino acid sequence that is at least 80% identical to SEQ ID NO: 51;
a V_{H} comprising an amino acid sequence that is at least 80% identical to SEQ ID NO: 53;
a V_{H} comprising an amino acid sequence that is at least 80% identical to SEQ ID NO: 54;
a V_{H} comprising an amino acid sequence that is at least 80% identical to SEQ ID NO: 56;
a V_{H} comprising an amino acid sequence that is at least 80% identical to SEQ ID NO: 57;
a V_{H} comprising an amino acid sequence that is at least 80% identical to SEQ ID NO: 59;
a V_{H} comprising an amino acid sequence that is at least 80% identical to SEQ ID NO: 61;
a V_{H} comprising an amino acid sequence that is at least 80% identical to SEQ ID NO: 63;
a V_{H} comprising an amino acid sequence that is at least 80% identical to SEQ ID NO: 97;
a V_{H} comprising an amino acid sequence that is at least 80% identical to SEQ ID NO: 101;
a V_{H} comprising an amino acid sequence that is at least 80% identical to SEQ ID NO: 105;
a V_{H} comprising an amino acid sequence that is at least 80% identical to SEQ ID NO: 109;
a V_{H} comprising an amino acid sequence that is at least 80% identical to SEQ ID NO: 113;
a V_{H} comprising an amino acid sequence that is at least 80% identical to SEQ ID NO: 117;
a V_{H} comprising an amino acid sequence that is at least 80% identical to SEQ ID NO: 121;
a V_{H} comprising an amino acid sequence that is at least 80% identical to SEQ ID NO: 125;
a V_{H} comprising an amino acid sequence that is at least 80% identical to SEQ ID NO: 129;
a V_{H} comprising an amino acid sequence that is at least 80% identical to SEQ ID NO: 133; and
a V_{H} comprising an amino acid sequence that is at least 80% identical to SEQ ID NO: 137.

14. The antibody-based molecule of any one of claims 1-13, wherein said antibody-based molecule comprises a variable light region (V_{L}), wherein said V_{L} comprises:
a complementarity-determining region 1 (CDR-L1) having an amino acid sequence of SEQ ID NO: 29 or a modified amino acid sequence of SEQ ID NO: 29, said modified sequence having at least 80% sequence identity to SEQ ID NO: 29;
a complementarity-determining region 2 (CDR-L2) having an amino acid sequence of SEQ ID NO: 30 or a modified amino acid sequence of SEQ ID NO: 30, said modified sequence having at least 80% sequence identity to SEQ ID NO: 30; and
a complementarity-determining region 3 (CDR-L3) having an amino acid sequence of any one of SEQ ID NOs: 31-34 or 82-91 or a modified amino acid sequence of any one of SEQ ID NOs: 31-34 or 82-91, said modified sequence having at least 80% sequence identity to SEQ ID NOs: 31-34 or 82-91.

15. The antibody-based molecule of any one of claims 1-13, wherein the antibody-based molecule comprises a V_{L} comprising the CDR-L1 of SEQ ID NO: 29, the CDR-L2 of SEQ ID NO: 30, and the CDR-L3 of SEQ ID NO: 94.

16. The antibody-based molecule of claim 15, wherein said VL is selected from the group consisting of:
a V_{L} comprising the CDR-L1 of SEQ ID NO: 29, the CDR-L2 of SEQ ID NO: 30, and the CDR-L3 of SEQ ID NO: 31;
a V_{L} comprising the CDR-L1 of SEQ ID NO: 29, the CDR-L2 of SEQ ID NO: 30, and the CDR-L3 of SEQ ID NO: 32;
a V_{L} comprising the CDR-L1 of SEQ ID NO: 29, the CDR-L2 of SEQ ID NO: 30, and the CDR-L3 of SEQ ID NO: 33;
a V_{L} comprising the CDR-L1 of SEQ ID NO: 29, the CDR-L2 of SEQ ID NO: 30, and the CDR-L3 of SEQ ID NO: 34;
a V_{L} comprising the CDR-L1 of SEQ ID NO: 29, the CDR-L2 of SEQ ID NO: 30, and the CDR-L3 of SEQ ID NO: 82;
a V_{L} comprising the CDR-L1 of SEQ ID NO: 29, the CDR-L2 of SEQ ID NO: 30, and the CDR-L3 of SEQ ID NO: 83;
a V_{L} comprising the CDR-L1 of SEQ ID NO: 29, the CDR-L2 of SEQ ID NO: 30, and the CDR-L3 of SEQ ID NO: 84;
a V_{L} comprising the CDR-L1 of SEQ ID NO: 29, the CDR-L2 of SEQ ID NO: 30, and the CDR-L3 of SEQ ID NO: 85;
a V_{L} comprising the CDR-L1 of SEQ ID NO: 29, the CDR-L2 of SEQ ID NO: 30, and the CDR-L3 of SEQ ID NO: 86;
a V_{L} comprising the CDR-L1 of SEQ ID NO: 29, the CDR-L2 of SEQ ID NO: 30, and the CDR-L3 of SEQ ID NO: 87;
a V_{L} comprising the CDR-L1 of SEQ ID NO: 29, the CDR-L2 of SEQ ID NO: 30, and the CDR-L3 of SEQ ID NO: 88;
a V_{L} comprising the CDR-L1 of SEQ ID NO: 29, the CDR-L2 of SEQ ID NO: 30, and the CDR-L3 of SEQ ID NO: 89;
a V_{L} comprising the CDR-L1 of SEQ ID NO: 29, the CDR-L2 of SEQ ID NO: 30, and the CDR-L3 of SEQ ID NO: 90; and
a V_{L} comprising the CDR-L1 of SEQ ID NO: 29, the CDR-L2 of SEQ ID NO: 30, and the CDR-L3 of SEQ ID NO: 91.

17. The antibody-based molecule of any one of claims 1-16, wherein said antibody or binding fragment thereof comprises:
a V_{H} comprising the CDR-H1 of SEQ ID NO: 3, the CDR-H2 of SEQ ID NO: 14, and the CDR-H3 of SEQ ID NO: 2, and a V_{L} comprising the CDR-L1 of SEQ ID NO: 29, the CDR-L2 of SEQ ID NO: 30, and the CDR-L3 of SEQ ID NO: 31 (11-1);
a V_{H} comprising the CDR-H1 of SEQ ID NO: 4, the CDR-H2 of SEQ ID NO: 15, and the CDR-H3 of SEQ ID NO: 2, and a V_{L} comprising the CDR-L1 of SEQ ID NO: 29, the CDR-L2 of SEQ ID NO: 30, and the CDR-L3 of SEQ ID NO: 31 (CH2);
a V_{H} comprising the CDR-H1 of SEQ ID NO: 5, the CDR-H2 of SEQ ID NO: 16, and the CDR-H3 of SEQ ID NO: 2, and a V_{L} comprising the CDR-L1 of SEQ ID NO: 29, the CDR-L2 of SEQ ID NO: 30, and the CDR-L3 of SEQ ID NO: 31 (CH10);
a V_{H} comprising the CDR-H1 of SEQ ID NO: 6, the CDR-H2 of SEQ ID NO: 17, and the CDR-H3 of SEQ ID NO: 2, and a V_{L} comprising the CDR-L1 of SEQ ID NO: 29, the CDR-L2 of SEQ ID NO: 30, and the CDR-L3 of SEQ ID NO: 31 (CH15);
a V_{H} comprising the CDR-H1 of SEQ ID NO: 7, the CDR-H2 of SEQ ID NO: 18, and the CDR-H3 of SEQ ID NO: 2, and a V_{L} comprising the CDR-L1 of SEQ ID NO: 29, the CDR-L2 of SEQ ID NO: 30, and the CDR-L3 of SEQ ID NO: 31 (CL1);
a V_{H} comprising the CDR-H1 of SEQ ID NO: 8, the CDR-H2 of SEQ ID NO: 19, and the CDR-H3 of SEQ ID NO: 2, and a V_{L} comprising the CDR-L1 of SEQ ID NO: 29, the CDR-L2 of SEQ ID NO: 30, and the CDR-L3 of SEQ ID NO: 31 (CL3);
a V_{H} comprising the CDR-H1 of SEQ ID NO: 9, the CDR-H2 of SEQ ID NO: 20, and the CDR-H3 of SEQ ID NO: 2, and a V_{L} comprising the CDR-L1 of SEQ ID NO: 29, the CDR-L2 of SEQ ID NO: 30, and the CDR-L3 of SEQ ID NO: 31 (CL6);
a V_{H} comprising the CDR-H1 of SEQ ID NO: 10, the CDR-H2 of SEQ ID NO: 21, and the CDR-H3 of SEQ ID NO: 2, and a V_{L} comprising the CDR-L1 of SEQ ID NO: 29, the CDR-L2 of SEQ ID NO: 30, and the CDR-L3 of SEQ ID NO: 31 (CL11);
a V_{H} comprising the CDR-H1 of SEQ ID NO: 6, the CDR-H2 of SEQ ID NO: 17, and the CDR-H3 of SEQ ID NO: 93 and a V_{L} comprising the CDR-L1 of SEQ ID NO: 29, the CDR-L2 of SEQ ID NO: 30, and the CDR-L3 of SEQ ID NO: 94 (CH15V, TL21-24);
a V_{H} comprising the CDR-H1 of SEQ ID NO: 75, the CDR-H2 of SEQ ID NO: 17, and the CDR-H3 of SEQ ID NO: 93 and a V_{L} comprising the CDR-L1 of SEQ ID NO: 29, the CDR-L2 of SEQ ID NO: 30, and the CDR-L3 of SEQ ID NO: 94 (TL1, TL16););
a V_{H} comprising the CDR-H1 of SEQ ID NO: 76, the CDR-H2 of SEQ ID NO: 17, and the CDR-H3 of SEQ ID NO: 93 and a V_{L} comprising the CDR-L1 of SEQ ID NO: 29, the CDR-L2 of SEQ ID NO: 30, and the CDR-L3 of SEQ ID NO: 94 (TL7, TL18, LL2, LL5).

18. The antibody-based molecule of any one of claims 1-16, wherein said antibody or binding fragment thereof comprises:
a V_{H} comprising the CDR-H1 of SEQ ID NO: 3, the CDR-H2 of SEQ ID NO: 14, and the CDR-H3 of SEQ ID NO: 25, and the VL comprising the CDR-L1 of SEQ ID NO: 29, the CDR-L2 of SEQ ID NO: 30, and the CDR-L3 of SEQ ID NO: 31 (11-1);
a V_{H} comprising the CDR-H1 of SEQ ID NO: 4, the CDR-H2 of SEQ ID NO: 15, and the CDR-H3 of SEQ ID NO: 25, and the VL comprising the CDR-L1 of SEQ ID NO: 29, the CDR-L2 of SEQ ID NO: 30, and the CDR-L3 of SEQ ID NO: 31 (CH2);
a V_{H} comprising the CDR-H1 of SEQ ID NO: 5, the CDR-H2 of SEQ ID NO: 16, and the CDR-H3 of SEQ ID NO: 25, and the VL comprising the CDR-L1 of SEQ ID NO: 29, the CDR-L2 of SEQ ID NO: 30, and the CDR-L3 of SEQ ID NO: 31 (CH10);
a V_{H} comprising the CDR-H1 of SEQ ID NO: 6, the CDR-H2 of SEQ ID NO: 17, and the CDR-H3 of SEQ ID NO: 25, and the VL comprising the CDR-L1 of SEQ ID NO: 29, the CDR-L2 of SEQ ID NO: 30, and the CDR-L3 of SEQ ID NO: 31 (CH15);
a V_{H} comprising the CDR-H1 of SEQ ID NO: 7, the CDR-H2 of SEQ ID NO: 18, and the CDR-H3 of SEQ ID NO: 25, and the VL comprising the CDR-L1 of SEQ ID NO: 29, the CDR-L2 of SEQ ID NO: 30, and the CDR-L3 of SEQ ID NO: 31 (CL1);
a V_{H} comprising the CDR-H1 of SEQ ID NO: 8, the CDR-H2 of SEQ ID NO: 19, and the CDR-H3 of SEQ ID NO: 25, and the VL comprising the CDR-L1 of SEQ ID NO: 29, the CDR-L2 of SEQ ID NO: 30, and the CDR-L3 of SEQ ID NO: 31 (CL3);
a V_{H} comprising the CDR-H1 of SEQ ID NO: 9, the CDR-H2 of SEQ ID NO: 20, and the CDR-H3 of SEQ ID NO: 25, and the VL comprising the CDR-L1 of SEQ ID NO: 29, the CDR-L2 of SEQ ID NO: 30, and the CDR-L3 of SEQ ID NO: 31 (CL6);
a V_{H} comprising the CDR-H1 of SEQ ID NO: 10, the CDR-H2 of SEQ ID NO: 21, and the CDR-H3 of SEQ ID NO: 25, and the VL comprising the CDR-L1 of SEQ ID NO: 29, the CDR-L2 of SEQ ID NO: 30, and the CDR-L3 of SEQ ID NO: 31 (CL11);
a V_{H} comprising the CDR-H1 of SEQ ID NO: 11, the CDR-H2 of SEQ ID NO: 22, and the CDR-H3 of SEQ ID NO: 26, and the VL comprising the CDR-L1 of SEQ ID NO: 29, the CDR-L2 of SEQ ID NO: 30, and the CDR-L3 of SEQ ID NO: 32 (7-20)
a V_{H} comprising the CDR-H1 of SEQ ID NO: 12, the CDR-H2 of SEQ ID NO: 23, and the CDR-H3 of SEQ ID NO: 27, and the VL comprising the CDR-L1 of SEQ ID NO: 29, the CDR-L2 of SEQ ID NO: 30, and the CDR-L3 of SEQ ID NO: 33 (10-20);
a V_{H} comprising the CDR-H1 of SEQ ID NO: 13, the CDR-H2 of SEQ ID NO: 24, and the CDR-H3 of SEQ ID NO: 28, and the VL comprising the CDR-L1 of SEQ ID NO: 29, the CDR-L2 of SEQ ID NO: 30, and the CDR-L3 of SEQ ID NO: 34 (25-20);
a V_{H} comprising the CDR-H1 of SEQ ID NO: 6, the CDR-H2 of SEQ ID NO: 17, and the CDR-H3 of SEQ ID NO: 74 and a V_{L} comprising the CDR-L1 of SEQ ID NO: 29, the CDR-L2 of SEQ ID NO: 30, and the CDR-L3 of SEQ ID NO: 31 (CH15V);
a V_{H} comprising the CDR-H1 of SEQ ID NO: 75, the CDR-H2 of SEQ ID NO: 17, and the CDR-H3 of SEQ ID NO: 77 and a V_{L} comprising the CDR-L1 of SEQ ID NO: 29, the CDR-L2 of SEQ ID NO: 30, and the CDR-L3 of SEQ ID NO: 82 (TL1);
a V_{H} comprising the CDR-H1 of SEQ ID NO: 76, the CDR-H2 of SEQ ID NO: 17, and the CDR-H3 of SEQ ID NO: 78 and a V_{L} comprising the CDR-L1 of SEQ ID NO: 29, the CDR-L2 of SEQ ID NO: 30, and the CDR-L3 of SEQ ID NO: 83 (TL7);
a V_{H} comprising the CDR-H1 of SEQ ID NO: 75, the CDR-H2 of SEQ ID NO: 17, and the CDR-H3 of SEQ ID NO: 79 and a V_{L} comprising the CDR-L1 of SEQ ID NO: 29, the CDR-L2 of SEQ ID NO: 30, and the CDR-L3 of SEQ ID NO: 84 (TL16);
a V_{H} comprising the CDR-H1 of SEQ ID NO: 76, the CDR-H2 of SEQ ID NO: 17, and the CDR-H3 of SEQ ID NO: 80 and a V_{L} comprising the CDR-L1 of SEQ ID NO: 29, the CDR-L2 of SEQ ID NO: 30, and the CDR-L3 of SEQ ID NO: 85 (TL18);
a V_{H} comprising the CDR-H1 of SEQ ID NO: 6, the CDR-H2 of SEQ ID NO: 17, and the CDR-H3 of SEQ ID NO: 81 and a V_{L} comprising the CDR-L1 of SEQ ID NO: 29, the CDR-L2 of SEQ ID NO: 30, and the CDR-L3 of SEQ ID NO: 86 (TL21);
a V_{H} comprising the CDR-H1 of SEQ ID NO: 6, the CDR-H2 of SEQ ID NO: 17, and the CDR-H3 of SEQ ID NO: 81 and a V_{L} comprising the CDR-L1 of SEQ ID NO: 29, the CDR-L2 of SEQ ID NO: 30, and the CDR-L3 of SEQ ID NO: 87 (TL22);
a V_{H} comprising the CDR-H1 of SEQ ID NO: 6, the CDR-H2 of SEQ ID NO: 17, and the CDR-H3 of SEQ ID NO: 81 and a V_{L} comprising the CDR-L1 of SEQ ID NO: 29, the CDR-L2 of SEQ ID NO: 30, and the CDR-L3 of SEQ ID NO: 88 (TL23); a V_{H} comprising the CDR-H1 of SEQ ID NO: 6, the CDR-H2 of SEQ ID NO: 17, and the CDR-H3 of SEQ ID NO: 81 and a V_{L} comprising the CDR-L1 of SEQ ID NO: 29, the CDR-L2 of SEQ ID NO: 30, and the CDR-L3 of SEQ ID NO: 89 (TL24);
a V_{H} comprising the CDR-H1 of SEQ ID NO: 76, the CDR-H2 of SEQ ID NO: 17, and the CDR-H3 of SEQ ID NO: 81 and a V_{L} comprising the CDR-L1 of SEQ ID NO: 29, the CDR-L2 of SEQ ID NO: 30, and the CDR-L3 of SEQ ID NO: 90 (LL2);
a V_{H} comprising the CDR-H1 of SEQ ID NO: 76, the CDR-H2 of SEQ ID NO: 17, and the CDR-H3 of SEQ ID NO: 81 and a V_{L} comprising the CDR-L1 of SEQ ID NO: 29, the CDR-L2 of SEQ ID NO: 30, and the CDR-L3 of SEQ ID NO: 91 (LL5);
a V_{H} comprising the CDR-H1 of SEQ ID NO: 75, the CDR-H2 of SEQ ID NO: 17, and the CDR-H3 of SEQ ID NO: 77, and a V_{L} comprising the CDR-L1 of SEQ ID NO: 29, the CDR-L2 of SEQ ID NO: 30, and the CDR-L3 of SEQ ID NO: 31 (sTL1);
a V_{H} comprising the CDR-H1 of SEQ ID NO: 76, the CDR-H2 of SEQ ID NO: 17, and the CDR-H3 of SEQ ID NO: 80 and a V_{L} comprising the CDR-L1 of SEQ ID NO: 29, the CDR-L2 of SEQ ID NO: 30, and the CDR-L3 of SEQ ID NO: 31 (sTL18); and
a V_{H} comprising the CDR-H1 of SEQ ID NO: 76, the CDR-H2 of SEQ ID NO: 17, and the CDR-H3 of SEQ ID NO: 78, and a V_{L} comprising the CDR-L1 of SEQ ID NO: 29, the CDR-L2 of SEQ ID NO: 30, and the CDR-L3 of SEQ ID NO: 31 (sLL2).

19. The antibody-based molecule of any one of claims 3-18, wherein said molecule comprises:
a V_{H} comprising an amino acid sequence that is at least 80% identical to SEQ ID NO: 35 and a V_{L} comprising an amino acid sequence that is at least 80% identical to SEQ ID NO: 37;
a V_{H} comprising an amino acid sequence that is at least 80% identical to SEQ ID NO: 36 and a V_{L} comprising an amino acid sequence that is at least 80% identical to SEQ ID NO: 37;
a V_{H} comprising an amino acid sequence that is at least 80% identical to SEQ ID NO: 38 and a V_{L} comprising an amino acid sequence that is at least 80% identical to SEQ ID NO: 40;
a V_{H} comprising an amino acid sequence that is at least 80% identical to SEQ ID NO: 39 and a V_{L} comprising an amino acid sequence that is at least 80% identical to SEQ ID NO: 40;
a V_{H} comprising an amino acid sequence that is at least 80% identical to SEQ ID NO: 41 and a V_{L} comprising an amino acid sequence that is at least 80% identical to SEQ ID NO: 43;
a V_{H} comprising an amino acid sequence that is at least 80% identical to SEQ ID NO: 42 and a V_{L} comprising an amino acid sequence that is at least 80% identical to SEQ ID NO: 43;
a V_{H} comprising an amino acid sequence that is at least 80% identical to SEQ ID NO: 44 and a V_{L} comprising an amino acid sequence that is at least 80% identical to SEQ ID NO: 46;
a V_{H} comprising an amino acid sequence that is at least 80% identical to SEQ ID NO: 45 and a V_{L} comprising an amino acid sequence that is at least 80% identical to SEQ ID NO: 46;
a V_{H} comprising an amino acid sequence that is at least 80% identical to SEQ ID NO: 47 and a V_{L} comprising an amino acid sequence that is at least 80% identical to SEQ ID NO: 49;
a V_{H} comprising an amino acid sequence that is at least 80% identical to SEQ ID NO: 48 and a V_{L} comprising an amino acid sequence that is at least 80% identical to SEQ ID NO: 49;
a V_{H} comprising an amino acid sequence that is at least 80% identical to SEQ ID NO: 50 and a V_{L} comprising an amino acid sequence that is at least 80% identical to SEQ ID NO: 52;
a V_{H} comprising an amino acid sequence that is at least 80% identical to SEQ ID NO: 51 and a V_{L} comprising an amino acid sequence that is at least 80% identical to SEQ ID NO: 52;
a V_{H} comprising an amino acid sequence that is at least 80% identical to SEQ ID NO: 53 and a V_{L} comprising an amino acid sequence that is at least 80% identical to SEQ ID NO: 55;
a V_{H} comprising an amino acid sequence that is at least 80% identical to SEQ ID NO: 54 and a V_{L} comprising an amino acid sequence that is at least 80% identical to SEQ ID NO: 55;
a V_{H} comprising an amino acid sequence that is at least 80% identical to SEQ ID NO: 56 and a V_{L} comprising an amino acid sequence that is at least 80% identical to SEQ ID NO: 58;
a V_{H} comprising an amino acid sequence that is at least 80% identical to SEQ ID NO: 57 and a V_{L} comprising an amino acid sequence that is at least 80% identical to SEQ ID NO: 58;
a V_{H} comprising an amino acid sequence that is at least 80% identical to SEQ ID NO: 59 and a V_{L} comprising an amino acid sequence that is at least 80% identical to SEQ ID NO: 60;
a V_{H} comprising an amino acid sequence that is at least 80% identical to SEQ ID NO: 61 and a V_{L} comprising an amino acid sequence that is at least 80% identical to SEQ ID NO: 62;
a V_{H} comprising an amino acid sequence that is at least 80% identical to SEQ ID NO: 63 and a V_{L} comprising an amino acid sequence that is at least 80% identical to SEQ ID NO: 64;
a V_{H} comprising an amino acid sequence that is at least 80% identical to SEQ ID NO: 97 and a V_{L} comprising an amino acid sequence that is at least 80% identical to SEQ ID NO: 99;
a V_{H} comprising an amino acid sequence that is at least 80% identical to SEQ ID NO: 101 and a V_{L} comprising an amino acid sequence that is at least 80% identical to SEQ ID NO: 103;
a V_{H} comprising an amino acid sequence that is at least 80% identical to SEQ ID NO: 105 and a V_{L} comprising an amino acid sequence that is at least 80% identical to SEQ ID NO: 107;
a V_{H} comprising an amino acid sequence that is at least 80% identical to SEQ ID NO: 109 and a V_{L} comprising an amino acid sequence that is at least 80% identical to SEQ ID NO: 111;
a V_{H} comprising an amino acid sequence that is at least 80% identical to SEQ ID NO: 113 and a V_{L} comprising an amino acid sequence that is at least 80% identical to SEQ ID NO: 115;
a V_{H} comprising an amino acid sequence that is at least 80% identical to SEQ ID NO: 117 and a V_{L} comprising an amino acid sequence that is at least 80% identical to SEQ ID NO: 119;
a V_{H} comprising an amino acid sequence that is at least 80% identical to SEQ ID NO: 121 and a V_{L} comprising an amino acid sequence that is at least 80% identical to SEQ ID NO: 123;
a V_{H} comprising an amino acid sequence that is at least 80% identical to SEQ ID NO: 125 and a V_{L} comprising an amino acid sequence that is at least 80% identical to SEQ ID NO: 127;
a V_{H} comprising an amino acid sequence that is at least 80% identical to SEQ ID NO: 129 and a V_{L} comprising an amino acid sequence that is at least 80% identical to SEQ ID NO: 131; and
a V_{H} comprising an amino acid sequence that is at least 80% identical to SEQ ID NO: 133 and a V_{L} comprising an amino acid sequence that is at least 80% identical to SEQ ID NO: 135;
a V_{H} comprising an amino acid sequence that is at least 80% identical to SEQ ID NO: 137 and a V_{L} comprising an amino acid sequence that is at least 80% identical to SEQ ID NO: 139;
a V_{H} comprising an amino acid sequence that is at least 80% identical to SEQ ID NO: 101 and a V_{L} comprising an amino acid sequence that is at least 80% identical to SEQ ID NO: 99;
a V_{H} comprising an amino acid sequence that is at least 80% identical to SEQ ID NO: 113 and a V_{L} comprising an amino acid sequence that is at least 80% identical to SEQ ID NO: 99; and
a V_{H} comprising an amino acid sequence that is at least 80% identical to SEQ ID NO: 101 and a V_{L} comprising an amino acid sequence that is at least 80% identical to SEQ ID NO: 99.

20. The antibody-based molecule of any one of claims 1-19, wherein said antibody-based molecule is a human antibody-based molecule or HER2 epitope-binding fragment thereof.

21. The antibody-based molecule of any one of claims 1-19, wherein said antibody-based molecule is an IgG antibody.

22. The antibody-based molecule of any one of claims 1-19, wherein said antibody activates human complement.

23. The antibody-based molecule of any one of claims 1-19, wherein said antibody activates human antibody dependent cellular cytotoxicity.

24. The antibody-based molecule of any one of claims 1-23, wherein said antibody binds to a dimerized form of HER2.

25. The antibody-based molecule of any one of claims 1-24, wherein said antibody-based molecule is a monoclonal antibody or an epitope-binding fragment thereof.

26. The antibody-based molecule of any one of claims 1-25 wherein said antibody-based molecule is a full-length antibody, an epitope-binding fragment of an antibody, or an antibody derivative.

27. The antibody-based molecule of claim 26, wherein said antibody-based molecule is an epitope binding fragment selected from a F(ab) fragment, a F(ab′) fragment, and a F(ab′)₂ fragment.

28. The antibody-based molecule of claim 26, where said antibody-based molecule is an antibody derivative selected from the group consisting of a scFv, a minibody, a diabody, a triabody, and a tetrabody.

29. The antibody-based molecule of any one of claim 1-24, wherein said antibody-based molecule is a bi-specific antibody or a tri-specific antibody.

30. A polynucleotide encoding the antibody-based molecule of any one of claims 1-29.

31. A vector comprising the polynucleotide of claim 30.

32. A host cell comprising the vector of claim 31.

33. A chimeric antigen receptor molecule comprising:
an antibody-based molecule of any one of claims 1-29;
a transmembrane domain; and
an activation domain.

34. The chimeric receptor molecule of claim 33, wherein the antibody-based molecule comprises a scFv.

35. The chimeric receptor molecule of claim 34, wherein the scFv comprises
a V_{H} comprising an amino acid sequence that is at least 80% identical to SEQ ID NO: 35 and a V_{L} comprising an amino acid sequence that is at least 80% identical to SEQ ID NO: 37;
a V_{H} comprising an amino acid sequence that is at least 80% identical to SEQ ID NO: 36 and a V_{L} comprising an amino acid sequence that is at least 80% identical to SEQ ID NO: 37;
a V_{H} comprising an amino acid sequence that is at least 80% identical to SEQ ID NO: 38 and a V_{L} comprising an amino acid sequence that is at least 80% identical to SEQ ID NO: 40;
a V_{H} comprising an amino acid sequence that is at least 80% identical to SEQ ID NO: 39 and a V_{L} comprising an amino acid sequence that is at least 80% identical to SEQ ID NO: 40;
a V_{H} comprising an amino acid sequence that is at least 80% identical to SEQ ID NO: 41 and a V_{L} comprising an amino acid sequence that is at least 80% identical to SEQ ID NO: 43;
a V_{H} comprising an amino acid sequence that is at least 80% identical to SEQ ID NO: 42 and a V_{L} comprising an amino acid sequence that is at least 80% identical to SEQ ID NO: 43;
a V_{H} comprising an amino acid sequence that is at least 80% identical to SEQ ID NO: 44 and a V_{L} comprising an amino acid sequence that is at least 80% identical to SEQ ID NO: 46;
a V_{H} comprising an amino acid sequence that is at least 80% identical to SEQ ID NO: 45 and a V_{L} comprising an amino acid sequence that is at least 80% identical to SEQ ID NO: 46;
a V_{H} comprising an amino acid sequence that is at least 80% identical to SEQ ID NO: 47 and a V_{L} comprising an amino acid sequence that is at least 80% identical to SEQ ID NO: 49;
a V_{H} comprising an amino acid sequence that is at least 80% identical to SEQ ID NO: 48 and a V_{L} comprising an amino acid sequence that is at least 80% identical to SEQ ID NO: 49;
a V_{H} comprising an amino acid sequence that is at least 80% identical to SEQ ID NO: 50 and a V_{L} comprising an amino acid sequence that is at least 80% identical to SEQ ID NO: 52;
a V_{H} comprising an amino acid sequence that is at least 80% identical to SEQ ID NO: 51 and a V_{L} comprising an amino acid sequence that is at least 80% identical to SEQ ID NO: 52;
a V_{H} comprising an amino acid sequence that is at least 80% identical to SEQ ID NO: 53 and a V_{L} comprising an amino acid sequence that is at least 80% identical to SEQ ID NO: 55;
a V_{H} comprising an amino acid sequence that is at least 80% identical to SEQ ID NO: 54 and a V_{L} comprising an amino acid sequence that is at least 80% identical to SEQ ID NO: 55;
a V_{H} comprising an amino acid sequence that is at least 80% identical to SEQ ID NO: 56 and a V_{L} comprising an amino acid sequence that is at least 80% identical to SEQ ID NO: 58;
a V_{H} comprising an amino acid sequence that is at least 80% identical to SEQ ID NO: 57 and a V_{L} comprising an amino acid sequence that is at least 80% identical to SEQ ID NO: 58;
a V_{H} comprising an amino acid sequence that is at least 80% identical to SEQ ID NO: 59 and a V_{L} comprising an amino acid sequence that is at least 80% identical to SEQ ID NO: 60;
a V_{H} comprising an amino acid sequence that is at least 80% identical to SEQ ID NO: 61 and a V_{L} comprising an amino acid sequence that is at least 80% identical to SEQ ID NO: 62;
a V_{H} comprising an amino acid sequence that is at least 80% identical to SEQ ID NO: 63 and a V_{L} comprising an amino acid sequence that is at least 80% identical to SEQ ID NO: 64;
a V_{H} comprising an amino acid sequence that is at least 80% identical to SEQ ID NO: 97 and a V_{L} comprising an amino acid sequence that is at least 80% identical to SEQ ID NO: 99;
a V_{H} comprising an amino acid sequence that is at least 80% identical to SEQ ID NO: 101 and a V_{L} comprising an amino acid sequence that is at least 80% identical to SEQ ID NO: 103;
a V_{H} comprising an amino acid sequence that is at least 80% identical to SEQ ID NO: 105 and a V_{L} comprising an amino acid sequence that is at least 80% identical to SEQ ID NO: 107;
a V_{H} comprising an amino acid sequence that is at least 80% identical to SEQ ID NO: 109 and a V_{L} comprising an amino acid sequence that is at least 80% identical to SEQ ID NO: 111;
a V_{H} comprising an amino acid sequence that is at least 80% identical to SEQ ID NO: 113 and a V_{L} comprising an amino acid sequence that is at least 80% identical to SEQ ID NO: 115;
a V_{H} comprising an amino acid sequence that is at least 80% identical to SEQ ID NO: 117 and a V_{L} comprising an amino acid sequence that is at least 80% identical to SEQ ID NO: 119;
a V_{H} comprising an amino acid sequence that is at least 80% identical to SEQ ID NO: 121 and a V_{L} comprising an amino acid sequence that is at least 80% identical to SEQ ID NO: 123;
a V_{H} comprising an amino acid sequence that is at least 80% identical to SEQ ID NO: 125 and a V_{L} comprising an amino acid sequence that is at least 80% identical to SEQ ID NO: 127;
a V_{H} comprising an amino acid sequence that is at least 80% identical to SEQ ID NO: 129 and a V_{L} comprising an amino acid sequence that is at least 80% identical to SEQ ID NO: 131; and
a V_{H} comprising an amino acid sequence that is at least 80% identical to SEQ ID NO: 133 and a V_{L} comprising an amino acid sequence that is at least 80% identical to SEQ ID NO: 135;
a V_{H} comprising an amino acid sequence that is at least 80% identical to SEQ ID NO: 137 and a V_{L} comprising an amino acid sequence that is at least 80% identical to SEQ ID NO: 139;
a V_{H} comprising an amino acid sequence that is at least 80% identical to SEQ ID NO: 101 and a V_{L} comprising an amino acid sequence that is at least 80% identical to SEQ ID NO: 99;
a V_{H} comprising an amino acid sequence that is at least 80% identical to SEQ ID NO: 113 and a V_{L} comprising an amino acid sequence that is at least 80% identical to SEQ ID NO: 99; and
a V_{H} comprising an amino acid sequence that is at least 80% identical to SEQ ID NO: 101 and a V_{L} comprising an amino acid sequence that is at least 80% identical to SEQ ID NO: 99.

36. A polynucleotide encoding the chimeric receptor molecule of any one of claims 33-35.

37. A vector comprising the isolated polynucleotide of claim 36.

38. A host cell expressing the polynucleotide of claim 36 or comprising the vector of claim 37.

39. The host cell of claim 38, wherein said host cell is a natural killer cell.

40. The host cell of claim 38, wherein said host cell is a T cell.

41. The host cell of claim 38, wherein said host cell is a macrophage.

42. An immunoconjugate comprising:
the antibody-based molecule of any one of claims 1-29, and
a cytotoxic agent, wherein said cytotoxic agent is coupled to said antibody-based molecule.

43. The immunoconjugate of claim 42 further comprising:
a linker coupling the cytotoxic agent to the antibody-based molecule.

44. The immunoconjugate of claim 42 or claim 43, wherein the cytotoxic agent is a chemotherapeutic.

45. The immunoconjugate of claim 42 or claim 43, wherein the cytotoxic agent is selected from auristatin, a maytansinoid, a calicheamicin, a pyrrolobenzodiazepine, a nemorubicin derivative, and a 1-(chloromethyl)-2,3-dihydro-1H-benzo[e]indole (CBI).

46. A multi-specific antibody or multi-specific binding fragment thereof comprising:
a first antigen-binding arm comprising the antibody-based molecule of any one of claims 1-29, and
a second antigen-binding arm that binds to a surface antigen selectively expressed on an immune cell surface.

47. The multi-specific antibody or multi-specific binding fragment thereof of claim 46, wherein said multi-specific antibody is a bi-specific antibody or bi-specific binding fragment thereof.

48. The multi-specific antibody or multi-specific binding fragment thereof of claim 46 or 47, wherein said first antigen-binding arm comprises a VH (V_{H}), said V_{H} comprising:
a complementarity-determining region 3 (CDR-H3) comprising an amino acid sequence of Y₁X₂X₃Y₄X₅M₆X₇X₈Y₉G₁₀S₁₁W₁₂X₁₃X₁₄M₁₅X₁₆X₁₇X₁₈D₁₉Y₂₀ (SEQ ID NO: 2), wherein X is any amino acid residue; or
a complementarity-determining region 3 (CDR-H3) comprising an amino acid X₁G₂X₃Y₄X₅X₆X₇X₈X₉G₁₀S₁₁W₁₂X₁₃X₁₄X₁₅P₁₆X₁₇X₁₈D₁₉Y₂₀ (SEQ ID NO: 93), where
X₁ can be any of Tyr (Y) or Phe (F);
X₃ can be any of Val (V), Glu (E), His (H), Ile (I), Leu (L), Met (M), Pro (P), Gln (Q), or Thy (T);
X₅ can be any of Thy (T), Ala (A), Glu (E), His (H), Ile (I), Lys (K), Leu (L), Met (M), Asn (N), Gln (Q), Arg (R), or Ser(S);
X₆ can be any of Met (M) or (L);
X₇ can be any of His (H), Ala (A), Lys (K), Met (M), Gln (Q), or Arg (R);
X₈ can be any of Gln (Q), Ala (A), Phe (F), His (H), Ile (I), Lys (K), Leu (L), Met (M), Asn (N), Arg (R), Ser(S), or Thy (T);
X₉ can be any of Tyr (Y), Ala (A), Phe (F), Gly (G), or Arg (R);
X₁₃ can be any of Glu (E), Gln (Q), or (V);
X₁₄ can be any of Gln (Q), Met (M), or Ser(S);
X₁₅ can be any of Met (M), Phe (F), Leu (L), Arg (R), or Thy (T);
X₁₇ can be any of Ala (A) or Tyr (Y); and
X₁₈ can be any of Phe (F), His (H), or Tyr (Y).

49. The multi-specific antibody or multi-specific binding fragment thereof of claim 46 or 47, wherein said first antigen-binding arm comprises a VH (V_{H}), said V_{H} comprising:
a complementarity-determining region 1 (CDR-H1) comprising an amino acid sequence of G₁X₂X₃₁₄H₅ (SEQ ID NO: 92), where
X₂ can be any of Asn (N), Ala (A), Cys (C), Phe (F), His (H), Leu (L), Met (M), or Ser(S); and
X₃ can be any of Tyr (Y), Glu (E), or Phe (F).

50. The multi-specific antibody or multi-specific binding fragment thereof of claim 49, wherein said V_{H} comprising:
a framework region 1 (FR1) adjacent to CDR-H1 that includes a pair of residues selected from [Phe (F)/Trp (W)/Tyr (Y)]-[Ser(S)/Ala (A)/Phe (F)/Gly (G)/His (H)/Ile (I)/Met (M)/Asn (N)/Gln (Q)/Arg (R)/Thy (T)/Val (V)/Trp (W)/Tyr (Y)]; or
a framework region 1 (FR1) adjacent to CDR-H1 that includes the amino acid sequence of EVOLVESGGGLVQPGGSLRLSCAASGFTX₂₉X₃₀ (SEQ ID NO: 95) where
X₂₉ can be any of Phe (F), Trp (W), or Tyr (Y); and
X₃₀ can be any of Ser(S), Ala (A), Phe (F), Gly (G), His (H), Ile (I), Met (M), Asn (N), Gln (Q), Arg (R), Thy (T), Val (V), Trp (W), or Tyr (Y).

51. The multi-specific antibody or multi-specific binding fragment thereof of claim 46 or 47, wherein said first antigen-binding arm comprises a VL (V_{L}), said V_{L} comprising:
a complementarity-determining region 3 (CDR-L3) comprising an amino acid comprises the amino acid sequence of Q₁Q₂X₃X₄-Z₅-X₆X₇X₈X₉T₁₀ (SEQ ID NO: 94) where
X₃ can be any of Tyr (Y), Ser(S) or Asp (D);
X₄ can be any of Ser(S), Asn (N), Trp (W), Leu (L), Glu (E), Asp (D), Gly (G),
Z₅ can be absent, in which case there is a direct bond between X₄ and X₆, or Z₅ can be a single residue or dipeptide selected from: Trp (W), Tyr (Y), Asp (D), Pro (P), -Trp-Glu-(-WE-), -Leu-Arg-(-LR-), or -Asn-Tyr-(-NY-);
X₆ can be any of Ser(S), Lys (K), Tyr (Y), Gly (G), Trp (W), Asn (N), or Glu (E);
X₇ can be any of Ser(S), Asp (D), Trp (W), Gly (G), Asp (D), or Pro (P);
X₈ can be any of Leu (L) or Pro (P); and
X₉ can be any of Ile (I), Leu (L), or Val (V).

52. The multi-specific antibody or multi-specific binding fragment of any one of claims 46-51, wherein said antibody or binding fragment thereof comprises:
a V_{H} comprising the CDR-H1 of SEQ ID NO: 3, the CDR-H2 of SEQ ID NO: 14, and the CDR-H3 of SEQ ID NO: 2, and a V_{L} comprising the CDR-L1 of SEQ ID NO: 29, the CDR-L2 of SEQ ID NO: 30, and the CDR-L3 of SEQ ID NO: 31 (11-1);
a V_{H} comprising the CDR-H1 of SEQ ID NO: 4, the CDR-H2 of SEQ ID NO: 15, and the CDR-H3 of SEQ ID NO: 2, and a V_{L} comprising the CDR-L1 of SEQ ID NO: 29, the CDR-L2 of SEQ ID NO: 30, and the CDR-L3 of SEQ ID NO: 31 (CH2);
a V_{H} comprising the CDR-H1 of SEQ ID NO: 5, the CDR-H2 of SEQ ID NO: 16, and the CDR-H3 of SEQ ID NO: 2, and a V_{L} comprising the CDR-L1 of SEQ ID NO: 29, the CDR-L2 of SEQ ID NO: 30, and the CDR-L3 of SEQ ID NO: 31 (CH10);
a V_{H} comprising the CDR-H1 of SEQ ID NO: 6, the CDR-H2 of SEQ ID NO: 17, and the CDR-H3 of SEQ ID NO: 2, and a V_{L} comprising the CDR-L1 of SEQ ID NO: 29, the CDR-L2 of SEQ ID NO: 30, and the CDR-L3 of SEQ ID NO: 31 (CH15);
a V_{H} comprising the CDR-H1 of SEQ ID NO: 7, the CDR-H2 of SEQ ID NO: 18, and the CDR-H3 of SEQ ID NO: 2, and a V_{L} comprising the CDR-L1 of SEQ ID NO: 29, the CDR-L2 of SEQ ID NO: 30, and the CDR-L3 of SEQ ID NO: 31 (CL1);
a V_{H} comprising the CDR-H1 of SEQ ID NO: 8, the CDR-H2 of SEQ ID NO: 19, and the CDR-H3 of SEQ ID NO: 2, and a V_{L} comprising the CDR-L1 of SEQ ID NO: 29, the CDR-L2 of SEQ ID NO: 30, and the CDR-L3 of SEQ ID NO: 31 (CL3);
a V_{H} comprising the CDR-H1 of SEQ ID NO: 9, the CDR-H2 of SEQ ID NO: 20, and the CDR-H3 of SEQ ID NO: 2, and a V_{L} comprising the CDR-L1 of SEQ ID NO: 29, the CDR-L2 of SEQ ID NO: 30, and the CDR-L3 of SEQ ID NO: 31 (CL6);
a V_{H} comprising the CDR-H1 of SEQ ID NO: 10, the CDR-H2 of SEQ ID NO: 21, and the CDR-H3 of SEQ ID NO: 2, and a V_{L} comprising the CDR-L1 of SEQ ID NO: 29, the CDR-L2 of SEQ ID NO: 30, and the CDR-L3 of SEQ ID NO: 31 (CL11);
a V_{H} comprising the CDR-H1 of SEQ ID NO: 6, the CDR-H2 of SEQ ID NO: 17, and the CDR-H3 of SEQ ID NO: 93 and a V_{L} comprising the CDR-L1 of SEQ ID NO: 29, the CDR-L2 of SEQ ID NO: 30, and the CDR-L3 of SEQ ID NO: 94 (CH15V, TL21-24);
a V_{H} comprising the CDR-H1 of SEQ ID NO: 75, the CDR-H2 of SEQ ID NO: 17, and the CDR-H3 of SEQ ID NO: 93 and a V_{L} comprising the CDR-L1 of SEQ ID NO: 29, the CDR-L2 of SEQ ID NO: 30, and the CDR-L3 of SEQ ID NO: 94 (TL1, TL16););
a V_{H} comprising the CDR-H1 of SEQ ID NO: 76, the CDR-H2 of SEQ ID NO: 17, and the CDR-H3 of SEQ ID NO: 93 and a V_{L} comprising the CDR-L1 of SEQ ID NO: 29, the CDR-L2 of SEQ ID NO: 30, and the CDR-L3 of SEQ ID NO: 94 (TL7, TL18, LL2, LL5).

53. The multi-specific antibody or multi-specific binding fragment of any one of claims 46-51, wherein said antibody or binding fragment thereof comprises:
a V_{H} comprising the CDR-H1 of SEQ ID NO: 3, the CDR-H2 of SEQ ID NO: 14, and the CDR-H3 of SEQ ID NO: 25, and a V_{L} comprising the CDR-L1 of SEQ ID NO: 29, the CDR-L2 of SEQ ID NO: 30, and the CDR-L3 of SEQ ID NO: 31 (11-1);
a V_{H} comprising the CDR-H1 of SEQ ID NO: 4, the CDR-H2 of SEQ ID NO: 15, and the CDR-H3 of SEQ ID NO: 25, and a V_{L} comprising the CDR-L1 of SEQ ID NO: 29, the CDR-L2 of SEQ ID NO: 30, and the CDR-L3 of SEQ ID NO: 31 (CH2);
a V_{H} comprising the CDR-H1 of SEQ ID NO: 5, the CDR-H2 of SEQ ID NO: 16, and the CDR-H3 of SEQ ID NO: 25, and a V_{L} comprising the CDR-L1 of SEQ ID NO: 29, the CDR-L2 of SEQ ID NO: 30, and the CDR-L3 of SEQ ID NO: 31 (CH10);
a V_{H} comprising the CDR-H1 of SEQ ID NO: 6, the CDR-H2 of SEQ ID NO: 17, and the CDR-H3 of SEQ ID NO: 25, and a V_{L} comprising the CDR-L1 of SEQ ID NO: 29, the CDR-L2 of SEQ ID NO: 30, and the CDR-L3 of SEQ ID NO: 31 (CH15);
a V_{H} comprising the CDR-H1 of SEQ ID NO: 7, the CDR-H2 of SEQ ID NO: 18, and the CDR-H3 of SEQ ID NO: 25, and a V_{L} comprising the CDR-L1 of SEQ ID NO: 29, the CDR-L2 of SEQ ID NO: 30, and the CDR-L3 of SEQ ID NO: 31 (CL1);
a V_{H} comprising the CDR-H1 of SEQ ID NO: 8, the CDR-H2 of SEQ ID NO: 19, and the CDR-H3 of SEQ ID NO: 25, and a V_{L} comprising the CDR-L1 of SEQ ID NO: 29, the CDR-L2 of SEQ ID NO: 30, and the CDR-L3 of SEQ ID NO: 31 (CL3);
a V_{H} comprising the CDR-H1 of SEQ ID NO: 9, the CDR-H2 of SEQ ID NO: 20, and the CDR-H3 of SEQ ID NO: 25, and a V_{L} comprising the CDR-L1 of SEQ ID NO: 29, the CDR-L2 of SEQ ID NO: 30, and the CDR-L3 of SEQ ID NO: 31 (CL6);
a V_{H} comprising the CDR-H1 of SEQ ID NO: 10, the CDR-H2 of SEQ ID NO: 21, and the CDR-H3 of SEQ ID NO: 25, and a V_{L} comprising the CDR-L1 of SEQ ID NO: 29, the CDR-L2 of SEQ ID NO: 30, and the CDR-L3 of SEQ ID NO: 31 (CL11);
a V_{H} comprising the CDR-H1 of SEQ ID NO: 11, the CDR-H2 of SEQ ID NO: 22, and the CDR-H3 of SEQ ID NO: 26, and a V_{L} comprising the CDR-L1 of SEQ ID NO: 29, the CDR-L2 of SEQ ID NO: 30, and the CDR-L3 of SEQ ID NO: 32 (7-20);
a V_{H} comprising the CDR-H1 of SEQ ID NO: 12, the CDR-H2 of SEQ ID NO: 23, and the CDR-H3 of SEQ ID NO: 27, and a V_{L} comprising the CDR-L1 of SEQ ID NO: 29, the CDR-L2 of SEQ ID NO: 30, and the CDR-L3 of SEQ ID NO: 33 (10-20);
a V_{H} comprising the CDR-H1 of SEQ ID NO: 13, the CDR-H2 of SEQ ID NO: 24, and the CDR-H3 of SEQ ID NO: 28, and a V_{L} comprising the CDR-L1 of SEQ ID NO: 29, the CDR-L2 of SEQ ID NO: 30, and the CDR-L3 of SEQ ID NO: 34 (25-20);
a V_{H} comprising the CDR-H1 of SEQ ID NO: 6, the CDR-H2 of SEQ ID NO: 17, and the CDR-H3 of SEQ ID NO: 74 and a V_{L} comprising the CDR-L1 of SEQ ID NO: 29, the CDR-L2 of SEQ ID NO: 30, and the CDR-L3 of SEQ ID NO: 31 (CH15V);
a V_{H} comprising the CDR-H1 of SEQ ID NO: 75, the CDR-H2 of SEQ ID NO: 17, and the CDR-H3 of SEQ ID NO: 77 and a V_{L} comprising the CDR-L1 of SEQ ID NO: 29, the CDR-L2 of SEQ ID NO: 30, and the CDR-L3 of SEQ ID NO: 82 (TL1);
V_{H} comprising the CDR-H1 of SEQ ID NO: 76, the CDR-H2 of SEQ ID NO: 17, and the CDR-H3 of SEQ ID NO: 78 and a V_{L} comprising the CDR-L1 of SEQ ID NO: 29, the CDR-L2 of SEQ ID NO: 30, and the CDR-L3 of SEQ ID NO: 83 (TL7);
a V_{H} comprising the CDR-H1 of SEQ ID NO: 75, the CDR-H2 of SEQ ID NO: 17, and the CDR-H3 of SEQ ID NO: 79 and a V_{L} comprising the CDR-L1 of SEQ ID NO: 29, the CDR-L2 of SEQ ID NO: 30, and the CDR-L3 of SEQ ID NO: 84 (TL16);
a V_{H} comprising the CDR-H1 of SEQ ID NO: 76, the CDR-H2 of SEQ ID NO: 17, and the CDR-H3 of SEQ ID NO: 80 and a V_{L} comprising the CDR-L1 of SEQ ID NO: 29, the CDR-L2 of SEQ ID NO: 30, and the CDR-L3 of SEQ ID NO: 85 (TL18);
a V_{H} comprising the CDR-H1 of SEQ ID NO: 6, the CDR-H2 of SEQ ID NO: 17, and the CDR-H3 of SEQ ID NO: 81 and a V_{L} comprising the CDR-L1 of SEQ ID NO: 29, the CDR-L2 of SEQ ID NO: 30, and the CDR-L3 of SEQ ID NO: 86 (TL21);
a V_{H} comprising the CDR-H1 of SEQ ID NO: 6, the CDR-H2 of SEQ ID NO: 17, and the CDR-H3 of SEQ ID NO: 81 and a V_{L} comprising the CDR-L1 of SEQ ID NO: 29, the CDR-L2 of SEQ ID NO: 30, and the CDR-L3 of SEQ ID NO: 87 (TL22);
a V_{H} comprising the CDR-H1 of SEQ ID NO: 6, the CDR-H2 of SEQ ID NO: 17, and the CDR-H3 of SEQ ID NO: 81 and a V_{L} comprising the CDR-L1 of SEQ ID NO: 29, the CDR-L2 of SEQ ID NO: 30, and the CDR-L3 of SEQ ID NO: 88 (TL23);
a V_{H} comprising the CDR-H1 of SEQ ID NO: 6, the CDR-H2 of SEQ ID NO: 17, and the CDR-H3 of SEQ ID NO: 81 and a V_{L} comprising the CDR-L1 of SEQ ID NO: 29, the CDR-L2 of SEQ ID NO: 30, and the CDR-L3 of SEQ ID NO: 89 (TL24);
a V_{H} comprising the CDR-H1 of SEQ ID NO: 76, the CDR-H2 of SEQ ID NO: 17, and the CDR-H3 of SEQ ID NO: 81 and a V_{L} comprising the CDR-L1 of SEQ ID NO: 29, the CDR-L2 of SEQ ID NO: 30, and the CDR-L3 of SEQ ID NO: 90 (LL2); and
a V_{H} comprising the CDR-H1 of SEQ ID NO: 76, the CDR-H2 of SEQ ID NO: 17, and the CDR-H3 of SEQ ID NO: 81 and a V_{L} comprising the CDR-L1 of SEQ ID NO: 29, the CDR-L2 of SEQ ID NO: 30, and the CDR-L3 of SEQ ID NO: 91 (LL5);
a V_{H} comprising the CDR-H1 of SEQ ID NO: 75, the CDR-H2 of SEQ ID NO: 17, and the CDR-H3 of SEQ ID NO: 77, and a V_{L} comprising the CDR-L1 of SEQ ID NO: 29, the CDR-L2 of SEQ ID NO: 30, and the CDR-L3 of SEQ ID NO: 31 (sTL1);
a V_{H} comprising the CDR-H1 of SEQ ID NO: 76, the CDR-H2 of SEQ ID NO: 17, and the CDR-H3 of SEQ ID NO: 80 and a V_{L} comprising the CDR-L1 of SEQ ID NO: 29, the CDR-L2 of SEQ ID NO: 30, and the CDR-L3 of SEQ ID NO: 31 (STL18); and
a V_{H} comprising the CDR-H1 of SEQ ID NO: 76, the CDR-H2 of SEQ ID NO: 17, and the CDR-H3 of SEQ ID NO: 78, and a V_{L} comprising the CDR-L1 of SEQ ID NO: 29, the CDR-L2 of SEQ ID NO: 30, and the CDR-L3 of SEQ ID NO: 31 (sLL2).

54. The multi-specific antibody or multi-specific binding fragment of any one of claims 46-51, wherein said molecule comprises:
a V_{H} comprising an amino acid sequence that is at least 80% identical to SEQ ID NO: 35 and a V_{L} comprising an amino acid sequence that is at least 80% identical to SEQ ID NO: 37;
a V_{H} comprising an amino acid sequence that is at least 80% identical to SEQ ID NO: 36 and a V_{L} comprising an amino acid sequence that is at least 80% identical to SEQ ID NO: 37;
a V_{H} comprising an amino acid sequence that is at least 80% identical to SEQ ID NO: 38 and a V_{L} comprising an amino acid sequence that is at least 80% identical to SEQ ID NO: 40;
a V_{H} comprising an amino acid sequence that is at least 80% identical to SEQ ID NO: 39 and a V_{L} comprising an amino acid sequence that is at least 80% identical to SEQ ID NO: 40;
a V_{H} comprising an amino acid sequence that is at least 80% identical to SEQ ID NO: 41 and a V_{L} comprising an amino acid sequence that is at least 80% identical to SEQ ID NO: 43;
a V_{H} comprising an amino acid sequence that is at least 80% identical to SEQ ID NO: 42 and a V_{L} comprising an amino acid sequence that is at least 80% identical to SEQ ID NO: 43;
a V_{H} comprising an amino acid sequence that is at least 80% identical to SEQ ID NO: 44 and a V_{L} comprising an amino acid sequence that is at least 80% identical to SEQ ID NO: 46;
a V_{H} comprising an amino acid sequence that is at least 80% identical to SEQ ID NO: 45 and a V_{L} comprising an amino acid sequence that is at least 80% identical to SEQ ID NO: 46;
a V_{H} comprising an amino acid sequence that is at least 80% identical to SEQ ID NO: 47 and a V_{L} comprising an amino acid sequence that is at least 80% identical to SEQ ID NO: 49;
a V_{H} comprising an amino acid sequence that is at least 80% identical to SEQ ID NO: 48 and a V_{L} comprising an amino acid sequence that is at least 80% identical to SEQ ID NO: 49;
a V_{H} comprising an amino acid sequence that is at least 80% identical to SEQ ID NO: 50 and a V_{L} comprising an amino acid sequence that is at least 80% identical to SEQ ID NO: 52;
a V_{H} comprising an amino acid sequence that is at least 80% identical to SEQ ID NO: 51 and a V_{L} comprising an amino acid sequence that is at least 80% identical to SEQ ID NO: 52;
a V_{H} comprising an amino acid sequence that is at least 80% identical to SEQ ID NO: 53 and a V_{L} comprising an amino acid sequence that is at least 80% identical to SEQ ID NO: 55;
a V_{H} comprising an amino acid sequence that is at least 80% identical to SEQ ID NO: 54 and a V_{L} comprising an amino acid sequence that is at least 80% identical to SEQ ID NO: 55;
a V_{H} comprising an amino acid sequence that is at least 80% identical to SEQ ID NO: 56 and a V_{L} comprising an amino acid sequence that is at least 80% identical to SEQ ID NO: 58;
a V_{H} comprising an amino acid sequence that is at least 80% identical to SEQ ID NO: 57 and a V_{L} comprising an amino acid sequence that is at least 80% identical to SEQ ID NO: 58;
a V_{H} comprising an amino acid sequence that is at least 80% identical to SEQ ID NO: 59 and a V_{L} comprising an amino acid sequence that is at least 80% identical to SEQ ID NO: 60;
a V_{H} comprising an amino acid sequence that is at least 80% identical to SEQ ID NO: 61 and a V_{L} comprising an amino acid sequence that is at least 80% identical to SEQ ID NO: 62;
a V_{H} comprising an amino acid sequence that is at least 80% identical to SEQ ID NO: 63 and a V_{L} comprising an amino acid sequence that is at least 80% identical to SEQ ID NO: 64;
a V_{H} comprising an amino acid sequence that is at least 80% identical to SEQ ID NO: 97 and a V_{L} comprising an amino acid sequence that is at least 80% identical to SEQ ID NO: 99;
a V_{H} comprising an amino acid sequence that is at least 80% identical to SEQ ID NO: 101 and a V_{L} comprising an amino acid sequence that is at least 80% identical to SEQ ID NO: 103;
a V_{H} comprising an amino acid sequence that is at least 80% identical to SEQ ID NO: 105 and a V_{L} comprising an amino acid sequence that is at least 80% identical to SEQ ID NO: 107;
a V_{H} comprising an amino acid sequence that is at least 80% identical to SEQ ID NO: 109 and a V_{L} comprising an amino acid sequence that is at least 80% identical to SEQ ID NO: 111;
a V_{H} comprising an amino acid sequence that is at least 80% identical to SEQ ID NO: 113 and a V_{L} comprising an amino acid sequence that is at least 80% identical to SEQ ID NO: 115;
a V_{H} comprising an amino acid sequence that is at least 80% identical to SEQ ID NO: 117 and a V_{L} comprising an amino acid sequence that is at least 80% identical to SEQ ID NO: 119;
a V_{H} comprising an amino acid sequence that is at least 80% identical to SEQ ID NO: 121 and a V_{L} comprising an amino acid sequence that is at least 80% identical to SEQ ID NO: 123;
a V_{H} comprising an amino acid sequence that is at least 80% identical to SEQ ID NO: 125 and a V_{L} comprising an amino acid sequence that is at least 80% identical to SEQ ID NO: 127;
a V_{H} comprising an amino acid sequence that is at least 80% identical to SEQ ID NO: 129 and a V_{L} comprising an amino acid sequence that is at least 80% identical to SEQ ID NO: 131; and
a V_{H} comprising an amino acid sequence that is at least 80% identical to SEQ ID NO: 133 and a V_{L} comprising an amino acid sequence that is at least 80% identical to SEQ ID NO: 135;
a V_{H} comprising an amino acid sequence that is at least 80% identical to SEQ ID NO: 137 and a V_{L} comprising an amino acid sequence that is at least 80% identical to SEQ ID NO: 139;
a V_{H} comprising an amino acid sequence that is at least 80% identical to SEQ ID NO: 101 and a V_{L} comprising an amino acid sequence that is at least 80% identical to SEQ ID NO: 99;
a V_{H} comprising an amino acid sequence that is at least 80% identical to SEQ ID NO: 113 and a V_{L} comprising an amino acid sequence that is at least 80% identical to SEQ ID NO: 99; and
a V_{H} comprising an amino acid sequence that is at least 80% identical to SEQ ID NO: 101 and a V_{L} comprising an amino acid sequence that is at least 80% identical to SEQ ID NO: 99.

55. The multi-specific antibody or multi-specific binding fragment according to any one of claims 46 to 54, wherein the second antigen-binding arm binds to a surface antigen selectively expressed on T cells.

56. The multi-specific antibody or multi-specific binding fragment thereof of claim 55, wherein the T cell surface antigen is CD3.

57. The multi-specific antibody or multi-specific binding fragment thereof of claim 56, wherein the multi-specific antibody or multi-specific binding fragment thereof is selected from:
CH15V_scDb
DIVRSDIQMTQSPSSLSASVGDRVTITCRASQSVSSAVAWYQQKPGKAPKLLIYSASSLYSGVPSRFS
GSRSGTDFTLTISSLQPEDFATYYCQQSSSSLITFGQGTKVEIKGGGGSEVQLQQSGPELVKPGASMK
ISCKASGYSFTGYTMNWVKQSHGKNLEWMGLINPYKGVSTYNQKFKDKATLTVDKSSSTAYMELLSLT
SEDSAVYYCARSGYYGDSDWYFDVWGQGTTLTVSSGGGGSGGGGSGGGGSDIQMTQTTSSLSASLGDR
VTISCRASQDIRNYLNWYQQKPDGTVKLLIYYTSRLHSGVPSKFSGSGSGTDYSLTISNLEQEDIATY
FCQQGNTLPWTFAGGTKLEIKGGGGSEVQLVESGGGLVQPGGSLRLSCAASGFTFSGNYIHWVRQAPG
KGLEWVASIYSAGGYTDYADSVKGRFTISADTSKNTAYLQMNSLRAEDTAVYYCARYGVYTMHQYGSW
EQMPAFDYWGQGTLVTVSSLEGGGGLNDIFEAQKIEWHESRHHHHHH
sLL2_scDb
DIVRSDIQMTQSPSSLSASVGDRVTITCRASQSVSSAVAWYQQKPGKAPKLLIYSASSLYSGVPSRFS
GSRSGTDFTLTISSLQPEDFATYYCQQSSSSLITFGQGTKVEIKGGGGSEVQLQQSGPELVKPGASMK
ISCKASGYSFTGYTMNWVKQSHGKNLEWMGLINPYKGVSTYNQKFKDKATLTVDKSSSTAYMELLSLT
SEDSAVYYCARSGYYGDSDWYFDVWGQGTTLTVSSGGGGSGGGGSGGGGSDIQMTQTTSSLSASLGDR
VTISCRASQDIRNYLNWYQQKPDGTVKLLIYYTSRLHSGVPSKFSGSGSGTDYSLTISNLEQEDIATY
FCQQGNTLPWTFAGGTKLEIKGGGGSEVQLVESGGGLVQPGGSLRLSCAASGFTWSGSYIHWVRQAPG
KGLEWVASIYSAGGYTDYADSVKGRFTISADTSKNTAYLQMNSLRAEDTAVYYCARYGVYTLASAGSW
ESLPAFDYWGQGTLVTVSSLEGGGGLNDIFEAQKIEWHESRHHHHHH
sTL1_scDb
DIVRSDIQMTQSPSSLSASVGDRVTITCRASQSVSSAVAWYQQKPGKAPKLLIYSASSLYSGVPSRFS
GSRSGTDFTLTISSLQPEDFATYYCQQSSSSLITFGQGTKVEIKGGGGSEVQLQQSGPELVKPGASMK
ISCKASGYSFTGYTMNWVKQSHGKNLEWMGLINPYKGVSTYNQKFKDKATLTVDKSSSTAYMELLSLT
SEDSAVYYCARSGYYGDSDWYFDVWGQGTTLTVSSGGGGSGGGGSGGGGSDIQMTQTTSSLSASLGDR
VTISCRASQDIRNYLNWYQQKPDGTVKLLIYYTSRLHSGVPSKFSGSGSGTDYSLTISNLEQEDIATY
FCQQGNTLPWTFAGGTKLEIKGGGGSEVQLVESGGGLVQPGGSLRLSCAASGFTWSGAYIHWVRQAPG
KGLEWVASIYSAGGYTDYADSVKGRFTISADTSKNTAYLQMNSLRAEDTAVYYCARYGTYELKSYGSW
ESLPAFDYWGQGTLVTVSSLEGGGGLNDIFEAQKIEWHESRHHHHHH
sTL18_scDb
DIVRSDIQMTQSPSSLSASVGDRVTITCRASQSVSSAVAWYQQKPGKAPKLLIYSASSLYSGVPSRFS
GSRSGTDFTLTISSLQPEDFATYYCQQSSSSLITFGQGTKVEIKGGGGSEVQLQQSGPELVKPGASMK
ISCKASGYSFTGYTMNWVKQSHGKNLEWMGLINPYKGVSTYNQKFKDKATLTVDKSSSTAYMELLSLT
SEDSAVYYCARSGYYGDSDWYFDVWGQGTTLTVSSGGGGSGGGGSGGGGSDIQMTQTTSSLSASLGDR
VTISCRASQDIRNYLNWYQQKPDGTVKLLIYYTSRLHSGVPSKFSGSGSGTDYSLTISNLEQEDIATY
FCQQGNTLPWTFAGGTKLEIKGGGGSEVQLVESGGGLVQPGGSLRLSCAASGFTEGGSYIHWVRQAPG
KGLEWVASIYSAGGYTDYADSVKGRFTISADTSKNTAYLQMNSLRAEDTAVYYCARYGVYTLHQYGSW
EQLPAFDYWGQGTLVTVSSLEGGGGLNDIFEAQKIEWHESRHHHHHH
sTL18_BiTE_HL
EVQLVESGGGLVQPGGSLRLSCAASGFTFGGSYIHWVRQAPGKGLEWVASIYSAGGYTDYADSVKGRFTISADT
SKNTAYLQMNSLRAEDTAVYYCARYGVYTLHQYGSWEQLPAFDYWGQGTLVTVSSGGGGSGGGGSGGGGSDIQM
TQSPSSLSASVGDRVTITCRASQSVSSAVAWYQQKPGKAPKLLIYSASSLYSGVPSRFSGSRSGTDFTLTISSL
QPEDFATYYCQQSSSSLITFGQGTKVEIKSGGGGSEVQLQQSGPELVKPGASMKISCKASGYSFTGYTMNWVKQ
SHGKNLEWMGLINPYKGVSTYNQKFKDKATLTVDKSSSTAYMELLSLTSEDSAVYYCARSGYYGDSDWYFDVWG
QGTTLTVSSGGGGSGGGGSGGGGSDIQMTQTTSSLSASLGDRVTISCRASQDIRNYLNWYQQKPDGTVKLLIYY
TSRLHSGVPSKFSGSGSGTDYSLTISNLEQEDIATYFCQQGNTLPWTFAGGTKLEIKLEGGGGLNDIFEAQKIE
WHESRHHHHHH
sTL18_BiTE_LH
DIQMTQSPSSLSASVGDRVTITCRASQSVSSAVAWYQQKPGKAPKLLIYSASSLYSGVPSRFSGSRSGTDFTLT
ISSLQPEDFATYYCQQSSSSLITFGQGTKVEIKGGGGSGGGGSGGGGSEVQLVESGGGLVQPGGSLRLSCAASG
FTFGGSYIHWVRQAPGKGLEWVASIYSAGGYTDYADSVKGRFTISADTSKNTAYLQMNSLRAEDTAVYYCARYG
VYTLHQYGSWEQLPAFDYWGQGTLVTVSSSGGGGSDIQMTQTTSSLSASLGDRVTISCRASQDIRNYLNWYQQK
PDGTVKLLIYYTSRLHSGVPSKFSGSGSGTDYSLTISNLEQEDIATYFCQQGNTLPWTFAGGTKLEIKGGGGSG
GGGSGGGGSEVQLQQSGPELVKPGASMKISCKASGYSFTGYTMNWVKQSHGKNLEWMGLINPYKGVSTYNQKFK
DKATLTVDKSSSTAYMELLSLTSEDSAVYYCARSGYYGDSDWYFDVWGQGTTLTVSSLEGGGGLNDIFEAQKIE
WHESRHHHHHH
sTL18_CrossMab having chains A, B, C and
>Chain_A (UCHT1VHCL)
EVQLQQSGPELVKPGASMKISCKASGYSFTGYTMNWVKQSHGKNLEWMGLINPYKGVSTYNQKFKDKATLTVDK
SSSTAYMELLSLTSEDSAVYYCARSGYYGDSDWYFDVWGQGTTLTVSSASVAAPSVFIFPPSDEQLKSGTASVV
CLLNNFYPREAKVQWKVDNALQSGNSQESVTEQDSKDSTYSLSSTLTLSKADYEKHKVYACEVTHQGLSSPVTK
SFNRGEC
>Chain_B (sTL18VHCH1_UCHT1VLCH1_Fcknob)
EVQLVESGGGLVQPGGSLRLSCAASGFTEGGSYIHWVRQAPGKGLEWVASIYSAGGYTDYADSVKGRFTISADT
SKNTAYLQMNSLRAEDTAVYYCARYGVYTLHQYGSWEQLPAFDYWGQGTLVTVSSASTKGPSVFPLAPSSKSTS
GGTAALGCLVEDYFPEPVTVSWNSGALTSGVHTFPAVLQSSGLYSLSSVVTVPSSSLGTQTYICNVNHKPSNTK
VDEKVEPKSCDGGGGSGGGGSDIQMTQTTSSLSASLGDRVTISCRASQDIRNYLNWYQQKPDGTVKLLIYYTSR
LHSGVPSKFSGSGSGTDYSLTISNLEQEDIATYFCQQGNTLPWTFAGGTKLEIKSSASTKGPSVFPLAPSSKST
SGGTAALGCLVKDYFPEPVTVSWNSGALTSGVHTFPAVLQSSGLYSLSSVVTVPSSSLGTQTYICNVNHKPSNT
KVDKKVEPKSCDKTHTCPPCPAPEAAGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALGAPIEKTISKAKGQPREPQVYTLPPCRDE
LTKNQVSLWCLVKGFYPSDIAVEWESNGQPENNYKTTPPVLDSDGSFFLYSKLTVDKSRWQQGNVFSCSVMHEA
LHNHYTQKSLSLSP
>Chain_C (sTL18VHCH1_Fchole)
EVQLVESGGGLVQPGGSLRLSCAASGFTEGGSYIHWVRQAPGKGLEWVASIYSAGGYTDYADSVKGRFTISADT
SKNTAYLQMNSLRAEDTAVYYCARYGVYTLHQYGSWEQLPAFDYWGQGTLVTVSSASTKGPSVFPLAPSSKSTS
GGTAALGCLVEDYFPEPVTVSWNSGALTSGVHTFPAVLQSSGLYSLSSVVTVPSSSLGTQTYICNVNHKPSNTK
VDEKVEPKSCDKTHTCPPCPAPEAAGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALGAPIEKTISKAKGQPREPQVCTLPPSRDEL
TKNQVSLSCAVKGFYPSDIAVEWESNGQPENNYKTTPPVLDSDGSFFLVSKLTVDKSRWQQGNVFSCSVMHEAL
HNHYTQKSLSLSP
>Chain_D (sTL18VLCL)
DIQMTQSPSSLSASVGDRVTITCRASQSVSSAVAWYQQKPGKAPKLLIYSASSLYSGVPSRFSGSRSGTDFTLT
ISSLQPEDFATYYCQQSSSSLITFGQGTKVEIKRTVAAPSVFIFPPSDRKLKSGTASVVCLLNNFYPREAKVQW
KVDNALQSGNSQESVTEQDSKDSTYSLSSTLTLSKADYEKHKVYACEVTHQGLSSPVTKSFNRGEC
sTL18 FabH-UCHT1 scFv having chains A and B
>sTL18VHCH1_UCHT1scFv (chain A)
EVQLVESGGGLVQPGGSLRLSCAASGFTEGGSYIHWVRQAPGKGLEWVASIYSAGGYTDYADSVKGRFTISADT
SKNTAYLQMNSLRAEDTAVYYCARYGVYTLHQYGSWEQLPAFDYWGQGTLVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKPSNTK
VDKRVEPKSCGGGGSEVQLQQSGPELVKPGASMKISCKASGYSFTGYTMNWVKQSHGKNLEWMGLINPYKGVST
YNQKFKDKATLTVDKSSSTAYMELLSLTSEDSAVYYCARSGYYGDSDWYFDVWGQGTTLTVSSGGGGSGGGGSG
GGGSDIQMTQTTSSLSASLGDRVTISCRASQDIRNYLNWYQQKPDGTVKLLIYYTSRLHSGVPSKFSGSGSGTD
YSLTISNLEQEDIATYFCQQGNTLPWTFAGGTKLEIK
>sTL18VLCL (chain B)
DIQMTQSPSSLSASVGDRVTITCRASQSVSSAVAWYQQKPGKAPKLLIYSASSLYSGVPSRFSGSRSGTDFTLT
ISSLQPEDFATYYCQQSSSSLITFGQGTKVEIKRTVAAPSVFIFPPSDEQLKSGTASVVCLLNNFYPREAKVQW
KVDNALQSGNSQESVTEQDSKDSTYSLSSTLTLSKADYEKHKVYACEVTHQGLSSPVTKSFNRGEC
sTL18 FabL-UCHT1 scFv having chains A and B
>sTIL18VHCH1 (chain A)
EVQLVESGGGLVQPGGSLRLSCAASGFTFGGSYIHWVRQAPGKGLEWVASIYSAGGYTDYADSVKGRFTISADT
SKNTAYLQMNSLRAEDTAVYYCARYGVYTLHQYGSWEQLPAFDYWGQGTLVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKPSNTK
VDKRVEPKSC
>sTL18VLCL_UCHT1scFv (chain B)
DIQMTQSPSSLSASVGDRVTITCRASQSVSSAVAWYQQKPGKAPKLLIYSASSLYSGVPSRFSGSRSGTDFTLT
ISSLQPEDFATYYCQQSSSSLITFGQGTKVEIKRTVAAPSVFIFPPSDEQLKSGTASVVCLLNNFYPREAKVQW
KVDNALQSGNSQESVTEQDSKDSTYSLSSTLTLSKADYEKHKVYACEVTHQGLSSPVTKSFNRGECGGGGSEVQ
LQQSGPELVKPGASMKISCKASGYSFTGYTMNWVKQSHGKNLEWMGLINPYKGVSTYNQKFKDKATLTVDKSSS
TAYMELLSLTSEDSAVYYCARSGYYGDSDWYFDVWGQGTTLTVSSGGGGSGGGGSGGGGSDIQMTQTTSSLSAS
LGDRVTISCRASQDIRNYLNWYQQKPDGTVKLLIYYTSRLHSGVPSKFSGSGSGTDYSLTISNLEQEDIATYFC
QQGNTLPWTFAGGTKLEIK

58. The multi-specific antibody or multi-specific binding fragment thereof according to any one of claims 46 to 54, wherein the second antigen-binding arm binds to a surface antigen selectively expressed on natural killer (NK) cells.

59. The multi-specific antibody or multi-specific binding fragment thereof of claim 58, wherein the NK cell specific surface antigen is selected from the group consisting of CD16A, NKG2D, CD94/NKG2C, NKp30, NKp44, and NKp46.

60. A polynucleotide encoding the multi-specific antibody or multi-specific binding fragment thereof of any one of claims 46 to 59.

61. A vector comprising the isolated polynucleotide of claim 60.

62. A host cell comprising the vector of claim 61.

63. A pharmaceutical composition comprising:
the antibody-based molecule of any one of claims 1-29, the immunoconjugate of any one of claims 42-45, or the multi-specific antibody or multi-specific binding fragment thereof of any one of claims 46 to 59, the polynucleotides of claims or 30, 36, or 60, or the vectors of claim 31, 37, or 61; and
a pharmaceutically acceptable carrier.

64. A delivery vehicle comprising the polynucleotide of claim 30, 36, or 60 or the vector of any one of claim 31, 37, or 61.

65. The delivery vehicle of claim 64, wherein the delivery vehicle is selected from a nanoparticle, a polymer-based particle, and a lipid-based particle.

66. A method of treating a subject having a HER2-positive cancer, said method comprising:
administering to the subject the pharmaceutical composition of claim 63 in an amount effective to treat the subject having the HER2-positive cancer.

67. A method of treating a subject having a cancer expressing a mutant HER-2, said method comprising:
administering to the subject a population of autologous immune cells expressing the chimeric antigen receptor of any one of claims 33-35 in an amount effective to treat the subject having the mutant HER-2 expressing cancer.

68. The method of claim 67, wherein the population of autologous immune cells is selected from a population of T cells, NK cells, and macrophages.

69. The method of claim 66 or claim 67, wherein the cancer expressing mutant HER2 is characterized by cancer cells expressing a S310 mutant HER2 protein.

70. The method of claim 69, wherein the S310 mutant HER2 is a S310F mutant HER2.

71. The method of claim 69, wherein the S310 mutant HER2 is a S310Y mutant HER2.

72. The method of any one of claims 66-71, wherein the cancer expressing mutant HER2 is selected from breast cancer, colorectal cancer, lung squamous cell carcinoma, lung small cell cancer, lung adenocarcinoma, bladder cancer, gastric cancer, glioblastoma, cutaneous squamous carcinoma, gallbladder cancer, head and neck squamous cell carcinoma, endometrial cancer, cholangiocarcinoma, cervical cancer, uterine cancer, glioma, prostate cancer, salivary gland cancer, and testicular cancer.

73. The method of claim 72, wherein the cancer expressing mutant HER2 is breast cancer.

74. The method of claim 73, wherein the breast cancer expressing mutant HER2 is early-stage breast cancer, metastatic breast cancer, recurrent breast cancer, advanced breast cancer, or non-resectable breast cancer.

75. A method of selectively killing of HER2-mutant cells in a population of cells, said method comprising:
contacting a population of cells comprising HER2-mutant cells with the antibody-based molecule of any one of claims 1-29, the immunoconjugate of any one of claims 42-45, or the multi-specific antibody or multi-specific binding fragment of any one of claims 46-59 in an amount effective to selectively kill the HER2-mutant cells in the population of cells.

76. The method of claim 75, wherein the HER2-mutant cells express a S310F mutant HER2.

77. The method of claim 75, wherein the HER2-mutant cells express a S310Y mutant HER2.

78. A diagnostic agent comprising:
the antibody-based molecule of any one of claim 1-29, and
a detectable label, wherein said detectable label is coupled to the antibody-based molecule.

79. The diagnostic agent of claim 78, wherein the label is a radiolabel, enzyme label, or fluorochrome label.

80. A method of detecting a cancer expressing mutant HER2 in a subject, said method comprising:
contacting a biological sample from the subject with the antibody-based molecule of any one of claims 1-29 or the diagnostic agent of claim 78 or 79, and
detecting a complex between HER2 mutant cancer cells and the antibody-based molecule or a complex between the HER2 mutant cancer cells and the diagnostic agent in the biological sample.

81. A HER2-Fc conjugate protein comprising the amino acid sequence of any one of SEQ ID NOs: 67, 68, or 69.
